(19) United States
(12) Patent Application Publication
(10) Pub. No.: US 2021/0054077 A1
(43) Pub. Date: Feb. 25, 2021

(54) CD3 ANTIGEN BINDING FRAGMENTS AND COMPOSITIONS COMPRISING SAME

(71) Applicant: Amunix Pharmaceuticals, Inc., Mountain View, CA (US)

(72) Inventors: Volker Schellenberger, Palo Alto, CA (US); Philipp Kuhn, Bremen (DE); André Frenzel, Braunschweig (DE); Darragh MacCann, Magherafelt (GB); James McClory, Ballynaskeagh Banbridge (GB)

(21) Appl. No.: 16/996,899

(22) Filed: Aug. 19, 2020

Related U.S. Application Data
(63) Continuation-in-part of application No. PCT/US2020/039673, filed on Jun. 25, 2020.
(60) Provisional application No. 63/041,059, filed on Jun. 18, 2020.
(60) Provisional application No. 62/866,746, filed on Jun. 26, 2019.

(51) Int. Cl. C07K 16/28

(52) U.S. Cl. CPC C07K 16/2809; C07K 2317/92; C07K 2317/31; C07K 2317/567; C07K 2317/94; C07K 2317/565

(57) ABSTRACT

This disclosure relates to compositions having an antibody binding fragment that specifically binds to CD3 or an epitope thereof. Some embodiments include compositions and antibody binding fragments with increased stability. Bispecific fusion proteins including such antibody-binding fragments are also disclosed.

### CROSS-REFERENCE STATEMENT

This application is a continuation-in-part application claiming the benefit of International Patent Application No. PCT/US2020/039673, entitled “CD3 ANTIGEN BINDING FRAGMENTS AND COMPOSITIONS COMPRISING SAME,” filed on Jun. 25, 2020, which claims the benefit of U.S. Provisional Application No. 62/866,746, entitled “CD3 ANTIGEN BINDING FRAGMENTS AND COMPOSITIONS COMPRISING SAME”, filed on Jun. 26, 2019, and U.S. Provisional Application No. 63/041,059, entitled “CD3 ANTIGEN BINDING FRAGMENTS AND COMPOSITIONS COMPRISING SAME”, filed on Jun. 18, 2020, all of which are incorporated herein in their entireties for all purposes.

### SEQUENCE LISTING

The instant application contains a Sequence Listing which has been submitted electronically in ASCII format and is hereby incorporated by reference in its entirety. Said ASCII copy, created on Jun. 24, 2020, is named 32808-775_601_SL.txt and is 3,106,733 bytes in size.

### BACKGROUND

Many approved cancer therapeutics are cytotoxic drugs that kill normal cells as well as tumor cells. The therapeutic benefit of these cytotoxic drugs depends on tumor cells being more sensitive than normal cells, thereby allowing clinical responses to be achieved using doses that do not result in unacceptable side effects. However, essentially all of these non-specific drugs result in some if not severe damage to normal tissues, which often limits treatment suitability.

Bispecific antibodies can offer a different approach to cytotoxic drugs by directing immune effector cells to kill cancer cells. Bispecific antibodies combine the benefits of different binding specificities derived from two monoclonal antibodies into a single composition, enabling approaches or combinations of coverages that are not possible with monospecific antibodies. In one embodiment, this approach relies on binding of one arm of the bispecific antibody to a tumor-associated antigen or marker, while the other arm, upon binding the CD3 molecule on T cells, triggers their cytotoxic activity by the release of effector molecules such as such as TNF-α, IFN-γ, interleukins 2, 4 and 10, perforin, and granzymes. Advances in antibody engineering have led to the development of a number of bispecific antibody formats and compositions for redirecting effector cells to tumor targets, including bispecifics that function by recruiting and activating polyclonal populations of T cells at tumor sites, and do so without the need for co-stimulation or conventional MHC recognition. There remains, however, the dual problems of certain patients experiencing serious side effects referred to as “cytokine storm” or “cytokine release syndrome” (Lee D W et al. Current concepts in the diagnosis and management of cytokine release syndrome. Blood. 2014 124(2):188-195) mediated by the release of TNF-α and IFN-γ, amongst other cytokines, in addition to the fact that some bispecific compositions have a very short half-life, necessitating continuous infusions of four to eight weeks in order to maintain circulating concentrations within the therapeutic window for sufficient time to achieve a therapeutic effect, or have a variable effect. Thus, there is an unmet need in the field for the development of effective bispecific antibodies for use in cancer treatment.

### SUMMARY

The present invention relates to anti-cluster of differentiation 3 (CD3) antigen binding fragments incorporated into chimeric fusion proteins and methods of using the same.

In one aspect, disclosed herein is a polypeptide comprising an antigen binding fragment, wherein the antigen binding fragment, comprises light chain complementarity-determining regions (CDR-L) and heavy chain complementarity-determining regions (CDR-H), and wherein the antigen binding fragment, a. specifically binds to cluster of differentiation 3 T cell receptor (CD3); and b. comprises CDR-H1, CDR-H2, and CDR-H3, having amino acid sequences of SEQ ID NOs: 8, 9, and 10, respectively.

In another aspect, disclosed herein is a polypeptide comprising an anti-CD3 antigen binding fragment, wherein the antigen binding fragment comprises light chain complementarity-determining regions (CDR-L) and heavy chain complementarity-determining regions (CDR-H), and wherein the antigen binding fragment a. specifically binds to CD3; b. comprises CDR-H1, CDR-H2, and CDR-H3, wherein CDR-H3 comprises an amino acid sequence of SEQ ID NO:10; and c. exhibits a higher thermal stability, as evidenced by in an in vitro assay, (i) a higher melting temperature (Tₘ) relative to that of an antigen binding fragment consisting of a sequence shown in SEQ ID NO:41, or (ii) upon incorporating said anti-CD3 antigen binding fragment into an anti-CD3 bispecific antibody, the bispecific antibody exhibits a higher Tm relative to a control bispecific antibody, wherein said anti-CD3 bispecific antibody comprises said anti-CD3 binding fragment and a reference antigen binding fragment that binds to an antigen other than CD3, and wherein said control bispecific antigen binding fragment consists of SEQ ID NO:41 and said reference antigen binding fragment.

In some embodiments, the Tₘ of the antigen binding fragment is at least 2° C. greater, or at least 3° C. greater, or at least 4° C. greater, or at least 5° C. greater than the Tₘ of an antigen binding fragment consisting of a sequence of SEQ ID NO:41.

In yet another aspect, disclosed herein is a polypeptide comprising an antigen binding fragment, wherein the antigen binding fragment comprises light chain complementarity-determining regions (CDR-L) and heavy chain complementarity-determining regions (CDR-H), wherein the antigen binding fragment a. specifically binds to CD3; b. comprises CDR-H1, CDR-H2, and CDR-H3, wherein CDR-H3 comprises an amino acid sequence of SEQ ID NO:10; and c. comprises FR-H1, FR-H2, FR-H3, FR-H4, each exhibiting at least 86%, 87%, 88%, 89%, 90%, 91%, 92%, 93%, 94%, 95%, 96%, 97%, 98%, 99% sequence identity or is identical to an amino acid of SEQ ID NOs: 22, 23, 25, and 26, respectively. In some embodiments, the antigen binding fragment disclosed herein is a chimeric or a humanized antigen binding fragment. In other embodiments, the antigen binding fragment is selected from the group consisting of Fv, Fab, Fab′, Fab′-SH, linear antibody, and single-chain variable fragment (scFv).

In some embodiments, the CDR-H1 and the CDR-H2 comprise amino acid sequences of SEQ ID NOs: 8 and 9, respectively. In certain embodiments, the CDR-L comprises: a CDR-L1 having an amino acid sequence of SEQ ID NOs: 1 or 2, a CDR-L2 having an amino acid sequence of SEQ ID NOs: 4 or 5, and a CDR-L3 having an amino acid sequence of SEQ ID NO:6. In another embodiment, the CDR-L comprises: a CDR-L1 having an amino acid sequence of SEQ ID NO:1; a CDR-L2 having an amino acid sequence of any one of SEQ ID NOs: 4 or 5; and a CDR-L3 having an amino acid sequence of SEQ ID NOs: 6 or 7. In yet another embodiment, the CDR-L comprises: a CDR-L1 having an amino acid sequence of SEQ ID NO:2; a CDR-L2 having an amino acid sequence of any one of SEQ ID NOS: 4 or 5; and a CDR-L3 having an amino acid sequence of SEQ ID NO:6. In one embodiment, the CDR-L comprises: a CDR-L1 having an amino acid sequence of SEQ ID NO: 1; a CDR-L2 having an amino acid sequence of SEQ ID NO: 4; a CDR-L3 having an amino acid sequence of SEQ ID NO: 6. In certain embodiments, the CDR-L comprises: a CDR-L1 having an amino acid sequence of SEQ ID NO:2; a CDR-L2 having an amino acid sequence of SEQ ID NO:5; and a CDR-L3 having an amino acid sequence of SEQ ID NO:6.

In certain embodiments, the antigen binding fragment further comprises FR-L1, FR-L2, FR-L3, FR-L4, each exhibiting at least 86%, 87%, 88%, 89%, 90%, 91%, 92%, 93%, 94%, 95%, 96%, 97%, 98%, 99% sequence identity or is identical to amino acid sequences of SEQ ID NOs: 12, 13, 18, and 19, respectively.

In other embodiments, the antigen binding fragment further comprises a light chain framework region (FR-L) and a heavy chain framework region (FR-H), and wherein the antigen binding fragment comprises: a. a FR-L1 having an amino acid sequence of SEQ ID NO:12; b. a FR-L2 having an amino acid sequence of SEQ ID NO:13; c. a FR-L3 having an amino acid sequence of any one of SEQ ID NOs:14-17; d. a FR-L4 having an amino acid sequence of SEQ ID NO:19; e. a FR-H1 having an amino acid sequence of SEQ ID NO:20 or SEQ ID NO:21; f a FR-H2 having an amino acid sequence of SEQ ID NO:23; e. a FR-H3 having an amino acid sequence of SEQ ID NO:24; and f. a FR-H4 having an amino acid sequence of any one of SEQ ID NO:26. In other embodiments, the antigen binding fragment comprises: a. a FR-L1 having an amino acid sequence of SEQ ID NO:12; b. a FR-L2 having an amino acid sequence of SEQ ID NO:13; c. a FR-L3 having an amino acid sequence of SEQ ID NO:14; d. a FR-L4 having an amino acid sequence of SEQ ID NO:19; e. a FR-H1 having an amino acid sequence of SEQ ID NO:20; f. a FR-H2 having an amino acid sequence of SEQ ID NO:23; g. a FR-H3 having an amino acid sequence of SEQ ID NO:24; and h. a FR-H4 having an amino acid sequence of SEQ ID NO:26. In another embodiment, the antigen binding fragment comprises: a. a FR-L1 having an amino acid sequence of SEQ ID NO:12; b. a FR-L2 having an amino acid sequence of SEQ ID NO:13; c. a FR-L3 having an amino acid sequence of SEQ ID NO:15; d. a FR-L4 having an amino acid sequence of SEQ ID NO:19; e. a FR-H1 having an amino acid sequence of SEQ ID NO:21; f. a FR-H2 having an amino acid sequence of SEQ ID NO:23; g. a FR-H3 having an amino acid sequence of SEQ ID NO:24; and h. a FR-H4 having an amino acid sequence of SEQ ID NO:26. In another embodiment, the antigen binding fragment comprises: a. a FR-L1 having an amino acid sequence of SEQ ID NO:12; b. a FR-L2 having an amino acid sequence of SEQ ID NO:13; c. a FR-L3 having an amino acid sequence of SEQ ID NO:16; d. a FR-L4 having an amino acid sequence of SEQ ID NO:19; e. a FR-H1 having an amino acid sequence of SEQ ID NO:21; f. a FR-H2 having an amino acid sequence of SEQ ID NO:23; g. a FR-H3 having an amino acid sequence of SEQ ID NO:24; and h. a FR-H4 having an amino acid sequence of SEQ ID NO:26. In certain embodiments, the antigen binding fragment comprises: a. a FR-L1 having an amino acid sequence of SEQ ID NO:12; b. a FR-L2 having an amino acid sequence of SEQ ID NO:13; c. a FR-L3 having an amino acid sequence of SEQ ID NO:17; d. a FR-L4 having an amino acid sequence of SEQ ID NO:19; e. a FR-H1 having an amino acid sequence of SEQ ID NO:21; f. a FR-H2 having an amino acid sequence of SEQ ID NO:23; g. a FR-H3 having an amino acid sequence of SEQ ID NO:24; and h. a FR-H4 having an amino acid sequence of SEQ ID NO:26.

In some embodiments, the antigen binding fragment comprises a variable heavy (VH) amino acid sequence having at least 90%, 91%, 92%, 93%, 94%, 95%, 96%, 97%, 98%, 99% sequence identity or is identical to an amino acid sequence of SEQ ID NO:28 or SEQ ID NO:31. In certain embodiments, the antigen binding fragment comprises a variable light (VL) amino acid sequence having at least 90%, 91%, 92%, 93%, 94%, 95%, 96%, 97%, 98%, 99% sequence identity or is identical to an amino acid sequence of any one of SEQ ID NOs: 27, 29, 30, 32, or 33. In other embodiments, the antigen binding fragment comprises an amino acid sequence having at least 95%, 96%, 97%, 98%, 99% sequence identity or is identical to an amino acid sequence of any one of SEQ ID NOs: 36-40.

In some embodiments, the antigen binding fragment specifically binds human or cynomolgus monkey (cyno) CD3. In other embodiments, the antigen binding fragment specifically binds human and cynomolgus monkey (cyno) CD3. In certain embodiments, the antigen binding fragment binds a CD3 complex subunit selected from CD3 epsilon, CD3 delta, CD3 gamma, CD3 zeta, CD3 alpha and CD3 beta epsilon unit of CD3. In other embodiments, the antigen binding fragment binds a CD3 epsilon fragment of CD3.

In certain embodiments, the antigen binding fragment specifically binds human or cyno CD3 with a dissociation constant (K_{d}) constant between about between about 10 nM and about 400 nM, as determined in an in vitro antigen-binding assay comprising a human or cyno CD3 antigen. In other embodiments, the antigen binding fragment specifically binds human or cyno CD3 with a dissociation constant (K_{d}) of less than about 10 nM, or less than about 50 nM, or less than about 100 nM, or less than about 150 nM, or less than about 200 nM, or less than about 250 nM, or less than about 300 nM, or less than about 350 nM, or less than about 400 nM as determined in an in vitro antigen-binding assay. In another embodiment, the antigen binding fragment exhibits a binding affinity to CD3 that is at least 2-fold, 3-fold, 4-fold, 5-fold, 6-fold, 7-fold, 8-fold, 9-fold, or at least 10-fold weaker relative to that of an antigen binding fragment consisting of an amino acid sequence of SEQ ID NO:41, as determined by the respective dissociation constants (K_{d}) in an in vitro antigen-binding assay.

In some embodiments, the antigen binding fragment exhibits an isoelectric point (pI) that is less than or equal to 6.6. In other embodiments, the antigen binding fragment exhibits a pI that is between 6.0 and 6.6, inclusive. In certain embodiments, the antigen binding fragment exhibits a pI that is at least 0.1, 0.2, 0.3, 0.4, 0.5, 0.6, 0.7, 0.8, 0.9, or 1.0 pH units lower than the pI of a reference antigen binding fragment consisting of a sequence shown in SEQ ID NO: 41.

In other embodiments, the polypeptide disclosed herein, further comprises a first release segment peptide (RS1), wherein the RS1 is a substrate for cleavage by a mammalian protease. In certain embodiments, the RS1 is a substrate for a protease selected from the group consisting of legumain, MMP-2, MMP-7, MMP-9, MMP-11, MMP-14, uPA, and matriptase. In another embodiment, the RS1 comprises an amino acid sequence having at least 90%, 91%, 92%, 93%, 94%, 95%, 96%, 97%, 98%, 99%, or 100% sequence identity to a sequence selected from SEQ ID NOs: 42-660. In certain embodiments, the RS1 comprises an amino acid sequence selected from the sequences of RSR-2089, RSR-2295, RSR-2298, RSR-2488, RSR-2599, RSR-2485, RSR-2486, RSR-2728, RSN-2089, RSN-2295, RSN-2298, RSN-2488, RSN-2599, RSN-2485, RSN-2486, RSN-2728, RSC-2089, RSC-2295, RSC-2298, RSC-2488, RSC-2599, RSC-2485, RSC-2486, and RSC-2728, each of which being forth in Table 5.

In some embodiments, the polypeptide disclosed herein further comprises a first extended recombinant polypeptide (XTEN1) wherein the XTEN1 is characterized in that a. it has at least about 36 amino acids or at least about 100 amino acids; b. at least 90%, 91%, 92%, 93%, 94%, 95%, 96%, 97%, 98%, 99% or 100% of the amino acid residues of the XTEN1 sequence are selected from glycine (G), alanine (A), serine (S), threonine (T), glutamate (E) and proline (P); and c. it has at least 4-6 different amino acids selected from G, A, S, T, E and P. In some embodiments, the XTEN1 have at least about 36 to about 1000 amino acids or at least about 100 to 1000 amino acids. In certain embodiments, the XTEN1 comprises an amino acid sequence selected from at least three of SEQ ID NOs: 661-664. In other embodiments, the XTEN1 comprises an amino acid sequence having at least about 90%, 91%, 92%, 93%, 94%, 95%, 96%, 97%, 98%, 99%, or 100% sequence identity to a sequence selected from SEQ ID NOs: 665-718 and 922-926. In another embodiment, the XTEN1 comprises an amino acid sequence having at least about 90%, 91%, 92%, 93%, 94%, 95%, 96%, 97%, 98%, 99%, or 100% sequence identity to a sequence selected from the sequences of AE144_1A, AE144_2A, AE144_2B, AE144_3A, AE144_3B, AE144_4A, AE144_4B, AE144_5A, AE144_6B, AE144_7A, AE284, AE288_1, AE288_2, AE288_3, AE292, AE293, AE300, AE576, AE584, AE864, AE864_2, AE865, AE866, AE867, and AE868, each of which being set forth in Table 7.

In certain embodiments, the polypeptide disclosed herein is expressed as a fusion protein, wherein the fusion protein, in an uncleaved state, has a structural arrangement from N-terminus to C-terminus of AF1-RS1-XTEN1 or XTEN1-RS1-AF1, wherein AF1 is a first antigen binding fragment.

In certain aspect, disclosed herein is a polypeptide comprising an RS1, RS2, AF1, AF2, XTEN1, and XTEN2, wherein: a. the RS1 and RS2 are each a substrate for cleavage by a mammalian protease and each comprise an amino acid sequence having at least 90%, 91%, 92%, 93%, 94%, 95%, 96%, 97%, 98%, 99%, or 100% sequence identity to a sequence selected from SEQ ID NOs:42-660; b. the AF1 is an antigen binding fragment of a monoclonal antibody having binding specificity to CD3; c. the AF2 is an antigen binding fragment comprising a VL and VH of a monoclonal antibody having binding affinity to a target cell marker; d. the XTEN1 comprises an amino acid sequence having at least about 90%, 91%, 92%, 93%, 94%, 95%, 96%, 97%, 98%, 99%, or 100% sequence identity to a sequence selected from SEQ ID NOs: 665-718 and 922-926; e. the XTEN2 comprises an amino acid sequence having at least about 90%, 91%, 92%, 93%, 94%, 95%, 96%, 97%, 98%, 99%, or 100% sequence identity to a sequence selected from SEQ ID NOs: 665-718 and 922-926; f the polypeptide has a structural arrangement from N-terminus to C-terminus as follows: XTEN1-RS1-AF2-AF1-RS2-XTEN2, XTEN1-RS1-AF1-AF2-RS2-XTEN2, XTEN2-RS2-AF2-AF1-RS1-XTEN1, XTEN2-RS2-AF1-AF2-RS1-XTEN1, or XTEN2-RS2-diabody-RS1-XTEN1, wherein the diabody comprises VL and VH of the AF1 and AF2; and g. the polypeptide exhibits a higher thermal stability, as determined by an increase in melting temperature (Tm) in an in vitro assay, relative to an antibody fragment consisting of a sequence shown in SEQ ID NO:41.

In some embodiments, the AF1 comprises heavy chain complementary determining regions (CDR-H) CDR-H1, CDR-H2, and CDR-H3, wherein CDR-H3 comprises an amino acid sequence of SEQ ID NO:10; and exhibits a higher thermal stability, as determined by an increased melting temperature (Tₘ) in an in vitro assay, relative to that of an antigen binding fragment consisting of a sequence shown in SEQ ID NO:41. In other embodiments, the AF1 comprises light chain complementarity-determining regions (CDR-L) and heavy chain complementarity-determining regions (CDR-H), wherein the AF1 comprises CDR-H1, CDR-H2, and CDR-H3, wherein CDR-H3 comprises an amino acid sequence of SEQ ID NO:10; and comprises FR-H1, FR-H2, FR-H3, FR-H4, each exhibiting at least 86%, 87%, 88%, 89%, 90%, 91%, 92%, 93%, 94%, 95%, 96%, 97%, 98%, 99% sequence identity or is identical to an amino acid of SEQ ID NOs: 20 or 21, 23, 24, and 26, respectively.

In certain embodiments, the CDR-H1 and the CDR-H2 comprise amino acid sequences of SEQ ID NOs: 8 and 9, respectively. In some embodiments, the CDR-L comprises: a CDR-L1 having an amino acid sequence of SEQ ID NO: 1 or 2; a CDR-L2 having an amino acid sequence of SEQ ID NO: 4 or 5; and a CDR-L3 having an amino acid sequence of SEQ ID NO:6. In another embodiment, the CDR-L comprises: a CDR-L1 having an amino acid sequence of SEQ ID NO:1; a CDR-L2 having an amino acid sequence of any one of SEQ ID NOs: 4 or 5; and a CDR-L3 having an amino acid sequence of SEQ ID NO:6. In other embodiments, the CDR-L comprises: a CDR-L1 having an amino acid sequence of SEQ ID NO:2; a CDR-L2 having an amino acid sequence of any one of SEQ ID NOs: 4 or 5; and a CDR-L3 having an amino acid sequence of SEQ ID NO:6. In other embodiments, the CDR-L comprises: a CDR-L1 having an amino acid sequence of SEQ ID NO: 1; a CDR-L2 amino acid sequence of any one of SEQ ID NO: 4; and a CDR-L3 amino acid sequence of SEQ ID NO: 6. In other embodiments, the CDR-L comprises: a CDR-L1 having an amino acid sequence of SEQ ID NO:2; a CDR-L2 having an amino acid sequence of any one of SEQ ID NO:5; and a CDR-L3 having an amino acid sequence of SEQ ID NO:6.

In some embodiments, the AF1 comprises a light chain framework region (FR-L) and a heavy chain framework region (FR-H), and wherein the AF1 comprises: a. a FR-L1 having an amino acid sequence of SEQ ID NO:12; b. a FR-L2 having an amino acid sequence of SEQ ID NO:13; c. a FR-L3 having an amino acid sequence of SEQ ID NO:14; d. a FR-L4 having an amino acid sequence of SEQ ID NO:19; e. a FR-H1 having an amino acid sequence of SEQ ID NO:20; f. a FR-H2 having an amino acid sequence of SEQ ID NO:23; g. a FR-H3 having an amino acid sequence of SEQ ID NO:24; and h. a FR-H4 having an amino acid sequence of SEQ ID NO:26. In one embodiment, the AF1 comprises: a. a FR-L1 having an amino acid sequence of SEQ ID NO:12; b. a FR-L2 having an amino acid sequence of SEQ ID NO:13; c. a FR-L3 having an amino acid sequence of SEQ ID NO:15; d. a FR-L4 having an amino acid sequence of SEQ ID NO:19; e. a FR-H1 having an amino acid sequence of SEQ ID NO:21; f. a FR-H2 having an amino acid sequence of SEQ ID NO:23; g. a FR-H3 having an amino acid sequence of SEQ ID NO:24; and h. a FR-H4 having an amino acid sequence of SEQ ID NO:26. In another embodiment, the AF1 comprises: a. a FR-L1 having an amino acid sequence of SEQ ID NO:12; b. a FR-L2 having an amino acid sequence of SEQ ID NO:13; c. a FR-L3 having an amino acid sequence of SEQ ID NO:16; d. a FR-L4 having an amino acid sequence of SEQ ID NO:19; e. a FR-H1 having an amino acid sequence of SEQ ID NO:21; f a FR-H2 having an amino acid sequence of SEQ ID NO:23; g. a FR-H3 having an amino acid sequence of SEQ ID NO:24; and h. a FR-H4 having an amino acid sequence of SEQ ID NO:26. In yet another embodiment, the AF1 comprises: a. a FR-L1 having an amino acid sequence of SEQ ID NO: 12; b. a FR-L2 having an amino acid sequence of SEQ ID NO:13; c. a FR-L3 having an amino acid sequence of SEQ ID NO:17; d. a FR-L4 having an amino acid sequence of SEQ ID NO:19; e. a FR-H1 having an amino acid sequence of SEQ ID NO:21; f a FR-H2 having an amino acid sequence of SEQ ID NO:23; g. a FR-H3 having an amino acid sequence of SEQ ID NO:24; and h. a FR-H4 having an amino acid sequence of SEQ ID NO:26.

In some other embodiments, the AF1 further comprises FR-L1, FR-L2, FR-L3, FR-L4, each exhibiting at least 86%, 87%, 88%, 89%, 90%, 91%, 92%, 93%, 94%, 95%, 96%, 97%, 98%, 99% sequence identity or is identical to amino acid sequences of SEQ ID NOs: 12, 13, 14-17, and 19, respectively.

In some embodiments, the AF1 comprises a variable heavy (VH) amino acid sequence having at least 90%, 91%, 92%, 93%, 94%, 95%, 96%, 97%, 98%, 99% sequence identity or is identical to an amino acid sequence of SEQ ID NO:28 or SEQ ID NO:31. In certain embodiments, the AF1 comprises a variable light (VL) amino acid sequence having at least 90%, 91%, 92%, 93%, 94%, 95%, 96%, 97%, 98%, 99% sequence identity or is identical to an amino acid sequence of any one of SEQ ID NOs: 27, 29, 30, 32, or 33. In certain embodiments, the AF1 comprises an amino acid sequence having at least 95%, 96%, 97%, 98%, 99% sequence identity or is identical to an amino acid sequence of any one of SEQ ID NOs:36-40.

In certain embodiments, the AF1 specifically binds human or cynomolgus monkey (cyno) CD3. In some embodiments, the AF1 specifically binds human and cynomolgus monkey (cyno) CD3. In some other embodiments, the AF1 binds CD3 complex subunits selected from CD3 epsilon, CD3 delta, CD3 gamma, CD3 zeta, CD3 alpha and CD3 beta epsilon fragment of CD3. In another embodiment, the AF1 binds CD3 epsilon. In another embodiment, the AF1 specifically binds human or cyno CD3 with a dissociation constant (K_{d}) constant between about between about 10 nM and about 400 nM, as determined in an in vitro antigen-binding assay. In certain embodiments, the AF1 specifically binds human or cyno CD3 with a dissociation constant (K_{d}) of less than about 3 nM, or less than about 10 nM, or less than about 50 nM, or less than about 100 nM, or less than about 150 nM, or less than about 200 nM, or less than about 250 nM, or less than about 300 nM, as determined in an in vitro antigen-binding assay. In other embodiments, the AF1 specifically binds human or cyno CD3 with at least 2-fold, 3-fold, 4-fold, 5-fold, 6-fold, 7-fold, 8-fold, 9-fold, or at least 10-fold less binding affinity than an AF1 consisting of an amino acid sequence of SEQ ID NO: 41, as determined by the respective dissociation constants (K_{d}) in an in vitro antigen-binding assays.

In some embodiments, the Tₘ of the AF1 is at least 2° C. greater, or at least 3° C. greater, or at least 4° C. greater, or at least 5° C. greater, or at least 6° C. greater, or at least 7° C. greater, or at least 8° C. greater, or at least 9° C. greater, or at least 10° C. greater than the Tₘ of an antigen binding fragment consisting of a sequence of SEQ ID NO:41, as determined by an increase in melting temperature in an in vitro assay.

In other embodiments, AF1 exhibits an isoelectric point (pI) that is less than or equal to 6.6. In certain embodiments, the AF1 exhibits a pI that is between 6.0 and 6.6, inclusive. In other embodiments, the AF1 exhibits a pI that is at least 0.1, 0.2, 0.3, 0.4, 0.5, 0.6, 0.7, 0.8, 0.9, or 1.0 lower than the pI of a reference antigen binding fragment consisting of a sequence shown in SEQ ID NO: 41.

In another embodiment, the polypeptide disclosed herein, further comprises a second antigen binding fragment (AF2) that specifically binds to a target cell marker other than CD3. In some embodiments, the AF2 is fused to the AF1 by a flexible peptide linker. In other embodiments, the flexible linker comprises 2 or 3 types of amino acids selected from the group consisting of glycine, serine, and proline. In certain embodiments, (1) the AF2 fragment is selected from the group consisting of Fv, Fab, Fab′, Fab′-SH, linear antibody, a single domain antibody, and single-chain variable fragment (scFv), or (2) the AF1 and AF2 are configured as an (Fab′)2 or a single chain diabody.

In some embodiments, the CDR of the AF2 is selected from the sequences of SEQ ID NOs: 719-918. In certain embodiments, the AF2 comprises VL and VH of a monoclonal antibody having binding affinity to the target cell marker. In other embodiments, the VL is selected from the sequences of SEQ ID NOs:819-918, and the VH of the AF2 is selected from the sequences of SEQ ID NOs:719-818.

In some embodiments, the target cell marker is a tumor antigen. In some embodiments, the target cell marker is selected from 1-40-β-amyloid, 4-1BB, 5AC, 5T4, 707-AP, A kinase anchor protein 4 (AKAP-4), activin receptor type-2B (ACVR2B), activin receptor-like kinase 1 (ALK1), adenocarcinoma antigen, adipophilin, adrenoceptor β3 (ADRB3), AGS-22M6, α folate receptor, α-fetoprotein (AFP), AIM-2, anaplastic lymphoma kinase (ALK), androgen receptor, angiopoietin 2, angiopoietin 3, angiopoietin-binding cell surface receptor 2 (Tie 2), anthrax toxin, AOC3 (VAP-1), B cell maturation antigen (BCMA), B7-H3 (CD276), Bacillus anthracis anthrax, B-cell activating factor (BAFF), B-lymphoma cell, bone marrow stromal cell antigen 2 (BST2), Brother of the Regulator of Imprinted Sites (BORIS), C242 antigen, C5, CA-125, cancer antigen 125 (CA-125 or MUC16), Cancer/testis antigen 1 (NY-ESO-1), Cancer/testis antigen 2 (LAGE-1a), carbonic anhydrase 9 (CA-IX), Carcinoembryonic antigen (CEA), cardiac myosin, CCCTC-Binding Factor (CTCF), CCL11 (eotaxin-1), CCR4, CCR5, CCR6, CCR7, CCR8, CCR9, CD11, CD123, CD125, CD140a, CD147 (basigin), CD15, CD152, CD154 (CD40L), CD171, CD179a, CD18, CD19, CD2, CD20, CD200, CD22, CD221, CD23 (IgE receptor), CD24, CD25 (a chain of IL-2 receptor), CD27, CD274, CD28, CD3, CD3 ε, CD30, CD300 molecule-like family member f (CD300LF), CD319 (SLAMF7), CD33, CD37, CD38, CD4, CD40, CD40 ligand, CD41, CD44 v7, CD44 v8, CD44 v6, CD5, CD51, CD52, CD56, CD6, CD70, CD72, CD74, CD79A, CD79B, CD80, CD97, CEA-related antigen, CFD, ch4D5, chromosome X open reading frame 61 (CXORF61), claudin 18.2 (CLDN18.2), claudin 6 (CLDN6), Clostridium difficile, clumping factor A, CLCA2, colony stimulating factor 1 receptor (CSF1R), CSF2, CTLA-4, C-type lectin domain family 12 member A (CLEC12A), C-type lectin-like molecule-1 (CLL-1 or CLECL1), C-X-C chemokine receptor type 4, cyclin B1, cytochrome P4501B1 (CYP1B1), cyp-B, cytomegalovirus, cytomegalovirus glycoprotein B, dabigatran, DLL4, DPP4, DR5, E. coli shiga toxin type-1, E. coli shiga toxin type-2, ecto-ADP-ribosyltransferase 4 (ART4), EGF-like module-containing mucin-like hormone receptor-like 2 (EMR2), EGF-like-domain multiple 7 (EGFL7), elongation factor 2 mutated (ELF2M), endotoxin, Ephrin A2, Ephrin B2, ephrin type-A receptor 2, epidermal growth factor receptor (EGFR), epidermal growth factor receptor variant III (EGFRvIII), episialin, epithelial cell adhesion molecule (EpCAM), epithelial glycoprotein 2 (EGP-2), epithelial glycoprotein 40 (EGP-40), ERBB2, ERBB3, ERBB4, ERG (transmembrane protease, serine 2 (TMPRSS2) ETS fusion gene), Escherichia coli, ETS translocation-variant gene 6, located on chromosome 12p (ETV6-AML), F protein of respiratory syncytial virus, FAP, Fc fragment of IgA receptor (FCAR or CD89), Fc receptor-like 5 (FCRLS), fetal acetylcholine receptor, fibrin II β chain, fibroblast activation protein α (FAP), fibronectin extra domain-B, FGF-5, Fms-Like Tyrosine Kinase 3 (FLT3), folate binding protein (FBP), folate hydrolase, folate receptor 1, folate receptor α, folate receptor β, Fos-related antigen 1, Frizzled receptor, Fucosyl GM1, G250, G protein-coupled receptor 20 (GPR20), G protein-coupled receptor class C group 5, member D (GPRC5D), ganglioside G2 (GD2), GD3 ganglioside, glycoprotein 100 (gp100), glypican-3 (GPC3), GMCSF receptor α-chain, GPNMB, GnT-V, growth differentiation factor 8, GUCY2C, heat shock protein 70-2 mutated (mut hsp70-2), hemagglutinin, Hepatitis A virus cellular receptor 1 (HAVCR1), hepatitis B surface antigen, hepatitis B virus, HER1, HER2/neu, HER3, hexasaccharide portion of globoH glycoceramide (GloboH), HGF, HHGFR, high molecular weight-melanoma-associated antigen (HMW-MAA), histone complex, HIV-1, HLA-DR, HNGF, Hsp90, HST-2 (FGF6), human papilloma virus E6 (HPV E6), human papilloma virus E7 (HPV E7), human scatter factor receptor kinase, human Telomerase reverse transcriptase (hTERT), human TNF, ICAM-1 (CD54), iCE, IFN-α, IFN-0, IFN-γ, IgE, IgE Fc region, IGF-1, IGF-1 receptor, IGHE, IL-12, IL-13, IL-17, IL-17A, IL-17F, IL-1β, IL-20, IL-22, IL-23, IL-31, IL-31RA, IL-4, IL-5, IL-6, IL-6 receptor, IL-9, immunoglobulin lambda-like polypeptide 1 (IGLL1), influenza A hemagglutinin, insulin-like growth factor 1 receptor (IGF-I receptor), insulin-like growth factor 2 (ILGF2), integrin α4β7, integrin β2, integrin α2, integrin α4, integrin α5β1, integrin α7β7, integrin αIIbβ3, integrin αvβ3, interferon α/β receptor, interferon γ-induced protein, Interleukin 11 receptor α (IL-11Rα), Interleukin-13 receptor subunit α-2 (IL-13Ra2 or CD213A2), intestinal carboxyl esterase, kinase domain region (KDR), KIR2D, KIT (CD117), L1-cell adhesion molecule (L1-CAM), legumain, leukocyte immunoglobulin-like receptor subfamily A member 2 (LILRA2), leukocyte-associated immunoglobulin-like receptor 1 (LAIR1), lymphocyte antigen 6 (Ly-6), Lewis-Y antigen, LFA-1 (CD11a), LINGO-1, lipoteichoic acid, LOXL2, L-selectin (CD62L), lymphocyte antigen 6 complex, locus K 9 (LY6K), lymphocyte antigen 75 (LY75), lymphocyte-specific protein tyrosine kinase (LCK), lymphotoxin-α (LT-α) or Tumor necrosis factor-β (TNF-β), Lysosomal Associated Membrane Protein 1 (LAMP1), macrophage migration inhibitory factor (MIF or MMIF), M-CSF, mammary gland differentiation antigen (NY-BR-1), MCP-1, melanoma cancer testis antigen-1 (MAD-CT-1), melanoma cancer testis antigen-2 (MAD-CT-2), melanoma inhibitor of apoptosis (ML-IAP), melanoma-associated antigen 1 (MAGE-A1), mesothelin, mucin 1, cell surface associated (MUC1), MUC-2, MUC3, MUC4, MUC5AC, MUC5B, MUC7, MUC16, mucin CanAg, myelin-associated glycoprotein, myostatin, N-Acetyl glucosaminyl-transferase V (NA17), NCA-90 (granulocyte antigen), Nectin-4, nerve growth factor (NGF), neural apoptosis-regulated proteinase 1, neural cell adhesion molecule (NCAM), neurite outgrowth inhibitor (e.g., NOGO-A, NOGO-B, NOGO-C), neuropilin-1 (NRP1), N-glycolylneuraminic acid, NKG2D, Notch receptor, o-acetyl-GD2 ganglioside (OAcGD2), olfactory receptor 51E2 (OR51E2), oncofetal antigen (h5T4), oncogene fusion protein consisting of breakpoint cluster region (BCR) and Abelson murine leukemia viral oncogene homolog 1 (Abl) (bcr-abl), Oryctolagus cuniculus, OX-40, oxLDL, p53 mutant, paired box protein Pax-3 (PAX3), paired box protein Pax-5 (PAXS), pannexin 3 (PANX3), P-cadherin, phosphate-sodium co-transporter, phosphatidylserine, placenta-specific 1 (PLAC1), platelet-derived growth factor receptor α (PDGF-R α), platelet-derived growth factor receptor β (PDGFR-β), polysialic acid, proacrosin binding protein sp32 (OY-TES1), programmed cell death protein 1 (PD-1), Programmed death-ligand 1 (PD-L1), proprotein convertase subtilisin/kexin type 9 (PCSK9), prostase, prostate carcinoma tumor antigen-1 (PCTA-1 or Galectin 8), melanoma antigen recognized by T cells 1 (MelanA or MART1), P15, P53, PRAME, prostate stem cell antigen (PSCA), prostate-specific membrane antigen (PSMA), prostatic acid phosphatase (PAP), prostatic carcinoma cells, prostein, Protease Serine 21 (Testisin or PRSS21), Proteasome (Prosome, Macropain) Subunit, β Type, 9 (LMP2), Pseudomonas aeruginosa, rabies virus glycoprotein, RAGE, Ras Homolog Family Member C (RhoC), receptor activator of nuclear factor kappa-B ligand (RANKL), Receptor for Advanced Glycation Endproducts (RAGE-1), receptor tyrosine kinase-like orphan receptor 1 (ROR1), renal ubiquitous 1 (RU1), renal ubiquitous 2 (RU2), respiratory syncytial virus, Rh blood group D antigen, Rhesus factor, sarcoma translocation breakpoints, sclerostin (SOST), selectin P, sialyl Lewis adhesion molecule (sLe), sperm protein 17 (SPA17), sphingosine-1-phosphate, squamous cell carcinoma antigen recognized by T Cells 1, 2, and 3 (SART1, SART2, and SART3), stage-specific embryonic antigen-4 (SSEA-4), Staphylococcus aureus, STEAP1, syndecan 1 (SDC1)+A314, SOX10, survivin, survivin-2B, synovial sarcoma, X breakpoint 2 (SSX2), T-cell receptor, TCR Γ Alternate Reading Frame Protein (TARP), telomerase, TEM1, tenascin C, TGF-β (e.g., TGF-β 1, TGF-β 2, TGF-β 3), thyroid stimulating hormone receptor (TSHR), tissue factor pathway inhibitor (TFPI), Tn antigen ((Tn Ag) or (GalNAcα-Ser/Thr)), TNF receptor family member B cell maturation (BCMA), TNF-α, TRAIL-R1, TRAIL-R2, TRG, transglutaminase 5 (TGS5), tumor antigen CTAA16.88, tumor endothelial marker 1 (TEM1/CD248), tumor endothelial marker 7-related (TEM7R), tumor protein p53 (p53), tumor specific glycosylation of MUC1, tumor-associated calcium signal transducer 2 (TROP-2), tumor-associated glycoprotein 72 (TAG72), tumor-associated glycoprotein 72 (TAG-72)+A327, TWEAK receptor, tyrosinase, tyrosinase-related protein 1 (TYRP1 or glycoprotein 75), tyrosinase-related protein 2 (TYRP2), uroplakin 2 (UPK2), vascular endothelial growth factor (e.g., VEGF-A, VEGF-B, VEGF-C, VEGF-D, PIGF), vascular endothelial growth factor receptor 1 (VEGFR1), vascular endothelial growth factor receptor 2 (VEGFR2), vimentin, v-myc avian myelocytomatosis viral oncogene neuroblastoma derived homolog (MYCN), von Willebrand factor (VWF), Wilms tumor protein (WT1), X Antigen Family, Member 1A (XAGE1), β-amyloid, κ-light chain, Fibroblast Growth Factor Receptor 2 (FGFR2), LIV-1 Protein, estrogen regulated (LIV1, aka SLC39A6), Neurotrophic Receptor Tyrosine Kinase 1 (NTRK1, aka TRK), Ret Proto-Oncogene (RET), B Cell Maturation Antigen (BCMA, aka TNFRSF17), Transferrin Receptor (TFRC, aka CD71), Activated Leukocyte Cell Adhesion Molecule (ALCAM, aka CD166), Somatostatin Receptor 2 (SSTR2), KIT Proto-Oncogene Receptor Tyrosine Kinase (cKIT), V-Set Immunoregulatory Receptor (VSIR, aka VISTA), Glycoprotein Nmb (GPNMB), Delta Like Canonical Notch Ligand 3 (DLL3), Interleukin 3 Receptor Subunit Alpha (IL3RA, aka CD123), Lysosomal Associated Membrane Protein 1 (LAMP1), Cadherin 3, Type 1, P-Cadherin (CDH3), Ephrin A4 (EFNA4), Protein Tyrosine Kinase 7 (PTK7), Solute Carrier Family 34 Member 2 (SLC34A2, aka NaPi-2b), Guanylyl Cyclase C (GCC), PLAUR Domain Containing 3 (LYPD3, aka LY6 or C4.4a), Mucin 17, Cell Surface Associated (MUC17), Fms Related Receptor Tyrosine Kinase 3 (FLT3), NKG2D ligands (e.g. ULBP1, ULBP2, ULBP3, H60, Rae-1α, Rae-1β, Rae-1δ, Rae-1γ, MICA, MICB, hHLA-A), SLAM Family Member 7 (SLAMF7), Interleukin 13 Receptor Subunit Alpha 2 (IL13RA2), C-Type Lectin Domain Family 12 Member A (CLEC12A aka CLL-1), CEA Cell Adhesion Molecule 5 (CEACAM aka CD66e), Interleukin 3 Receptor Subunit Alpha (IL3RA), CD5 Molecule (CD5), UL16 Binding Protein 1 (ILBP1), V-Set Domain Containing T Cell Activation Inhibitor 1 (VTCN1 aka B7-H4), Chondroitin Sulfate Proteoglycan 4 (CSPG4), Syndecan 1 (SDC1 aka CD138), Interleukin 1 Receptor Accessory Protein (IL1RAP), Baculoviral IAP Repeat Containing 5 (BIRC5 aka Survivin), CD74 Molecule (CD74), Hepatitis A Virus Cellular Receptor 1 (HAVCR1 aka TIM1), SLIT and NTRK Like Family Member 6 (SILTRK6), CD37 Molecule (CD37), Coagulation Factor III, Tissue Factor (CD142 aka F3), AXL Receptor Tyrosine Kinase (AXL), Endothelin Receptor Type B (EDNRB aka ETBR), Cadherin 6 (CDH6), Fibroblast Growth Factor Receptor 3 (FGFR3), Carbonic Anhydrase 6 (CA6), CanAg glycoform of MUC1, Integrin Subunit Alpha V (ITGAV), Teratocarcinoma-Derived Growth Factor 1 (TDGF1, aka Crypto 1), SLAM Family Member 6 (SLAMF6 aka CD352), and Notch Receptor 3 (NOTCH3).

In some embodiments, the CDR of the AF2 is selected from a CDR sequence of the sequences of SEQ ID NOs:719-918. In certain embodiments, the AF2 comprises VL and VH of a monoclonal antibody having binding affinity to the target cell marker. In certain embodiments, the VL sequences are selected from the sequences of SEQ ID NOs:719-818 and VH sequences are selected from the sequences of SEQ ID NOs:819-918.

In some embodiments, the AF2 specifically binds the target cell marker with a K_{d} between about 0.1 nM and about 100 nM, as determined in an in vitro antigen-binding assay comprising the target cell marker. In certain embodiments, the binding affinity of the AF2 to the target cell marker is at least 10-fold greater, or at least 100-fold greater, or at least 1000-fold greater than the binding affinity of the AF1 to CD3, as measured in an in vitro antigen-binding assay. In certain embodiments, the AF2 comprises a CDR of a monoclonal antibody having binding affinity to the target cell marker.

In certain embodiments, the polypeptide disclosed herein, further comprises a second release segment (RS2), wherein the RS2 is a substrate for cleavage by a mammalian protease. In some embodiments, the RS2 is a substrate for a protease selected from legumain, MMP-2, MMP-7, MMP-9, MMP-11, MMP-14, uPA, and matriptase. In other embodiments, the RS2 comprises an amino acid sequence having at least 90%, 91%, 92%, 93%, 94%, 95%, 96%, 97%, 98%, 99% sequence identity to a sequence selected from SEQ ID NOs:42-660. In another embodiment, the sequences of RS1 and RS2 are identical. In yet another embodiment, the sequences of RS1 and RS2 are not identical. In some embodiments, the RS1 and RS2 are each a substrate for cleavage by multiple proteases at one, two, or three cleavage sites within each release segment sequence.

In some embodiments, the polypeptide disclosed herein further comprises a second extended recombinant polypeptide (XTEN2) wherein the XTEN2 is characterized in that a. it has at least about 36 amino acids or at least about 100 amino acids; b. at least 90%, 91%, 92%, 93%, 94%, 95%, 96%, 97%, 98%, 99% or 100% of the amino acid residues of the XTEN1 sequence are selected from glycine (G), alanine (A), serine (S), threonine (T), glutamate (E) and proline (P); and c. it has at least 4-6 different amino acids selected from G, A, S, T, E and P. In other embodiments, the XTEN2 comprises an amino acid sequence, wherein at least 90%, 91%, 92%, 93%, 94%, 95%, 96%, 97%, 98%, 99% or 100% of the amino acid sequence comprises non-overlapping sequences selected from at least three of SEQ ID NOs:661-664. In another embodiment, the XTEN2 comprises an amino acid sequence having at least about 90%, 91%, 92%, 93%, 94%, 95%, 96%, 97%, 98%, 99%, or 100% sequence identity to a sequence selected from SEQ ID NOs: 665-718 and 922-926. In certain embodiments, the XTEN2 comprises an amino acid sequence having at least about 90%, 91%, 92%, 93%, 94%, 95%, 96%, 97%, 98%, 99%, or 100% sequence identity to a sequence selected from the sequences of AE144_1A, AE144_2A, AE144_2B, AE144_3A, AE144_3B, AE144_4A, AE144_4B, AE144_5A, AE144_6B, AE144_7A, AE284, AE288_1, AE288_2, AE288_3, AE292, AE293, AE300, AE576, AE584, AE864, AE864_2, AE865, AE866, AE867, and AE868, each of which being set forth in Table 7.

In other embodiments, the polypeptide has a structural arrangement from N-terminus to C-terminus as follows: XTEN1-RS1-AF2-AF1-RS2-XTEN2, XTEN1-RS1-AF1-AF2-RS2-XTEN2, XTEN2-RS2-AF2-AF1-RS1-XTEN1, XTEN2-RS2-AF1-AF2-RS1-XTEN1, XTEN2-RS2-diabody-RS1-XTEN1, or XTEN1-RS1-diabody-RS2-XTEN2, wherein the diabody comprises VL and VH of the AF1 and AF2, wherein the AF1 specifically binds CD3 and AF2 specifically binds a target cell marker, and wherein XTEN 1 and XTEN2 are of different amino acid length or sequence.

In some other embodiments, the AF1 is fused to the AF2 by a flexible peptide linker wherein a. the AF2 specifically binds to a second reference antigen other than CD3 such that the polypeptide is a bispecific antigen binding fragment capable of binding both CD3 and the second reference antigen; b. the bispecific antigen binding fragment exhibits a higher thermal stability, as determined by an increase in melting temperature (Tₘ) in an in vitro assay relative to a control bispecific antigen binding fragment wherein said control bispecific antigen binding fragment comprises SEQ ID NO:41 and AF2.

In certain embodiments, the second reference antigen is a target cell marker selected from 1-40-β-amyloid, 4-1BB, 5AC, 5T4, 707-AP, A kinase anchor protein 4 (AKAP-4), activin receptor type-2B (ACVR2B), activin receptor-like kinase 1 (ALK1), adenocarcinoma antigen, adipophilin, adrenoceptor β3 (ADRB3), AGS-22M6, α folate receptor, α-fetoprotein (AFP), AIM-2, anaplastic lymphoma kinase (ALK), androgen receptor, angiopoietin 2, angiopoietin 3, angiopoietin-binding cell surface receptor 2 (Tie 2), anthrax toxin, AOC3 (VAP-1), B cell maturation antigen (BCMA), B7-H3 (CD276), Bacillus anthracis anthrax, B-cell activating factor (BAFF), B-lymphoma cell, bone marrow stromal cell antigen 2 (BST2), Brother of the Regulator of Imprinted Sites (BORIS), C242 antigen, C5, CA-125, cancer antigen 125 (CA-125 or MUC16), Cancer/testis antigen 1 (NY-ESO-1), Cancer/testis antigen 2 (LAGE-1a), carbonic anhydrase 9 (CA-IX), Carcinoembryonic antigen (CEA), cardiac myosin, CCCTC-Binding Factor (CTCF), CCL11 (eotaxin-1), CCR4, CCR5, CCR6, CCR7, CCR8, CCR9, CD11, CD123, CD125, CD140a, CD147 (basigin), CD15, CD152, CD154 (CD40L), CD171, CD179a, CD18, CD19, CD2, CD20, CD200, CD22, CD221, CD23 (IgE receptor), CD24, CD25 (α chain of IL-2 receptor), CD27, CD274, CD28, CD3, CD3 ε, CD30, CD300 molecule-like family member f (CD300LF), CD319 (SLAMF7), CD33, CD37, CD38, CD4, CD40, CD40 ligand, CD41, CD44 v7, CD44 v8, CD44 v6, CD5, CD51, CD52, CD56, CD6, CD70, CD72, CD74, CD79A, CD79B, CD80, CD97, CEA-related antigen, CFD, ch4D5, chromosome X open reading frame 61 (CXORF61), claudin 18.2 (CLDN18.2), claudin 6 (CLDN6), Clostridium difficile, clumping factor A, CLCA2, colony stimulating factor 1 receptor (CSF1R), CSF2, CTLA-4, C-type lectin domain family 12 member A (CLEC12A), C-type lectin-like molecule-1 (CLL-1 or CLECL1), C-X-C chemokine receptor type 4, cyclin B1, cytochrome P4501B1 (CYP1B1), cyp-B, cytomegalovirus, cytomegalovirus glycoprotein B, dabigatran, DLL4, DPP4, DR5, E. coli shiga toxin type-1, E. coli shiga toxin type-2, ecto-ADP-ribosyltransferase 4 (ART4), EGF-like module-containing mucin-like hormone receptor-like 2 (EMR2), EGF-like-domain multiple 7 (EGFL7), elongation factor 2 mutated (ELF2M), endotoxin, Ephrin A2, Ephrin B2, ephrin type-A receptor 2, epidermal growth factor receptor (EGFR), epidermal growth factor receptor variant III (EGFRvIII), episialin, epithelial cell adhesion molecule (EpCAM), epithelial glycoprotein 2 (EGP-2), epithelial glycoprotein 40 (EGP-40), ERBB2, ERBB3, ERBB4, ERG (transmembrane protease, serine 2 (TMPRSS2) ETS fusion gene), Escherichia coli, ETS translocation-variant gene 6, located on chromosome 12p (ETV6-AML), F protein of respiratory syncytial virus, FAP, Fc fragment of IgA receptor (FCAR or CD89), Fc receptor-like 5 (FCRLS), fetal acetylcholine receptor, fibrin II β chain, fibroblast activation protein α (FAP), fibronectin extra domain-B, FGF-5, Fms-Like Tyrosine Kinase 3 (FLT3), folate binding protein (FBP), folate hydrolase, folate receptor 1, folate receptor α, folate receptor β, Fos-related antigen 1, Frizzled receptor, Fucosyl GM1, G250, G protein-coupled receptor 20 (GPR20), G protein-coupled receptor class C group 5, member D (GPRC5D), ganglioside G2 (GD2), GD3 ganglioside, glycoprotein 100 (gp100), glypican-3 (GPC3), GMCSF receptor α-chain, GPNMB, GnT-V, growth differentiation factor 8, GUCY2C, heat shock protein 70-2 mutated (mut hsp70-2), hemagglutinin, Hepatitis A virus cellular receptor 1 (HAVCR1), hepatitis B surface antigen, hepatitis B virus, HER1, HER2/neu, HER3, hexasaccharide portion of globoH glycoceramide (GloboH), HGF, HHGFR, high molecular weight-melanoma-associated antigen (HMW-MAA), histone complex, HIV-1, HLA-DR, HNGF, Hsp90, HST-2 (FGF6), human papilloma virus E6 (HPV E6), human papilloma virus E7 (HPV E7), human scatter factor receptor kinase, human Telomerase reverse transcriptase (hTERT), human TNF, ICAM-1 (CD54), iCE, IFN-α, IFN-β, IFN-γ, IgE, IgE Fc region, IGF-1, IGF-1 receptor, IGHE, IL-12, IL-13, IL-17, IL-17A, IL-17F, IL-1β, IL-20, IL-22, IL-23, IL-31, IL-31RA, IL-4, IL-5, IL-6, IL-6 receptor, IL-9, immunoglobulin lambda-like polypeptide 1 (IGLL1), influenza A hemagglutinin, insulin-like growth factor 1 receptor (IGF-I receptor), insulin-like growth factor 2 (ILGF2), integrin α4β7, integrin β2, integrin α2, integrin α4, integrin α5β1, integrin α7β7, integrin αIIbβ3, integrin αvβ3, interferon α/β receptor, interferon γ-induced protein, Interleukin 11 receptor α (IL-11Rα), Interleukin-13 receptor subunit α-2 (IL-13Ra2 or CD213A2), intestinal carboxyl esterase, kinase domain region (KDR), KIR2D, KIT (CD117), L1-cell adhesion molecule (L1-CAM), legumain, leukocyte immunoglobulin-like receptor subfamily A member 2 (LILRA2), leukocyte-associated immunoglobulin-like receptor 1 (LAIR1), lymphocyte antigen 6 (Ly-6), Lewis-Y antigen, LFA-1 (CD11a), LINGO-1, lipoteichoic acid, LOXL2, L-selectin (CD62L), lymphocyte antigen 6 complex, locus K 9 (LY6K), lymphocyte antigen 75 (LY75), lymphocyte-specific protein tyrosine kinase (LCK), lymphotoxin-α (LT-α) or Tumor necrosis factor-0 (TNF-β), Lysosomal Associated Membrane Protein 1 (LAMP1), macrophage migration inhibitory factor (MIF or MMIF), M-CSF, mammary gland differentiation antigen (NY-BR-1), MCP-1, melanoma cancer testis antigen-1 (MAD-CT-1), melanoma cancer testis antigen-2 (MAD-CT-2), melanoma inhibitor of apoptosis (ML-IAP), melanoma-associated antigen 1 (MAGE-A1), mesothelin, mucin 1, cell surface associated (MUC1), MUC-2, MUC3, MUC4, MUC5AC, MUC5B, MUC7, MUC16, mucin CanAg, myelin-associated glycoprotein, myostatin, N-Acetyl glucosaminyl-transferase V (NA17), NCA-90 (granulocyte antigen), Nectin-4, nerve growth factor (NGF), neural apoptosis-regulated proteinase 1, neural cell adhesion molecule (NCAM), neurite outgrowth inhibitor (e.g., NOGO-A, NOGO-B, NOGO-C), neuropilin-1 (NRP1), N-glycolylneuraminic acid, NKG2D, Notch receptor, o-acetyl-GD2 ganglioside (OAcGD2), olfactory receptor 51E2 (OR51E2), oncofetal antigen (h5T4), oncogene fusion protein consisting of breakpoint cluster region (BCR) and Abelson murine leukemia viral oncogene homolog 1 (Abl) (bcr-abl), Oryctolagus cuniculus, OX-40, oxLDL, p53 mutant, paired box protein Pax-3 (PAX3), paired box protein Pax-5 (PAXS), pannexin 3 (PANX3), P-cadherin, phosphate-sodium co-transporter, phosphatidylserine, placenta-specific 1 (PLAC1), platelet-derived growth factor receptor α (PDGF-R α), platelet-derived growth factor receptor β (PDGFR-β), polysialic acid, proacrosin binding protein sp32 (OY-TES1), programmed cell death protein 1 (PD-1), Programmed death-ligand 1 (PD-L1), proprotein convertase subtilisin/kexin type 9 (PCSK9), prostase, prostate carcinoma tumor antigen-1 (PCTA-1 or Galectin 8), melanoma antigen recognized by T cells 1 (MelanA or MART1), P15, P53, PRAME, prostate stem cell antigen (PSCA), prostate-specific membrane antigen (PSMA), prostatic acid phosphatase (PAP), prostatic carcinoma cells, prostein, Protease Serine 21 (Testisin or PRSS21), Proteasome (Prosome, Macropain) Subunit, β Type, 9 (LMP2), Pseudomonas aeruginosa, rabies virus glycoprotein, RAGE, Ras Homolog Family Member C (RhoC), receptor activator of nuclear factor kappa-B ligand (RANKL), Receptor for Advanced Glycation Endproducts (RAGE-1), receptor tyrosine kinase-like orphan receptor 1 (ROR1), renal ubiquitous 1 (RU1), renal ubiquitous 2 (RU2), respiratory syncytial virus, Rh blood group D antigen, Rhesus factor, sarcoma translocation breakpoints, sclerostin (SOST), selectin P, sialyl Lewis adhesion molecule (sLe), sperm protein 17 (SPA17), sphingosine-1-phosphate, squamous cell carcinoma antigen recognized by T Cells 1, 2, and 3 (SART1, SART2, and SART3), stage-specific embryonic antigen-4 (SSEA-4), Staphylococcus aureus, STEAP1, syndecan 1 (SDC1)+A314, SOX10, survivin, survivin-2B, synovial sarcoma, X breakpoint 2 (SSX2), T-cell receptor, TCR F Alternate Reading Frame Protein (TARP), telomerase, TEM1, tenascin C, TGF-β (e.g., TGF-β1, TGF-β2, TGF-β3), thyroid stimulating hormone receptor (TSHR), tissue factor pathway inhibitor (TFPI), Tn antigen ((Tn Ag) or (GalNAcα-Ser/Thr)), TNF receptor family member B cell maturation (BCMA), TNF-α, TRAIL-R1, TRAIL-R2, TRG, transglutaminase 5 (TGS5), tumor antigen CTAA16.88, tumor endothelial marker 1 (TEM1/CD248), tumor endothelial marker 7-related (TEM7R), tumor protein p53 (p53), tumor specific glycosylation of MUC1, tumor-associated calcium signal transducer 2 (TROP-2), tumor-associated glycoprotein 72 (TAG72), tumor-associated glycoprotein 72 (TAG-72)+A327, TWEAK receptor, tyrosinase, tyrosinase-related protein 1 (TYRP1 or glycoprotein 75), tyrosinase-related protein 2 (TYRP2), uroplakin 2 (UPK2), vascular endothelial growth factor (e.g., VEGF-A, VEGF-B, VEGF-C, VEGF-D, PIGF), vascular endothelial growth factor receptor 1 (VEGFR1), vascular endothelial growth factor receptor 2 (VEGFR2), vimentin, v-myc avian myelocytomatosis viral oncogene neuroblastoma derived homolog (MYCN), von Willebrand factor (VWF), Wilms tumor protein (WT1), X Antigen Family, Member 1A (XAGE1), β-amyloid, κ-light chain, Fibroblast Growth Factor Receptor 2 (FGFR2), LIV-1 Protein, estrogen regulated (LIV1, aka SLC39A6), Neurotrophic Receptor Tyrosine Kinase 1 (NTRK1, aka TRK), Ret Proto-Oncogene (RET), B Cell Maturation Antigen (BCMA, aka TNFRSF17), Transferrin Receptor (TFRC, aka CD71), Activated Leukocyte Cell Adhesion Molecule (ALCAM, aka CD166), Somatostatin Receptor 2 (SSTR2), KIT Proto-Oncogene Receptor Tyrosine Kinase (cKIT), V-Set Immunoregulatory Receptor (VSIR, aka VISTA), Glycoprotein Nmb (GPNMB), Delta Like Canonical Notch Ligand 3 (DLL3), Interleukin 3 Receptor Subunit Alpha (IL3RA, aka CD123), Lysosomal Associated Membrane Protein 1 (LAMP1), Cadherin 3, Type 1, P-Cadherin (CDH3), Ephrin A4 (EFNA4), Protein Tyrosine Kinase 7 (PTK7), Solute Carrier Family 34 Member 2 (SLC34A2, aka NaPi-2b), Guanylyl Cyclase C (GCC), PLAUR Domain Containing 3 (LYPD3, aka LY6 or C4.4a), Mucin 17, Cell Surface Associated (MUC17), Fms Related Receptor Tyrosine Kinase 3 (FLT3), NKG2D ligands (e.g. ULBP1, ULBP2, ULBP3, H60, Rae-1α, Rae-1β, Rae-1δ, Rae-1γ, MICA, MICB, hHLA-A), SLAM Family Member 7 (SLAMF7), Interleukin 13 Receptor Subunit Alpha 2 (IL13RA2), C-Type Lectin Domain Family 12 Member A (CLEC12A aka CLL-1), CEA Cell Adhesion Molecule 5 (CEACAM aka CD66e), Interleukin 3 Receptor Subunit Alpha (IL3RA), CD5 Molecule (CD5), UL16 Binding Protein 1 (ILBP1), V-Set Domain Containing T Cell Activation Inhibitor 1 (VTCN1 aka B7-H4), Chondroitin Sulfate Proteoglycan 4 (CSPG4), Syndecan 1 (SDC1 aka CD138), Interleukin 1 Receptor Accessory Protein (IL1RAP), Baculoviral IAP Repeat Containing 5 (BIRC5 aka Survivin), CD74 Molecule (CD74), Hepatitis A Virus Cellular Receptor 1 (HAVCR1 aka TIM1), SLIT and NTRK Like Family Member 6 (SILTRK6), CD37 Molecule (CD37), Coagulation Factor III, Tissue Factor (CD142 aka F3), AXL Receptor Tyrosine Kinase (AXL), Endothelin Receptor Type B (EDNRB aka ETBR), Cadherin 6 (CDH6), Fibroblast Growth Factor Receptor 3 (FGFR3), Carbonic Anhydrase 6 (CA6), CanAg glycoform of MUC1, Integrin Subunit Alpha V (ITGAV), Teratocarcinoma-Derived Growth Factor 1 (TDGF1, aka Crypto 1), SLAM Family Member 6 (SLAMF6 aka CD352), and Notch Receptor 3 (NOTCH3).

In some embodiments, (1) the AF2 fragment disclosed herein is selected from the group consisting of Fv, Fab, Fab′, Fab′-SH, linear antibody, a single domain antibody, and single-chain variable fragment (scFv), or (2) the AF1 and AF2 disclosed herein are configured as an (Fab′)2 or a single chain diabody.

In certain embodiments, the binding affinity of the AF2 to the target cell marker is at least 10-fold greater, or at least 100-fold greater, or at least 1000-fold greater than the binding affinity of the AF1 to CD3, as measured in an in vitro antigen-binding assay.

In some other embodiments, the AF1 and AF2 each exhibit an isoelectric point (pI) that is less than or equal to 6.6. In another embodiment, the AF1 and AF2 each exhibit a pI that is between 5.5 and 6.6, inclusive. In certain embodiments, the pI of AF1 is within 0.1, 0.2, 0.3, 0.4, 0.5, 0.6, 0.7, 0.8, 0.9, 1.0, 1.1, 1.2, 1.3, 1.4, or 1.5 pH units of the pI of the AF2.

In yet another aspect, disclosed herein is a polypeptide comprising an antigen binding fragment, wherein the antigen binding fragment comprises light chain complementarity-determining regions (CDR-L) and heavy chain complementarity-determining regions (CDR-H), wherein the antigen binding fragment a. specifically binds to the epsilon subunit of CD3; and b. comprises a VH amino acid sequence comprising SEQ ID NO: 920. In some embodiments, the antigen binding fragment comprises a VL amino acid sequence comprising SEQ ID NO: 919. In certain embodiments, the antigen binding fragment consists of SEQ ID NO: 921.

In one aspect, disclosed herein is a pharmaceutical composition comprising the polypeptide disclosed herein and one or more pharmaceutically suitable excipients. In some embodiments, the pharmaceutical composition is formulated for intradermal, subcutaneous, intravenous, intra-arterial, intraabdominal, intraperitoneal, intrathecal, or intramuscular administration. In another embodiment, the pharmaceutical composition is in a liquid form. In another embodiment, the pharmaceutical composition is in a pre-filled syringe for a single injection. In yet another embodiment, the pharmaceutical composition is formulated as a lyophilized powder to be reconstituted prior to administration.

In another aspect, disclosed herein is use of the polypeptide disclosed herein in the preparation of a medicament for the treatment of a disease in a subject. In some embodiments, the disease is selected from the group consisting of carcinomas, Hodgkin's lymphoma, non-Hodgkin's lymphoma, B cell lymphoma, diffuse large B cell lymphoma, T-cell lymphoma, follicular lymphoma, mantle cell lymphoma, blastoma, breast cancer, colon cancer, prostate cancer, head and neck cancer, any form of skin cancer, melanoma, genito-urinary tract cancer, ovarian cancer, ovarian cancer with malignant ascites, vaginal cancer, vulvar cancer, Ewing sarcoma, peritoneal carcinomatosis, uterine serous carcinoma, parathyroid cancer, endometrial cancer, cervical cancer, colorectal cancer, an epithelia intraperitoneal malignancy with malignant ascites, uterine cancer, mesothelioma in the peritoneum kidney cancers, lung cancer, laryngeal cancer, small-cell lung cancer, non-small cell lung cancer, gastric cancer, esophageal cancer, stomach cancer, small intestine cancer, liver cancer, hepatocarcinoma, retinoblastoma, hepatoblastoma, liposarcoma, pancreatic cancer, gall bladder cancer, testicular cancer, cancers of the bile duct, cancers of the bone, salivary gland carcinoma, thyroid cancer, craniopharyngioma, carcinoid tumor, epithelial cancer, arrhenoblastoma, adenocarcinoma, sarcomas of any origin, primary hematologic malignancies including acute or chronic lymphocytic leukemias, acute or chronic myelogenous leukemias, B-cell derived chronic lymphatic leukemia, hairy cell leukemia, myeloproliferative neoplastic disorders, or myelodysplastic disorders, myasthenia gravis, Morbus Basedow, Kaposi sarcoma, neuroblastoma, Hashimoto thyroiditis, Wilms tumor, or Goodpasture syndrome.

In yet another aspect, disclosed herein is a method of treating a disease in a subject, comprising administering to the subject in need thereof one or more therapeutically effective doses of the pharmaceutical composition disclosed herein. In certain embodiments, the subject is selected from the group consisting of mouse, rat, monkey, and human. In some embodiments, the disease is selected from the group consisting of carcinomas, Hodgkin's lymphoma, non-Hodgkin's lymphoma, B cell lymphoma, T-cell lymphoma, follicular lymphoma, mantle cell lymphoma, blastoma, breast cancer, colon cancer, prostate cancer, head and neck cancer, any form of skin cancer, melanoma, genito-urinary tract cancer, ovarian cancer, ovarian cancer with malignant ascites, peritoneal carcinomatosis, uterine serous carcinoma, endometrial cancer, cervical cancer, colorectal cancer, an epithelia intraperitoneal malignancy with malignant ascites, uterine cancer, mesothelioma in the peritoneum kidney cancers, lung cancer, small-cell lung cancer, non-small cell lung cancer, gastric cancer, esophageal cancer, stomach cancer, small intestine cancer, liver cancer, hepatocarcinoma, hepatoblastoma, liposarcoma, pancreatic cancer, gall bladder cancer, cancers of the bile duct, salivary gland carcinoma, thyroid cancer, epithelial cancer, adenocarcinoma, sarcomas of any origin, primary hematologic malignancies including acute or chronic lymphocytic leukemias, acute or chronic myelogenous leukemias, myeloproliferative neoplastic disorders, or myelodysplastic disorders, myasthenia gravis, Morbus Basedow, Hashimoto thyroiditis, or Goodpasture syndrome.

In other embodiments, the pharmaceutical composition is administered to the subject as one or more therapeutically effective doses administered twice weekly, once a week, every two weeks, every three weeks, every four weeks, or monthly. In certain embodiments, the pharmaceutical composition is administered to the subject as one or more therapeutically effective doses over a period of at least two weeks, or at least one month, or at least two months, or at least three months, or at least four months, or at least five months, or at least six months. In some embodiments, the dose is administered intradermally, subcutaneously, intravenously, intra-arterially, intra-abdominally, intraperitoneally, intrathecally, or intramuscularly.

In one aspect, disclosed herein is an isolated nucleic acid, the nucleic acid comprising (a) a polynucleotide encoding a polypeptide disclosed herein; or (b) the complement of the polynucleotide of (a).

In a related aspect, disclosed herein is an expression vector comprising the polynucleotide sequence disclosed herein and a recombinant regulatory sequence operably linked to the polynucleotide sequence.

In another aspect, disclosed herein is an isolated host cell, comprising the expression vector disclosed herein. In some embodiments, the host cell is a prokaryote. In one embodiment, the host cell is E. coli.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various features of this disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 (FIG. 1) depicts the individual components of a bispecific antigen binding fragment composition. FIG. 1A depicts an antigen binding fragment having affinity to a target cell marker. FIG. 1B depicts an antigen binding fragment having affinity to an effector cell. FIGS. 1C and 1D depict XTEN polypeptides of different length. FIG. 1E depicts a cleavable release segment.

FIG. 2(FIG. 2) depicts two different forms of the polypeptide compositions described herein. FIG. 2A depicts, on the left side, an antigen binding fragment to an effector cell fused with a release segment and an XTEN, while the arrow depicts the action of a protease to cleave the release segment leading to, on the right hand side, the release of the XTEN from the antigen binding fragment of the polypeptide, such that the antigen-binding fragment regains its full binding affinity potential as it is no longer shielded by the XTEN. FIG. 2B depicts, on the left side, a bispecific composition having an antigen binding fragment to an effector cell fused to an antigen binding fragment having binding affinity to a target cell marker. A release segment and an XTEN are also fused to the antigen binding fragment having affinity to the effector cell, while the arrow depicts the action of a protease to cleave the release segment leading to, on the right-hand side, the release of the XTEN and the fused antigen binding fragments from the polypeptide, which would then regain their full binding affinity potential as they are no longer shielded by the XTEN.

FIG. 3 (FIG. 3) depicts two different forms of a bispecific antigen binding polypeptide. On the left side, a bispecific composition having an antigen binding fragment to an effector cell is fused to an antigen binding fragment having binding affinity to a target cell marker with the release segment (with the scissors indicating susceptibility to protease cleavage) and the XTEN is fused to the antigen binding fragment having binding affinity to an effector cell, while on the right hand side, a bispecific composition having an antigen binding fragment to an effector cell is fused to an antigen binding fragment having binding affinity to a target cell marker, with the release segment and the XTEN fused to the antigen binding fragment having binding affinity to the target cell marker.

FIG. 4 (FIG. 4) depicts three different forms of a bispecific antigen-binding polypeptide. FIG. 4A depicts a bispecific composition having an scFv antigen binding fragment to an effector cell fused to an scFv antigen binding fragment having binding affinity to a target cell marker with a release segment (with the scissors indicating susceptibility to protease cleavage) and an XTEN fused to each antigen binding fragment. FIGS. 4B and 4C are variations of 4A in which the antigen binding fragments are in a diabody configuration, with the release segments (with the scissors indicating susceptibility to protease cleavage) and XTENs fused to the antigen binding fragment to an effector cell or the target cell marker, respectively.

FIG. 5 (FIG. 5) shows schematic representations of a bispecific antigen binding polypeptide in proximity to tumor tissue (on the top) and normal tissue (on the bottom). The bispecific antigen binding polypeptide is preferentially cleaved at the tumor tissue to release one or more XTEN moieties as compared to that in the normal tissue. The cleaved bispecific antigen binding polypeptide is capable of binding to a T cell and a tumor cell expressing a tumor-specific marker.

FIG. 6 (FIG. 6) depicts the amino acid sequence of the control release segment RSR-1517 (SEQ ID NO:42), showing the sites of peptide cleavage for the listed proteases.

FIG. 7 (FIG. 7) shows in vitro cytotoxic activity of an N- and C-terminally XTENylated anti-HER2-anti-CD3 XPAT construct (“HER2-XPAT”) versus the same construct non-XTENylated (“HER2-PAT”) in a PBMC/BT-474 cell cytotoxicity assay. Cytotoxicity was assessed by caspase 3/7 assay or luminescent ATP assay, respectively.

FIGS. 8A-8B (FIGS. 8A-8B) shows that in vitro toxicity of non-XTENylated HER2-CD3 construct correlates to HER2 expression. (A) shows dose response of non-XTENylated HER2-CD3 construct (“HER2-PAT”) in cell lines with varying HER2 expression in the presence of PBMCs. (B) shows dose response of non-XTENylated HER2-CD3 construct (“HER2-PAT”) vs XTENylated HER2-CD3 construct (“HER2-XPAT”) in select cell lines with varying HER2 expression in the presence of PBMCs.

FIG. 8C illustrates that relatively low levels of unmasking of an exemplary XTENylated HER2-CD3 construct (“HER2-XPAT”) are required to elicit a significant increase in cytotoxicity. For example, FIG. 8C shows that, at 10 nM total concentration, 1% unmasked PAT (0.1 nM) and 99% masked XPAT yields maximal activity equivalent to maximal activity of 100% PAT.

FIG. 9A (FIG. 9A) illustrates a proposed model of how the HER2-PAT molecule forms an immunological synapse between T-cell and HER2-positive cell.

FIG. 9B (FIG. 9B) shows that HER2-PAT and HER2-XPAT constructs are capable of inducing conventional markers of T-cell activation in “natural” T-cells (supplied as PBMCs). FIGURE B shows a dose response of non-XTENylated HER2-CD3 construct (“HER2-PAT”) versus XTENylated HER2-CD3 construct (“HER2-XPAT”) on upregulation of CD69+ T-cells (an early marker of T-cell activation) in PBMCs in the presence of HER2+ cells (SK-OV-3 cells) as assessed by flow cytometry.

FIG. 9C (FIG. 9C) shows PD-1+ T cell activation upon exposure to HER2-PAT and HER2-XPAT constructs. More specifically, FIG. 9C shows a dose-dependent response of non-XTENylated HER2-CD3 construct (“HER2-PAT”) versus XTENylated HER2-CD3 construct (“HER2-XPAT”) on PD-1+ expression in T-cells in PBMCs in the presence of HER2+ cells (SK-OV-3 cells) as assessed by flow cytometry.

FIG. 9D (FIG. 9D) shows TNF-alpha secretion upon exposure to HER2-PAT and HER2-XPAT constructs. More specifically, FIG. 9D shows a dose-dependent response of non-XTENylated HER2-CD3 construct (“HER2-PAT”) versus XTENylated HER2-CD3 construct (“HER2-XPAT”) on TNF-alpha secretion when a mixture of PBMCs and SKOV3 cells are exposed to such constructs as assessed by flow cytometry.

FIG. 9E (FIG. 9E) shows IL-2 secretion upon exposure to HER2-PAT and HER2-XPAT constructs. More specifically, FIG. 9E shows a dose-dependent response of non-XTENylated HER2-CD3 construct (“HER2-PAT”) versus XTENylated HER2-CD3 construct (“HER2-XPAT”) on IL-2 secretion when a mixture of PBMCs and SKOV3 cells are exposed to such constructs as assessed by flow cytometry.

FIG. 9F (FIG. 9F) shows IL-6 secretion upon exposure to HER2-PAT and HER2-XPAT constructs. More specifically, FIG. 9F shows a dose-dependent response of non-XTENylated HER2-CD3 construct (“HER2-PAT”) versus XTENylated HER2-CD3 construct (“HER2-XPAT”) on IL-6 secretion when a mixture of PBMCs and SKOV3 cells are exposed to such constructs as assessed by flow cytometry.

FIG. 10A (FIG. 10A) shows that HER2-XPAT shows significantly decreased cytotoxicity in an in vitro PBMC/cardiomyocyte model versus non-XTENylated HER2-PAT.

FIGS. 11A-11B (FIGS. 11A-11B) shows that T-cell activation by the HER2-PAT and HER2-XPAT constructs is dependent on engagement of HER2-positive cells. FIG. 11(A) shows activation of T-cells by HER2-PAT or HER2-XPAT in the presence or absence of BT-474 HER2+ cells in an in vitro Jurkat T cell/BT-474 model as measured by luciferase activity driven by NFAT response elements in Jurkat T cells. FIG. 11(B) shows similar data showing activation of T-cells by HER2-PAT or HER2-XPAT in the presence or absence of SK-OV-3 cells in an in vitro Jurkat T cell/SK-OV-3 model.

FIG. 12 (FIG. 12) shows that single (N- or C-) terminus XTENylation causes intermediate reduction of Jurkat cell activation by XTENylated HER2-CD3 molecules in the presence of HER2 positive cells. (A) shows dose response of Jurkat cell activation non-XTENylated HER2-CD3 construct, single terminus XTENylated HER2-CD3 construct, or N- and C-terminally XTENylated HER2-CD3 molecule in the presence of BT-474 cells. (B) shows dose response of Jurkat cell activation by non-XTENylated HER2-CD3 construct, single terminus XTENylated HER2-CD3 construct, or N- and C-terminally XTENylated HER2-CD3 molecule in the presence of SK-OV-3 cells.

FIG. 13 (FIG. 13) shows that an N- and C-terminally XTENylated anti-HER2-anti-CD3 molecule (“XPAT”) and non-XTENylated HER2-CD3 molecule (“PAT”) are effective at decreasing tumor burden in a BT-474/human PBMC xenograft model, and that the anti-tumor activity of XTENylated HER2-CD3 molecule (“XPAT”) depends on cleavage of the XTEN molecules. (A) shows tumor volume post treatment with vehicle +/− PBMCs, XTENylated HER2-CD3 molecule (“XPAT”) and non-XTENylated HER2-CD3 molecule (“PAT”) over 25 days. (B) shows tumor volume post treatment with vehicle +PBMCs, XTENylated HER2-CD3 molecule (“XPAT”) and XTENylated HER2-CD3 molecule with non-cleavable XTEN over 25 days.

FIGS. 14A-14D (FIGS. 14A-14D) shows that N- and C-terminally XTENylated HER2-CD3 molecule (“HER2-XPAT”) and non-XTENylated HER2-CD3 molecule (“HER2-PAT”) are effective at increasing populations of activated CD4+ and CD8+ tumor infiltrating lymphocytes in tumor tissue taken from a BT-474/human PBMC xenograft model post-treatment. FIG. 14(A) shows percentage of “activated” (e.g. as measured by CD25+ expression) CD4+ cells in tumors as assessed by flow cytometry after treatment with vehicle, HER2-XPAT, or HER2-PAT. (B) shows percentage of “activated” (e.g. as measured by CD25+ expression) CD8+ cells in tumors as assessed by flow cytometry after treatment with vehicle, HER2-XPAT, or HER2-PAT. FIG. 14(C) shows percentage of “activated” (e.g. as measured by CD69+ expression) CD4+ cells in tumors as assessed by flow cytometry after treatment with vehicle, HER2-XPAT, or HER2-PAT. FIG. (D) shows percentage of “activated” (e.g. as measured by CD69+ expression) CD8+ cells in tumors as assessed by flow cytometry after treatment with vehicle, HER2-XPAT, or HER2-PAT.

FIGS. 14E-14H (FIGS. 14E-14H) illustrate minimal XPAT activation in the blood periphery in the same experimental setup as described in FIGS. 14A-14D above. For example, no appreciable level of activation of blood T cells was observed after treatment with XTENylated HER2-CD3 molecule (“HER2-XPAT”) or treatment with non-XTENylated HER2-CD3 molecule (“HER2-PAT”), as measured by CD25+ expression in CD4+ T cells (FIG. 14E), CD25+ expression in CD8+ T cells (FIG. 14F), CD69+ expression in CD4+ T cells (FIG. 14G), or CD69+ expression in CD8+ T cells (FIG. 14H) from humanized BT-474 xenograft mice.

FIG. 14I (FIG. 14I) illustrates representative levels of CD3+ T cells per mass of tumor after treatment with vehicle, HER2-XPAT, or HER2-PAT, as assessed by flow cytometry.

FIG. 14J (FIG. 14J) shows that an alternate dosing schedule of HER2-XPAT is effective at reducing tumor burden in humanized BT-474 xenograft mice.

FIG. 15 (FIG. 15) illustrates that XTENylation of a HER2-CD3 molecule decreases toxicity of the HER2-CD3 construct in cynomolgus monkeys. (A) shows schemes for maximum-tolerated dose trials of XTENylated (“HER2-XPAT”) or non-XTENylated (“HER2-PAT”) in monkeys. (B) shows plasma levels of XTENylated or non-XTENylated molecules post-dosing in monkeys, showing that the maximum tolerated dose of the XTENylated construct is >1000× the non-XTENylated molecules.

FIGS. 16A-16B (FIGS. 16A-16B) shows that in cynomolgus monkeys, HER2-XPAT constructs induce T cell margination at doses greater than 2.5 mpk, but fail to activate peripheral T cells at doses as high as 50 mpk. FIG. 16(A) shows effects on total lymphocytes, showing that XTENylated constructs induce reduction in total blood lymphocytes at doses greater than 2.5 mpk. FIG. 16(B) shows effects on activated CD4+ and CD8+ T cell populations (e.g., CD69+ or CD25+) for HER2-XPAT 2275 and the corresponding construct without any XTENs, showing that, for HER2-XPAT 2275, the post-dose effect on systemic T cell activation is within pre-dose ranges, while HER2-PAT induced CD25 expression on CD4+ and CD8+ T cells.

FIG. 17 (FIG. 17) illustrates that XTENyation of a HER2-CD3 molecule decreases cytokine release syndrome when the agent is administered in cynomolgus monkeys. (A), (B), and (C) show maximal concentrations of serum IL-6, TNFalpha, or IFNgamma induced by dose series of HER2-PAT or HER2-XPAT in cynomolgus monkeys, showing that HER2-XPAT does not induce appreciable cytokine release at tested doses.

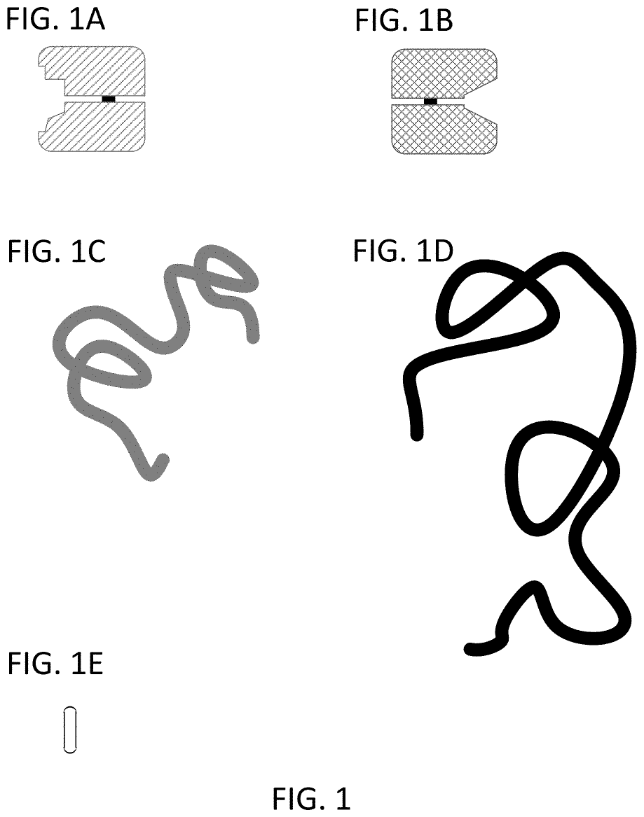

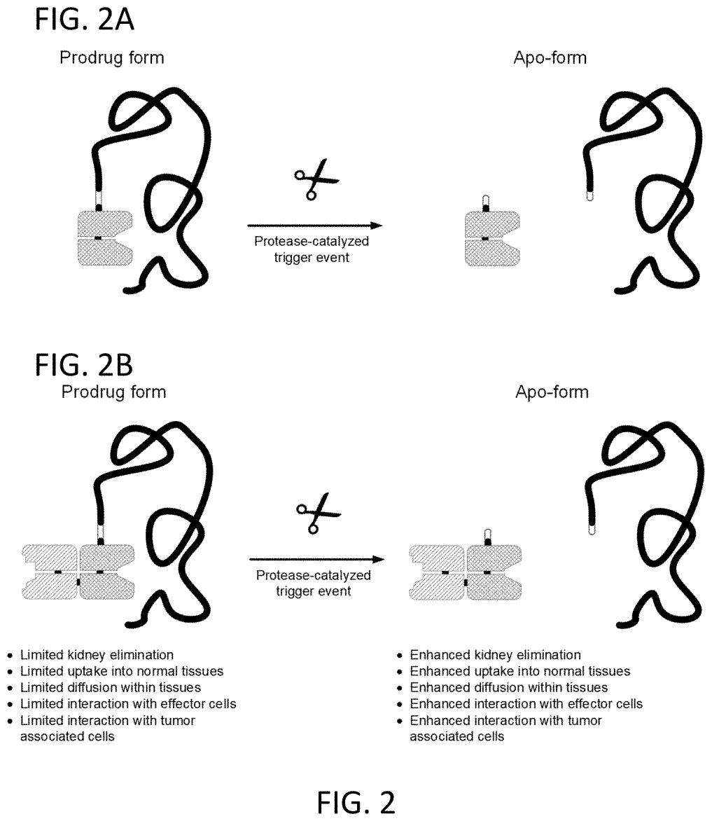

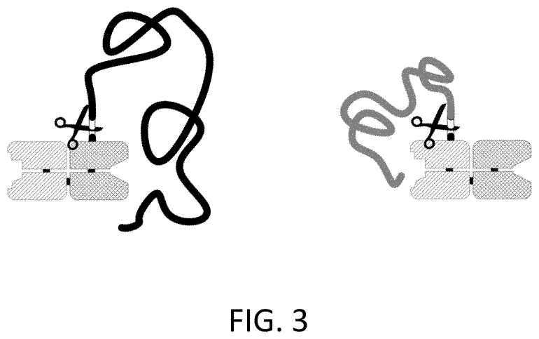

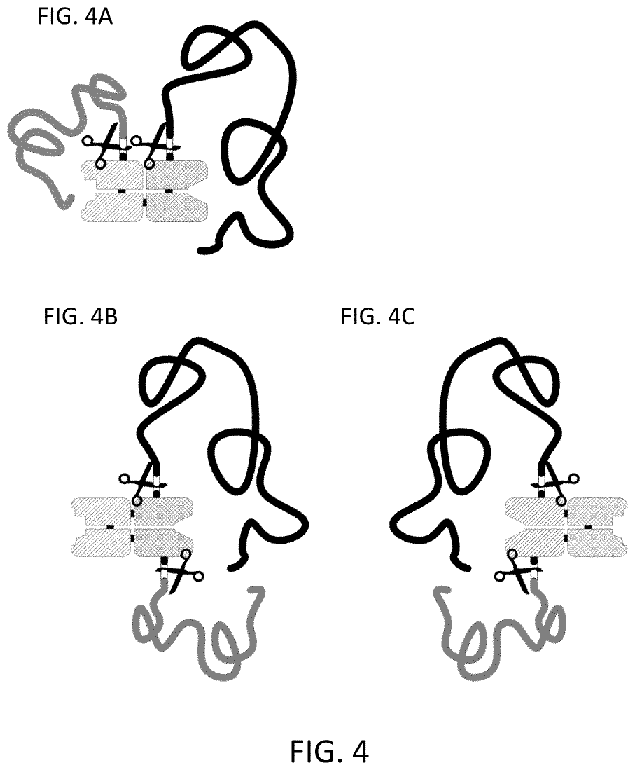

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

In the context of the present application, the following terms have the meanings ascribed to them unless specified otherwise:

As used throughout the specification and claims, the terms “a”, “an” and “the” are used in the sense that they mean “at least one”, “at least a first”, “one or more” or “a plurality” of the referenced components or steps, except in instances wherein an upper limit is thereafter specifically stated. Therefore, a “release segment”, as used herein, means “at least a first release segment” but includes a plurality of release segments. The operable limits and parameters of combinations, as with the amounts of any single agent, will be known to those of ordinary skill in the art in light of the present disclosure.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

The term “monomeric” as applied to a polypeptide refers to the state of the polypeptide as being a single continuous amino acid sequence substantially unassociated with one or more additional polypeptides of the same or different sequence.

As used herein, the term “amino acid” refers to either natural and/or unnatural or synthetic amino acids, including but not limited to both the D or L optical isomers, and amino acid analogs and peptidomimetics. Standard single or three letter codes may be used to designate amino acids.

The term “natural L-amino acid” or “L-amino acid” means the L optical isomer forms of glycine (G), proline (P), alanine (A), valine (V), leucine (L), isoleucine (I), methionine (M), cysteine (C), phenylalanine (F), tyrosine (Y), tryptophan (W), histidine (H), lysine (K), arginine (R), glutamine (Q), asparagine (N), glutamic acid (E), aspartic acid (D), serine (S), and threonine (T).

The term “antibody” is used herein in the broadest sense and encompasses various antibody structures, including but not limited to monoclonal antibodies, polyclonal antibodies, multispecific antibodies (e.g., bispecific antibodies), nanobodies, VHH antibodies, and antibody fragments so long as they exhibit the desired antigen-binding activity or immunological activity. The term “immunoglobulin” (Ig) is used interchangeably with antibody herein. The full-length antibodies may be, for example, monoclonal, recombinant, chimeric, deimmunized, humanized and human antibodies. Antibodies represent a large family of molecules that include several types of molecules, such as IgD, IgG, IgA, IgM and IgE. The term “immunoglobulin molecule” includes, for example, hybrid antibodies, or altered antibodies, and fragments thereof. It has been shown that the antigen binding function of an antibody can be performed by fragments of a naturally-occurring antibody or monoclonal antibody.

A “humanized” antibody refers to a chimeric antibody comprising amino acid residues from non-human complementarity-determining regions (CDRs) and amino acid residues from human framework regions (FRs). In certain embodiments, a humanized antibody will comprise substantially all of at least one, and typically two, variable domains, in which all or substantially all of the CDRs correspond to those of a non-human antibody (which may include amino acid substitutions), and all or substantially all of the FRs correspond to those of a human antibody (which may include amino acid substitutions).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being known in the art or described herein.

An “antigen binding fragment” as used herein refers to an immunoglobulin molecule and immunologically active portions of immunoglobulin molecule, i.e., a molecule that contains an antigen-binding site which specifically binds (“immunoreacts with”) an antigen. Examples include but are not limited to Fv, Fab, Fab′, Fab′-SH, F(ab′)2, diabodies, linear antibodies (see U.S. Pat. No. 5,641,870), a single domain antibody, a single domain camelid antibody, single-chain fragment variable (scFv) antibody molecules, and multispecific antibodies formed from antibody fragments that retain the ability to specifically bind to antigen. Also encompassed within the term “antigen binding fragment” is any polypeptide chain-containing molecular structure that has a specific shape which fits to and recognizes and binds to an epitope, where one or more non-covalent binding interactions stabilize the complex between the molecular structure and the epitope. An antigen binding fragment “specifically binds to” or is “immunoreactive with” an antigen if it binds with greater affinity or avidity than it binds to other reference antigens including polypeptides or other substances.

“scFv” or “single chain fragment variable” are used interchangeably herein to refer to an antibody fragment format comprising variable regions of heavy (“VH”) and light (“VL”) chains or two copies of a VH or VL chain of an antibody, which are joined together by a short flexible peptide linker which enables the scFv to form the desired structure for antigen binding. The scFv is a fusion protein of the variable regions of the heavy (VH) and light chains (VL) of immunoglobulins and can be easily expressed in functional form in E. coli or other host cells.

“Diabodies” refers to small antibody fragments prepared by constructing scFv fragments with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the VH and VL domains of the two antibodies are present on different polypeptide chains. Diabodies are described more fully in, for example, U.S. Pat. No. 7,635,475.

The term “bispecific antigen-binding fragment” is to be understood as an antigen binding fragment that has binding specificities for at least two different antigens

The terms “antigen”, “target antigen” and “immunogen” are used interchangeably herein to refer to the structure or binding determinant that an antibody, antibody fragment or an antibody fragment-based molecule binds to or has specificity against. The target antigen may be polypeptide, carbohydrate, nucleic acid, lipid, hapten or other naturally occurring or synthetic compound or portions thereof. An antigen is also a ligand for those antibodies or antibody fragments that have binding affinity for the antigen. Non-limiting exemplary antigens included CD3, HER2, EGFR, and EpCAM (and portions thereof) from human, non-human primates, murine, and other homologues thereof.

The term “CD3 antigen binding fragment” refers to an antigen binding fragment that is capable of binding cluster of differentiation 3 (CD3) or a member of the CD3 complex with sufficient affinity such that the antigen binding fragment is useful as a diagnostic and/or therapeutic agent in targeting CD3.

A “target cell marker” refers to a molecule expressed by a target cell including but not limited to cell-surface receptors, antigens, glycoproteins, oligonucleotides, enzymatic substrates, antigenic determinants, or binding sites that may be present in or on the surface of a target tissue or cell that may serve as ligands for antibodies.

A “target tissue” or “target cell” refers to a tissue or cell that is the cause of or is part of a disease condition such as, but not limited to cancer or inflammatory conditions. Sources of diseased target tissue or cells include a body organ, a tumor, a cancerous cell or population of cancerous cells or cells that form a matrix or are found in association with a population of cancerous cells, bone, skin, cells that produce cytokines or factors contributing to a disease condition.

The term “epitope” refers to the particular site on an antigen molecule to which an antibody, antibody fragment, or binding domain binds. An epitope is a ligand of an antibody or antibody fragment.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}). As used herein “a greater binding affinity” means a lower K_{d} value; e.g., 1×10⁻⁹ M is a greater binding affinity than 1×10⁻⁸ M. An antibody which binds an antigen of interest, e.g., a tumor-associated target cell antigen, is one that binds the antigen with sufficient affinity such that the antibody is useful as a diagnostic and/or therapeutic agent in targeting a cell or tissue expressing the antigen and does not significantly cross-react with other proteins.

“Dissociation constant”, or “K_{d}”, are used interchangeably and refer to the affinity between a ligand “L” and a protein “P”; i.e. how tightly a ligand binds to a particular protein. It can be calculated using the formula K_{d}=[L][P]/[LP], where [P], [L] and [LP] represent molar concentrations of the protein, ligand and complex, respectively.

The term “hypervariable region,” “HVR,” or “CDR”, when used herein, interchangeably refer to the regions of an antibody variable domain which are hypervariable in sequence and/or form structurally defined loops, and/or are involved in antigen recognition. Generally, antibodies comprise six hypervariable regions; three in the VH (H1, H2, H3), and three in the VL (L1, L2, L3). A number of CDR delineations are in use and are encompassed herein; e.g., CDR-L1 refers to the first hypervariable CDR region of the light chain, CDR-H2 refers to the second hypervariable CDR region of the heavy chain, etc. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)).

“Framework” or “FR” residues are those variable domain residues in antigen binding fragments other than the hypervariable region residues as herein defined, and are generally located between or that flank CDR. A number of FR delineations are in use and are encompassed herein; e.g., FR-L1 refers to the first FR region of the light chain, FR-H2 refers to the second FR region of the heavy chain, etc.

“Isoelectric point” or “pI” are used interchangeably herein to refer to the pH at which a particular molecule carries no net electrical charge or is electrically neutral in the statistical mean. The standard nomenclature to represent the isoelectric point is pH, such that the units are pH units; e.g., an antigen binding fragment with a pI of 6.3 would have a neutral charge in solution at pH 6.3. The isoelectric point can be determined mathematically, including a number of algorithms for estimating isoelectric points of peptides and proteins; e.g., the Henderson-Hasselbalch equation with different pK values. The isoelectric point can also be determined experimentally by in vitro assays such as capillary electrophoresis focusing.

The term “release segment” or “RS” refers to a peptide in the subject compositions having one or more sites within the sequence that can be recognized and cleaved by one or more proteases, effecting release of the antigen binding fragments and XTEN from the composition. As used herein, “mammalian protease” means a protease that normally exists in the body fluids, cells, tissues, and may be found in higher levels in certain target tissues or cells, e.g., in diseased tissues (e.g., tumor) of a mammal. RS sequences can be engineered to be cleaved by various mammalian proteases or multiple mammalian proteases that are present in or proximal to target tissues in a subject or are introduced in an in vitro assay. Other equivalent proteases (endogenous or exogenous) that are capable of recognizing a defined cleavage site can be utilized. It is specifically contemplated that the RS sequence can be adjusted and tailored to the protease utilized and can incorporate linker amino acids to join to adjacent polypeptides

The term “cleavage site” refers to that location between adjacent amino acids in a peptide or polypeptide that can be broken or cleaved by enzymes such as proteases; the breaking of the peptide bonds between the adjacent amino acids.

The term “within”, when referring to a first polypeptide being linked to a second polypeptide, encompasses linking or fusion of an additional component that connects the N-terminus of the first or second polypeptide to the C-terminus of the second or first polypeptide, respectively, as well as insertion of the first polypeptide into the sequence of the second polypeptide. For example, when an RS component is linked “within” a chimeric polypeptide assembly, the RS may be linked to the N-terminus, the C-terminus, or may be inserted between any two amino acids of an XTEN polypeptide.

“Activity” as applied to form(s) of a composition provided herein, refers to an action or effect, including but not limited to antigen binding, antagonist activity, agonist activity, a cellular or physiologic response, cell lysis, cell death, or an effect generally known in the art for the effector component of the composition, whether measured by an in vitro, ex vivo or in vivo assay or a clinical effect.

“Effector cell”, as used herein, includes any eukaryotic cells capable of conferring an effect on a target cell. For example, an effector cell can induce loss of membrane integrity, pyknosis, karyorrhexis, apoptosis, lysis, and/or death of a target cell. In another example, an effector cell can induce division, growth, differentiation of a target cell or otherwise altering signal transduction of a target cell. Non-limiting examples of effector cells include plasma cell, T cell, CD4 cell, CD8 cell, B cell, cytokine induced killer cell (CIK cell), master cell, dendritic cell, regulatory T cell (RegT cell), helper T cell, myeloid cell, macrophage, and NK cell.

An “effector cell antigen” refers to molecules expressed by an effector cell, including without limitation cell surface molecules such as proteins, glycoproteins or lipoproteins. Exemplary effector cell antigens include proteins of the CD3 complex or the T cell receptor (TCR), CD4, CD8, CD25, CD38, CD69, CD45RO, CD57, CD95, CD107, and CD154, as well as effector molecules such as cytokines in association with, bound to, expressed within, or expressed and released by, an effector cell. An effector cell antigen can serve as the binding counterpart of a binding domain of the subject chimeric polypeptide assembly.

As used herein, “CD3” or “cluster of differentiation 3” means the T cell surface antigen CD3 complex, which includes in individual form or independently combined form all known CD3 subunits, for example CD3 epsilon, CD3 delta, CD3 gamma, CD3 zeta, CD3 alpha and CD3 beta. The extracellular domains of CD3 epsilon, gamma and delta contain an immunoglobulin-like domain, so are therefore considered part of the immunoglobulin superfamily. CD3 includes, for example, human CD3 epsilon protein (NCBI RefSeq No. NP_000724), which is 207 amino acids in length, and human CD3 gamma protein (NCBI RefSeq No. NP_000064), which is 182 amino acids in length.

As used herein, the term “ELISA” refers to an enzyme-linked immunosorbent assay as described herein or as otherwise known in the art.

A “host cell” includes an individual cell or cell culture which can be or has been a recipient for the subject vectors into which exogenous nucleic acid has been introduced, such as those described herein. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

“Isolated,” when used to describe the various polypeptides disclosed herein, means a polypeptide that has been identified and separated and/or recovered from a component of its natural environment or from a more complex mixture (such as during protein purification). Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated nucleic acid” is a nucleic acid molecule that is identified and separated from at least one contaminant nucleic acid molecule with which it is ordinarily associated in the natural source of the polypeptide-encoding nucleic acid. For example, an isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

A “chimeric” protein or polypeptide contains at least one fusion polypeptide comprising at least one region in a different position in the sequence than that which occurs in nature. The regions may normally exist in separate proteins and are brought together in the fusion polypeptide; or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

“Fused,” and “fusion” are used interchangeably herein, and refers to the joining together of two or more peptide or polypeptide sequences by recombinant means. A “fusion protein” or “chimeric protein” comprises a first amino acid sequence linked to a second amino acid sequence with which it is not naturally linked in nature.

“XTENylated” is used to denote a peptide or polypeptide that has been modified by the linking or fusion of one or more XTEN polypeptides (described, below) to the peptide or polypeptide, whether by recombinant or chemical cross-linking means.

“Operably linked” means that the DNA sequences being linked are in reading phase or in-frame. An “in-frame fusion” refers to the joining of two or more open reading frames (ORFs) to form a continuous longer ORF, in a manner that maintains the correct reading frame of the original ORFs. For example, a promoter or enhancer is operably linked to a coding sequence for a polypeptide if it affects the transcription of the polypeptide sequence. Thus, the resulting recombinant fusion protein is a single protein containing two or more segments that correspond to polypeptides encoded by the original ORFs (which segments are not normally so joined in nature).

In the context of polypeptides, a “linear sequence” or a “sequence” is an order of amino acids in a polypeptide in an amino to carboxyl terminus (N- to C-terminus) direction in which residues that neighbor each other in the sequence are contiguous in the primary structure of the polypeptide. A “partial sequence” is a linear sequence of part of a polypeptide that is known to comprise additional residues in one or both directions.

“Heterologous” means derived from a genotypically distinct entity from the rest of the entity to which it is being compared. For example, a glycine-rich sequence removed from its native coding sequence and operatively linked to a coding sequence other than the native sequence is a heterologous glycine-rich sequence. The term “heterologous” as applied to a polynucleotide, a polypeptide, means that the polynucleotide or polypeptide is derived from a genotypically distinct entity from that of the rest of the entity to which it is being compared.

The terms “polynucleotides”, “nucleic acids”, “nucleotides,” and “oligonucleotides” are used interchangeably. They refer to nucleotides of any length, encompassing a singular nucleic acid as well as plural nucleic acids, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “complement of a polynucleotide” denotes a polynucleotide molecule having a complementary base sequence and reverse orientation as compared to a reference sequence, such that it could hybridize with a reference sequence with complete fidelity.

“Recombinant” as applied to a polynucleotide means that the polynucleotide is the product of various combinations of recombination steps which may include cloning, restriction and/or ligation steps, and other procedures that result in expression of a recombinant protein in a host cell.

The terms “gene” and “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

As used herein, a “coding region” or “coding sequence” is a portion of polynucleotide which consists of codons translatable into amino acids. Although a “stop codon” (TAG, TGA, or TAA) is typically not translated into an amino acid, it may be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions of the present invention can be present in a single polynucleotide construct, e.g., on a single vector, or in separate polynucleotide constructs, e.g., on separate (different) vectors. It follows, then, that a single vector can contain just a single coding region, or comprise two or more coding regions, e.g., a single vector can separately encode a binding domain-A and a binding domain-B as described below. In addition, a vector, polynucleotide, or nucleic acid of the invention can encode heterologous coding regions, either fused or unfused to a nucleic acid encoding a binding domain of the invention. Heterologous coding regions include without limitation specialized elements or motifs, such as a secretory signal peptide or a heterologous functional domain.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

“Homology” or “homologous” refers to sequence similarity or interchangeability between two or more polynucleotide sequences or between two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity compared to those sequences. Polypeptides that are homologous preferably have sequence identities that are at least 70%, preferably at least 80%, even more preferably at least 90%, even more preferably at least 95-99% identical when optimally aligned over sequences of comparable length.

“Ligation” as applied to polynucleic acids refers to the process of forming phosphodiester bonds between two nucleic acid fragments or genes, linking them together. To ligate the DNA fragments or genes together, the ends of the DNA must be compatible with each other. In some cases, the ends will be directly compatible after endonuclease digestion. However, it may be necessary to first convert the staggered ends commonly produced after endonuclease digestion to blunt ends to make them compatible for ligation.

The terms “stringent conditions” or “stringent hybridization conditions” include reference to conditions under which a polynucleotide will hybridize to its target sequence, to a detectably greater degree than other sequences (e.g., at least 2-fold over background). Generally, stringency of hybridization is expressed, in part, with reference to the temperature and salt concentration under which the wash step is carried out. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short polynucleotides (e.g., 10 to 50 nucleotides) and at least about 60° C. for long polynucleotides (e.g., greater than 50 nucleotides)—for example, “stringent conditions” can include hybridization in 50% formamide, 1 M NaCl, 1% SDS at 37° C., and three washes for 15 min each in 0.1×SSC/1% SDS at 60° C. to 65° C. Alternatively, temperatures of about 65° C., 60° C., 55° C., or 42° C. may be used. SSC concentration may be varied from about 0.1 to 2×SSC, with SDS being present at about 0.1%. Such wash temperatures are typically selected to be about 5° C. to 20° C. lower than the thermal melting point for the specific sequence at a defined ionic strength and pH. The Tm is the temperature (under defined ionic strength and pH) at which 50% of the target sequence hybridizes to a perfectly matched probe. An equation for calculating Tm and conditions for nucleic acid hybridization are well known and can be found in Sambrook, J. et al., “Molecular Cloning: A Laboratory Manual,” 3^{rd} edition, Cold Spring Harbor Laboratory Press, 2001. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art.

The terms “percent identity,” percentage of sequence identity,” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, or may be measured over a shorter length, for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured. The percentage of sequence identity is calculated by comparing two optimally aligned sequences over the window of comparison, determining the number of matched positions (at which identical residues occur in both polypeptide sequences), dividing the number of matched positions by the total number of positions in the window of comparison (i.e., the window size), and multiplying the result by 100 to yield the percentage of sequence identity. When sequences of different length are to be compared, the shortest sequence defines the length of the window of comparison. Conservative substitutions are not considered when calculating sequence identity.

The terms “percent identity,” percentage of sequence identity,” and “% identity,” with respect to the polypeptide sequences identified herein, is defined as the percentage of amino acid residues in a query sequence that are identical with the amino acid residues of a second, reference polypeptide sequence of comparable length or a portion thereof, after aligning the sequences and introducing gaps, if necessary, to achieve the maximum percent sequence identity, and not considering any conservative substitutions as part of the sequence identity, thereby resulting in optimal alignment. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve optimal alignment over the full length of the sequences being compared. Percent identity may be measured over the length of an entire defined polypeptide sequence, or may be measured over a shorter length, for example, over the length of a fragment taken from a larger, defined polypeptide sequence, for instance, a fragment of at least 10, at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Repetitiveness” used in the context of polynucleotide sequences refers to the degree of internal homology in the sequence such as, for example, the frequency of identical nucleotide sequences of a given length. Repetitiveness can, for example, be measured by analyzing the frequency of identical sequences.

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

A “vector” or “expression vector” are used interchangeably and refers to a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

“Serum degradation resistance,” as applied to a polypeptide, refers to the ability of the polypeptides to withstand degradation in blood or components thereof, which typically involves proteases in the serum or plasma. The serum degradation resistance can be measured by combining the protein with human (or mouse, rat, dog, monkey, as appropriate) serum or plasma, typically for a range of days (e.g. 0.25, 0.5, 1, 2, 4, 8, 16 days), typically at about 37° C. The samples for these time points can be run on a Western blot assay and the protein is detected with an antibody. The antibody can be to a tag in the protein. If the protein shows a single band on the western, where the protein's size is identical to that of the injected protein, then no degradation has occurred. In this exemplary method, the time point where 50% of the protein is degraded, as judged by Western blots or equivalent techniques, is the serum degradation half-life or “serum half-life” of the protein.

The terms “t_{1/2}”, “half-life”, “terminal half-life”, “elimination half-life” and “circulating half-life” are used interchangeably herein and, as used herein means the terminal half-life calculated as ln(2)/Kₑ₁. Kₑ₁ is the terminal elimination rate constant calculated by linear regression of the terminal linear portion of the log concentration vs. time curve. Half-life typically refers to the time required for half the quantity of an administered substance deposited in a living organism to be metabolized or eliminated by normal biological processes. When a clearance curve of a given polypeptide is constructed as a function of time, the curve is usually biphasic with a rapid α-phase and longer β-phase. The typical β-phase half-life of a human antibody in humans is 21 days. Half-life can be measured using timed samples from anybody fluid but is most typically measured in plasma samples.

The term “molecular weight” generally refers to the sum of atomic weights of the constituent atoms in a molecule. Molecular weight can be determined theoretically by summing the atomic masses of the constituent atoms in a molecule. When applied in the context of a polypeptide, the molecular weight is calculated by adding, based on amino acid composition, the molecular weight of each type of amino acid in the composition or by estimation from comparison to molecular weight standards in an SDS electrophoresis gel. The calculated molecular weight of a molecule can differ from the “apparent molecular weight” of a molecule, which generally refers to the molecular weight of a molecule as determined by one or more analytical techniques. “Apparent molecular weight factor” and “apparent molecular weight” are related terms and when used in the context of a polypeptide, the terms refer to a measure of the relative increase or decrease in apparent molecular weight exhibited by a particular amino acid or polypeptide sequence. The apparent molecular weight can be determined, for example, using size exclusion chromatography (SEC) or similar methods by comparing to globular protein standards, as measured in “apparent kD” units. The apparent molecular weight factor is the ratio between the apparent molecular weight and the “molecular weight”; the latter is calculated by adding, based on amino acid composition as described above, or by estimation from comparison to molecular weight standards in an SDS electrophoresis gel. The determination of apparent molecular weight and apparent molecular weight factor is described in U.S. Pat. No. 8,673,860.

A “defined medium” refers to a medium comprising nutritional and hormonal requirements necessary for the survival and/or growth of the cells in culture such that the components of the medium are known. Traditionally, the defined medium has been formulated by the addition of nutritional and growth factors necessary for growth and/or survival. Typically, the defined medium provides at least one component from one or more of the following categories: a) all essential amino acids, and usually the basic set of twenty amino acids plus cysteine; b) an energy source, usually in the form of a carbohydrate such as glucose; c) vitamins and/or other organic compounds required at low concentrations; d) free fatty acids; and e) trace elements, where trace elements are defined as inorganic compounds or naturally occurring elements that are typically required at very low concentrations, usually in the micromolar range. The defined medium may also optionally be supplemented with one or more components from any of the following categories: a) one or more mitogenic agents; b) salts and buffers as, for example, calcium, magnesium, and phosphate; c) nucleosides and bases such as, for example, adenosine and thymidine, hypoxanthine; and d) protein and tissue hydrolysates.

The term “agonist” is used in the broadest sense and includes any molecule that mimics a biological activity of a native polypeptide disclosed herein. Suitable agonist molecules specifically include agonist antibodies or antibody fragments, fragments or amino acid sequence variants of native polypeptides, peptides, small organic molecules, etc. Methods for identifying agonists of a native polypeptide may comprise contacting a native polypeptide with a candidate agonist molecule and measuring a detectable change in one or more biological activities normally associated with the native polypeptide.

As used herein, “treatment” or “treating,” or “palliating,” or “ameliorating” are used interchangeably herein. These terms refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms or improvement in one or more clinical parameters associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

A “therapeutic effect” or “therapeutic benefit,” as used herein, refers to a physiologic effect, including but not limited to the mitigation, amelioration, or prevention of disease or an improvement in one or more clinical parameters associated with the underlying disorder in a subject, or to otherwise enhance physical or mental wellbeing of a subject, resulting from administration of a polypeptide of the invention other than the ability to induce the production of an antibody against an antigenic epitope possessed by the biologically active protein. For prophylactic benefit, the compositions may be administered to a subject at risk of developing a particular disease, a recurrence of a former disease, condition or symptom of the disease, or to a subject reporting one or more of the physiological symptoms of a disease, even though a diagnosis of this disease may not have been made.

The terms “therapeutically effective amount” and “therapeutically effective dose”, as used herein, refer to an amount of a drug or a biologically active protein, either alone or as a part of a composition, that is capable of having any detectable, beneficial effect on any symptom, aspect, measured parameter or characteristics of a disease state or condition when administered in one or repeated doses to a subject. Such effect need not be absolute to be beneficial. Determination of a therapeutically effective amount is well within the capability of those skilled in the art, especially in light of the detailed disclosure provided herein.

The term “therapeutically effective and non-toxic dose” as used herein refers to a tolerable dose of the compositions as defined herein that is high enough to cause depletion of tumor or cancer cells, tumor elimination, tumor shrinkage or stabilization of disease without or essentially without major toxic effects in the subject. Such therapeutically effective and non-toxic doses may be determined by dose escalation studies described in the art and should be below the dose inducing severe adverse side effects.

The term “therapeutic index”, as used herein, refers to the ratio of the blood concentration at which a drug becomes toxic and the concentration at which the drug is effective. One exemplary ratio of therapeutic index is LD₅₀:ED₅₀, wherein LD₅₀ is the dose resulting in 50% mortality in a populations of subjects and ED₅₀ is the dose resulting in effectiveness in a population of subjects.

The term “dose regimen”, as used herein, refers to a schedule for consecutively administered multiple doses (i.e., at least two or more) of a composition, wherein the doses are given in therapeutically effective amounts to result in sustained beneficial effect on any symptom, aspect, measured parameter, endpoint, or characteristic of a disease state or condition in a subject.

As used herein, “administering” is meant a method of giving a dosage of a compound (e.g., an anti-CD3 antibody of the invention) or a composition (e.g., a pharmaceutical composition including an anti-CD3 antibody of the invention) to a subject.

A “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the subject or individual is a human.

The terms “cancer” and “cancerous” refer to or describe the physiological condition in mammals that is typically characterized by unregulated cell growth/proliferation. Examples of cancer include, but are not limited to, carcinomas, Hodgkin's lymphoma, non-Hodgkin's lymphoma, B cell lymphoma, T-cell lymphoma, follicular lymphoma, mantle cell lymphoma, blastoma, breast cancer, colon cancer, prostate cancer, head and neck cancer, any form of skin cancer, melanoma, genito-urinary tract cancer, ovarian cancer, ovarian cancer with malignant ascites, peritoneal carcinomatosis, uterine serous carcinoma, endometrial cancer, cervical cancer, colorectal cancer, an epithelia intraperitoneal malignancy with malignant ascites, uterine cancer, mesothelioma in the peritoneum kidney cancers, lung cancer, small-cell lung cancer, non-small cell lung cancer, gastric cancer, esophageal cancer, stomach cancer, small intestine cancer, liver cancer, hepatocarcinoma, hepatoblastoma, liposarcoma, pancreatic cancer, gall bladder cancer, cancers of the bile duct, salivary gland carcinoma, thyroid cancer, epithelial cancer, adenocarcinoma, sarcomas of any origin, primary hematologic malignancies including acute or chronic lymphocytic leukemias, acute or chronic myelogenous leukemias, myeloproliferative neoplastic disorders, or myelodysplastic disorders, myasthenia gravis, Morbus Basedow, Hashimoto thyroiditis, or Goodpasture syndrome.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder,” and “tumor” are not mutually exclusive as used herein.

“Tumor-specific marker” as used herein, refers to an antigen that is found on or in a cancer cell that may be, but is not necessarily, found in higher numbers in or on the cancer cell relative to normal cells or tissues.

### I). General Techniques

The practice of the present invention employs, unless otherwise indicated, conventional techniques of immunology, biochemistry, chemistry, molecular biology, microbiology, cell biology, genomics and recombinant DNA, which are within the skill of the art. See Sambrook, J. et al., “Molecular Cloning: A Laboratory Manual,” 3^{rd} edition, Cold Spring Harbor Laboratory Press, 2001; “Current protocols in molecular biology”, F. M. Ausubel, et al. eds., 1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory, 1988; “Goodman & Gilman's The Pharmacological Basis of Therapeutics,” 11th Edition, McGraw-Hill, 2005; and Freshney, R.I., “Culture of Animal Cells: A Manual of Basic Technique,” 4th edition, John Wiley & Sons, Somerset, N J, 2000, the contents of which are incorporated in their entirety herein by reference.

Host cells can be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing eukaryotic cells. In addition, animal cells can be grown in a defined medium that lacks serum but is supplemented with hormones, growth factors or any other factors necessary for the survival and/or growth of a particular cell type. Whereas a defined medium supporting cell survival maintains the viability, morphology, capacity to metabolize and potentially, capacity of the cell to differentiate, a defined medium promoting cell growth provides all chemicals necessary for cell proliferation or multiplication. The general parameters governing mammalian cell survival and growth in vitro are well established in the art. Physicochemical parameters which may be controlled in different cell culture systems are, e.g., pH, pO₂, temperature, and osmolarity. The nutritional requirements of cells are usually provided in standard media formulations developed to provide an optimal environment. Nutrients can be divided into several categories: amino acids and their derivatives, carbohydrates, sugars, fatty acids, complex lipids, nucleic acid derivatives and vitamins. Apart from nutrients for maintaining cell metabolism, most cells also require one or more hormones from at least one of the following groups: steroids, prostaglandins, growth factors, pituitary hormones, and peptide hormones to proliferate in serum-free media (Sato, G. H., et al. in “Growth of Cells in Hormonally Defined Media”, Cold Spring Harbor Press, N.Y., 1982). In addition to hormones, cells may require transport proteins such as transferrin (plasma iron transport protein), ceruloplasmin (a copper transport protein), and high-density lipoprotein (a lipid carrier) for survival and growth in vitro. The set of optimal hormones or transport proteins will vary for each cell type. Most of these hormones or transport proteins have been added exogenously or, in a rare case, a mutant cell line has been found which does not require a particular factor. Those skilled in the art will know of other factors required for maintaining a cell culture without undue experimentation.

Growth media for growth of prokaryotic host cells include nutrient broths (liquid nutrient medium) or LB medium (Luria Bertani). Suitable media include defined and undefined media. In general, media contains a carbon source such as glucose needed for bacterial growth, water, and salts. Media may also include a source of amino acids and nitrogen, for example beef or yeast extract (in an undefined medium) or known quantities of amino acids (in a defined medium). In some embodiments, the growth medium is LB broth, for example LB Miller broth or LB Lennox broth. LB broth comprises peptone (enzymatic digestion product of casein), yeast extract and sodium chloride. In some embodiments, a selective medium is used which comprises an antibiotic. In this medium, only the desired cells possessing resistance to the antibiotic will grow.

### II). CD3 Antigen Binding Fragment Compositions

In a first aspect, the disclosure provides polypeptides comprising an antigen binding fragment (AF1) having specific binding affinity for an effector cell antigen expressed on the surface of an effector cell selected from a plasma cell, a T cell, a B cell, a cytokine induced killer cell (CIK cell), a mast cell, a dendritic cell, a regulatory T cell (RegT cell), a helper T cell, a myeloid cell, and a NK cell. In one embodiment, the antigen binding fragment has binding affinity for an effector cell antigen expressed on the surface of a T cell. In another embodiment, the present disclosure provides polypeptides comprising antigen binding fragment having binding affinity for CD3. In another embodiment, the antigen binding fragment has binding affinity for a member of the CD3 complex, which includes in individual form or independently combined form all known CD3 subunits of the CD3 complex; for example, CD3 epsilon, CD3 delta, CD3 gamma, CD3 zeta, CD3 alpha and CD3 beta.

The antigen binding fragments that bind CD3 antigens have particular utility for pairing with a second antigen binding fragment (AF2) with binding affinity to a target cell marker or antigen of a diseased cell or tissue in composition formats in order to effect cell killing of the diseased cell or tissue. Binding specificity can be determined by complementarity determining regions, or CDRs, such as light chain CDRs or heavy chain CDRs. In many cases, binding specificity is determined by light chain CDRs and heavy chain CDRs. A given combination of heavy chain CDRs and light chain CDRs provides a given binding pocket that confers greater affinity and/or specificity towards CD3 as compared to other reference antigens.

The origin of the antigen binding fragments contemplated by the disclosure can be derived from a naturally occurring antibody or fragment thereof, a non-naturally occurring antibody or fragment thereof, a humanized antibody or fragment thereof, a synthetic antibody or fragment thereof, a hybrid antibody or fragment thereof, or an engineered antibody or fragment thereof. Methods for generating an antibody for a given target marker are well known in the art. For example, the monoclonal antibodies may be made using the hybridoma method described by Kohler et al., Nature, 256:495 (1975), or may be made by recombinant DNA methods (U.S. Pat. No. 4,816,567). The structure of antibodies and fragments thereof, variable regions of heavy and light chains of an antibody (VH and VL), single chain variable regions (scFv), complementarity determining regions (CDR), and domain antibodies (dAbs) are well understood. Methods for generating a polypeptide having a desired antigen binding fragment of a target cell marker are known in the art.

Certain CD3 binding antigen binding fragments of the disclosure have been specifically modified to enhance their stability in the polypeptide embodiments described herein relative to CD3 antibodies and antigen binding fragments known in the art. Protein aggregation of monoclonal and other antibodies continues to be a significant problem in their developability and remains a major area of focus in antibody production. Antibody aggregation can be triggered by partial unfolding of its domains, leading to monomer-monomer association followed by nucleation and aggregate growth. Although the aggregation propensities of antibodies and antibody-based proteins can be affected by the external experimental conditions, they are strongly dependent on the intrinsic antibody properties as determined by their sequences and structures. Although it is well known that proteins are only marginally stable in their folded states, it is often less well appreciated that most proteins are inherently aggregation-prone in their unfolded or partially unfolded states, and the resulting aggregates can be extremely stable and long-lived. Reduction in aggregation propensity has also been shown to be accompanied by an increase in expression titer, showing that reducing protein aggregation is beneficial throughout the development process and can lead to a more efficient path to clinical studies. For therapeutic proteins, aggregates are a significant risk factor for deleterious immune responses in patients and can form via a variety of mechanisms. Controlling aggregation can improve protein stability, manufacturability, attrition rates, safety, formulation, titers, immunogenicity, and solubility. The intrinsic properties of proteins such as size, hydrophobicity, electrostatics, and charge distribution play important roles in protein solubility. Low solubility of therapeutic proteins due to surface hydrophobicity has been shown to render formulation development more difficult and may lead to poor bio-distribution, undesirable pharmacokinetics behavior, and immunogenicity in vivo. Decreasing the overall surface hydrophobicity of candidate monoclonal antibodies can also provide benefits and cost savings relating to purification and dosing regimens. Individual amino acids can be identified by structural analysis as being contributory to aggregation potential in an antibody and can be located in CDR as well as framework regions. In particular, residues can be predicted to be at high risk of causing hydrophobicity issues in a given antibody. In one embodiment, the present disclosure provides an AF1 having the capability to specifically bind CD3 in which the AF1 has at least one amino acid substitution of a hydrophobic amino acid in a framework region relative to the parental antibody or antibody fragment wherein the hydrophobic amino acid is selected from isoleucine, leucine or methionine. In another embodiment, the CD3 AF1 has at least two amino acid substitutions of hydrophobic amino acids in one or more framework regions wherein the hydrophobic amino acids are selected from isoleucine, leucine or methionine.

In the context of the subject antigen binding fragments, the isoelectric point (pI) is the pH at which the antibody fragment has no net electrical charge. If the pH is below the pI of an antibody fragment, then it will have a net positive charge. A greater positive charge tends to correlate with increased blood clearance and tissue retention, with a generally shorter half-life. If the pH is greater than the pI of an antibody fragment it will have a negative charge. A negative charge generally results in decreased tissue uptake and a longer half-life. It is possible to manipulate this charge through mutations to the framework residues. These considerations informed the design of the sequences of the antigen binding fragments of the embodiments described herein wherein individual amino acid substitutions were made relative to the parental antibody utilized as the starting point. The isoelectric point of a polypeptide can be determined mathematically or experimentally in an in vitro assay. The isoelectric point (pI) is the pH at which a protein has a net charge of zero and can be calculated using the charges for the specific amino acids in the protein sequence. Estimated values for the charges are called acid dissociation constants or pKa values and are used to calculate the pI. The pI can be determined in vitro by methods such as capillary isoelectric focusing (see Datta-Mannan, A., et al. The interplay of non-specific binding, target-mediated clearance and FcRn interactions on the pharmacokinetics of humanized antibodies. mAbs 7:1084 (2015); Li, B., et al. Framework selection can influence pharmacokinetics of a humanized therapeutic antibody through differences in molecule charge. mAbs 6, 1255-1264 (2014)) or other methods known in the art.

In some aspects of any of the embodiments disclosed herein, a subject polypeptide comprising an AF1 comprises light chain complementarity-determining regions (CDR-L) and heavy chain complementarity-determining regions (CDR-H), wherein the AF1 (a) specifically binds to cluster of differentiation 3 T cell receptor (CD3), which can include, in individual form or independently combined form, all known CD3 subunits, for example CD3 epsilon, CD3 delta, CD3 gamma, CD3 zeta, CD3 alpha and CD3 beta. In one embodiment, the antigen binding fragments of any of the subject composition embodiments described herein is a chimeric or a humanized antigen binding fragment. In another embodiment, the antigen binding fragments of any of the subject composition embodiments described herein is selected from the group consisting of Fv, Fab, Fab′, Fab′-SH, linear antibody, and single-chain variable fragment (scFv). The antigen binding fragments having CDR-H and CDR-L can be configured in a (CDR-H)-(CDR-L) or a (CDR-H)-(CDR-L) orientation, N-terminus to C-terminus.

In one embodiment, the present disclosure provides polypeptides comprising an AF1 comprising CDR-L and CDR-H, wherein the AF1 (a) specifically binds to cluster of differentiation 3T cell receptor (CD3); and (b) comprises CDR-H3 having the amino acid sequence of SEQ ID NO: 10. In some embodiments of the present disclosure, the AF1 comprises CDR-H1, CDR-H2, and CDR-H3 having amino acid sequences of SEQ ID NOs: 8, 9, and 10, respectively. In another embodiment, the polypeptides of any of the subject composition embodiments described herein can comprise an AF1 wherein the AF1 comprises CDR-L and CDR-H, wherein the AF1: (a) specifically binds to CD3; (b) comprises CDR-H1, CDR-H2, and CDR-H3, wherein CDR-H3 comprises an amino acid sequence of SEQ ID NO:10; and (c) comprises heavy chain framework regions (FR-H) FR-H1, FR-H2, FR-H3, FR-H4, each exhibiting at least 86%, 87%, 88%, 89%, 90%, 91%, 92%, 93%, 94%, 95%, 96%, 97%, 98%, 99% sequence identity or is identical to amino acid sequences of SEQ ID NOs: 22, 23, 25, and 26, respectively. In another embodiment, the present disclosure provides polypeptides comprising an AF1, wherein the AF1 comprises CDR-L and CDR-H, wherein the AF1: (a) specifically binds to CD3; (b) comprises CDR-H1, CDR-H2, and CDR-H3, wherein CDR-H3 comprises an amino acid sequence of SEQ ID NO:10; and (c) comprises heavy chain framework regions (FR-H) FR-H1, FR-H2, FR-H3, FR-H4, each exhibiting at least 86%, 87%, 88%, 89%, 90%, 91%, 92%, 93%, 94%, 95%, 96%, 97%, 98%, 99% sequence identity or is identical to amino acid sequences of SEQ ID NOs: 22, 23, 25, and 26, respectively, and further comprises light chain framework regions (FR-L) FR-L1, FR-L2, FR-L3, FR-L4, each exhibiting at least 86%, 87%, 88%, 89%, 90%, 91%, 92%, 93%, 94%, 95%, 96%, 97%, 98%, 99% sequence identity or is identical to amino acid sequences of SEQ ID NOs: 12, 13, 18, and 19, respectively.

In another embodiment, a polypeptide of a subject composition embodiment described herein comprises an AF1, wherein the AF1 comprises CDR-L and CDR-H, wherein the AF1 (a) specifically binds to CD3; and (b) comprises CDR-H1, CDR-H2, and CDR-H3, wherein the CDR-H1, CDR-H2 and CDR-H3 comprises amino acid sequences of SEQ ID NOs:8, 9 and 10, respectively. In another embodiment of the foregoing, the polypeptide comprising an AF1 that further comprises (a) a CDR-L1 having an amino acid sequence of SEQ ID NOs: 1 or 2, (b) a CDR-L2 having an amino acid sequence of SEQ ID NOs: 4 or 5, and (c) a CDR-L3 having an amino acid sequence of SEQ ID NO:6. In yet another embodiment, the polypeptides of any of the subject composition embodiments described herein can comprise an AF1 that comprises CDR-L and CDR-H, wherein the AF1 (a) specifically binds to CD3; (b) comprises CDR-H1, CDR-H2, and CDR-H3, wherein the CDR-H1, CDR-H2 and CDR-H3 comprises amino acid sequences of SEQ ID NOs:8, 9 and 10, respectively and further comprise (c) a CDR-L1 having an amino acid sequence of SEQ ID NO:1; (d) a CDR-L2 having an amino acid sequence of any one of SEQ ID NOs: 4 or 5; and (e) a CDR-L3 having an amino acid sequence of SEQ ID NOs: 6 or 7. In yet another embodiment, the present disclosure provides polypeptides comprising an AF1 that comprises CDR-L and CDR-H, wherein the AF1 (a) specifically binds to CD3; (b) comprises CDR-H1, CDR-H2, and CDR-H3, wherein the CDR-H1, CDR-H2 and CDR-H3 comprises amino acid sequences of SEQ ID NOs:8, 9 and 10, respectively and further comprise (c) a CDR-L1 having an amino acid sequence of SEQ ID NO:2; (d) a CDR-L2 having an amino acid sequence of any one of SEQ ID NOs: 4 or 5; and (e) a CDR-L3 having an amino acid sequence of SEQ ID NO:6. In another embodiment, the polypeptides of any of the subject composition embodiments described herein can comprise an AF1 that comprises CDR-L and CDR-H, wherein the AF1 (a) specifically binds to CD3; (b) comprises CDR-H1, CDR-H2, and CDR-H3, wherein the CDR-H1, CDR-H2 and CDR-H3 comprises amino acid sequences of SEQ ID NOs:8, 9 and 10, respectively and further comprise (c) a CDR-L1 having an amino acid sequence of SEQ ID NO:1; (d) a CDR-L2 having an amino acid sequence of any one of SEQ ID NO: 4; and (e) a CDR-L3 having an amino acid sequence of SEQ ID NO: 6. In another embodiment, the present disclosure provides polypeptides comprising an AF1 that comprises CDR-L and CDR-H, wherein the AF1 (a) specifically binds to CD3; (b) comprises CDR-H1, CDR-H2, and CDR-H3, wherein the CDR-H1, CDR-H2 and CDR-H3 comprises amino acid sequences of SEQ ID NOs:8, 9 and 10, respectively and further comprise (c) a CDR-L1 having an amino acid sequence of SEQ ID NO:2; (d) a CDR-L2 having an amino acid sequence of any one of SEQ ID NO:5; and (e) a CDR-L3 having an amino acid sequence of SEQ ID NO:6. In the foregoing embodiments of the paragraph, the AF1 can further comprise light chain framework regions (FR-L) and heavy chain framework regions (FR-H) that link the respective CDR regions. In some cases of the foregoing embodiments of the paragraph, the AF1 further comprises: a FR-L1 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:12; a FR-L2 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:13; a FR-L3 exhibiting at least 86%, 87%, 88%, 89%, 90%, 91%, 92%, 93%, 94%, 95%, 96%, 97%, 98%, 99% sequence identity or is identical to the amino acid sequence of any one of SEQ ID NOs:14-17; a FR-L4 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:19; a FR-H1 exhibiting at least 86%, 87%, 88%, 89%, 90%, 91%, 92%, 93%, 94%, 95%, 96%, 97%, 98%, 99% sequence identity or is identical to the amino acid sequences of any one of SEQ ID NO:20, SEQ ID NO:21; a FR-H2 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:23; a FR-H3 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:24; and a FR-H4 exhibiting at least 86%, 87%, 88%, 89%, 90%, 91%, 92%, 93%, 94%, 95%, 96%, 97%, 98%, 99% sequence identity or is identical to the amino acid sequence of any one of SEQ ID NO:26. In other cases of the foregoing embodiments of the paragraph, the AF1 further comprises: a FR-L1 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:12; a FR-L2 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:13; a FR-L3 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:14; a FR-L4 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:19; a FR-H1 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:20; a FR-H2 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:23; a FR-H3 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:24; and a FR-H4 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:26. In other cases of the foregoing embodiments of the paragraph, the AF1 further comprises: a FR-L1 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:12; a FR-L2 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:13; a FR-L3 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:15; a FR-L4 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:19; a FR-H1 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:21; a FR-H2 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:23; a FR-H3 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:24; and a FR-H4 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:26. In other cases of the foregoing embodiments of the paragraph, the AF1 comprises: a FR-L1 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:12; a FR-L2 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:13; a FR-L3 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:16; a FR-L4 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:19; a FR-H1 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:21; a FR-H2 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:23; a FR-H3 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:24; and a FR-H4 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:26. In still other cases of the foregoing embodiments of the paragraph, the polypeptide comprising an AF1 comprises: a FR-L1 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:12; a FR-L2 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:13; a FR-L3 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:17; a FR-L4 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:19; a FR-H1 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:21; a FR-H2 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:23; a FR-H3 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:24; and a FR-H4 exhibiting at least 86%, 87%, 88%, 89%, 90%, 91%, 92%, 93%, 94%, 95%, 96%, 97%, 98%, 99% sequence identity or is identical to the amino acid sequence of SEQ ID NO:26.

In some aspects of any of the embodiments disclosed herein, a subject polypeptide can comprise an AF1 that binds to CD3, wherein the AF1 comprises VL regions and VH regions that confer the capability to specifically bind CD3. The AF1s can be configured in a VL-VH or VH-VL orientation and are fused by a linker peptide.

In one case, the present disclosure provides polypeptides comprising an AF1 comprising a VH amino acid sequence having at least 90%, 91%, 92%, 93%, 94%, 95%, 96%, 97%, 98%, 99% sequence identity or is identical to an amino acid sequence of SEQ ID NO:28 or SEQ ID NO:31. In another case, the present disclosure provides polypeptides comprising an AF1 comprising a VL amino acid sequence having at least 90%, 91%, 92%, 93%, 94%, 95%, 96%, 97%, 98%, 99% sequence identity or is identical to an amino acid sequence of any one of SEQ ID NOs: 27, 29, 30, 32, or 33. In another case, the polypeptides of any of the subject composition embodiments described herein comprise an AF1 that binds to CD3, wherein the AF1 comprises VL regions and VH regions that confer the capability to specifically bind CD3 and each has at least about 90%, 91%, 92%, 93%, 94%, 95%, 96%, 97%, 98%, 99% sequence identity or is identical to an amino acid sequence of SEQ ID NOs: 27 and 28, respectively. In other cases, the present disclosure provides polypeptides comprising an AF1 that binds to CD3, wherein the AF1 comprises VL regions and VH regions that confer the capability to specifically bind CD3 and each has at least about 90%, 91%, 92%, 93%, 94%, 95%, 96%, 97%, 98%, 99% sequence identity or is identical to an amino acid sequence of SEQ ID NOs: 29 and 28, respectively. In another case, the present disclosure provides polypeptides comprising an AF1 that binds to CD3, wherein the AF1 comprises VL regions and VH regions that confer the capability to specifically bind CD3 and each has at least about 90%, 91%, 92%, 93%, 94%, 95%, 96%, 97%, 98%, 99% sequence identity or is identical to an amino acid sequence of SEQ ID NOs: 30 and 31, respectively. In yet another case, the polypeptides of any of the subject composition embodiments described herein comprise an AF1 that binds to CD3, wherein the AF1 comprises VL regions and VH regions that confer the capability to specifically bind CD3 and each has at least about 90%, 91%, 92%, 93%, 94%, 95%, 96%, 97%, 98%, 99% sequence identity or is identical to an amino acid sequence of SEQ ID NOs: 32 and 31, respectively. In other cases, the present disclosure provides polypeptides comprising an AF1 that binds to CD3, wherein the AF1 comprises VL regions and VH regions that confer the capability to specifically bind CD3 and each has at least about 90%, 91%, 92%, 93%, 94%, 95%, 96%, 97%, 98%, 99% sequence identity or is identical to an amino acid sequence of SEQ ID NOs: 33 and 31, respectively.

In some aspects of any of the embodiments disclosed herein, a subject polypeptide comprises an AF1 that binds to CD3, wherein the AF1 is configured as an scFv having the capability to specifically bind CD3. In one embodiment, the AF1 comprises an amino acid sequence having at least 95%, 96%, 97%, 98%, 99% sequence identity or is identical to an amino acid sequence of any one of SEQ ID NOs:36-40.

In some cases, the CD3 AF1 of the polypeptide embodiments described herein specifically bind human or cynomolgus monkey (cyno) CD3. In other cases, the CD3 AF1 of the polypeptide embodiments described herein specifically binds human and cynomolgus monkey (cyno) CD3. In one embodiment, the CD3 AF1 of the polypeptide embodiments described herein binds a CD3 complex subunit selected from CD3 epsilon, CD3 delta, CD3 gamma, CD3 zeta, CD3 alpha and CD3 beta epsilon unit of CD3. In one embodiment, the AF1 of the polypeptide embodiments described herein binds a CD3 epsilon fragment of CD3.

In another aspect, the present disclosure provides polypeptides comprising an AF1 that binds to the CD3 protein complex and that has enhanced stability compared to CD3 binding antibodies or AF1s known in the art. Additionally, certain CD3 AF1 of the disclosure are designed to confer a higher degree of stability on the chimeric bispecific antigen binding compositions into which they are integrated, which may lead to improved expression and recovery of the fusion protein, increased shelf-life, and enhanced stability when administered to a subject. In one approach, certain CD3 AF1s of the present disclosure are designed to have a higher degree of thermal stability compared to certain CD3-binding antibodies and antigen binding fragments known in the art. As a result, the CD3 AF1 utilized as components of the chimeric bispecific antigen binding fragment compositions into which they are integrated exhibit favorable pharmaceutical properties, including high thermostability and low aggregation propensity, resulting in improved expression and recovery during manufacturing and storage, as well promoting long serum half-life. Biophysical properties such as thermostability are often limited by the antibody variable domains, which differ greatly in their intrinsic properties. High thermal stability is often associated with high expression levels and other desired properties, including being less susceptible to aggregation (Buchanan A, et al. Engineering a therapeutic IgG molecule to address cysteinylation, aggregation and enhance thermal stability and expression. MAbs 2013; 5:255). Thermal stability is determined by measuring the “melting temperature” (Tₘ), which is defined as the temperature at which half of the molecules are denatured. The melting temperature of each heterodimer is indicative of its thermal stability. In vitro assays to determine Tₘ are known in the art, including methods described in the Examples, below. The melting point of the heterodimer may be measured using techniques such as differential scanning calorimetry (Chen et al (2003) Pharm Res 20:1952-60; Ghirlando et al (1999) Immunol Lett 68:47-52). Alternatively, the thermal stability of the heterodimer may be measured using circular dichroism (Murray et al. (2002) J. Chromatogr Sci 40:343-9).

Thermal denaturation curves of the CD3 binding fragments and the anti-CD3 bispecific antibodies comprising said anti-CD3 binding fragment and a reference binding of the present disclosure show that various constructs of the present disclosure are more resistant to thermal denaturation than the antigen binding fragment consisting of a sequence shown in SEQ ID NO:41 or a control bispecific antibody wherein said control bispecific antigen binding fragment comprises SEQ ID NO:41 and a reference antigen binding fragment that binds to an antigen other than CD3. In one embodiment, the polypeptides of embodiments described herein comprise an anti-CD3 AF1, wherein the AF1 comprises CDR-L and CDR-H, and wherein the AF1: specifically binds to CD3; comprises CDR-H1, CDR-H2, and CDR-H3, wherein CDR-H3 comprises an amino acid sequence of SEQ ID NO:10, and exhibits a higher thermal stability, as evidenced by in an in vitro assay, wherein (i) the polypeptide exhibits a higher melting temperature (Tₘ) relative to that of an antigen binding fragment consisting of a sequence shown in SEQ ID NO:41, or (ii) upon incorporating said anti-CD3 AF1 into an anti-CD3 bispecific antibody, the bispecific antibody exhibits a higher Tₘ relative to a control bispecific antibody, wherein said anti-CD3 bispecific antibody comprises said anti-CD3 binding fragment and a reference antigen binding fragment that binds to an antigen other than CD3, and wherein said control bispecific antigen binding fragment consists of SEQ ID NO:41 and said reference antigen binding fragment. For instance, in some circumstances, the control bispecific antibody is identical to the subject polypeptide except that the AF1 is replaced with the antigen-binding fragment of SEQ ID NO:41). The reference antigen binding fragment of the embodiments is intended to include antigen binding fragments that bind any of the target cell markers described herein, including but not limited to EGFR, HER2, EpCAM, and CD19, amongst the other disclosed target cell markers. In one embodiment, the present disclosure provides a polypeptide comprising an anti-CD3 AF1, wherein the Tₘ of the AF1 is at least 2° C. greater, or at least 3° C. greater, or at least 4° C. greater, or at least 5° C. greater, or at least 6° C. greater, or at least 7° C. greater, or at least 8° C. greater, or at least 9° C. greater, or at least 10° C. greater than the Tₘ of an antigen binding fragment consisting of a sequence of SEQ ID NO:41. In another embodiment, the present disclosure provides a polypeptide comprising an anti-CD3 AF1, wherein the Tₘ of the AF1 is at least 2-10° C. greater, or at least 3-9° C. greater, or at least 4-8° C. greater, or at least 5-7° C. greater than the Tₘ of an antigen binding fragment consisting of the sequence of SEQ ID NO:41. In yet another embodiment, the disclosure provides bispecific antigen binding polypeptides comprising an anti-CD3 AF1, wherein the AF1 comprises CDR-L and CDR-H, and wherein the AF1: specifically binds to CD3; comprises CDR-H1, CDR-H2, and CDR-H3, wherein CDR-H3 comprises an amino acid sequence of SEQ ID NO:10, and a second antigen binding fragment that binds to an antigen other than CD3, and exhibits a higher thermal stability, as evidenced by in an in vitro assay, wherein the bispecific antigen binding polypeptide exhibits a higher melting temperature (Tₘ) relative to that of a control bispecific antibody control comprising a sequence shown in SEQ ID NO:41 and a reference antigen binding fragment that binds to an antigen other than CD3.

In a related aspect, the present disclosure provides various polypeptides comprising an AF1 that binds to CD3 that are incorporated into chimeric, bispecific antigen binding fragment compositions that are designed to have an isoelectric point (pI) that confer enhanced stability on the compositions of the disclosure compared to corresponding compositions comprising CD3 binding antibodies or antigen binding fragments known in the art. In one embodiment, polypeptide embodiments described herein can comprise antigen binding fragments that bind to CD3 wherein the AF1 exhibits a pI that is between 5.8 and 6.6, inclusive. In another embodiment, the present disclosure provides polypeptides comprising AF1 that bind to CD3 wherein the AF1 exhibits a pI that is at least 0.1, 0.2, 0.3, 0.4, 0.5, 0.6, 0.7, 0.8, 0.9, 1.0, 1.1, 1.2, 1.3, 1.4 or 1.5 pH units lower than the pI of a reference antigen binding fragment consisting of a sequence shown in SEQ ID NO: 41. In another embodiment, a polypeptide of any of the subject composition embodiments described herein can comprise an AF1 that binds to CD3 fused to a second antigen binding fragment that binds to an antigen other than CD3 wherein the CD3 AF1 exhibits a pI that is within at least 0.1, 0.2, 0.3, 0.4, 0.5, 0.6, 0.7, 0.8, 0.9, 1.0, 1.1, 1.2, 1.3, 1.4, or 1.5 pH units of the pI of the antigen binding fragment that does not binds to CD3. In another embodiment, the present disclosure provides polypeptides comprising an AF1 that binds to CD3 fused to a second antigen binding fragment that binds to an antigen other than CD3 wherein the CD3 AF1 exhibits a pI that is within at least about 0.1 to about 1.5, or at least about 0.3 to about 1.2, or at least about 0.5 to about 1.0, or at least about 0.7 to about 0.9 pH units of the pI of the second antigen binding fragment, as evidenced by calculation (see examples) or an in vitro assay. In one embodiment, the second antigen binding fragment has specific binding affinity to a non-CD3 antigen selected from the group consisting of EpCAM, EGFR, HER2, CD19, or any of the target cell marker embodiments disclosed herein, including but not limited to the target cell markers of Table 8. It is specifically intended that by such design wherein the pI of the two antigen binding fragments are within such ranges, the resulting fused antigen binding fragments will confer a higher degree of stability on the chimeric bispecific antigen binding fragment compositions into which they are integrated, leading to improved expression and enhanced recovery of the fusion protein in soluble, non-aggregated form, increased shelf-life of the formulated chimeric bispecific polypeptide compositions, and enhanced stability when the composition is administered to a subject.

In some aspects of any of the embodiments disclosed herein, a subject polypeptide comprises an AF1 that specifically binds human or cyno CD3 with a dissociation constant (K_{d}) constant between about between about 10 nM and about 400 nM, or between about 50 nM and about 350 nM, or between about 100 nM and 300 nM, as determined in an in vitro antigen-binding assay comprising a human or cyno CD3 antigen. In another embodiment, a polypeptide of any of the subject composition embodiments described herein can comprise an AF1 that specifically binds human or cyno CD3 with a dissociation constant (K_{d}) weaker than about 10 nM, or about 50 nM, or about 100 nM, or about 150 nM, or about 200 nM, or about 250 nM, or about 300 nM, or about 350 nM, or weaker than about 400 nM as determined in an in vitro antigen-binding assay. For clarity, an antigen binding fragment with a K_{d} of 400 nM binds its ligand more weakly than one with a K_{d} of 10 nM.

In another embodiment, the present disclosure provides polypeptides comprising an AF1 that exhibits a binding affinity to CD3 that is at least 2-fold, 3-fold, 4-fold, 5-fold, 6-fold, 7-fold, 8-fold, 9-fold, or at least 10-fold weaker relative to that of an antigen binding fragment consisting of an amino acid sequence of SEQ ID NO:41, as determined by the respective dissociation constants (K_{d}) in an in vitro antigen-binding assay. In another embodiment, the present disclosure provides polypeptides comprising an AF1 that exhibits a binding affinity to CD3 that is at least 2-fold, 3-fold, 4-fold, 5-fold, 6-fold, 7-fold, 8-fold, 9-fold, 10-fold, 20-fold, 50-fold, 100-fold, or at least 1000-fold at weaker relative to that of a second antigen binding fragment incorporated into the polypeptide that specifically binds an antigen other than CD3, as determined by the respective dissociation constants (K_{d}) in an in vitro antigen-binding assay. In the foregoing embodiment, the antigen other than CD3 is selected from, but not be limited to HER2, EGFR, EpCAM, or CD19, or any of the target cell marker embodiments disclosed herein, including but not limited to the target cell markers of Table 8. The binding affinity of the subject compositions for the target ligands can be assayed using binding or competitive binding assays, such as Biacore assays with chip-bound receptors or binding proteins or ELISA assays, as described in U.S. Pat. No. 5,534,617, assays described in the Examples herein, radio-receptor assays, or other assays known in the art. The binding affinity constant can then be determined using standard methods, such as Scatchard analysis, as described by van Zoelen, et al., Trends Pharmacol Sciences (1998) 19)12):487, or other methods known in the art. The same methodologies would be employed to make bispecific antigen binding fragment constructs having antigen binding fragments against CD3 and target cell markers described herein, in any combination or orientation (i.e., AF1-AF2 or AF2-AF1 in an N- to C-terminal orientation).

TABLE 1
CD3 CDR SEQUENCES

| | CDR | | |
|---|---|---|---|
| Construct | REGION | Amino Acid Sequence | SEQ ID NO: |
| 3.23, 3.30, 3.31, 3.32 | CDR-L1 | RSSNGAVTSSNYAN | 1 |
| 3.24 | CDR-L1 | RSSNGEVTTSNYAN | 2 |
| 3.9 | CDR-L1 | RSSTGAVTTSNYAN | 3 |
| 3.23, 3.30, 3.31, 3.32, 3.9 | CDR-L2 | GTNKRAP | 4 |
| 3.24 | CDR-L2 | GTIKRAP | 5 |
| 3.23, 3.24, 3.30, 3.31, 3.32 | CDR-L3 | ALWYPNLWVF | 6 |
| 3.9 | CDR-L3 | ALWYSNLWVF | 7 |
| 3.23, 3.24, 3.30, 3.31, 3.32, 3.9 | CDR-H1 | GFTFNTYAMN | 8 |
| 3.23, 3.24, 3.30, 3.31, 3.32, 3.9 | CDR-H2 | RIRSKYNNYATYYADSVKD | 9 |
| 3.23. 3.24, 3.30, 3.31, 3.32 | CDR-H3 | HENFGNSYVSWFAH | 10 |
| 3.9 | CDR-H3 | HGNFGNSYVSWFAY | 11 |

TABLE 2
CD3 FR SEQUENCES

| | FR | | SEQ ID |
|---|---|---|---|
| Construct | REGION | Amino Acid Sequence | NO: |
| 3.23, 3.24, 3.30, 3.31, 3.32, | FR-L1 | ELVVTQEPSLTVSPGGTVTLTC | 12 |
| 3.9 | | | |
| 3.23, 3.24, 3.30, 3.31, 3.32, | FR-L2 | WVQQKPGQAPRGLIG | 13 |
| 3.9 | | | |
| 3.23, 3.24 | FR-L3 | GTPARFSGSLLGGKAALTLSGVQPEDEAVYYC | 14 |
| 3.30 | FR-L3 | GTPARFSGSSLGGKAALTLSGVQPEDEAVYYC | 15 |
| 3.31 | FR-L3 | GTPARFSGSLLGGSAALTLSGVQPEDEAVYYC | 16 |
| 3.32 | FR-L3 | GTPARFSGSSLGGSAALTLSGVQPEDEAVYYC | 17 |
| 3.9 | FR-L3 | GTPARFSGSLLGGKAALTLSGVQPEDEAEYYC | 18 |
| 3.23, 3.24, 3.30, 3.31, 3.32, | FR-L4 | GGGTKLTVL | 19 |
| 3.9 | | | |
| 3.23, 3.24 | FR-H1 | EVQLLESGGGIVQPGGSLKLSCAAS | 20 |
| 3.30, 3.31, 3.32 | FR-H1 | EVQLQESGGGIVQPGGSLKLSCAAS | 21 |
| 3.9 | FR-H1 | EVQLLESGGGLVQPGGSLKLSCAAS | 22 |
| 3.23, 3.24, 3.30, 3.31, 3.32, | FR-H2 | WVRQAPGKGLEWVA | 23 |
| 3.9 | | | |
| 3.23, 3.24, 3.30, 3.31, 3.32 | FR-H3 | RFTISRDDSKNTVYLQMNNLKTEDTAVYYCVR | 24 |
| 3.9 | FR-H3 | RFTISRDDSKNTAYLQMNNLKTEDTAVYYCVR | 25 |
| 3.23, 3.24, 3.30, 3.31, 3.32, | FR-H4 | WGQGTLVTVSS | 26 |
| 3.9 | | | |

TABLE 3
VL & VH SEQUENCES

| | | | SEQ ID |
|---|---|---|---|
| Construct | REGION | Amino Acid Sequence | NO: |
| 3.23 | VL | ELVVTQEPSLTVSPGGTVTLTCRSSNGAVTSSNYANWVQQ | 27 |
| | | KPGQAPRGLIGGTNKRAPGTPARFSGSLLGGKAALTLSGV | |
| | | QPEDEAVYYCALWYPNLWVFGGGTKLTVL | |
| 3.23, 3.24 | VH | EVQLLESGGGIVQPGGSLKLSCAASGFTFNTYAMNWVRQA | 28 |
| | | PGKGLEWVARIRSKYNNYATYYADSVKDRFTISRDDSKNT | |
| | | VYLQMNNLKTEDTAVYYCVRHENFGNSYVSWFAHWGQGTL | |
| | | VTVSS | |
| 3.24 | VL | ELVVTQEPSLTVSPGGTVTLTCRSSNGEVTTSNYANWVQQ | 29 |
| | | KPGQAPRGLIGGTIKRAPGTPARFSGSLLGGKAALTLSGV | |
| | | QPEDEAVYYCALWYPNLWVFGGGTKLTVL | |
| 3.30 | VL | ELVVTQEPSLTVSPGGTVTLTCRSSNGAVTSSNYANWVQQ | 30 |
| | | KPGQAPRGLIGGTNKRAPGTPARFSGSSLGGKAALTLSGV | |
| | | QPEDEAVYYCALWYPNLWVFGGGTKLTVL | |
| 3.30, 3.31, | VH | EVQLQESGGGIVQPGGSLKLSCAASGFTFNTYAMNWVRQA | 31 |
| 3.32 | | PGKGLEWVARIRSKYNNYATYYADSVKDRFTISRDDSKNT | |
| | | VYLQMNNLKTEDTAVYYCVRHENFGNSYVSWFAHWGQGTL | |
| | | VTVSS | |
| 3.31 | VL | ELVVTQEPSLTVSPGGTVTLTCRSSNGAVTSSNYANWVQQ | 32 |
| | | KPGQAPRGLIGGTNKRAPGTPARFSGSLLGGSAALTLSGV | |
| | | QPEDEAVYYCALWYPNLWVFGGGTKLTVL | |
| 3.32 | VL | ELVVTQEPSLTVSPGGTVTLTCRSSNGAVTSSNYANWVQQ | 33 |
| | | KPGQAPRGLIGGTNKRAPGTPARFSGSSLGGSAALTLSGV | |
| | | QPEDEAVYYCALWYPNLWVFGGGTKLTVL | |
| 3.9 | VL | ELVVTQEPSLTVSPGGTVTLTCRSSTGAVTTSNYANWVQQ | 34 |
| | | KPGQAPRGLIGGTNKRAPGTPARFSGSLLGGKAALTLSGV | |
| | | QPEDEAEYYCALWYSNLWVFGGGTKLTVL | |
| 3.9 | VH | EVQLLESGGGLVQPGGSLKLSCAASGFTFNTYAMNWVRQA | 35 |
| | | PGKGLEWVARIRSKYNNYATYYADSVKDRFTISRDDSKNT | |
| | | AYLQMNNLKTEDTAVYYCVRHGNFGNSYVSWFAYWGQGTL | |
| | | VTVSS | |
| 3.33 | VL | ELVVTQEPSLTVSPGGTVTLTCRSSTGAVTTSNYANWVQQ | 919 |
| | | KPGQAPRGLIGGTNKRAPGTPARFSGSSLGGSAALTLSGV | |
| | | QPEDEAEYYCALWYSNLWVFGGGTKLTVL | |
| 3.33 | VH | EVQLQESGGGLVQPGGSLKLSCAASGFTFNTYAMNWVRQA | 920 |
| | | PGKGLEWVARIRSKYNNYATYYADSVKDRFTISRDDSKNT | |
| | | AYLQMNNLKTEDTAVYYCVRHGNFGNSYVSWFAYWGQGTL | |
| | | VTVSS | |

TABLE 4
scFv sequences

| Construct | Amino Acid Sequence | SEQ ID NO: |
|---|---|---|
| 3.23 | ELVVTQEPSLTVSPGGTVTLTCRSSNGAVTSSNYANWVQQKPGQAPRGLIG | 36 |
| | GTNKRAPGTPARFSGSLLGGKAALTLSGVQPEDEAVYYCALWYPNLWVFGG | |
| | GTKLTVLGATPPETGAETESPGETTGGSAESEPPGEGEVQLLESGGGIVQP | |
| | GGSLKLSCAASGFTFNTYAMNWVRQAPGKGLEWVARIRSKYNNYATYYADS | |
| | VKDRFTISRDDSKNTVYLQMNNLKTEDTAVYYCVRHENFGNSYVSWFAHWG | |
| | QGTLVTVSS | |
| 3.24 | ELVVTQEPSLTVSPGGTVTLTCRSSNGEVTTSNYANWVQQKPGQAPRGLIG | 37 |
| | GTIKRAPGTPARFSGSLLGGKAALTLSGVQPEDEAVYYCALWYPNLWVFGG | |
| | GTKLTVLGATPPETGAETESPGETTGGSAESEPPGEGEVQLLESGGGIVQP | |
| | GGSLKLSCAASGFTFNTYAMNWVRQAPGKGLEWVARIRSKYNNYATYYADS | |
| | VKDRFTISRDDSKNTVYLQMNNLKTEDTAVYYCVRHENFGNSYVSWFAHWG | |
| | QGTLVTVSS | |
| 3.30 | ELVVTQEPSLTVSPGGTVTLTCRSSNGAVTSSNYANWVQQKPGQAPRGLIG | 38 |
| | GTNKRAPGTPARFSGSSLGGKAALTLSGVQPEDEAVYYCALWYPNLWVFGG | |
| | GTKLTVLGATPPETGAETESPGETTGGSAESEPPGEGEVQLQESGGGIVQP | |
| | GGSLKLSCAASGFTFNTYAMNWVRQAPGKGLEWVARIRSKYNNYATYYADS | |
| | VKDRFTISRDDSKNTVYLQMNNLKTEDTAVYYCVRHENFGNSYVSWFAHWG | |
| | QGTLVTVSS | |
| 3.31 | ELVVTQEPSLTVSPGGTVTLTCRSSNGAVTSSNYANWVQQKPGQAPRGLIG | 39 |
| | GTNKRAPGTPARFSGSLLGGSAALTLSGVQPEDEAVYYCALWYPNLWVFGG | |
| | GTKLTVLGATPPETGAETESPGETTGGSAESEPPGEGEVQLQESGGGIVQP | |
| | GGSLKLSCAASGFTFNTYAMNWVRQAPGKGLEWVARIRSKYNNYATYYADS | |
| | VKDRFTISRDDSKNTVYLQMNNLKTEDTAVYYCVRHENFGNSYVSWFAHWG | |
| | QGTLVTVSS | |
| 3.32 | ELVVTQEPSLTVSPGGTVTLTCRSSNGAVTSSNYANWVQQKPGQAPRGLIG | 40 |
| | GTNKRAPGTPARFSGSSLGGSAALTLSGVQPEDEAVYYCALWYPNLWVFGG | |
| | GTKLTVLGATPPETGAETESPGETTGGSAESEPPGEGEVQLQESGGGIVQP | |
| | GGSLKLSCAASGFTFNTYAMNWVRQAPGKGLEWVARIRSKYNNYATYYADS | |
| | VKDRFTISRDDSKNTVYLQMNNLKTEDTAVYYCVRHENFGNSYVSWFAHWG | |
| | QGTLVTVSS | |
| 3.9 | ELVVTQEPSLTVSPGGTVTLTCRSSTGAVTTSNYANWVQQKPGQAPRGLIG | 41 |
| | GTNKRAPGTPARFSGSLLGGKAALTLSGVQPEDEAEYYCALWYSNLWVFGG | |
| | GTKLTVLGATPPETGAETESPGETTGGSAESEPPGEGEVQLLESGGGLVQP | |
| | GGSLKLSCAASGFTFNTYAMNWVRQAPGKGLEWVARIRSKYNNYATYYADS | |
| | VKDRFTISRDDSKNTAYLQMNNLKTEDTAVYYCVRHGNFGNSYVSWFAYWG | |
| | QGTLVTVSS | |
| 3.33 | ELVVTQEPSLTVSPGGTVTLTCRSSTGAVTTSNYANWVQQKPGQAPRGLIG | 921 |
| | GTNKRAPGTPARFSGSSLGGSAALTLSGVQPEDEAEYYCALWYSNLWVFGG | |
| | GTKLTVLGATPPETGAETESPGETTGGSAESEPPGEGEVQLQESGGGLVQP | |
| | GGSLKLSCAASGFTFNTYAMNWVRQAPGKGLEWVARIRSKYNNYATYYADS | |
| | VKDRFTISRDDSKNTAYLQMNNLKTEDTAVYYCVRHGNFGNSYVSWFAYWG | |
| | QGTLVTVSS | |
| 4.11 | QSVLTQPPSASGTPGQRVTISCSGSSSNIGSNYVYWYQQLPGTAPKLLIYR | 1160 |
| | NNQRPSGVPDRFSGSKSGTSASLAISGLRSEDEADYYCAAWDDSLSGLWVF | |
| | GGGTKLTVLGATPPETGAETESPGETTGGSAESEPPGEGQVQLQQWGGGLV | |
| | KPGGSLRLSCAASGFTFSSYSMNWVRQAPGKGLEWVSRINSDGSSTNYADS | |
| | VKGRFTISRDNAKNTLYLQMNSLRAEDTAVYYCARELRWGNWGQGTLVTVS | |
| | S | |
| 4.12 | QAGLTQPPSASGTPGQRVTLSCSGSYSNIGTYYVYWYQQLPGTAPKLLIYS | 1161 |
| | NDQRLSGVPDRFSGSKSGTSASLAISGLQSEDEAAYYCAAWDDSLNGWAFG | |
| | GGTKLTVLGATPPETGAETESPGETTGGSAESEPPGEGQVQLQQWGGGLVK | |
| | PGGSLRLSCAASGFTFSSYSMNWVRQAPGKGLEWVSRINSDGSSTNYADSV | |
| | KGRFTISRDNAKNTLYLQMNSLRAEDTAVYYCARELRWGNWGQGTLVTVSS | |
| 4.13 | QPGLTQPPSASGTPGQRVTLSCSGRSSNIGSYYVYWYQHLPGMAPKLLIYR | 1162 |
| | NSRRPSGVPDRFSGSKSGTSASLVISGLQSDDEADYYCAAWDDSLKSWVFG | |
| | GGTKLTVLGATPPETGAETESPGETTGGSAESEPPGEGQVQLQQWGGGLVK | |
| | PGGSLRLSCAASGFTFSSYSMNWVRQAPGKGLEWVSRINSDGSSTNYADSV | |
| | KGRFTISRDNAKNTLYLQMNSLRAEDTAVYYCARELRWGNWGQGTLVTVSS | |
| 4.14 | QSVLTQPPSASGTPGQRVTISCSGSSSNIGTNYVYWYQQFPGTAPKLLIYS | 1163 |
| | NNQRPSGVPDRFSGSKSGTSGSLAISGLQSEDEADYSCAAWDDSLNGWVFG | |
| | GGTKLTVLGATPPETGAETESPGETTGGSAESEPPGEGQVQLVQWGGGLVK | |
| | PGGSLRLSCAASGFTFSSYSMNWVRQAPGKGLEWVSRINSDGSSTNYADSV | |
| | KGRFTISRDNAKNTLYLQMNSLRAEDTAVYYCARELRWGNWGQGTLVTVSS | |
| 4.15 | QPGLTQPPSASGTPGQRVTISCSGSSSNIGSNYVYWYQQLPGTAPKLLIYR | 1164 |
| | NNQRPSGVPDRLSGSKSGTSASLAISGLRSEDEADYYCAAWDDSLSGWVFG | |
| | GGTKLTVLGATPPETGAETESPGETTGGSAESEPPGEGQVQLVQWGGGLVK | |
| | PGGSLRLSCAASGFTFSSYSMNWVRQAPGKGLEWVSRINSDGSSTNYADSV | |
| | KGRFTISRDNAKNTLYLQMNSLRAEDTAVYYCARELRWGNWGQGTLVTVSS | |
| 4.16 | QAVLTQPPSASGTPGQRVTISCSGSSSNIGSYYVYWYQQVPGAAPKLLMRL | 1165 |
| | NNQRPSGVPDRFSGAKSGTSASLVISGLRSEDEADYYCAAWDDSLSGQWVF | |
| | GGGTKLTVLGATPPETGAETESPGETTGGSAESEPPGEGQVQLQQWGGGLV | |
| | KPGGSLRLSCAASGFTFSSYSMNWVRQAPGKGLEWVSRINSDGSSTNYADS | |
| | VKGRFTISRDNAKNTLYLQMNSLRAEDTAVYYCARELRWGNWGQGTLVTVS | |
| | S | |
| 4.17 | QAGLTQPPSASGTPGQRVTISCSGSSSNIGSNYVYWYQQLPGTAPKLLIYR | 1166 |
| | NNQRPSGVPDRFSGSKSGTSASLAISGLRSEDEADYYCATWDASLSGWVFG | |
| | GGTKLTVLGATPPETGAETESPGETTGGSAESEPPGEGEVQLVQWGGGLVK | |
| | PGGSLRLSCAASGFTFSSYSMNWVRQAPGKGLEWVSRINSDGSSTNYADSV | |
| | KGRFTISRDNAKNTLYLQMNSLRAEDTAVYYCARELRWGNWGQGTLVTVSS | |

### III). Release Segments

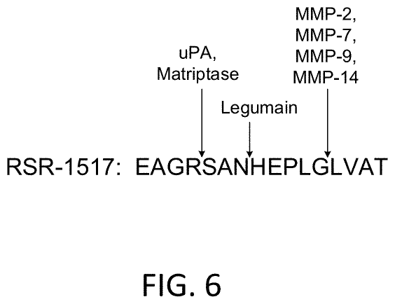

In another aspect, the disclosure relates to release segment (RS) peptides suitable for inclusion in the subject compositions described herein that are substrates for one or more mammalian proteases associated with or produced by disease tissues or cells found in proximity to disease tissues. Such proteases can include, but not be limited to the classes of proteases such as metalloproteinases, cysteine proteases, aspartate proteases, and serine proteases. The RS are useful for, amongst other things, conferring a prodrug format on the subject compositions that can be activated by the cleavage of the RS by mammalian proteases. As described herein, the RS are incorporated into the subject composition embodiments described herein, linking the incorporated antigen binding fragment to the XTEN (the configurations of which are described more fully, below) such that upon cleavage of the RS by action of the one or more proteases for which the RS are substrates, the antigen binding fragments and XTEN are released from the composition and the antigen binding fragments, no longer shielded by the XTEN, regain their full potential to bind their respective ligands. In a particular feature, the RS serve as substrates for proteases found in close association with or are co-localized with disease tissues or cells, such as but not limited to tumors, cancer cells, and inflammatory tissues, and upon cleavage of the RS, the antigen binding fragments that are otherwise shielded by the XTEN of the subject compositions (and thus have a lower binding affinity for their respective ligands) are released from the composition and regain their full potential to bind the target and/or effector cell ligands. In another embodiment, the RS of the subject polypeptide compositions comprise an amino acid sequence that is a substrate for a cellular protease located within a targeted cell. In another particular feature of the subject compositions described herein, the RS that are substrates for two or three classes of proteases were designed with sequences that are capable of being cleaved in different locations of the RS sequence by the different proteases, with a representative example depicted in FIG. 6. Thus, the RS that are substrates for two, three, or more classes of proteases have two, three, or a plurality of distinct cleavage sites in the RS sequence, but cleavage by a single protease nevertheless results in the release of the antigen binding fragments and the XTEN from the composition comprising the RS.

In one embodiment, the disclosure provides an activatable polypeptide comprising one or more release segments wherein the release segment is a substrate for cleavage by one or more mammalian proteases. In another embodiment, the present disclosure provides a polypeptide comprising a first release segment (RS1) sequence wherein the RS1 is a substrate for cleavage by a mammalian protease wherein the RS1 is a substrate for a protease selected from the group consisting of legumain, MMP-2, MMP-7, MMP-9, MMP-11, MMP-14, uPA, and matriptase. In other cases, the polypeptides of any of the subject composition embodiments described herein comprise a first release segment (RS1) sequence wherein the RS1 is a substrate for cleavage by one or more mammalian proteases selected from the group consisting of meprin, neprilysin (CD10), PSMA, BMP-1, A disintegrin and metalloproteinases (ADAMs), ADAMS, ADAMS, ADAM10, ADAM12, ADAM15, ADAM17 (TACE), ADAM19, ADAM28 (MDC-L), ADAM with thrombospondin motifs (ADAMTS), ADAMTS1, ADAMTS4, ADAMTS5, MMP-1 (collagenase 1), matrix metalloproteinase-1 (MMP-1), matrix metalloproteinase-2 (MMP-2, gelatinase A), matrix metalloproteinase-3 (MMP-3, stromelysin 1), matrix metalloproteinase-7 (MMP-7, Matrilysin 1), matrix metalloproteinase-8 (MMP-8, collagenase 2), matrix metalloproteinase-9 (MMP-9, gelatinase B), matrix metalloproteinase-10 (MMP-10, stromelysin 2), matrix metalloproteinase-11 (MMP-11, stromelysin 3), matrix metalloproteinase-12 (MMP-12, macrophage elastase), matrix metalloproteinase-13 (MMP-13, collagenase 3), matrix metalloproteinase-14 (MMP-14, MT1-MMP), matrix metalloproteinase-15 (MMP-15, MT2-MMP), matrix metalloproteinase-19 (MMP-19), matrix metalloproteinase-23 (MMP-23, CA-MMP), matrix metalloproteinase-24 (MMP-24, MT5-MMP), matrix metalloproteinase-26 (MMP-26, matrilysin 2), matrix metalloproteinase-27 (MMP-27, CMMP), legumain, cathepsin B, cathepsin C, cathepsin K, cathepsin L, cathepsin S, cathepsin X, cathepsin D, cathepsin E, secretase, urokinase (uPA), tissue-type plasminogen activator (tPA), plasmin, thrombin, prostate-specific antigen (PSA, KLK3), human neutrophil elastase (HNE), elastase, tryptase, Type II transmembrane serine proteases (TTSPs), DESC1, hepsin (HPN), matriptase, matriptase-2, TMPRSS2, TMPRSS3, TMPRSS4 (CAP2), fibroblast activation protein (FAP), kallikrein-related peptidase (KLK family), KLK4, KLK5, KLK6, KLK7, KLK8, KLK10, KLK11, KLK13, and KLK14.

In another embodiment, the present disclosure provides polypeptides comprising a first release segment (RS1) sequence for incorporation into the subject polypeptide compositions described herein wherein the RS1 is a substrate for cleavage by one or more mammalian proteases wherein the RS1 comprises an amino acid sequence having at least 90%, 91%, 92%, 93%, 94%, 95%, 96%, 97%, 98%, 99%, or 100% sequence identity to a sequence selected from SEQ ID NOs:42-660. In another embodiment, the RS1 comprises an amino acid sequence selected from the sequences of RSR-2089, RSR-2295, RSR-2298, RSR-2488, RSR-2599, RSR-2485, RSR-2486, RSR-2728, RSN-2089, RSN-2295, RSN-2298, RSN-2488, RSN-2599, RSN-2485, RSN-2486, RSN-2728, RSC-2089, RSC-2295, RSC-2298, RSC-2488, RSC-2599, RSC-2485, RSC-2486, and RSC-2728, each of which being forth in Table 5. As described more fully in descriptions of the configurations and properties of the subject polypeptide compositions, below, the release segment is fused between the antigen binding fragment and an XTEN polypeptide such that upon cleavage of the release segment, the XTEN is released from the composition.

In other embodiments, the disclosure provides polypeptides comprising a first release segment (RS1) sequence and a second release segment (RS2) for incorporation into the subject polypeptide compositions described herein wherein the RS1 and the RS2 are identical. In another embodiment, the present disclosure provides polypeptides comprising a first release segment (RS1) sequence and a second release segment (RS2) for incorporation into the subject polypeptide compositions wherein the RS1 and the RS2 are different. In some cases of the foregoing embodiments, the RS1 and the RS2 are each a substrate for cleavage by a mammalian protease selected from the group consisting of legumain, MMP-2, MMP-7, MMP-9, MMP-11, MMP-14, uPA, and matriptase. In another embodiment, the disclosure provides polypeptides comprising an RS1 and an RS2 sequence for incorporation into the subject polypeptide compositions described herein wherein the RS1 and RS2 are each a substrate for cleavage by one or more mammalian protease wherein the RS1 and RS2 each comprise an amino acid sequence having at least 90%, 91%, 92%, 93%, 94%, 95%, 96%, 97%, 98%, 99%, or 100% sequence identity to a sequence selected from SEQ ID NOs:42-660. In another embodiment, the RS1 and RS2 each comprise an amino acid sequence selected from the sequences of RSR-2089, RSR-2295, RSR-2298, RSR-2488, RSR-2599, RSR-2485, RSR-2486, RSR-2728, RSN-2089, RSN-2295, RSN-2298, RSN-2488, RSN-2599, RSN-2485, RSN-2486, RSN-2728, RSC-2089, RSC-2295, RSC-2298, RSC-2488, RSC-2599, RSC-2485, RSC-2486, and RSC-2728, each of which being set forth in Table 5. As described more fully in paragraphs related to the descriptions of the configurations and properties of the subject polypeptide compositions, below, the release segments are fused between the antigen binding fragment and an XTEN polypeptide such that upon cleavage of each release segment, the adjoining XTEN is released from the composition.

TABLE 5
Release Segments and Amino Acid Sequences

| | | SEQ |
|---|---|---|
| | | ID |
| Name | Amino Acid Sequence | NO: |
| RSR-1517 | EAGRSANHEPLGLVAT | 42 |
| BSRS-A1 | ASGRSTNAGPSGLAGP | 43 |
| BSRS-A2 | ASGRSTNAGPQGLAGQ | 44 |
| BSRS-A3 | ASGRSTNAGPPGLTGP | 45 |
| VP-1 | ASSRGTNAGPAGLTGP | 46 |
| RSR-1752 | ASSRTTNTGPSTLTGP | 47 |
| RSR-1512 | AAGRSDNGTPLELVAP | 48 |
| RSR-1517 | EAGRSANHEPLGLVAT | 42 |
| VP-2 | ASGRGTNAGPAGLTGP | 49 |
| RSR-1018 | LFGRNDNHEPLELGGG | 50 |
| RSR-1053 | TAGRSDNLEPLGLVFG | 51 |
| RSR-1059 | LDGRSDNFHPPELVAG | 52 |
| RSR-1065 | LEGRSDNEEPENLVAG | 53 |
| RSR-1167 | LKGRSDNNAPLALVAG | 54 |
| RSR-1201 | VYSRGTNAGPHGLTGR | 55 |
| RSR-1218 | ANSRGTNKGFAGLIGP | 56 |
| RSR-1226 | ASSRLTNEAPAGLTIP | 57 |
| RSR-1254 | DQSRGTNAGPEGLTDP | 58 |
| RSR-1256 | ESSRGTNIGQGGLTGP | 59 |
| RSR-1261 | SSSRGTNQDPAGLTIP | 60 |
| RSR-1293 | ASSRGQNHSPMGLTGP | 61 |
| RSR-1309 | AYSRGPNAGPAGLEGR | 62 |
| RSR-1326 | ASERGNNAGPANLTGF | 63 |
| RSR-1345 | ASHRGTNPKPAILTGP | 64 |
| RSR-1354 | MSSRRTNANPAQLTGP | 65 |
| RSR-1426 | GAGRTDNHEPLELGAA | 66 |
| RSR-1478 | LAGRSENTAPLELTAG | 67 |
| RSR-1479 | LEGRPDNHEPLALVAS | 68 |
| RSR-1496 | LSGRSDNEEPLALPAG | 69 |
| RSR-1508 | EAGRTDNHEPLELSAP | 70 |
| RSR-1513 | EGGRSDNHGPLELVSG | 71 |
| RSR-1516 | LSGRSDNEAPLELEAG | 72 |
| RSR-1524 | LGGRADNHEPPELGAG | 73 |
| RSR-1622 | PPSRGTNAEPAGLTGE | 74 |
| RSR-1629 | ASTRGENAGPAGLEAP | 75 |
| RSR-1664 | ESSRGTNGAPEGLTGP | 76 |
| RSR-1667 | ASSRATNESPAGLTGE | 77 |
| RSR-1709 | ASSRGENPPPGGLTGP | 78 |
| RSR-1712 | AASRGTNTGPAELTGS | 79 |
| RSR-1727 | AGSRTTNAGPGGLEGP | 80 |
| RSR-1754 | APSRGENAGPATLTGA | 81 |
| RSR-1819 | ESGRAANTGPPTLTAP | 82 |
| RSR-1832 | NPGRAANEGPPGLPGS | 83 |
| RSR-1855 | ESSRAANLTPPELTGP | 84 |
| RSR-1911 | ASGRAANETPPGLTGA | 85 |
| RSR-1929 | NSGRGENLGAPGLTGT | 86 |
| RSR-1951 | TTGRAANLTPAGLTGP | 87 |
| RSR-2295 | EAGRSANHTPAGLTGP | 88 |
| RSR-2298 | ESGRAANTTPAGLTGP | 89 |
| RSR-2038 | TTGRATEAANLTPAGLTGP | 90 |
| RSR-2072 | TTGRAEEAANLTPAGLTGP | 91 |
| RSR-2089 | TTGRAGEAANLTPAGLTGP | 92 |
| RSR-2302 | TTGRATEAANATPAGLTGP | 93 |
| RSR-3047 | TTGRAGEAEGATSAGATGP | 94 |
| RSR-3052 | TTGEAGEAANATSAGATGP | 95 |
| RSR-3043 | TTGEAGEAAGLTPAGLTGP | 96 |
| RSR-3041 | TTGAAGEAANATPAGLTGP | 97 |
| RSR-3044 | TTGRAGEAAGLTPAGLTGP | 98 |
| RSR-3057 | TTGRAGEAANATSAGATGP | 99 |
| RSR-3058 | TTGEAGEAAGATSAGATGP | 100 |
| RSR-2485 | ESGRAANTEPPELGAG | 101 |
| RSR-2486 | ESGRAANTAPEGLTGP | 102 |
| RSR-2488 | EPGRAANHEPSGLTEG | 103 |
| RSR-2599 | ESGRAANHTGAPPGGLTGP | 104 |
| RSR-2706 | TTGRTGEGANATPGGLTGP | 105 |
| RSR-2707 | RTGRSGEAANETPEGLEGP | 106 |
| RSR-2708 | RTGRTGESANETPAGLGGP | 107 |
| RSR-2709 | STGRTGEPANETPAGLSGP | 108 |
| RSR-2710 | TTGRAGEPANATPTGLSGP | 109 |
| RSR-2711 | RTGRPGEGANATPTGLPGP | 110 |
| RSR-2712 | RTGRGGEAANATPSGLGGP | 111 |
| RSR-2713 | STGRSGESANATPGGLGGP | 112 |
| RSR-2714 | RTGRTGEEANATPAGLPGP | 113 |
| RSR-2715 | ATGRPGEPANTTPEGLEGP | 114 |
| RSR-2716 | STGRSGEPANATPGGLTGP | 115 |
| RSR-2717 | PTGRGGEGANTTPTGLPGP | 116 |
| RSR-2718 | PTGRSGEGANATPSGLTGP | 117 |
| RSR-2719 | TTGRASEGANSTPAPLTEP | 118 |
| RSR-2720 | TYGRAAEAANTTPAGLTAP | 119 |
| RSR-2721 | TTGRATEGANATPAELTEP | 120 |
| RSR-2722 | TVGRASEEANTTPASLTGP | 121 |
| RSR-2723 | TTGRAPEAANATPAPLTGP | 122 |
| RSR-2724 | TWGRATEPANATPAPLTSP | 123 |
| RSR-2725 | TVGRASESANATPAELTSP | 124 |
| RSR-2726 | TVGRAPEGANSTPAGLTGP | 125 |
| RSR-2727 | TWGRATEAPNLEPATLTTP | 126 |
| RSR-2728 | TTGRATEAPNLTPAPLTEP | 127 |
| RSR-2729 | TQGRATEAPNLSPAALTSP | 128 |
| RSR-2730 | TQGRAAEAPNLTPATLTAP | 129 |
| RSR-2731 | TSGRAPEATNLAPAPLTGP | 130 |
| RSR-2732 | TQGRAAEAANLTPAGLTEP | 131 |
| RSR-2733 | TTGRAGSAPNLPPTGLTTP | 132 |
| RSR-2734 | TTGRAGGAENLPPEGLTAP | 133 |
| RSR-2735 | TTSRAGTATNLTPEGLTAP | 134 |
| RSR-2736 | TTGRAGTATNLPPSGLTTP | 135 |
| RSR-2737 | TTARAGEAENLSPSGLTAP | 136 |
| RSR-2738 | TTGRAGGAGNLAPGGLTEP | 137 |
| RSR-2739 | TTGRAGTATNLPPEGLTGP | 138 |
| RSR-2740 | TTGRAGGAANLAPTGLTEP | 139 |
| RSR-2741 | TTGRAGTAENLAPSGLTTP | 140 |
| RSR-2742 | TTGRAGSATNLGPGGLTGP | 141 |
| RSR-2743 | TTARAGGAENLTPAGLTEP | 142 |
| RSR-2744 | TTARAGSAENLSPSGLTGP | 143 |
| RSR-2745 | TTARAGGAGNLAPEGLTTP | 144 |
| RSR-2746 | TTSRAGAAENLTPTGLTGP | 145 |
| RSR-2747 | TYGRTTTPGNEPPASLEAE | 146 |
| RSR-2748 | TYSRGESGPNEPPPGLTGP | 147 |
| RSR-2749 | AWGRTGASENETPAPLGGE | 148 |
| RSR-2750 | RWGRAETTPNTPPEGLETE | 149 |
| RSR-2751 | ESGRAANHTGAEPPELGAG | 150 |
| RSR-2754 | TTGRAGEAANLTPAGLTES | 151 |
| RSR-2755 | TTGRAGEAANLTPAALTES | 152 |
| RSR-2756 | TTGRAGEAANLTPAPLTES | 153 |
| RSR-2757 | TTGRAGEAANLTPEPLTES | 154 |
| RSR-2758 | TTGRAGEAANLTPAGLTGA | 155 |
| RSR-2759 | TTGRAGEAANLTPEGLTGA | 156 |
| RSR-2760 | TTGRAGEAANLTPEPLTGA | 157 |
| RSR-2761 | TTGRAGEAANLTPAGLTEA | 158 |
| RSR-2762 | TTGRAGEAANLTPEGLTEA | 159 |
| RSR-2763 | TTGRAGEAANLTPAPLTEA | 160 |
| RSR-2764 | TTGRAGEAANLTPEPLTEA | 161 |
| RSR-2765 | TTGRAGEAANLTPEPLTGP | 162 |
| RSR-2766 | TTGRAGEAANLTPAGLTGG | 163 |
| RSR-2767 | TTGRAGEAANLTPEGLTGG | 164 |
| RSR-2768 | TTGRAGEAANLTPEALTGG | 165 |
| RSR-2769 | TTGRAGEAANLTPEPLTGG | 166 |
| RSR-2770 | TTGRAGEAANLTPAGLTEG | 167 |
| RSR-2771 | TTGRAGEAANLTPEGLTEG | 168 |
| RSR-2772 | TTGRAGEAANLTPAPLTEG | 169 |
| RSR-2773 | TTGRAGEAANLTPEPLTEG | 170 |
| RSN-0001 | GSAPGSAGGYAELRMGGAIATSGSETPGT | 171 |
| RSN-0002 | GSAPGTGGGYAPLRMGGGAATSGSETPGT | 172 |
| RSN-0003 | GSAPGAEGGYAALRMGGEIATSGSETPGT | 173 |
| RSN-0004 | GSAPGGPGGYALLRMGGPAATSGSETPGT | 174 |
| RSN-0005 | GSAPGEAGGYAFLRMGGSIATSGSETPGT | 175 |
| RSN-0006 | GSAPGPGGGYASLRMGGTAATSGSETPGT | 176 |
| RSN-0007 | GSAPGSEGGYATLRMGGAIATSGSETPGT | 177 |
| RSN-0008 | GSAPGTPGGYANLRMGGGAATSGSETPGT | 178 |
| RSN-0009 | GSAPGASGGYAHLRMGGEIATSGSETPGT | 179 |
| RSN-0010 | GSAPGGTGGYGELRMGGPAATSGSETPGT | 180 |
| RSN-0011 | GSAPGEAGGYPELRMGGSIATSGSETPGT | 181 |
| RSN-0012 | GSAPGPGGGYVELRMGGTAATSGSETPGT | 182 |
| RSN-0013 | GSAPGSEGGYLELRMGGAIATSGSETPGT | 183 |
| RSN-0014 | GSAPGTPGGYSELRMGGGAATSGSETPGT | 184 |
| RSN-0015 | GSAPGASGGYTELRMGGEIATSGSETPGT | 185 |
| RSN-0016 | GSAPGGTGGYQELRMGGPAATSGSETPGT | 186 |
| RSN-0017 | GSAPGEAGGYEELRMGGSIATSGSETPGT | 187 |
| RSN-0018 | GSAPGPGIGPAELRMGGTAATSGSETPGT | 188 |
| RSN-0019 | GSAPGSEIGAAELRMGGAIATSGSETPGT | 189 |
| RSN-0020 | GSAPGTPIGSAELRMGGGAATSGSETPGT | 190 |
| RSN-0021 | GSAPGASIGTAELRMGGEIATSGSETPGT | 191 |
| RSN-0022 | GSAPGGTIGNAELRMGGPAATSGSETPGT | 192 |
| RSN-0023 | GSAPGEAIGQAELRMGGSIATSGSETPGT | 193 |
| RSN-0024 | GSAPGPGGPYAELRMGGTAATSGSETPGT | 194 |
| RSN-0025 | GSAPGSEGAYAELRMGGAIATSGSETPGT | 195 |
| RSN-0026 | GSAPGTPGVYAELRMGGGAATSGSETPGT | 196 |
| RSN-0027 | GSAPGASGLYAELRMGGEIATSGSETPGT | 197 |
| RSN-0028 | GSAPGGTGIYAELRMGGPAATSGSETPGT | 198 |
| RSN-0029 | GSAPGEAGFYAELRMGGSIATSGSETPGT | 199 |
| RSN-0030 | GSAPGPGGYYAELRMGGTAATSGSETPGT | 200 |
| RSN-0031 | GSAPGSEGSYAELRMGGAIATSGSETPGT | 201 |
| RSN-0032 | GSAPGTPGNYAELRMGGGAATSGSETPGT | 202 |
| RSN-0033 | GSAPGASGEYAELRMGGEIATSGSETPGT | 203 |
| RSN-0034 | GSAPGGTGHYAELRMGGPAATSGSETPGT | 204 |
| RSN-0035 | GSAPGEAGGYAEARMGGSIATSGSETPGT | 205 |
| RSN-0036 | GSAPGPGGGYAEVRMGGTAATSGSETPGT | 206 |
| RSN-0037 | GSAPGSEGGYAEIRMGGAIATSGSETPGT | 207 |
| RSN-0038 | GSAPGTPGGYAEFRMGGGAATSGSETPGT | 208 |
| RSN-0039 | GSAPGASGGYAEYRMGGEIATSGSETPGT | 209 |
| RSN-0040 | GSAPGGTGGYAESRMGGPAATSGSETPGT | 210 |
| RSN-0041 | GSAPGEAGGYAETRMGGSIATSGSETPGT | 211 |
| RSN-0042 | GSAPGPGGGYAELAMGGTRATSGSETPGT | 212 |
| RSN-0043 | GSAPGSEGGYAELVMGGARATSGSETPGT | 213 |
| RSN-0044 | GSAPGTPGGYAELLMGGGRATSGSETPGT | 214 |
| RSN-0045 | GSAPGASGGYAELIMGGERATSGSETPGT | 215 |
| RSN-0046 | GSAPGGTGGYAELWMGGPRATSGSETPGT | 216 |
| RSN-0047 | GSAPGEAGGYAELSMGGSRATSGSETPGT | 217 |
| RSN-0048 | GSAPGPGGGYAELTMGGTRATSGSETPGT | 218 |
| RSN-0049 | GSAPGSEGGYAELQMGGARATSGSETPGT | 219 |
| RSN-0050 | GSAPGTPGGYAELNMGGGRATSGSETPGT | 220 |
| RSN-0051 | GSAPGASGGYAELEMGGERATSGSETPGT | 221 |
| RSN-0052 | GSAPGGTGGYAELRPGGPIATSGSETPGT | 222 |
| RSN-0053 | GSAPGEAGGYAELRAGGSAATSGSETPGT | 223 |
| RSN-0054 | GSAPGPGGGYAELRLGGTIATSGSETPGT | 224 |
| RSN-0055 | GSAPGSEGGYAELRIGGAAATSGSETPGT | 225 |
| RSN-0056 | GSAPGTPGGYAELRSGGGIATSGSETPGT | 226 |
| RSN-0057 | GSAPGASGGYAELRNGGEAATSGSETPGT | 227 |
| RSN-0058 | GSAPGGTGGYAELRQGGPIATSGSETPGT | 228 |
| RSN-0059 | GSAPGEAGGYAELRDGGSAATSGSETPGT | 229 |
| RSN-0060 | GSAPGPGGGYAELREGGTIATSGSETPGT | 230 |
| RSN-0061 | GSAPGSEGGYAELRHGGAAATSGSETPGT | 231 |
| RSN-0062 | GSAPGTPGGYAELRMPGGIATSGSETPGT | 232 |
| RSN-0063 | GSAPGASGGYAELRMAGEAATSGSETPGT | 233 |
| RSN-0064 | GSAPGGTGGYAELRMVGPIATSGSETPGT | 234 |
| RSN-0065 | GSAPGEAGGYAELRMLGSAATSGSETPGT | 235 |
| RSN-0066 | GSAPGPGGGYAELRMIGTIATSGSETPGT | 236 |
| RSN-0067 | GSAPGSEGGYAELRMYGAIATSGSETPGT | 237 |
| RSN-0068 | GSAPGTPGGYAELRMSGGAATSGSETPGT | 238 |
| RSN-0069 | GSAPGASGGYAELRMNGEIATSGSETPGT | 239 |
| RSN-0070 | GSAPGGTGGYAELRMQGPAATSGSETPGT | 240 |
| RSN-0071 | GSAPGANHTPAGLTGPGARATSGSETPGT | 241 |
| RSN-0072 | GSAPGANTAPEGLTGPSTRATSGSETPGT | 242 |
| RSN-0073 | GSAPGTGAPPGGLTGPGTRATSGSETPGT | 243 |
| RSN-0074 | GSAPGANHEPSGLTEGSPRATSGSETPGT | 244 |
| RSN-0075 | GSAPGANTEPPELGAGTERATSGSETPGT | 245 |
| RSN-0076 | GSAPGASGPPPGLTGPPGRATSGSETPGT | 246 |
| RSN-0077 | GSAPGASGTPAPLGGEPGRATSGSETPGT | 247 |
| RSN-0078 | GSAPGPAGPPEGLETEAGRATSGSETPGT | 248 |
| RSN-0079 | GSAPGPTSGQGGLTGPESRATSGSETPGT | 249 |
| RSN-0080 | GSAPGSAGGAANLVRGGAIATSGSETPGT | 250 |
| RSN-0081 | GSAPGTGGGAAPLVRGGGAATSGSETPGT | 251 |
| RSN-0082 | GSAPGAEGGAAALVRGGEIATSGSETPGT | 252 |
| RSN-0083 | GSAPGGPGGAALLVRGGPAATSGSETPGT | 253 |
| RSN-0084 | GSAPGEAGGAAFLVRGGSIATSGSETPGT | 254 |
| RSN-0085 | GSAPGPGGGAASLVRGGTAATSGSETPGT | 255 |
| RSN-0086 | GSAPGSEGGAATLVRGGAIATSGSETPGT | 256 |
| RSN-0087 | GSAPGTPGGAAGLVRGGGAATSGSETPGT | 257 |
| RSN-0088 | GSAPGASGGAADLVRGGEIATSGSETPGT | 258 |
| RSN-0089 | GSAPGGTGGAGNLVRGGPAATSGSETPGT | 259 |
| RSN-0090 | GSAPGEAGGAPNLVRGGSIATSGSETPGT | 260 |
| RSN-0091 | GSAPGPGGGAVNLVRGGTAATSGSETPGT | 261 |
| RSN-0092 | GSAPGSEGGALNLVRGGAIATSGSETPGT | 262 |
| RSN-0093 | GSAPGTPGGASNLVRGGGAATSGSETPGT | 263 |
| RSN-0094 | GSAPGASGGATNLVRGGEIATSGSETPGT | 264 |
| RSN-0095 | GSAPGGTGGAQNLVRGGPAATSGSETPGT | 265 |
| RSN-0096 | GSAPGEAGGAENLVRGGSIATSGSETPGT | 266 |
| RSN-1517 | GSAPEAGRSANHEPLGLVATATSGSETPGT | 267 |
| BSRS-A1-2 | GSAPASGRSTNAGPSGLAGPATSGSETPGT | 268 |
| BSRS-A2-2 | GSAPASGRSTNAGPQGLAGQATSGSETPGT | 269 |
| BSRS-A3-2 | GSAPASGRSTNAGPPGLTGPATSGSETPGT | 270 |
| VP-1-2 | GSAPASSRGTNAGPAGLTGPATSGSETPGT | 271 |
| RSN-1752 | GSAPASSRTTNTGPSTLTGPATSGSETPGT | 272 |
| RSN-1512 | GSAPAAGRSDNGTPLELVAPATSGSETPGT | 273 |
| RSN-1517 | GSAPEAGRSANHEPLGLVATATSGSETPGT | 267 |
| VP-2-2 | GSAPASGRGTNAGPAGLTGPATSGSETPGT | 274 |
| RSN-1018 | GSAPLFGRNDNHEPLELGGGATSGSETPGT | 275 |
| RSN-1053 | GSAPTAGRSDNLEPLGLVFGATSGSETPGT | 276 |
| RSN-1059 | GSAPLDGRSDNFHPPELVAGATSGSETPGT | 277 |
| RSN-1065 | GSAPLEGRSDNEEPENLVAGATSGSETPGT | 278 |
| RSN-1167 | GSAPLKGRSDNNAPLALVAGATSGSETPGT | 279 |
| RSN-1201 | GSAPVYSRGTNAGPHGLTGRATSGSETPGT | 280 |
| RSN-1218 | GSAPANSRGTNKGFAGLIGPATSGSETPGT | 281 |
| RSN-1226 | GSAPASSRLTNEAPAGLTIPATSGSETPGT | 282 |
| RSN-1254 | GSAPDQSRGTNAGPEGLTDPATSGSETPGT | 283 |
| RSN-1256 | GSAPESSRGTNIGQGGLTGPATSGSETPGT | 284 |
| RSN-1261 | GSAPSSSRGTNQDPAGLTIPATSGSETPGT | 285 |
| RSN-1293 | GSAPASSRGQNHSPMGLTGPATSGSETPGT | 286 |
| RSN-1309 | GSAPAYSRGPNAGPAGLEGRATSGSETPGT | 287 |
| RSN-1326 | GSAPASERGNNAGPANLTGFATSGSETPGT | 288 |
| RSN-1345 | GSAPASHRGTNPKPAILTGPATSGSETPGT | 289 |
| RSN-1354 | GSAPMSSRRTNANPAQLTGPATSGSETPGT | 290 |
| RSN-1426 | GSAPGAGRTDNHEPLELGAAATSGSETPGT | 291 |
| RSN-1478 | GSAPLAGRSENTAPLELTAGATSGSETPGT | 292 |
| RSN-1479 | GSAPLEGRPDNHEPLALVASATSGSETPGT | 293 |
| RSN-1496 | GSAPLSGRSDNEEPLALPAGATSGSETPGT | 294 |
| RSN-1508 | GSAPEAGRTDNHEPLELSAPATSGSETPGT | 295 |
| RSN-1513 | GSAPEGGRSDNHGPLELVSGATSGSETPGT | 296 |
| RSN-1516 | GSAPLSGRSDNEAPLELEAGATSGSETPGT | 297 |
| RSN-1524 | GSAPLGGRADNHEPPELGAGATSGSETPGT | 298 |
| RSN-1622 | GSAPPPSRGTNAEPAGLTGEATSGSETPGT | 299 |
| RSN-1629 | GSAPASTRGENAGPAGLEAPATSGSETPGT | 300 |
| RSN-1664 | GSAPESSRGTNGAPEGLTGPATSGSETPGT | 301 |
| RSN-1667 | GSAPASSRATNESPAGLTGEATSGSETPGT | 302 |
| RSN-1709 | GSAPASSRGENPPPGGLTGPATSGSETPGT | 303 |
| RSN-1712 | GSAPAASRGTNTGPAELTGSATSGSETPGT | 304 |
| RSN-1727 | GSAPAGSRTTNAGPGGLEGPATSGSETPGT | 305 |
| RSN-1754 | GSAPAPSRGENAGPATLTGAATSGSETPGT | 306 |
| RSN-1819 | GSAPESGRAANTGPPTLTAPATSGSETPGT | 307 |
| RSN-1832 | GSAPNPGRAANEGPPGLPGSATSGSETPGT | 308 |
| RSN-1855 | GSAPESSRAANLTPPELTGPATSGSETPGT | 309 |
| RSN-1911 | GSAPASGRAANETPPGLTGAATSGSETPGT | 310 |
| RSN-1929 | GSAPNSGRGENLGAPGLTGTATSGSETPGT | 311 |
| RSN-1951 | GSAPTTGRAANLTPAGLTGPATSGSETPGT | 312 |
| RSN-2295 | GSAPEAGRSANHTPAGLTGPATSGSETPGT | 313 |
| RSN-2298 | GSAPESGRAANTTPAGLTGPATSGSETPGT | 314 |
| RSN-2038 | GSAPTTGRATEAANLTPAGLTGPATSGSETPGT | 315 |
| RSN-2072 | GSAPTTGRAEEAANLTPAGLTGPATSGSETPGT | 316 |
| RSN-2089 | GSAPTTGRAGEAANLTPAGLTGPATSGSETPGT | 317 |
| RSN-2302 | GSAPTTGRATEAANATPAGLTGPATSGSETPGT | 318 |
| RSN-3047 | GSAPTTGRAGEAEGATSAGATGPATSGSETPGT | 319 |
| RSN-3052 | GSAPTTGEAGEAANATSAGATGPATSGSETPGT | 320 |
| RSN-3043 | GSAPTTGEAGEAAGLTPAGLTGPATSGSETPGT | 321 |
| RSN-3041 | GSAPTTGAAGEAANATPAGLTGPATSGSETPGT | 322 |
| RSN-3044 | GSAPTTGRAGEAAGLTPAGLTGPATSGSETPGT | 323 |
| RSN-3057 | GSAPTTGRAGEAANATSAGATGPATSGSETPGT | 324 |
| RSN-3058 | GSAPTTGEAGEAAGATSAGATGPATSGSETPGT | 325 |
| RSN-2485 | GSAPESGRAANTEPPELGAGATSGSETPGT | 326 |
| RSN-2486 | GSAPESGRAANTAPEGLTGPATSGSETPGT | 327 |
| RSN-2488 | GSAPEPGRAANHEPSGLTEGATSGSETPGT | 328 |
| RSN-2599 | GSAPESGRAANHTGAPPGGLTGPATSGSETPGT | 329 |
| RSN-2706 | GSAPTTGRTGEGANATPGGLTGPATSGSETPGT | 330 |
| RSN-2707 | GSAPRTGRSGEAANETPEGLEGPATSGSETPGT | 331 |
| RSN-2708 | GSAPRTGRTGESANETPAGLGGPATSGSETPGT | 332 |
| RSN-2709 | GSAPSTGRTGEPANETPAGLSGPATSGSETPGT | 333 |
| RSN-2710 | GSAPTTGRAGEPANATPTGLSGPATSGSETPGT | 334 |
| RSN-2711 | GSAPRTGRPGEGANATPTGLPGPATSGSETPGT | 335 |
| RSN-2712 | GSAPRTGRGGEAANATPSGLGGPATSGSETPGT | 336 |
| RSN-2713 | GSAPSTGRSGESANATPGGLGGPATSGSETPGT | 337 |
| RSN-2714 | GSAPRTGRTGEEANATPAGLPGPATSGSETPGT | 338 |
| RSN-2715 | GSAPATGRPGEPANTTPEGLEGPATSGSETPGT | 339 |
| RSN-2716 | GSAPSTGRSGEPANATPGGLTGPATSGSETPGT | 340 |
| RSN-2717 | GSAPPTGRGGEGANTTPTGLPGPATSGSETPGT | 341 |
| RSN-2718 | GSAPPTGRSGEGANATPSGLTGPATSGSETPGT | 342 |
| RSN-2719 | GSAPTTGRASEGANSTPAPLTEPATSGSETPGT | 343 |
| RSN-2720 | GSAPTYGRAAEAANTTPAGLTAPATSGSETPGT | 344 |
| RSN-2721 | GSAPTTGRATEGANATPAELTEPATSGSETPGT | 345 |
| RSN-2722 | GSAPTVGRASEEANTTPASLTGPATSGSETPGT | 346 |
| RSN-2723 | GSAPTTGRAPEAANATPAPLTGPATSGSETPGT | 347 |
| RSN-2724 | GSAPTWGRATEPANATPAPLTSPATSGSETPGT | 348 |
| RSN-2725 | GSAPTVGRASESANATPAELTSPATSGSETPGT | 349 |
| RSN-2726 | GSAPTVGRAPEGANSTPAGLTGPATSGSETPGT | 350 |
| RSN-2727 | GSAPTWGRATEAPNLEPATLTTPATSGSETPGT | 351 |
| RSN-2728 | GSAPTTGRATEAPNLTPAPLTEPATSGSETPGT | 352 |
| RSN-2729 | GSAPTQGRATEAPNLSPAALTSPATSGSETPGT | 353 |
| RSN-2730 | GSAPTQGRAAEAPNLTPATLTAPATSGSETPGT | 354 |
| RSN-2731 | GSAPTSGRAPEATNLAPAPLTGPATSGSETPGT | 355 |
| RSN-2732 | GSAPTQGRAAEAANLTPAGLTEPATSGSETPGT | 356 |
| RSN-2733 | GSAPTTGRAGSAPNLPPTGLTTPATSGSETPGT | 357 |
| RSN-2734 | GSAPTTGRAGGAENLPPEGLTAPATSGSETPGT | 358 |
| RSN-2735 | GSAPTTSRAGTATNLTPEGLTAPATSGSETPGT | 359 |
| RSN-2736 | GSAPTTGRAGTATNLPPSGLTTPATSGSETPGT | 360 |
| RSN-2737 | GSAPTTARAGEAENLSPSGLTAPATSGSETPGT | 361 |
| RSN-2738 | GSAPTTGRAGGAGNLAPGGLTEPATSGSETPGT | 362 |
| RSN-2739 | GSAPTTGRAGTATNLPPEGLTGPATSGSETPGT | 363 |
| RSN-2740 | GSAPTTGRAGGAANLAPTGLTEPATSGSETPGT | 364 |
| RSN-2741 | GSAPTTGRAGTAENLAPSGLTTPATSGSETPGT | 365 |
| RSN-2742 | GSAPTTGRAGSATNLGPGGLTGPATSGSETPGT | 366 |
| RSN-2743 | GSAPTTARAGGAENLTPAGLTEPATSGSETPGT | 367 |
| RSN-2744 | GSAPTTARAGSAENLSPSGLTGPATSGSETPGT | 368 |
| RSN-2745 | GSAPTTARAGGAGNLAPEGLTTPATSGSETPGT | 369 |
| RSN-2746 | GSAPTTSRAGAAENLTPTGLTGPATSGSETPGT | 370 |
| RSN-2747 | GSAPTYGRTTTPGNEPPASLEAEATSGSETPGT | 371 |
| RSN-2748 | GSAPTYSRGESGPNEPPPGLTGPATSGSETPGT | 372 |
| RSN-2749 | GSAPAWGRTGASENETPAPLGGEATSGSETPGT | 373 |
| RSN-2750 | GSAPRWGRAETTPNTPPEGLETEATSGSETPGT | 374 |
| RSN-2751 | GSAPESGRAANHTGAEPPELGAGATSGSETPGT | 375 |
| RSN-2754 | GSAPTTGRAGEAANLTPAGLTESATSGSETPGT | 376 |
| RSN-2755 | GSAPTTGRAGEAANLTPAALTESATSGSETPGT | 377 |
| RSN-2756 | GSAPTTGRAGEAANLTPAPLTESATSGSETPGT | 378 |
| RSN-2757 | GSAPTTGRAGEAANLTPEPLTESATSGSETPGT | 379 |
| RSN-2758 | GSAPTTGRAGEAANLTPAGLTGAATSGSETPGT | 380 |
| RSN-2759 | GSAPTTGRAGEAANLTPEGLTGAATSGSETPGT | 381 |
| RSN-2760 | GSAPTTGRAGEAANLTPEPLTGAATSGSETPGT | 382 |
| RSN-2761 | GSAPTTGRAGEAANLTPAGLTEAATSGSETPGT | 383 |
| RSN-2762 | GSAPTTGRAGEAANLTPEGLTEAATSGSETPGT | 384 |
| RSN-2763 | GSAPTTGRAGEAANLTPAPLTEAATSGSETPGT | 385 |
| RSN-2764 | GSAPTTGRAGEAANLTPEPLTEAATSGSETPGT | 386 |
| RSN-2765 | GSAPTTGRAGEAANLTPEPLTGPATSGSETPGT | 387 |
| RSN-2766 | GSAPTTGRAGEAANLTPAGLTGGATSGSETPGT | 388 |
| RSN-2767 | GSAPTTGRAGEAANLTPEGLTGGATSGSETPGT | 389 |
| RSN-2768 | GSAPTTGRAGEAANLTPEALTGGATSGSETPGT | 390 |
| RSN-2769 | GSAPTTGRAGEAANLTPEPLTGGATSGSETPGT | 391 |
| RSN-2770 | GSAPTTGRAGEAANLTPAGLTEGATSGSETPGT | 392 |
| RSN-2771 | GSAPTTGRAGEAANLTPEGLTEGATSGSETPGT | 393 |
| RSN-2772 | GSAPTTGRAGEAANLTPAPLTEGATSGSETPGT | 394 |
| RSN-2773 | GSAPTTGRAGEAANLTPEPLTEGATSGSETPGT | 395 |
| RSN-3047 | GSAPTTGRAGEAEGATSAGATGPATSGSETPGT | 319 |
| RSN-2783 | GSAPEAGRSAEATSAGATGPATSGSETPGT | 396 |
| RSN-3107 | GSAPSASGTYSRGESGPGSPATSGSETPGT | 397 |
| RSN-3103 | GSAPSASGEAGRTDTHPGSPATSGSETPGT | 398 |
| RSN-3102 | GSAPSASGEPGRAAEHPGSPATSGSETPGT | 399 |
| RSN-3119 | GSAPSPAGESSRGTTTAGSPATSGSETPGT | 400 |
| RSN-3043 | GSAPTTGEAGEAAGLTPAGLTGPATSGSETPGT | 321 |
| RSN-2789 | GSAPEAGESAGATPAGLTGPATSGSETPGT | 401 |
| RSN-3109 | GSAPSASGAPLELEAGPGSPATSGSETPGT | 402 |
| RSN-3110 | GSAPSASGEPPELGAGPGSPATSGSETPGT | 403 |
| RSN-3111 | GSAPSASGEPSGLTEGPGSPATSGSETPGT | 404 |
| RSN-3112 | GSAPSASGTPAPLTEPPGSPATSGSETPGT | 405 |
| RSN-3113 | GSAPSASGTPAELTEPPGSPATSGSETPGT | 406 |
| RSN-3114 | GSAPSASGPPPGLTGPPGSPATSGSETPGT | 407 |
| RSN-3115 | GSAPSASGTPAPLGGEPGSPATSGSETPGT | 408 |
| RSN-3125 | GSAPSPAGAPEGLTGPAGSPATSGSETPGT | 409 |
| RSN-3126 | GSAPSPAGPPEGLETEAGSPATSGSETPGT | 410 |
| RSN-3127 | GSAPSPTSGQGGLTGPGSEPATSGSETPGT | 411 |
| RSN-3131 | GSAPSESAPPEGLETESTEPATSGSETPGT | 412 |
| RSN-3132 | GSAPSEGSEPLELGAASETPATSGSETPGT | 413 |
| RSN-3133 | GSAPSEGSGPAGLEAPSETPATSGSETPGT | 414 |
| RSN-3138 | GSAPSEPTPPASLEAEPGSPATSGSETPGT | 415 |
| RSC-0001 | GTAEAASASGGSAGGYAELRMGGAIPGSP | 416 |
| RSC-0002 | GTAEAASASGGTGGGYAPLRMGGGAPGSP | 417 |
| RSC-0003 | GTAEAASASGGAEGGYAALRMGGEIPGSP | 418 |
| RSC-0004 | GTAEAASASGGGPGGYALLRMGGPAPGSP | 419 |
| RSC-0005 | GTAEAASASGGEAGGYAFLRMGGSIPGSP | 420 |
| RSC-0006 | GTAEAASASGGPGGGYASLRMGGTAPGSP | 421 |
| RSC-0007 | GTAEAASASGGSEGGYATLRMGGAIPGSP | 422 |
| RSC-0008 | GTAEAASASGGTPGGYANLRMGGGAPGSP | 423 |
| RSC-0009 | GTAEAASASGGASGGYAHLRMGGEIPGSP | 424 |
| RSC-0010 | GTAEAASASGGGTGGYGELRMGGPAPGSP | 425 |
| RSC-0011 | GTAEAASASGGEAGGYPELRMGGSIPGSP | 426 |
| RSC-0012 | GTAEAASASGGPGGGYVELRMGGTAPGSP | 427 |
| RSC-0013 | GTAEAASASGGSEGGYLELRMGGAIPGSP | 428 |
| RSC-0014 | GTAEAASASGGTPGGYSELRMGGGAPGSP | 429 |
| RSC-0015 | GTAEAASASGGASGGYTELRMGGEIPGSP | 430 |
| RSC-0016 | GTAEAASASGGGTGGYQELRMGGPAPGSP | 431 |
| RSC-0017 | GTAEAASASGGEAGGYEELRMGGSIPGSP | 432 |
| RSC-0018 | GTAEAASASGGPGIGPAELRMGGTAPGSP | 433 |
| RSC-0019 | GTAEAASASGGSEIGAAELRMGGAIPGSP | 434 |
| RSC-0020 | GTAEAASASGGTPIGSAELRMGGGAPGSP | 435 |
| RSC-0021 | GTAEAASASGGASIGTAELRMGGEIPGSP | 436 |
| RSC-0022 | GTAEAASASGGGTIGNAELRMGGPAPGSP | 437 |
| RSC-0023 | GTAEAASASGGEAIGQAELRMGGSIPGSP | 438 |
| RSC-0024 | GTAEAASASGGPGGPYAELRMGGTAPGSP | 439 |
| RSC-0025 | GTAEAASASGGSEGAYAELRMGGAIPGSP | 440 |
| RSC-0026 | GTAEAASASGGTPGVYAELRMGGGAPGSP | 441 |
| RSC-0027 | GTAEAASASGGASGLYAELRMGGEIPGSP | 442 |
| RSC-0028 | GTAEAASASGGGTGIYAELRMGGPAPGSP | 443 |
| RSC-0029 | GTAEAASASGGEAGFYAELRMGGSIPGSP | 444 |
| RSC-0030 | GTAEAASASGGPGGYYAELRMGGTAPGSP | 445 |
| RSC-0031 | GTAEAASASGGSEGSYAELRMGGAIPGSP | 446 |
| RSC-0032 | GTAEAASASGGTPGNYAELRMGGGAPGSP | 447 |
| RSC-0033 | GTAEAASASGGASGEYAELRMGGEIPGSP | 448 |
| RSC-0034 | GTAEAASASGGGTGHYAELRMGGPAPGSP | 449 |
| RSC-0035 | GTAEAASASGGEAGGYAEARMGGSIPGSP | 450 |
| RSC-0036 | GTAEAASASGGPGGGYAEVRMGGTAPGSP | 451 |
| RSC-0037 | GTAEAASASGGSEGGYAEIRMGGAIPGSP | 452 |
| RSC-0038 | GTAEAASASGGTPGGYAEFRMGGGAPGSP | 453 |
| RSC-0039 | GTAEAASASGGASGGYAEYRMGGEIPGSP | 454 |
| RSC-0040 | GTAEAASASGGGTGGYAESRMGGPAPGSP | 455 |
| RSC-0041 | GTAEAASASGGEAGGYAETRMGGSIPGSP | 456 |
| RSC-0042 | GTAEAASASGGPGGGYAELAMGGTRPGSP | 457 |
| RSC-0043 | GTAEAASASGGSEGGYAELVMGGARPGSP | 458 |
| RSC-0044 | GTAEAASASGGTPGGYAELLMGGGRPGSP | 459 |
| RSC-0045 | GTAEAASASGGASGGYAELIMGGERPGSP | 460 |
| RSC-0046 | GTAEAASASGGGTGGYAELWMGGPRPGSP | 461 |
| RSC-0047 | GTAEAASASGGEAGGYAELSMGGSRPGSP | 462 |
| RSC-0048 | GTAEAASASGGPGGGYAELTMGGTRPGSP | 463 |
| RSC-0049 | GTAEAASASGGSEGGYAELQMGGARPGSP | 464 |
| RSC-0050 | GTAEAASASGGTPGGYAELNMGGGRPGSP | 465 |
| RSC-0051 | GTAEAASASGGASGGYAELEMGGERPGSP | 466 |
| RSC-0052 | GTAEAASASGGGTGGYAELRPGGPIPGSP | 467 |
| RSC-0053 | GTAEAASASGGEAGGYAELRAGGSAPGSP | 468 |
| RSC-0054 | GTAEAASASGGPGGGYAELRLGGTIPGSP | 469 |
| RSC-0055 | GTAEAASASGGSEGGYAELRIGGAAPGSP | 470 |
| RSC-0056 | GTAEAASASGGTPGGYAELRSGGGIPGSP | 471 |
| RSC-0057 | GTAEAASASGGASGGYAELRNGGEAPGSP | 472 |
| RSC-0058 | GTAEAASASGGGTGGYAELRQGGPIPGSP | 473 |
| RSC-0059 | GTAEAASASGGEAGGYAELRDGGSAPGSP | 474 |
| RSC-0060 | GTAEAASASGGPGGGYAELREGGTIPGSP | 475 |
| RSC-0061 | GTAEAASASGGSEGGYAELRHGGAAPGSP | 476 |
| RSC-0062 | GTAEAASASGGTPGGYAELRMPGGIPGSP | 477 |
| RSC-0063 | GTAEAASASGGASGGYAELRMAGEAPGSP | 478 |
| RSC-0064 | GTAEAASASGGGTGGYAELRMVGPIPGSP | 479 |
| RSC-0065 | GTAEAASASGGEAGGYAELRMLGSAPGSP | 480 |
| RSC-0066 | GTAEAASASGGPGGGYAELRMIGTIPGSP | 481 |
| RSC-0067 | GTAEAASASGGSEGGYAELRMYGAIPGSP | 482 |
| RSC-0068 | GTAEAASASGGTPGGYAELRMSGGAPGSP | 483 |
| RSC-0069 | GTAEAASASGGASGGYAELRMNGEIPGSP | 484 |
| RSC-0070 | GTAEAASASGGGTGGYAELRMQGPAPGSP | 485 |
| RSC-0071 | GTAEAASASGGANHTPAGLTGPGARPGSP | 486 |
| RSC-0072 | GTAEAASASGGANTAPEGLTGPSTRPGSP | 487 |
| RSC-0073 | GTAEAASASGGTGAPPGGLTGPGTRPGSP | 488 |
| RSC-0074 | GTAEAASASGGANHEPSGLTEGSPRPGSP | 489 |
| RSC-0075 | GTAEAASASGGANTEPPELGAGTERPGSP | 490 |
| RSC-0076 | GTAEAASASGGASGPPPGLTGPPGRPGSP | 491 |
| RSC-0077 | GTAEAASASGGASGTPAPLGGEPGRPGSP | 492 |
| RSC-0078 | GTAEAASASGGPAGPPEGLETEAGRPGSP | 493 |
| RSC-0079 | GTAEAASASGGPTSGQGGLTGPESRPGSP | 494 |
| RSC-0080 | GTAEAASASGGSAGGAANLVRGGAIPGSP | 495 |
| RSC-0081 | GTAEAASASGGTGGGAAPLVRGGGAPGSP | 496 |
| RSC-0082 | GTAEAASASGGAEGGAAALVRGGEIPGSP | 497 |
| RSC-0083 | GTAEAASASGGGPGGAALLVRGGPAPGSP | 498 |
| RSC-0084 | GTAEAASASGGEAGGAAFLVRGGSIPGSP | 499 |
| RSC-0085 | GTAEAASASGGPGGGAASLVRGGTAPGSP | 500 |
| RSC-0086 | GTAEAASASGGSEGGAATLVRGGAIPGSP | 501 |
| RSC-0087 | GTAEAASASGGTPGGAAGLVRGGGAPGSP | 502 |
| RSC-0088 | GTAEAASASGGASGGAADLVRGGEIPGSP | 503 |
| RSC-0089 | GTAEAASASGGGTGGAGNLVRGGPAPGSP | 504 |
| RSC-0090 | GTAEAASASGGEAGGAPNLVRGGSIPGSP | 505 |
| RSC-0091 | GTAEAASASGGPGGGAVNLVRGGTAPGSP | 506 |
| RSC-0092 | GTAEAASASGGSEGGALNLVRGGAIPGSP | 507 |
| RSC-0093 | GTAEAASASGGTPGGASNLVRGGGAPGSP | 508 |
| RSC-0094 | GTAEAASASGGASGGATNLVRGGEIPGSP | 509 |
| RSC-0095 | GTAEAASASGGGTGGAQNLVRGGPAPGSP | 510 |
| RSC-0096 | GTAEAASASGGEAGGAENLVRGGSIPGSP | 511 |
| RSC-1517 | GTAEAASASGEAGRSANHEPLGLVATPGSP | 512 |
| BSRS-A1-3 | GTAEAASASGASGRSTNAGPSGLAGPPGSP | 513 |
| BSRS-A2-3 | GTAEAASASGASGRSTNAGPQGLAGQPGSP | 514 |
| BSRS-A3-3 | GTAEAASASGASGRSTNAGPPGLTGPPGSP | 515 |
| VP-1-2 | GTAEAASASGASSRGTNAGPAGLTGPPGSP | 516 |
| RSC-1752-2 | GTAEAASASGASSRTTNTGPSTLTGPPGSP | 517 |
| RSC-1512 | GTAEAASASGAAGRSDNGTPLELVAPPGSP | 518 |
| RSC-1517 | GTAEAASASGEAGRSANHEPLGLVATPGSP | 512 |
| VP-2-2 | GTAEAASASGASGRGTNAGPAGLTGPPGSP | 519 |
| RSC-1018 | GTAEAASASGLFGRNDNHEPLELGGGPGSP | 520 |
| RSC-1053 | GTAEAASASGTAGRSDNLEPLGLVFGPGSP | 521 |
| RSC-1059 | GTAEAASASGLDGRSDNFHPPELVAGPGSP | 522 |
| RSC-1065 | GTAEAASASGLEGRSDNEEPENLVAGPGSP | 523 |
| RSC-1167 | GTAEAASASGLKGRSDNNAPLALVAGPGSP | 524 |
| RSC-1201 | GTAEAASASGVYSRGTNAGPHGLTGRPGSP | 525 |
| RSC-1218 | GTAEAASASGANSRGTNKGFAGLIGPPGSP | 526 |
| RSC-1226 | GTAEAASASGASSRLTNEAPAGLTIPPGSP | 527 |
| RSC-1254 | GTAEAASASGDQSRGTNAGPEGLTDPPGSP | 528 |
| RSC-1256 | GTAEAASASGESSRGTNIGQGGLTGPPGSP | 529 |
| RSC-1261 | GTAEAASASGSSSRGTNQDPAGLTIPPGSP | 530 |
| RSC-1293 | GTAEAASASGASSRGQNHSPMGLTGPPGSP | 531 |
| RSC-1309 | GTAEAASASGAYSRGPNAGPAGLEGRPGSP | 532 |
| RSC-1326 | GTAEAASASGASERGNNAGPANLTGFPGSP | 533 |
| RSC-1345 | GTAEAASASGASHRGTNPKPAILTGPPGSP | 534 |
| RSC-1354 | GTAEAASASGMSSRRTNANPAQLTGPPGSP | 535 |
| RSC-1426 | GTAEAASASGGAGRTDNHEPLELGAAPGSP | 536 |
| RSC-1478 | GTAEAASASGLAGRSENTAPLELTAGPGSP | 537 |
| RSC-1479 | GTAEAASASGLEGRPDNHEPLALVASPGSP | 538 |
| RSC-1496 | GTAEAASASGLSGRSDNEEPLALPAGPGSP | 539 |
| RSC-1508 | GTAEAASASGEAGRTDNHEPLELSAPPGSP | 540 |
| RSC-1513 | GTAEAASASGEGGRSDNHGPLELVSGPGSP | 541 |
| RSC-1516 | GTAEAASASGLSGRSDNEAPLELEAGPGSP | 542 |
| RSC-1524 | GTAEAASASGLGGRADNHEPPELGAGPGSP | 543 |
| RSC-1622 | GTAEAASASGPPSRGTNAEPAGLTGEPGSP | 544 |
| RSC-1629 | GTAEAASASGASTRGENAGPAGLEAPPGSP | 545 |
| RSC-1664 | GTAEAASASGESSRGTNGAPEGLTGPPGSP | 546 |
| RSC-1667 | GTAEAASASGASSRATNESPAGLTGEPGSP | 547 |
| RSC-1709 | GTAEAASASGASSRGENPPPGGLTGPPGSP | 548 |
| RSC-1712 | GTAEAASASGAASRGTNTGPAELTGSPGSP | 549 |
| RSC-1727 | GTAEAASASGAGSRTTNAGPGGLEGPPGSP | 550 |
| RSC-1754 | GTAEAASASGAPSRGENAGPATLTGAPGSP | 551 |
| RSC-1819 | GTAEAASASGESGRAANTGPPTLTAPPGSP | 552 |
| RSC-1832 | GTAEAASASGNPGRAANEGPPGLPGSPGSP | 553 |
| RSC-1855 | GTAEAASASGESSRAANLTPPELTGPPGSP | 554 |
| RSC-1911 | GTAEAASASGASGRAANETPPGLTGAPGSP | 555 |
| RSC-1929 | GTAEAASASGNSGRGENLGAPGLTGTPGSP | 556 |
| RSC-1951 | GTAEAASASGTTGRAANLTPAGLTGPPGSP | 557 |
| RSC-2295 | GTAEAASASGEAGRSANHTPAGLTGPPGSP | 558 |
| RSC-2298 | GTAEAASASGESGRAANTTPAGLTGPPGSP | 559 |
| RSC-2038 | GTAEAASASGTTGRATEAANLTPAGLTGPPGSP | 560 |
| RSC-2072 | GTAEAASASGTTGRAEEAANLTPAGLTGPPGSP | 561 |
| RSC-2089 | GTAEAASASGTTGRAGEAANLTPAGLTGPPGSP | 562 |
| RSC-2302 | GTAEAASASGTTGRATEAANATPAGLTGPPGSP | 563 |
| RSC-3047 | GTAEAASASGTTGRAGEAEGATSAGATGPPGSP | 564 |
| RSC-3052 | GTAEAASASGTTGEAGEAANATSAGATGPPGSP | 565 |
| RSC-3043 | GTAEAASASGTTGEAGEAAGLTPAGLTGPPGSP | 566 |
| RSC-3041 | GTAEAASASGTTGAAGEAANATPAGLTGPPGSP | 567 |
| RSC-3044 | GTAEAASASGTTGRAGEAAGLTPAGLTGPPGSP | 568 |
| RSC-3057 | GTAEAASASGTTGRAGEAANATSAGATGPPGSP | 569 |
| RSC-3058 | GTAEAASASGTTGEAGEAAGATSAGATGPPGSP | 570 |
| RSC-2485 | GTAEAASASGESGRAANTEPPELGAGPGSP | 571 |
| RSC-2486 | GTAEAASASGESGRAANTAPEGLTGPPGSP | 572 |
| RSC-2488 | GTAEAASASGEPGRAANHEPSGLTEGPGSP | 573 |
| RSC-2599 | GTAEAASASGESGRAANHTGAPPGGLTGPPGSP | 574 |
| RSC-2706 | GTAEAASASGTTGRTGEGANATPGGLTGPPGSP | 575 |
| RSC-2707 | GTAEAASASGRTGRSGEAANETPEGLEGPPGSP | 576 |
| RSC-2708 | GTAEAASASGRTGRTGESANETPAGLGGPPGSP | 577 |
| RSC-2709 | GTAEAASASGSTGRTGEPANETPAGLSGPPGSP | 578 |
| RSC-2710 | GTAEAASASGTTGRAGEPANATPTGLSGPPGSP | 579 |
| RSC-2711 | GTAEAASASGRTGRPGEGANATPTGLPGPPGSP | 580 |
| RSC-2712 | GTAEAASASGRTGRGGEAANATPSGLGGPPGSP | 581 |
| RSC-2713 | GTAEAASASGSTGRSGESANATPGGLGGPPGSP | 582 |
| RSC-2714 | GTAEAASASGRTGRTGEEANATPAGLPGPPGSP | 583 |
| RSC-2715 | GTAEAASASGATGRPGEPANTTPEGLEGPPGSP | 584 |
| RSC-2716 | GTAEAASASGSTGRSGEPANATPGGLTGPPGSP | 585 |
| RSC-2717 | GTAEAASASGPTGRGGEGANTTPTGLPGPPGSP | 586 |
| RSC-2718 | GTAEAASASGPTGRSGEGANATPSGLTGPPGSP | 587 |
| RSC-2719 | GTAEAASASGTTGRASEGANSTPAPLTEPPGSP | 588 |
| RSC-2720 | GTAEAASASGTYGRAAEAANTTPAGLTAPPGSP | 589 |
| RSC-2721 | GTAEAASASGTTGRATEGANATPAELTEPPGSP | 590 |
| RSC-2722 | GTAEAASASGTVGRASEEANTTPASLTGPPGSP | 591 |
| RSC-2723 | GTAEAASASGTTGRAPEAANATPAPLTGPPGSP | 592 |
| RSC-2724 | GTAEAASASGTWGRATEPANATPAPLTSPPGSP | 593 |
| RSC-2725 | GTAEAASASGTVGRASESANATPAELTSPPGSP | 594 |
| RSC-2726 | GTAEAASASGTVGRAPEGANSTPAGLTGPPGSP | 595 |
| RSC-2727 | GTAEAASASGTWGRATEAPNLEPATLTTPPGSP | 596 |
| RSC-2728 | GTAEAASASGTTGRATEAPNLTPAPLTEPPGSP | 597 |
| RSC-2729 | GTAEAASASGTQGRATEAPNLSPAALTSPPGSP | 598 |
| RSC-2730 | GTAEAASASGTQGRAAEAPNLTPATLTAPPGSP | 599 |
| RSC-2731 | GTAEAASASGTSGRAPEATNLAPAPLTGPPGSP | 600 |
| RSC-2732 | GTAEAASASGTQGRAAEAANLTPAGLTEPPGSP | 601 |
| RSC-2733 | GTAEAASASGTTGRAGSAPNLPPTGLTTPPGSP | 602 |
| RSC-2734 | GTAEAASASGTTGRAGGAENLPPEGLTAPPGSP | 603 |
| RSC-2735 | GTAEAASASGTTSRAGTATNLTPEGLTAPPGSP | 604 |
| RSC-2736 | GTAEAASASGTTGRAGTATNLPPSGLTTPPGSP | 605 |
| RSC-2737 | GTAEAASASGTTARAGEAENLSPSGLTAPPGSP | 606 |
| RSC-2738 | GTAEAASASGTTGRAGGAGNLAPGGLTEPPGSP | 607 |
| RSC-2739 | GTAEAASASGTTGRAGTATNLPPEGLTGPPGSP | 608 |
| RSC-2740 | GTAEAASASGTTGRAGGAANLAPTGLTEPPGSP | 609 |
| RSC-2741 | GTAEAASASGTTGRAGTAENLAPSGLTTPPGSP | 610 |
| RSC-2742 | GTAEAASASGTTGRAGSATNLGPGGLTGPPGSP | 611 |
| RSC-2743 | GTAEAASASGTTARAGGAENLTPAGLTEPPGSP | 612 |
| RSC-2744 | GTAEAASASGTTARAGSAENLSPSGLTGPPGSP | 613 |
| RSC-2745 | GTAEAASASGTTARAGGAGNLAPEGLTTPPGSP | 614 |
| RSC-2746 | GTAEAASASGTTSRAGAAENLTPTGLTGPPGSP | 615 |
| RSC-2747 | GTAEAASASGTYGRTTTPGNEPPASLEAEPGSP | 616 |
| RSC-2748 | GTAEAASASGTYSRGESGPNEPPPGLTGPPGSP | 617 |
| RSC-2749 | GTAEAASASGAWGRTGASENETPAPLGGEPGSP | 618 |
| RSC-2750 | GTAEAASASGRWGRAETTPNTPPEGLETEPGSP | 619 |
| RSC-2751 | GTAEAASASGESGRAANHTGAEPPELGAGPGSP | 620 |
| RSC-2754 | GTAEAASASGTTGRAGEAANLTPAGLTESPGSP | 621 |
| RSC-2755 | GTAEAASASGTTGRAGEAANLTPAALTESPGSP | 622 |
| RSC-2756 | GTAEAASASGTTGRAGEAANLTPAPLTESPGSP | 623 |
| RSC-2757 | GTAEAASASGTTGRAGEAANLTPEPLTESPGSP | 624 |
| RSC-2758 | GTAEAASASGTTGRAGEAANLTPAGLTGAPGSP | 625 |
| RSC-2759 | GTAEAASASGTTGRAGEAANLTPEGLTGAPGSP | 626 |
| RSC-2760 | GTAEAASASGTTGRAGEAANLTPEPLTGAPGSP | 627 |
| RSC-2761 | GTAEAASASGTTGRAGEAANLTPAGLTEAPGSP | 628 |
| RSC-2762 | GTAEAASASGTTGRAGEAANLTPEGLTEAPGSP | 629 |
| RSC-2763 | GTAEAASASGTTGRAGEAANLTPAPLTEAPGSP | 630 |
| RSC-2764 | GTAEAASASGTTGRAGEAANLTPEPLTEAPGSP | 631 |
| RSC-2765 | GTAEAASASGTTGRAGEAANLTPEPLTGPPGSP | 632 |
| RSC-2766 | GTAEAASASGTTGRAGEAANLTPAGLTGGPGSP | 633 |
| RSC-2767 | GTAEAASASGTTGRAGEAANLTPEGLTGGPGSP | 634 |
| RSC-2768 | GTAEAASASGTTGRAGEAANLTPEALTGGPGSP | 635 |
| RSC-2769 | GTAEAASASGTTGRAGEAANLTPEPLTGGPGSP | 636 |
| RSC-2770 | GTAEAASASGTTGRAGEAANLTPAGLTEGPGSP | 637 |
| RSC-2771 | GTAEAASASGTTGRAGEAANLTPEGLTEGPGSP | 638 |
| RSC-2772 | GTAEAASASGTTGRAGEAANLTPAPLTEGPGSP | 639 |
| RSC-2773 | GTAEAASASGTTGRAGEAANLTPEPLTEGPGSP | 640 |
| RSC-3047 | GTAEAASASGTTGRAGEAEGATSAGATGPPGSP | 564 |
| RSC-2783 | GTAEAASASGEAGRSAEATSAGATGPPGSP | 641 |
| RSC-3107 | GTAEAASASGSASGTYSRGESGPGSPPGSP | 642 |
| RSC-3103 | GTAEAASASGSASGEAGRTDTHPGSPPGSP | 643 |
| RSC-3102 | GTAEAASASGSASGEPGRAAEHPGSPPGSP | 644 |
| RSC-3119 | GTAEAASASGSPAGESSRGTTIAGSPPGSP | 645 |
| RSC-3043 | GTAEAASASGTTGEAGEAAGLTPAGLTGPPGSP | 566 |
| RSC-2789 | GTAEAASASGEAGESAGATPAGLTGPPGSP | 646 |
| RSC-3109 | GTAEAASASGSASGAPLELEAGPGSPPGSP | 647 |
| RSC-3110 | GTAEAASASGSASGEPPELGAGPGSPPGSP | 648 |
| RSC-3111 | GTAEAASASGSASGEPSGLTEGPGSPPGSP | 649 |
| RSC-3112 | GTAEAASASGSASGTPAPLTEPPGSPPGSP | 650 |
| RSC-3113 | GTAEAASASGSASGTPAELTEPPGSPPGSP | 651 |
| RSC-3114 | GTAEAASASGSASGPPPGLTGPPGSPPGSP | 652 |
| RSC-3115 | GTAEAASASGSASGTPAPLGGEPGSPPGSP | 653 |
| RSC-3125 | GTAEAASASGSPAGAPEGLTGPAGSPPGSP | 654 |
| RSC-3126 | GTAEAASASGSPAGPPEGLETEAGSPPGSP | 655 |
| RSC-3127 | GTAEAASASGSPTSGQGGLTGPGSEPPGSP | 656 |
| RSC-3131 | GTAEAASASGSESAPPEGLETESTEPPGSP | 657 |
| RSC-3132 | GTAEAASASGSEGSEPLELGAASETPPGSP | 658 |
| RSC-3133 | GTAEAASASGSEGSGPAGLEAPSETPPGSP | 659 |
| RSC-3138 | GTAEAASASGSEPTPPASLEAEPGSPPGSP | 660 |

In another aspect, the release segments (either RS1 and/or RS2) for incorporation into the polypeptides of any of the subject composition embodiments described herein can be designed to be selectively sensitive in order to have different rates of cleavage and different cleavage efficiencies to the various proteases for which they are substrates. As a given protease may be found in different concentrations in diseased tissues, including but not limited to a tumor, a blood cancer, or an inflammatory tissue or site of inflammation compared to healthy tissues or in the circulation, the disclosure provides RS that have had the individual amino acid sequences engineered to have a higher or lower cleavage efficiency for a given protease in order to ensure that the polypeptide is preferentially converted from the prodrug form to the active form (i.e., by the separation and release of the antigen binding fragments and XTEN from the polypeptide after cleavage of the release segment) when in proximity to the target cell or tissue and its co-localized proteases compared to the rate of cleavage of the release segment in healthy tissue or the circulation such that the released antigen binding fragments have a greater ability to bind to ligands in the diseased tissues compared to the prodrug form that remains in circulation. By such selective designs, the therapeutic index of the resulting compositions can be improved, resulting in reduced side effects relative to convention therapeutics that do not incorporate such site-specific activation.

As used herein cleavage efficiency is defined as the loge value of the ratio of the percentage of the test substrate comprising the release segment cleaved to the percentage of the control substrate AC1611 cleaved when each is subjected to the protease enzyme in biochemical assays (further detailed in the Examples) in which the reaction is conducted wherein the initial substrate concentration is 6 μM, the reactions are incubated at 37° C. for 2 hours before being stopped by adding EDTA, with the amount of digestion products and uncleaved substrate analyzed by non-reducing SDS-PAGE to establish the ratio of the percentage of the release segments cleaved. The cleavage efficiency is calculated as follows:

$${Log}_{2} ( \frac{\% Cleaved for substrate of interest}{\% cleaved for AC 1611 in the same experiment} )$$

$${Log}_{2} ( \frac{\% Cleaved for substrate of interest}{\% cleaved for AC 1611 in the same experiment} )$$

Thus, a cleavage efficiency of −1 means that the amount of test substrate cleaved was 50% compared to that of the control substrate, while a cleavage efficiency of +1 means that the amount of test substrate cleaved was 200% compared to that of the control substrate. A higher rate of cleavage by the test protease relative to the control would result in a higher cleavage efficiency, and a slower rate of cleavage by the test protease relative to the control would result in a lower cleavage efficiency. As detailed in the Examples, a control RS sequence AC1611 (RSR-1517), having the amino acid sequence EAGRSANHEPLGLVAT (SEQ ID NO: 42), was established as having an appropriate baseline cleavage efficiency by the proteases legumain, MMP-2, MMP-7, MMP-9, MMP-14, uPA, and matriptase, when tested in in vitro biochemical assays for rates of cleavage by the individual proteases. By selective substitution of amino acids at individual locations in the RS peptides, libraries of RS were created and evaluated against the panel of the 7 proteases (detailed more fully in the Examples), resulting in profiles that were used to establish guidelines for appropriate amino acid substitutions in order to achieve RS with desired cleavage efficiencies. In making RS with desired cleavage efficiencies, substitutions using the hydrophilic amino acids A, E, G, P, S, and T are preferred, however other L-amino acids can be substituted at given positions in order to adjust the cleavage efficiency so long as the release segment retains at least some susceptibility to cleavage by a protease.

### IV). XTEN Polypeptides

In another aspect, the disclosure relates to polypeptides comprising at least a first extended recombinant polypeptide (XTEN) that is incorporated into the subject composition embodiments described herein, thereby increasing the mass and size of the construct and also serving to greatly reduce the ability of the antigen binding fragments to bind their ligands when the molecule is in the intact, uncleaved state, as described more fully below. In some embodiments, the disclosure provides a polypeptide comprising a single XTEN fused to the terminus of the RS that is located between the antigen binding fragment and the XTEN. In other embodiments, the disclosure provides a polypeptide comprising a first and a second XTEN (XTEN1 and XTEN2) fused to the N- and C-terminus of an RS1 and RS2, respectively, that are located between each antigen binding fragment and the XTEN.

Without being bound by theory, the incorporation of the XTEN can be incorporated into the design of the subject compositions to confer certain properties: 1) provide polypeptide compositions with an XTEN that shields the antigen binding fragments and reduces their binding affinity for the target cell markers and effector cell antigens when the composition is in its intact, prodrug form; ii) provide polypeptide compositions with an XTEN that provides enhanced half-life when administered to a subject, iii) contribute to the solubility and stability of the intact composition, thereby enhancing the pharmaceutical properties of the subject compositions; and iv) provide polypeptide compositions with an XTEN that reduces extravasation in normal tissues and organs yet permits a degree of extravasation in diseased tissues (e.g., a tumor) with larger pore sizes in the vasculature, yet could be released from the composition by action of certain mammalian proteases, thereby permitting the antigen binding fragments of the composition to more readily penetrate into the diseased tissues, e.g. a tumor, and to bind to and link together the target cell markers on the effector cell and tumor cell. To meet these needs, the disclosure provides compositions comprising one or more XTEN in which the XTEN provides increased mass and hydrodynamic radius to the resulting composition. The XTEN polypeptides of the embodiments provide certain advantages in the design of the subject compositions in that is provides not only provides increased mass and hydrodynamic radius to the composition, but its flexible, unstructured characteristics can provide a shielding effect over the antigen binding fragments of the composition, thereby reducing the binding to antigens in normal tissues or the vasculature of normal tissues that don't express or express reduced levels of target cell markers and/or effector cell antigens. Additionally, the incorporation of XTEN into the subject compositions can enhance the solubility and proper folding of the single chain antibody binding fragments during their expression and recovery.

XTEN are polypeptides with non-naturally occurring, substantially non-repetitive sequences having a low degree or no secondary or tertiary structure under physiologic conditions, as well as one or more additional properties described in the paragraphs that follow. In some embodiments, the present disclosure provides polypeptides comprising one or more XTEN having from at least about 36, 72, 96, 100, 144, 200, 288, 292, 293, 300, 576, 584, 800, 864, 867, 868, 900, or at least about 1000 or more amino acids. In one embodiment, the present disclosure provides a polypeptide comprising an XTEN1 wherein the XTEN1 is characterized in that it has at least about 36 or 100 amino acid residues wherein at least 90%, 91%, 92%, 93%, 94%, 95%, 96%, 97%, 98%, 99% or 100% of the amino acid residues of the XTEN1 sequence are selected from glycine (G), alanine (A), serine (S), threonine (T), glutamate (E) and proline (P) and it has at least 4-6 different amino acids selected from G, A, S, T, E and P. In some embodiments, the present disclosure provides polypeptides comprising an XTEN1 having at least about 36 to about 1000, at least about 100 to 1000, or at least 100 to about 900, or at least about 144 to about 868, or at least about 288-868 amino acid residues. In other cases, the present disclosure provides polypeptides comprising an XTEN1 having at least about 36 to about 1000, at least about 100 to about 1000, or at least 100 to about 900, or at least about 144 to about 868, or at least about 288-868 amino acid residues wherein 90%, 91%, 92%, 93%, 94%, 95%, 96%, 97%, 98%, 99%, or 100% of the amino acid residues are selected from 4-6 types of amino acids selected from the group consisting of glycine (G), alanine (A), serine (S), threonine (T), glutamate (E) and proline (P). In other cases, the present disclosure provides polypeptides comprising an XTEN1 wherein the XTEN1 is characterized in that it has at least about 36 to about 1000 amino acid residues or at least about 100 to about 1000 amino acid residues, at least 90%, 91%, 92%, 93%, 94%, 95%, 96%, 97%, 98%, 99% or 100% of the amino acid residues of the XTEN1 sequence are selected from six types of amino acids selected from glycine (G), alanine (A), serine (S), threonine (T), glutamate (E) and proline (P).

In another embodiment, the present disclosure provides polypeptides of any of the embodiments described herein comprising an XTEN1 wherein the XTEN1 is characterized in that it has at least about 36 to about 1000, at least about 100 to about 1000, or at least about 100 to about 900, or at least 144 to about 868 amino acid residues, wherein at least 90%, 91%, 92%, 93%, 94%, 95%, 96%, 97%, 98%, 99% or 100% of the amino acid residues of the XTEN1 sequence are selected from at least three of the sequences of SEQ ID NOs: 661-664. In some cases, the XTEN 1 sequence can be assembled by any combination of the 12 amino acid units of SEQ ID NOs: 661-664 such that any length of at least 36 amino acids or longer, in 12 amino acid increments, can be achieved; e.g., 36, 48, 60, 72, 84, 96 amino acids, etc. In other cases, the polypeptides of any of the subject composition embodiments described herein can comprise an XTEN1 wherein the XTEN1 is characterized in that it has at least about 36 to about 1000, at least about 100 to about 1000, or at least about 100 to about 900, or at least 144 to about 868 amino acid residues, wherein at least 90%, 91%, 92%, 93%, 94%, 95%, 96%, 97%, 98%, 99% or 100% of the amino acid residues of the XTEN1 sequence are selected from the sequences of SEQ ID NOs: 665-718 and 922-926. In another embodiment, the XTEN of any of the subject composition embodiments described herein can have an affinity tag of HHHHHH (SEQ ID NO: 1150), HHHHHHHH (SEQ ID NO: 1151), or the sequence EPEA (SEQ ID NO: 1149) appended to the N- or C-terminus of the XTEN of the composition to facilitate the purification of the composition to at least 90%, 91%, 92%, 93%, 94%, 95%, 96%, 97%, 98%, or at least 99% purity by chromatography methods known in the art; e.g., IMAC chromatography or C-tagXL chromatography, or methods described in the Examples, below.

In another embodiment, the present disclosure provides a polypeptide comprising an XTEN1 wherein the XTEN1 comprises an amino acid sequence having at least about 90%, 91%, 92%, 93%, 94%, 95%, 96%, 97%, 98%, 99%, or 100% sequence identity to an AE36 (comprising a sequence selected from any three of the sequences of SEQ ID NOs: 661-664), or a sequence selected from the sequences of AE144_1A, AE144_2A, AE144_2B, AE144_3A, AE144_3B, AE144_4A, AE144_4B, AE144_5A, AE144_6B, AE144_7A, AE284, AE288_1, AE288_2, AE288_3, AE292, AE293, AE576, AE584, AE864, AE864_2, AE865, AE866, AE867, and AE868, each of which being set forth in Table 7.

In some aspects of any of the embodiments disclosed herein, a subject polypeptide comprises an XTEN1 and an XTEN2. The configurations of the polypeptides comprising XTEN1 and XTEN2, amongst the other components, are described herein, below. In one embodiment, the present disclosure provides a polypeptide comprising an XTEN1 and an XTEN2 wherein the XTEN 1 and XTEN2 are each characterized in that it has at least about 36 to about 1000 amino acid residues or at least about 100 to about 1000 amino acid residues, wherein at least 90%, 91%, 92%, 93%, 94%, 95%, 96%, 97%, 98%, 99% or 100% of the amino acid residues of the XTEN 1 and XTEN2 sequences are selected from at least three of the sequences of SEQ ID NOs: 661-664. In another embodiment, the present disclosure provides a polypeptide comprising an XTEN1 and an XTEN2 wherein the XTEN 1 and the XTEN2 are each characterized in that each has at least about 36 to about 1000 amino acid residues or at least about 100 to about 1000 amino acid residues, wherein at least 90%, 91%, 92%, 93%, 94%, 95%, 96%, 97%, 98%, 99% or 100% of the amino acid residues of the XTEN 1 and XTEN2 sequences are selected from the sequences of SEQ ID NOs: 665-718 and 922-926. In another embodiment, the polypeptides of any of the subject composition embodiments described herein can comprise an XTEN1 and an XTEN2 wherein the XTEN 1 and XTEN2 each comprises an amino acid sequence having at least about 90%, 91%, 92%, 93%, 94%, 95%, 96%, 97%, 98%, 99%, or 100% sequence identity to a sequence selected from the sequences of AE144_1A, AE144_2A, AE144_2B, AE144_3A, AE144_3B, AE144_4A, AE144_4B, AE144_5A, AE144_6B, AE144_7A, AE284, AE288_1, AE288_2, AE288_3, AE292, AE293, AE576, AE584, AE864, AE864_2, AE865, AE866, AE867, and AE868, each of which being set forth in Table 7. In some cases of the foregoing embodiments of the paragraph, the XTEN1 and XTEN 2 are identical. In other cases of the foregoing embodiments of the paragraph, the XTEN1 and XTEN2 of the foregoing embodiments of the paragraph have different amino acid sequences. In some cases, the XTEN1 of any of the polypeptide composition embodiments having 2 XTENs is fused to the C-terminus of the polypeptide and is selected from the group consisting of AE293, AE300, AE584 and AE868. In other cases, the XTEN2 of any of the polypeptide composition embodiments having 2 XTENs is fused to the N-terminus of the polypeptide and is selected from the group consisting of AE144_7A, AE292, AE576, and AE864. In other cases, the XTEN1 of any of the polypeptide composition embodiments having 2 XTENs is fused to the C-terminus of the polypeptide and is selected from the group consisting of AE293, AE300, AE584 and AE868 and the XTEN 2 is fused to the N-terminus and is selected from the group consisting of AE144_7A, AE292, AE576, and AE864.

TABLE 6
XTEN Sequence Motifs

| Motif Name | Amino Acid Sequence | SEQ ID NO: |
|---|---|---|
| AE1 | GSPAGSPTSTEE | 661 |
| AE2 | GSEPATSGSETP | 662 |
| AE3 | GTSESATPESGP | 663 |
| AE4 | GTSTEPSEGSAP | 664 |

TABLE 7
XTEN Sequences

| | | SEQ |
|---|---|---|
| XTEN | | ID |
| Name | Amino Acid Sequence | NO: |
| AE144 | GSEPATSGSETPGTSESATPESGPGSEPATSGSETPGSPAGSPTSTEEGTSTE | 665 |
| | PSEGSAPGSEPATSGSETPGSEPATSGSETPGSEPATSGSETPGTSTEPSEGS | |
| | APGTSESATPESGPGSEPATSGSETPGTSTEPSEGSAP | |
| AE144_1A | SPAGSPTSTEEGTSESATPESGPGTSTEPSEGSAPGSPAGSPTSTEEGTSTEP | 666 |
| | SEGSAPGTSTEPSEGSAPGTSESATPESGPGSEPATSGSETPGSEPATSGSET | |
| | PGSPAGSPTSTEEGTSESATPESGPGTSTEPSEGSAPG | |
| AE144_2A | TSTEPSEGSAPGSPAGSPTSTEEGTSTEPSEGSAPGTSTEPSEGSAPGTSESA | 667 |
| | TPESGPGTSTEPSEGSAPGTSESATPESGPGSEPATSGSETPGTSTEPSEGSA | |
| | PGTSTEPSEGSAPGTSESATPESGPGTSESATPESGPG | |
| AE144_2B | TSTEPSEGSAPGSPAGSPTSTEEGTSTEPSEGSAPGTSTEPSEGSAPGTSESA | 668 |
| | TPESGPGTSTEPSEGSAPGTSESATPESGPGSEPATSGSETPGTSTEPSEGSA | |
| | PGTSTEPSEGSAPGTSESATPESGPGTSESATPESGPG | |
| AE144_3A | SPAGSPTSTEEGTSESATPESGPGSEPATSGSETPGTSESATPESGPGTSTEP | 669 |
| | SEGSAPGTSTEPSEGSAPGTSTEPSEGSAPGTSTEPSEGSAPGTSTEPSEGSA | |
| | PGTSTEPSEGSAPGSPAGSPTSTEEGTSTEPSEGSAPG | |
| AE144_3B | SPAGSPTSTEEGTSESATPESGPGSEPATSGSETPGTSESATPESGPGTSTEP | 670 |
| | SEGSAPGTSTEPSEGSAPGTSTEPSEGSAPGTSTEPSEGSAPGTSTEPSEGSA | |
| | PGTSTEPSEGSAPGSPAGSPTSTEEGTSTEPSEGSAPG | |
| AE144_4A | TSESATPESGPGSEPATSGSETPGTSESATPESGPGSEPATSGSETPGTSESA | 671 |
| | TPESGPGTSTEPSEGSAPGTSESATPESGPGSPAGSPTSTEEGSPAGSPTSTE | |
| | EGSPAGSPTSTEEGTSESATPESGPGTSTEPSEGSAPG | |
| AE144_4B | TSESATPESGPGSEPATSGSETPGTSESATPESGPGSEPATSGSETPGTSESA | 672 |
| | TPESGPGTSTEPSEGSAPGTSESATPESGPGSPAGSPTSTEEGSPAGSPTSTE | |
| | EGSPAGSPTSTEEGTSESATPESGPGTSTEPSEGSAPG | |
| AE144_5A | TSESATPESGPGSEPATSGSETPGTSESATPESGPGSEPATSGSETPGTSESA | 673 |
| | TPESGPGTSTEPSEGSAPGSPAGSPTSTEEGTSESATPESGPGSEPATSGSET | |
| | PGTSESATPESGPGSPAGSPTSTEEGSPAGSPTSTEEG | |
| AE144_6B | TSTEPSEGSAPGTSESATPESGPGTSESATPESGPGTSESATPESGPGSEPAT | 674 |
| | SGSETPGSEPATSGSETPGSPAGSPTSTEEGTSTEPSEGSAPGTSTEPSEGSA | |
| | PGSEPATSGSETPGTSESATPESGPGTSTEPSEGSAPG | |
| AE2881 | GTSESATPESGPGSEPATSGSETPGTSESATPESGPGSEPATSGSETPGTSES | 675 |
| | ATPESGPGTSTEPSEGSAPGSPAGSPTSTEEGTSESATPESGPGSEPATSGSE | |
| | TPGTSESATPESGPGSPAGSPTSTEEGSPAGSPTSTEEGTSTEPSEGSAPGTS | |
| | ESATPESGPGTSESATPESGPGTSESATPESGPGSEPATSGSETPGSEPATSG | |
| | SETPGSPAGSPTSTEEGTSTEPSEGSAPGTSTEPSEGSAPGSEPATSGSETPG | |
| | TSESATPESGPGTSTEPSEGSAP | |
| AE288_2 | GSPAGSPTSTEEGTSESATPESGPGSEPATSGSETPGTSESATPESGPGTSTE | 676 |
| | PSEGSAPGTSTEPSEGSAPGTSTEPSEGSAPGTSTEPSEGSAPGTSTEPSEGS | |
| | APGTSTEPSEGSAPGSPAGSPTSTEEGTSTEPSEGSAPGTSESATPESGPGSE | |
| | PATSGSETPGTSESATPESGPGSEPATSGSETPGTSESATPESGPGTSTEPSE | |
| | GSAPGTSESATPESGPGSPAGSPTSTEEGSPAGSPTSTEEGSPAGSPTSTEEG | |
| | TSESATPESGPGTSTEPSEGSAP | |
| AE576 | GSPAGSPTSTEEGTSESATPESGPGTSTEPSEGSAPGSPAGSPTSTEEGTSTE | 677 |
| | PSEGSAPGTSTEPSEGSAPGTSESATPESGPGSEPATSGSETPGSEPATSGSE | |
| | TPGSPAGSPTSTEEGTSESATPESGPGTSTEPSEGSAPGTSTEPSEGSAPGSP | |
| | AGSPTSTEEGTSTEPSEGSAPGTSTEPSEGSAPGTSESATPESGPGTSTEPSE | |
| | GSAPGTSESATPESGPGSEPATSGSETPGTSTEPSEGSAPGTSTEPSEGSAPG | |
| | TSESATPESGPGTSESATPESGPGSPAGSPTSTEEGTSESATPESGPGSEPAT | |
| | SGSETPGTSESATPESGPGTSTEPSEGSAPGTSTEPSEGSAPGTSTEPSEGSA | |
| | PGTSTEPSEGSAPGTSTEPSEGSAPGTSTEPSEGSAPGSPAGSPTSTEEGTST | |
| | EPSEGSAPGTSESATPESGPGSEPATSGSETPGTSESATPESGPGSEPATSGS | |
| | ETPGTSESATPESGPGTSTEPSEGSAPGTSESATPESGPGSPAGSPTSTEEGS | |
| | PAGSPTSTEEGSPAGSPTSTEEGTSESATPESGPGTSTEPSEGSAP | |
| AE624 | MAEPAGSPTSTEEGTPGSGTASSSPGSSTPSGATGSPGASPGTSSTGSPGSPA | 678 |
| | GSPTSTEEGTSESATPESGPGTSTEPSEGSAPGSPAGSPTSTEEGTSTEPSEG | |
| | SAPGTSTEPSEGSAPGTSESATPESGPGSEPATSGSETPGSEPATSGSETPGS | |
| | PAGSPTSTEEGTSESATPESGPGTSTEPSEGSAPGTSTEPSEGSAPGSPAGSP | |
| | TSTEEGTSTEPSEGSAPGTSTEPSEGSAPGTSESATPESGPGTSTEPSEGSAP | |
| | GTSESATPESGPGSEPATSGSETPGTSTEPSEGSAPGTSTEPSEGSAPGTSES | |
| | ATPESGPGTSESATPESGPGSPAGSPTSTEEGTSESATPESGPGSEPATSGSE | |
| | TPGTSESATPESGPGTSTEPSEGSAPGTSTEPSEGSAPGTSTEPSEGSAPGTS | |
| | TEPSEGSAPGTSTEPSEGSAPGTSTEPSEGSAPGSPAGSPTSTEEGTSTEPSE | |
| | GSAPGTSESATPESGPGSEPATSGSETPGTSESATPESGPGSEPATSGSETPG | |
| | TSESATPESGPGTSTEPSEGSAPGTSESATPESGPGSPAGSPTSTEEGSPAGS | |
| | PTSTEEGSPAGSPTSTEEGTSESATPESGPGTSTEPSEGSAP | |
| AE864 | GSPAGSPTSTEEGTSESATPESGPGTSTEPSEGSAPGSPAGSPTSTEEGTSTE | 679 |
| | PSEGSAPGTSTEPSEGSAPGTSESATPESGPGSEPATSGSETPGSEPATSGSE | |
| | TPGSPAGSPTSTEEGTSESATPESGPGTSTEPSEGSAPGTSTEPSEGSAPGSP | |
| | AGSPTSTEEGTSTEPSEGSAPGTSTEPSEGSAPGTSESATPESGPGTSTEPSE | |
| | GSAPGTSESATPESGPGSEPATSGSETPGTSTEPSEGSAPGTSTEPSEGSAPG | |
| | TSESATPESGPGTSESATPESGPGSPAGSPTSTEEGTSESATPESGPGSEPAT | |
| | SGSETPGTSESATPESGPGTSTEPSEGSAPGTSTEPSEGSAPGTSTEPSEGSA | |
| | PGTSTEPSEGSAPGTSTEPSEGSAPGTSTEPSEGSAPGSPAGSPTSTEEGTST | |
| | EPSEGSAPGTSESATPESGPGSEPATSGSETPGTSESATPESGPGSEPATSGS | |
| | ETPGTSESATPESGPGTSTEPSEGSAPGTSESATPESGPGSPAGSPTSTEEGS | |
| | PAGSPTSTEEGSPAGSPTSTEEGTSESATPESGPGTSTEPSEGSAPGTSESAT | |
| | PESGPGSEPATSGSETPGTSESATPESGPGSEPATSGSETPGTSESATPESGP | |
| | GTSTEPSEGSAPGSPAGSPTSTEEGTSESATPESGPGSEPATSGSETPGTSES | |
| | ATPESGPGSPAGSPTSTEEGSPAGSPTSTEEGTSTEPSEGSAPGTSESATPES | |
| | GPGTSESATPESGPGTSESATPESGPGSEPATSGSETPGSEPATSGSETPGSP | |
| | AGSPTSTEEGTSTEPSEGSAPGTSTEPSEGSAPGSEPATSGSETPGTSESATP | |
| | ESGPGTSTEPSEGSAP | |
| AE865 | GGSPAGSPTSTEEGTSESATPESGPGTSTEPSEGSAPGSPAGSPTSTEEGTST | 680 |
| | EPSEGSAPGTSTEPSEGSAPGTSESATPESGPGSEPATSGSETPGSEPATSGS | |
| | ETPGSPAGSPTSTEEGTSESATPESGPGTSTEPSEGSAPGTSTEPSEGSAPGS | |
| | PAGSPTSTEEGTSTEPSEGSAPGTSTEPSEGSAPGTSESATPESGPGTSTEPS | |
| | EGSAPGTSESATPESGPGSEPATSGSETPGTSTEPSEGSAPGTSTEPSEGSAP | |
| | GTSESATPESGPGTSESATPESGPGSPAGSPTSTEEGTSESATPESGPGSEPA | |
| | TSGSETPGTSESATPESGPGTSTEPSEGSAPGTSTEPSEGSAPGTSTEPSEGS | |
| | APGTSTEPSEGSAPGTSTEPSEGSAPGTSTEPSEGSAPGSPAGSPTSTEEGTS | |
| | TEPSEGSAPGTSESATPESGPGSEPATSGSETPGTSESATPESGPGSEPATSG | |
| | SETPGTSESATPESGPGTSTEPSEGSAPGTSESATPESGPGSPAGSPTSTEEG | |
| | SPAGSPTSTEEGSPAGSPTSTEEGTSESATPESGPGTSTEPSEGSAPGTSESA | |
| | TPESGPGSEPATSGSETPGTSESATPESGPGSEPATSGSETPGTSESATPESG | |
| | PGTSTEPSEGSAPGSPAGSPTSTEEGTSESATPESGPGSEPATSGSETPGTSE | |
| | SATPESGPGSPAGSPTSTEEGSPAGSPTSTEEGTSTEPSEGSAPGTSESATPE | |
| | SGPGTSESATPESGPGTSESATPESGPGSEPATSGSETPGSEPATSGSETPGS | |
| | PAGSPTSTEEGTSTEPSEGSAPGTSTEPSEGSAPGSEPATSGSETPGTSESAT | |
| | PESGPGTSTEPSEGSAP | |
| AE866 | PGSPAGSPTSTEEGTSESATPESGPGTSTEPSEGSAPGSPAGSPTSTEEGTST | 681 |
| | EPSEGSAPGTSTEPSEGSAPGTSESATPESGPGSEPATSGSETPGSEPATSGS | |
| | ETPGSPAGSPTSTEEGTSESATPESGPGTSTEPSEGSAPGTSTEPSEGSAPGS | |
| | PAGSPTSTEEGTSTEPSEGSAPGTSTEPSEGSAPGTSESATPESGPGTSTEPS | |
| | EGSAPGTSESATPESGPGSEPATSGSETPGTSTEPSEGSAPGTSTEPSEGSAP | |
| | GTSESATPESGPGTSESATPESGPGSPAGSPTSTEEGTSESATPESGPGSEPA | |
| | TSGSETPGTSESATPESGPGTSTEPSEGSAPGTSTEPSEGSAPGTSTEPSEGS | |
| | APGTSTEPSEGSAPGTSTEPSEGSAPGTSTEPSEGSAPGSPAGSPTSTEEGTS | |
| | TEPSEGSAPGTSESATPESGPGSEPATSGSETPGTSESATPESGPGSEPATSG | |
| | SETPGTSESATPESGPGTSTEPSEGSAPGTSESATPESGPGSPAGSPTSTEEG | |
| | SPAGSPTSTEEGSPAGSPTSTEEGTSESATPESGPGTSTEPSEGSAPGTSESA | |
| | TPESGPGSEPATSGSETPGTSESATPESGPGSEPATSGSETPGTSESATPESG | |
| | PGTSTEPSEGSAPGSPAGSPTSTEEGTSESATPESGPGSEPATSGSETPGTSE | |
| | SATPESGPGSPAGSPTSTEEGSPAGSPTSTEEGTSTEPSEGSAPGTSESATPE | |
| | SGPGTSESATPESGPGTSESATPESGPGSEPATSGSETPGSEPATSGSETPGS | |
| | PAGSPTSTEEGTSTEPSEGSAPGTSTEPSEGSAPGSEPATSGSETPGTSESAT | |
| | PESGPGTSTEPSEGSAPG | |
| AE1152 | GSPAGSPTSTEEGTSESATPESGPGTSTEPSEGSAPGSPAGSPTSTEEGTSTE | 682 |
| | PSEGSAPGTSTEPSEGSAPGTSESATPESGPGSEPATSGSETPGSEPATSGSE | |
| | TPGSPAGSPTSTEEGTSESATPESGPGTSTEPSEGSAPGTSTEPSEGSAPGSP | |
| | AGSPTSTEEGTSTEPSEGSAPGTSTEPSEGSAPGTSESATPESGPGTSTEPSE | |
| | GSAPGTSESATPESGPGSEPATSGSETPGTSTEPSEGSAPGTSTEPSEGSAPG | |
| | TSESATPESGPGTSESATPESGPGSPAGSPTSTEEGTSESATPESGPGSEPAT | |
| | SGSETPGTSESATPESGPGTSTEPSEGSAPGTSTEPSEGSAPGTSTEPSEGSA | |
| | PGTSTEPSEGSAPGTSTEPSEGSAPGTSTEPSEGSAPGSPAGSPTSTEEGTST | |
| | EPSEGSAPGTSESATPESGPGSEPATSGSETPGTSESATPESGPGSEPATSGS | |
| | ETPGTSESATPESGPGTSTEPSEGSAPGTSESATPESGPGSPAGSPTSTEEGS | |
| | PAGSPTSTEEGSPAGSPTSTEEGTSESATPESGPGTSTEPSEGSAPGTSESAT | |
| | PESGPGSEPATSGSETPGTSESATPESGPGSEPATSGSETPGTSESATPESGP | |
| | GTSTEPSEGSAPGSPAGSPTSTEEGTSESATPESGPGSEPATSGSETPGTSES | |
| | ATPESGPGSPAGSPTSTEEGSPAGSPTSTEEGTSTEPSEGSAPGTSESATPES | |
| | GPGTSESATPESGPGTSESATPESGPGSEPATSGSETPGSEPATSGSETPGSP | |
| | AGSPTSTEEGTSTEPSEGSAPGTSTEPSEGSAPGSEPATSGSETPGTSESATP | |
| | ESGPGTSTEPSEGSAPGTSESATPESGPGSEPATSGSETPGTSESATPESGPG | |
| | SEPATSGSETPGTSESATPESGPGTSTEPSEGSAPGSPAGSPTSTEEGTSESA | |
| | TPESGPGSEPATSGSETPGTSESATPESGPGSPAGSPTSTEEGSPAGSPTSTE | |
| | EGTSTEPSEGSAPGTSESATPESGPGTSESATPESGPGTSESATPESGPGSEP | |
| | ATSGSETPGSEPATSGSETPGSPAGSPTSTEEGTSTEPSEGSAPGTSTEPSEG | |
| | SAPGSEPATSGSETPGTSESATPESGPGTSTEPSEGSAP | |
| AE144A | STEPSEGSAPGSPAGSPTSTEEGTSTEPSEGSAPGTSESATPESGPGSEPATS | 683 |
| | GSETPGTSESATPESGPGSEPATSGSETPGTSESATPESGPGTSTEPSEGSAP | |
| | GTSESATPESGPGSPAGSPTSTEEGSPAGSPTSTEEGS | |
| AE144B | SEPATSGSETPGTSESATPESGPGSEPATSGSETPGTSESATPESGPGTSTEP | 684 |
| | SEGSAPGSPAGSPTSTEEGTSESATPESGPGSEPATSGSETPGTSESATPESG | |
| | PGSPAGSPTSTEEGSPAGSPTSTEEGTSTEPSEGSAPG | |
| AE180A | TSTEEGTSESATPESGPGSEPATSGSETPGTSESATPESGPGSPAGSPTSTEE | 685 |
| | GSPAGSPTSTEEGTSTEPSEGSAPGTSESATPESGPGTSESATPESGPGTSES | |
| | ATPESGPGSEPATSGSETPGSEPATSGSETPGSPAGSPTSTEEGTSTEPSEGS | |
| | APGTSTEPSEGSAPGSEPATS | |
| AE216A | PESGPGTSTEPSEGSAPGSPAGSPTSTEEGTSESATPESGPGSEPATSGSETP | 686 |
| | GTSESATPESGPGSPAGSPTSTEEGSPAGSPTSTEEGTSTEPSEGSAPGTSES | |
| | ATPESGPGTSESATPESGPGTSESATPESGPGSEPATSGSETPGSEPATSGSE | |
| | TPGSPAGSPTSTEEGTSTEPSEGSAPGTSTEPSEGSAPGSEPATSGSETPGTS | |
| | ESAT | |
| AE252A | ESGPGSEPATSGSETPGTSESATPESGPGTSTEPSEGSAPGSPAGSPTSTEEG | 687 |
| | TSESATPESGPGSEPATSGSETPGTSESATPESGPGSPAGSPTSTEEGSPAGS | |
| | PTSTEEGTSTEPSEGSAPGTSESATPESGPGTSESATPESGPGTSESATPESG | |
| | PGSEPATSGSETPGSEPATSGSETPGSPAGSPTSTEEGTSTEPSEGSAPGTST | |
| | EPSEGSAPGSEPATSGSETPGTSESATPESGPGTSTEPSE | |
| AE288A | TPESGPGTSTEPSEGSAPGTSESATPESGPGSEPATSGSETPGTSESATPESG | 688 |
| | PGSEPATSGSETPGTSESATPESGPGTSTEPSEGSAPGSPAGSPTSTEEGTSE | |
| | SATPESGPGSEPATSGSETPGTSESATPESGPGSPAGSPTSTEEGSPAGSPTS | |
| | TEEGTSTEPSEGSAPGTSESATPESGPGTSESATPESGPGTSESATPESGPGS | |
| | EPATSGSETPGSEPATSGSETPGSPAGSPTSTEEGTSTEPSEGSAPGTSTEPS | |
| | EGSAPGSEPATSGSETPGTSESA | |
| AE324A | PESGPGSPAGSPTSTEEGSPAGSPTSTEEGSPAGSPTSTEEGTSESATPESGP | 689 |
| | GTSTEPSEGSAPGTSESATPESGPGSEPATSGSETPGTSESATPESGPGSEPA | |
| | TSGSETPGTSESATPESGPGTSTEPSEGSAPGSPAGSPTSTEEGTSESATPES | |
| | GPGSEPATSGSETPGTSESATPESGPGSPAGSPTSTEEGSPAGSPTSTEEGTS | |
| | TEPSEGSAPGTSESATPESGPGTSESATPESGPGTSESATPESGPGSEPATSG | |
| | SETPGSEPATSGSETPGSPAGSPTSTEEGTSTEPSEGSAPGTSTEPSEGSAPG | |
| | SEPATS | |
| AE360A | PESGPGTSTEPSEGSAPGTSESATPESGPGSPAGSPTSTEEGSPAGSPTSTEE | 690 |
| | GSPAGSPTSTEEGTSESATPESGPGTSTEPSEGSAPGTSESATPESGPGSEPA | |
| | TSGSETPGTSESATPESGPGSEPATSGSETPGTSESATPESGPGTSTEPSEGS | |
| | APGSPAGSPTSTEEGTSESATPESGPGSEPATSGSETPGTSESATPESGPGSP | |
| | AGSPTSTEEGSPAGSPTSTEEGTSTEPSEGSAPGTSESATPESGPGTSESATP | |
| | ESGPGTSESATPESGPGSEPATSGSETPGSEPATSGSETPGSPAGSPTSTEEG | |
| | TSTEPSEGSAPGTSTEPSEGSAPGSEPATSGSETPGTSESAT | |
| AE396A | PESGPGSEPATSGSETPGTSESATPESGPGTSTEPSEGSAPGTSESATPESGP | 691 |
| | GSPAGSPTSTEEGSPAGSPTSTEEGSPAGSPTSTEEGTSESATPESGPGTSTE | |
| | PSEGSAPGTSESATPESGPGSEPATSGSETPGTSESATPESGPGSEPATSGSE | |
| | TPGTSESATPESGPGTSTEPSEGSAPGSPAGSPTSTEEGTSESATPESGPGSE | |
| | PATSGSETPGTSESATPESGPGSPAGSPTSTEEGSPAGSPTSTEEGTSTEPSE | |
| | GSAPGTSESATPESGPGTSESATPESGPGTSESATPESGPGSEPATSGSETPG | |
| | SEPATSGSETPGSPAGSPTSTEEGTSTEPSEGSAPGTSTEPS | |
| AE432A | EGSAPGSPAGSPTSTEEGTSTEPSEGSAPGTSESATPESGPGSEPATSGSETP | 692 |
| | GTSESATPESGPGSEPATSGSETPGTSESATPESGPGTSTEPSEGSAPGTSES | |
| | ATPESGPGSPAGSPTSTEEGSPAGSPTSTEEGSPAGSPTSTEEGTSESATPES | |
| | GPGTSTEPSEGSAPGTSESATPESGPGSEPATSGSETPGTSESATPESGPGSE | |
| | PATSGSETPGTSESATPESGPGTSTEPSEGSAPGSPAGSPTSTEEGTSESATP | |
| | ESGPGSEPATSGSETPGTSESATPESGPGSPAGSPTSTEEGSPAGSPTSTEEG | |
| | TSTEPSEGSAPGTSESATPESGPGTSESATPESGPGTSESATPESGPGSEPAT | |
| | SGSETPGSEPATSGSETPGSPAGSPTSTEEGTSTEPSEGSAPGTSTEPSEGSA | |
| | PGSEPATS | |
| AE468A | EGSAPGTSTEPSEGSAPGTSTEPSEGSAPGSPAGSPTSTEEGTSTEPSEGSAP | 693 |
| | GTSESATPESGPGSEPATSGSETPGTSESATPESGPGSEPATSGSETPGTSES | |
| | ATPESGPGTSTEPSEGSAPGTSESATPESGPGSPAGSPTSTEEGSPAGSPTST | |
| | EEGSPAGSPTSTEEGTSESATPESGPGTSTEPSEGSAPGTSESATPESGPGSE | |
| | PATSGSETPGTSESATPESGPGSEPATSGSETPGTSESATPESGPGTSTEPSE | |
| | GSAPGSPAGSPTSTEEGTSESATPESGPGSEPATSGSETPGTSESATPESGPG | |
| | SPAGSPTSTEEGSPAGSPTSTEEGTSTEPSEGSAPGTSESATPESGPGTSESA | |
| | TPESGPGTSESATPESGPGSEPATSGSETPGSEPATSGSETPGSPAGSPTSTE | |
| | EGTSTEPSEGSAPGTSTEPSEGSAPGSEPATSGSETPGTSESAT | |
| AE504A | EGSAPGTSTEPSEGSAPGTSTEPSEGSAPGTSTEPSEGSAPGTSTEPSEGSAP | 694 |
| | GSPAGSPTSTEEGTSTEPSEGSAPGTSESATPESGPGSEPATSGSETPGTSES | |
| | ATPESGPGSEPATSGSETPGTSESATPESGPGTSTEPSEGSAPGTSESATPES | |
| | GPGSPAGSPTSTEEGSPAGSPTSTEEGSPAGSPTSTEEGTSESATPESGPGTS | |
| | TEPSEGSAPGTSESATPESGPGSEPATSGSETPGTSESATPESGPGSEPATSG | |
| | SETPGTSESATPESGPGTSTEPSEGSAPGSPAGSPTSTEEGTSESATPESGPG | |
| | SEPATSGSETPGTSESATPESGPGSPAGSPTSTEEGSPAGSPTSTEEGTSTEP | |
| | SEGSAPGTSESATPESGPGTSESATPESGPGTSESATPESGPGSEPATSGSET | |
| | PGSEPATSGSETPGSPAGSPTSTEEGTSTEPSEGSAPGTSTEPSEGSAPGSEP | |
| | ATSGSETPGTSESATPESGPGTSTEPS | |
| AE540A | TPESGPGSPAGSPTSTEEGTSESATPESGPGSEPATSGSETPGTSESATPESG | 695 |
| | PGTSTEPSEGSAPGTSTEPSEGSAPGTSTEPSEGSAPGTSTEPSEGSAPGTST | |
| | EPSEGSAPGTSTEPSEGSAPGSPAGSPTSTEEGTSTEPSEGSAPGTSESATPE | |
| | SGPGSEPATSGSETPGTSESATPESGPGSEPATSGSETPGTSESATPESGPGT | |
| | STEPSEGSAPGTSESATPESGPGSPAGSPTSTEEGSPAGSPTSTEEGSPAGSP | |
| | TSTEEGTSESATPESGPGTSTEPSEGSAPGTSESATPESGPGSEPATSGSETP | |
| | GTSESATPESGPGSEPATSGSETPGTSESATPESGPGTSTEPSEGSAPGSPAG | |
| | SPTSTEEGTSESATPESGPGSEPATSGSETPGTSESATPESGPGSPAGSPTST | |
| | EEGSPAGSPTSTEEGTSTEPSEGSAPGTSESATPESGPGTSESATPESGPGTS | |
| | ESATPESGPGSEPATSGSETPGSEPATSGSETPGSPAGSPTSTEEGTSTEPSE | |
| | GSAPGTSTEP | |
| AE576A | TPESGPGTSESATPESGPGSPAGSPTSTEEGTSESATPESGPGSEPATSGSET | 696 |
| | PGTSESATPESGPGTSTEPSEGSAPGTSTEPSEGSAPGTSTEPSEGSAPGTST | |
| | EPSEGSAPGTSTEPSEGSAPGTSTEPSEGSAPGSPAGSPTSTEEGTSTEPSEG | |
| | SAPGTSESATPESGPGSEPATSGSETPGTSESATPESGPGSEPATSGSETPGT | |
| | SESATPESGPGTSTEPSEGSAPGTSESATPESGPGSPAGSPTSTEEGSPAGSP | |
| | TSTEEGSPAGSPTSTEEGTSESATPESGPGTSTEPSEGSAPGTSESATPESGP | |
| | GSEPATSGSETPGTSESATPESGPGSEPATSGSETPGTSESATPESGPGTSTE | |
| | PSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTS | |
| | ESATPESGPGTSESATPESGPGSEPATSGSETPGSEPATSGSETPGSPAGSPT | |
| | STEEGTSTEPSEGSAPGTSTEPSEGSAPGSEPATSGSETPGTSESA | |
| AE612A | GSETPGTSTEPSEGSAPGTSTEPSEGSAPGTSESATPESGPGTSESATPESGP | 697 |
| | GSPAGSPTSTEEGTSESATPESGPGSEPATSGSETPGTSESATPESGPGTSTE | |
| | PSEGSAPGTSTEPSEGSAPGTSTEPSEGSAPGTSTEPSEGSAPGTSTEPSEGS | |
| | APGTSTEPSEGSAPGSPAGSPTSTEEGTSTEPSEGSAPGTSESATPESGPGSE | |
| | PATSGSETPGTSESATPESGPGSEPATSGSETPGTSESATPESGPGTSTEPSE | |
| | GSAPGTSESATPESGPGSPAGSPTSTEEGSPAGSPTSTEEGSPAGSPTSTEEG | |
| | TSESATPESGPGTSTEPSEGSAPGTSESATPESGPGSEPATSGSETPGTSESA | |
| | TPESGPGSEPATSGSETPGTSESATPESGPGTSTEPSEGSAPGSPAGSPTSTE | |
| | EGTSESATPESGPGSEPATSGSETPGTSESATPESGPGSPAGSPTSTEEGSPA | |
| | GSPTSTEEGTSTEPSEGSAPGTSESATPESGPGTSESATPESGPGTSESATPE | |
| | SGPGSEPATSGSETPGSEPATSGSETPGSPAGSPTSTEEGTSTEPSEGSAPGT | |
| | STEPSEGSAPGSEPATSGSETPGTSESAT | |
| AE648A | PESGPGTSTEPSEGSAPGTSESATPESGPGSEPATSGSETPGTSTEPSEGSAP | 698 |
| | GTSTEPSEGSAPGTSESATPESGPGTSESATPESGPGSPAGSPTSTEEGTSES | |
| | ATPESGPGSEPATSGSETPGTSESATPESGPGTSTEPSEGSAPGTSTEPSEGS | |
| | APGTSTEPSEGSAPGTSTEPSEGSAPGTSTEPSEGSAPGTSTEPSEGSAPGSP | |
| | AGSPTSTEEGTSTEPSEGSAPGTSESATPESGPGSEPATSGSETPGTSESATP | |
| | ESGPGSEPATSGSETPGTSESATPESGPGTSTEPSEGSAPGTSESATPESGPG | |
| | SPAGSPTSTEEGSPAGSPTSTEEGSPAGSPTSTEEGTSESATPESGPGTSTEP | |
| | SEGSAPGTSESATPESGPGSEPATSGSETPGTSESATPESGPGSEPATSGSET | |
| | PGTSESATPESGPGTSTEPSEGSAPGSPAGSPTSTEEGTSESATPESGPGSEP | |
| | ATSGSETPGTSESATPESGPGSPAGSPTSTEEGSPAGSPTSTEEGTSTEPSEG | |
| | SAPGTSESATPESGPGTSESATPESGPGTSESATPESGPGSEPATSGSETPGS | |
| | EPATSGSETPGSPAGSPTSTEEGTSTEPSEGSAPGTSTEPSEGSAPGSEPATS | |
| | GSETPGTSESAT | |
| AE684A | EGSAPGSPAGSPTSTEEGTSTEPSEGSAPGTSTEPSEGSAPGTSESATPESGP | 699 |
| | GTSTEPSEGSAPGTSESATPESGPGSEPATSGSETPGTSTEPSEGSAPGTSTE | |
| | PSEGSAPGTSESATPESGPGTSESATPESGPGSPAGSPTSTEEGTSESATPES | |
| | GPGSEPATSGSETPGTSESATPESGPGTSTEPSEGSAPGTSTEPSEGSAPGTS | |
| | TEPSEGSAPGTSTEPSEGSAPGTSTEPSEGSAPGTSTEPSEGSAPGSPAGSPT | |
| | STEEGTSTEPSEGSAPGTSESATPESGPGSEPATSGSETPGTSESATPESGPG | |
| | SEPATSGSETPGTSESATPESGPGTSTEPSEGSAPGTSESATPESGPGSPAGS | |
| | PTSTEEGSPAGSPTSTEEGSPAGSPTSTEEGTSESATPESGPGTSTEPSEGSA | |
| | PGTSESATPESGPGSEPATSGSETPGTSESATPESGPGSEPATSGSETPGTSE | |
| | SATPESGPGTSTEPSEGSAPGSPAGSPTSTEEGTSESATPESGPGSEPATSGS | |
| | ETPGTSESATPESGPGSPAGSPTSTEEGSPAGSPTSTEEGTSTEPSEGSAPGT | |
| | SESATPESGPGTSESATPESGPGTSESATPESGPGSEPATSGSETPGSEPATS | |
| | GSETPGSPAGSPTSTEEGTSTEPSEGSAPGTSTEPSEGSAPGSEPATS | |
| AE720A | TSGSETPGSEPATSGSETPGSPAGSPTSTEEGTSESATPESGPGTSTEPSEGS | 700 |
| | APGTSTEPSEGSAPGSPAGSPTSTEEGTSTEPSEGSAPGTSTEPSEGSAPGTS | |
| | ESATPESGPGTSTEPSEGSAPGTSESATPESGPGSEPATSGSETPGTSTEPSE | |
| | GSAPGTSTEPSEGSAPGTSESATPESGPGTSESATPESGPGSPAGSPTSTEEG | |
| | TSESATPESGPGSEPATSGSETPGTSESATPESGPGTSTEPSEGSAPGTSTEP | |
| | SEGSAPGTSTEPSEGSAPGTSTEPSEGSAPGTSTEPSEGSAPGTSTEPSEGSA | |
| | PGSPAGSPTSTEEGTSTEPSEGSAPGTSESATPESGPGSEPATSGSETPGTSE | |
| | SATPESGPGSEPATSGSETPGTSESATPESGPGTSTEPSEGSAPGTSESATPE | |
| | SGPGSPAGSPTSTEEGSPAGSPTSTEEGSPAGSPTSTEEGTSESATPESGPGT | |
| | STEPSEGSAPGTSESATPESGPGSEPATSGSETPGTSESATPESGPGSEPATS | |
| | GSETPGTSESATPESGPGTSTEPSEGSAPGSPAGSPTSTEEGTSESATPESGP | |
| | GSEPATSGSETPGTSESATPESGPGSPAGSPTSTEEGSPAGSPTSTEEGTSTE | |
| | PSEGSAPGTSESATPESGPGTSESATPESGPGTSESATPESGPGSEPATSGSE | |
| | TPGSEPATSGSETPGSPAGSPTSTEEGTSTE | |
| AE756A | TSGSETPGSEPATSGSETPGSPAGSPTSTEEGTSESATPESGPGTSTEPSEGS | 701 |
| | APGTSTEPSEGSAPGSPAGSPTSTEEGTSTEPSEGSAPGTSTEPSEGSAPGTS | |
| | ESATPESGPGTSTEPSEGSAPGTSESATPESGPGSEPATSGSETPGTSTEPSE | |
| | GSAPGTSTEPSEGSAPGTSESATPESGPGTSESATPESGPGSPAGSPTSTEEG | |
| | TSESATPESGPGSEPATSGSETPGTSESATPESGPGTSTEPSEGSAPGTSTEP | |
| | SEGSAPGTSTEPSEGSAPGTSTEPSEGSAPGTSTEPSEGSAPGTSTEPSEGSA | |
| | PGSPAGSPTSTEEGTSTEPSEGSAPGTSESATPESGPGSEPATSGSETPGTSE | |
| | SATPESGPGSEPATSGSETPGTSESATPESGPGTSTEPSEGSAPGTSESATPE | |
| | SGPGSPAGSPTSTEEGSPAGSPTSTEEGSPAGSPTSTEEGTSESATPESGPGT | |
| | STEPSEGSAPGTSESATPESGPGSEPATSGSETPGTSESATPESGPGSEPATS | |
| | GSETPGTSESATPESGPGTSTEPSEGSAPGSPAGSPTSTEEGTSESATPESGP | |
| | GSEPATSGSETPGTSESATPESGPGSPAGSPTSTEEGSPAGSPTSTEEGTSTE | |
| | PSEGSAPGTSESATPESGPGTSESATPESGPGTSESATPESGPGSEPATSGSE | |
| | TPGSEPATSGSETPGSPAGSPTSTEEGTSTEPSEGSAPGTSTEPSEGSAPGSE | |
| | PATSGSETPGTSES | |
| AE792A | EGSAPGTSESATPESGPGSEPATSGSETPGSEPATSGSETPGSPAGSPTSTEE | 702 |
| | GTSESATPESGPGTSTEPSEGSAPGTSTEPSEGSAPGSPAGSPTSTEEGTSTE | |
| | PSEGSAPGTSTEPSEGSAPGTSESATPESGPGTSTEPSEGSAPGTSESATPES | |
| | GPGSEPATSGSETPGTSTEPSEGSAPGTSTEPSEGSAPGTSESATPESGPGTS | |
| | ESATPESGPGSPAGSPTSTEEGTSESATPESGPGSEPATSGSETPGTSESATP | |
| | ESGPGTSTEPSEGSAPGTSTEPSEGSAPGTSTEPSEGSAPGTSTEPSEGSAPG | |
| | TSTEPSEGSAPGTSTEPSEGSAPGSPAGSPTSTEEGTSTEPSEGSAPGTSESA | |
| | TPESGPGSEPATSGSETPGTSESATPESGPGSEPATSGSETPGTSESATPESG | |
| | PGTSTEPSEGSAPGTSESATPESGPGSPAGSPTSTEEGSPAGSPTSTEEGSPA | |
| | GSPTSTEEGTSESATPESGPGTSTEPSEGSAPGTSESATPESGPGSEPATSGS | |
| | ETPGTSESATPESGPGSEPATSGSETPGTSESATPESGPGTSTEPSEGSAPGS | |
| | PAGSPTSTEEGTSESATPESGPGSEPATSGSETPGTSESATPESGPGSPAGSP | |
| | TSTEEGSPAGSPTSTEEGTSTEPSEGSAPGTSESATPESGPGTSESATPESGP | |
| | GTSESATPESGPGSEPATSGSETPGSEPATSGSETPGSPAGSPTSTEEGTSTE | |
| | PSEGSAPGTSTEPSEGSAPGSEPATSGSETPGTSESATPESGPGTSTEPS | |
| AE828A | PESGPGTSTEPSEGSAPGSPAGSPTSTEEGTSTEPSEGSAPGTSTEPSEGSAP | 703 |
| | GTSESATPESGPGSEPATSGSETPGSEPATSGSETPGSPAGSPTSTEEGTSES | |
| | ATPESGPGTSTEPSEGSAPGTSTEPSEGSAPGSPAGSPTSTEEGTSTEPSEGS | |
| | APGTSTEPSEGSAPGTSESATPESGPGTSTEPSEGSAPGTSESATPESGPGSE | |
| | PATSGSETPGTSTEPSEGSAPGTSTEPSEGSAPGTSESATPESGPGTSESATP | |
| | ESGPGSPAGSPTSTEEGTSESATPESGPGSEPATSGSETPGTSESATPESGPG | |
| | TSTEPSEGSAPGTSTEPSEGSAPGTSTEPSEGSAPGTSTEPSEGSAPGTSTEP | |
| | SEGSAPGTSTEPSEGSAPGSPAGSPTSTEEGTSTEPSEGSAPGTSESATPESG | |
| | PGSEPATSGSETPGTSESATPESGPGSEPATSGSETPGTSESATPESGPGTST | |
| | EPSEGSAPGTSESATPESGPGSPAGSPTSTEEGSPAGSPTSTEEGSPAGSPTS | |
| | TEEGTSESATPESGPGTSTEPSEGSAPGTSESATPESGPGSEPATSGSETPGT | |
| | SESATPESGPGSEPATSGSETPGTSESATPESGPGTSTEPSEGSAPGSPAGSP | |
| | TSTEEGTSESATPESGPGSEPATSGSETPGTSESATPESGPGSPAGSPTSTEE | |
| | GSPAGSPTSTEEGTSTEPSEGSAPGTSESATPESGPGTSESATPESGPGTSES | |
| | ATPESGPGSEPATSGSETPGSEPATSGSETPGSPAGSPTSTEEGTSTEPSEGS | |
| | APGTSTEPSEGSAPGSEPATSGSETPGTSESAT | |
| AE869 | GSPGSPAGSPTSTEEGTSESATPESGPGTSTEPSEGSAPGSPAGSPTSTEEGT | 704 |
| | STEPSEGSAPGTSTEPSEGSAPGTSESATPESGPGSEPATSGSETPGSEPATS | |
| | GSETPGSPAGSPTSTEEGTSESATPESGPGTSTEPSEGSAPGTSTEPSEGSAP | |
| | GSPAGSPTSTEEGTSTEPSEGSAPGTSTEPSEGSAPGTSESATPESGPGTSTE | |
| | PSEGSAPGTSESATPESGPGSEPATSGSETPGTSTEPSEGSAPGTSTEPSEGS | |
| | APGTSESATPESGPGTSESATPESGPGSPAGSPTSTEEGTSESATPESGPGSE | |
| | PATSGSETPGTSESATPESGPGTSTEPSEGSAPGTSTEPSEGSAPGTSTEPSE | |
| | GSAPGTSTEPSEGSAPGTSTEPSEGSAPGTSTEPSEGSAPGSPAGSPTSTEEG | |
| | TSTEPSEGSAPGTSESATPESGPGSEPATSGSETPGTSESATPESGPGSEPAT | |
| | SGSETPGTSESATPESGPGTSTEPSEGSAPGTSESATPESGPGSPAGSPTSTE | |
| | EGSPAGSPTSTEEGSPAGSPTSTEEGTSESATPESGPGTSTEPSEGSAPGTSE | |
| | SATPESGPGSEPATSGSETPGTSESATPESGPGSEPATSGSETPGTSESATPE | |
| | SGPGTSTEPSEGSAPGSPAGSPTSTEEGTSESATPESGPGSEPATSGSETPGT | |
| | SESATPESGPGSPAGSPTSTEEGSPAGSPTSTEEGTSTEPSEGSAPGTSESAT | |
| | PESGPGTSESATPESGPGTSESATPESGPGSEPATSGSETPGSEPATSGSETP | |
| | GSPAGSPTSTEEGTSTEPSEGSAPGTSTEPSEGSAPGSEPATSGSETPGTSES | |
| | ATPESGPGTSTEPSEGSAPGR | |
| AE144_R1 | SAGSPGSPAGSPTSTEEGTSESATPESGPGTSTEPSEGSAPGSPAGSPTSTEE | 705 |
| | GTSTEPSEGSAPGTSTEPSEGSAPGTSESATPESGPGSEPATSGSETPGSEPA | |
| | TSGSETPGSPAGSPTSTEEGTSESATPESGPGTESASR | |
| AE288_R1 | SAGSPTGPGSEPATSGSETPGTSESATPESGPGSEPATSGSETPGTSESATPE | 706 |
| | SGPGTSTEPSEGSAPGSPAGSPTSTEEGTSESATPESGPGSEPATSGSETPGT | |
| | SESATPESGPGSPAGSPTSTEEGSPAGSPTSTEEGTSTEPSEGSAPGTSESAT | |
| | PESGPGTSESATPESGPGTSESATPESGPGSEPATSGSETPGSEPATSGSETP | |
| | GSPAGSPTSTEEGTSTEPSEGSAPGTSTEPSEGSAPGSEPATSGSETPGTSES | |
| | ATPESGPGTSTEPSEGSAPSASR | |
| AE432_R1 | SAGSPGSPAGSPTSTEEGTSESATPESGPGTSTEPSEGSAPGSPAGSPTSTEE | 707 |
| | GTSTEPSEGSAPGTSTEPSEGSAPGTSESATPESGPGSEPATSGSETPGSEPA | |
| | TSGSETPGSPAGSPTSTEEGTSESATPESGPGTSTEPSEGSAPGTSTEPSEGS | |
| | APGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPSE | |
| | GSAPGTSESATPESGPGTSESATPESGPGSPAGSPTSTEEGTSESATPESGPG | |
| | SEPATSGSETPGTSESATPESGPGTSTEPSEGSAPGTSTEPSEGSAPGTSTEP | |
| | SEGSAPGTSTEPSEGSAPGTSTEPSEGSAPGTSTEPSEGSAPGSPAGSPTSTE | |
| | EGTESASR | |
| AE576_R1 | SAGSPTEEGTSESATPESGPGSEPATSGSETPGTSESATPESGPGTSTEPSEG | 708 |
| | SAPGTSTEPSEGSAPGTSTEPSEGSAPGTSTEPSEGSAPGTSTEPSEGSAPGT | |
| | STEPSEGSAPGSPAGSPTSTEEGTSTEPSEGSAPGTSESATPESGPGSEPATS | |
| | GSETPGTSESATPESGPGSEPATSGSETPGTSESATPESGPGTSTEPSEGSAP | |
| | GTSESATPESGPGSPAGSPTSTEEGSPAGSPTSTEEGSPAGSPTSTEEGTSES | |
| | ATPESGPGTSTEPSEGSAPGTSESATPESGPGSEPATSGSETPGTSESATPES | |
| | GPGSEPATSGSETPGTSESATPESGPGTSTEPSEGSAPGSPAGSPTSTEEGTS | |
| | ESATPESGPGSEPATSGSETPGTSESATPESGPGSPAGSPTSTEEGSPAGSPT | |
| | STEEGTSTEPSEGSAPGTSESATPESGPGTSESATPESGPGTSESATPESGPG | |
| | SEPATSGSETPGSEPATSGSETPGSPAGSPTSTEEGTSTEPSEGSAPGTSTEP | |
| | SEGSAPGSEPATSGSETPGTSESATPESGPGTSTEPSEGSAPSASR | |
| AE864_R1 | SAGSPGSPAGSPTSTEEGTSESATPESGPGTSTEPSEGSAPGSPAGSPTSTEE | 709 |
| | GTSTEPSEGSAPGTSTEPSEGSAPGTSESATPESGPGSEPATSGSETPGSEPA | |
| | TSGSETPGSPAGSPTSTEEGTSESATPESGPGTSTEPSEGSAPGTSTEPSEGS | |
| | APGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPSE | |
| | GSAPGTSESATPESGPGTSESATPESGPGSPAGSPTSTEEGTSESATPESGPG | |
| | SEPATSGSETPGTSESATPESGPGTSTEPSEGSAPGTSTEPSEGSAPGTSTEP | |
| | SEGSAPGTSTEPSEGSAPGTSTEPSEGSAPGTSTEPSEGSAPGSPAGSPTSTE | |
| | EGTSTEPSEGSAPGTSESATPESGPGSEPATSGSETPGTSESATPESGPGSEP | |
| | ATSGSETPGTSESATPESGPGTSTEPSEGSAPGTSESATPESGPGSPAGSPTS | |
| | TEEGSPAGSPTSTEEGSPAGSPTSTEEGTSESATPESGPGTSTEPSEGSAPGT | |
| | SESATPESGPGSEPATSGSETPGTSESATPESGPGSEPATSGSETPGTSESAT | |
| | PESGPGTSTEPSEGSAPGSPAGSPTSTEEGTSESATPESGPGSEPATSGSETP | |
| | GTSESATPESGPGSPAGSPTSTEEGSPAGSPTSTEEGTSTEPSEGSAPGTSES | |
| | ATPESGPGTSESATPESGPGTSESATPESGPGSEPATSGSETPGSEPATSGSE | |
| | TPGSPAGSPTSTEEGTSTEPSEGSAPGTSTEPSEGSAPGSEPATSGSETPGTS | |
| | ESATPESGPGTESASR | |
| AE712 | PGSPAGSPTSTEEGTSESATPESGPGTSTEPSEGSAPGSPAGSPTSTEEGTST | 710 |
| | EPSEGSAPGTSTEPSEGSAPGTSESATPESGPGSEPATSGSETPGSEPATSGS | |
| | ETPGSPAGSPTSTEEGTSESATPESGPGTSTEPSEGSAPGTSTEPSEGSAPGS | |
| | PAGSPTSTEEGTSTEPSEGSAPGTSTEPSEGSAPGTSESATPESGPGTSTEPS | |
| | EGSAPGTSESATPESGPGSEPATSGSETPGTSTEPSEGSAPGTSTEPSEGSAP | |
| | GTSESATPESGPGTSESATPESGPGSPAGSPTSTEEGTSESATPESGPGSEPA | |
| | TSGSETPGTSESATPESGPGTSTEPSEGSAPGTSTEPSEGSAPGTSTEPSEGS | |
| | APGTSTEPSEGSAPGTSTEPSEGSAPGTSTEPSEGSAPGSPAGSPTSTEEGTS | |
| | TEPSEGSAPGTSESATPESGPGSEPATSGSETPGTSESATPESGPGSEPATSG | |
| | SETPGTSESATPESGPGTSTEPSEGSAPGTSESATPESGPGSPAGSPTSTEEG | |
| | SPAGSPTSTEEGSPAGSPTSTEEGTSESATPESGPGTSTEPSEGSAPGTSESA | |
| | TPESGPGSEPATSGSETPGTSESATPESGPGSEPATSGSETPGTSESATPESG | |
| | PGTSTEPSEGSAPGSPAGSPTSTEEGTSESATPESGPGSEPATSGSETPGTSE | |
| | SATPESGPGSPAGSPTSTEAHHH | |
| AE864_R2 | GSPGAGSPAGSPTSTEEGTSESATPESGPGTSTEPSEGSAPGSPAGSPTSTEE | 711 |
| | GTSTEPSEGSAPGTSTEPSEGSAPGTSESATPESGPGSEPATSGSETPGSEPA | |
| | TSGSETPGSPAGSPTSTEEGTSESATPESGPGTSTEPSEGSAPGTSTEPSEGS | |
| | APGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPSE | |
| | GSAPGTSESATPESGPGTSESATPESGPGSPAGSPTSTEEGTSESATPESGPG | |
| | SEPATSGSETPGTSESATPESGPGTSTEPSEGSAPGTSTEPSEGSAPGTSTEP | |
| | SEGSAPGTSTEPSEGSAPGTSTEPSEGSAPGTSTEPSEGSAPGSPAGSPTSTE | |
| | EGTSTEPSEGSAPGTSESATPESGPGSEPATSGSETPGTSESATPESGPGSEP | |
| | ATSGSETPGTSESATPESGPGTSTEPSEGSAPGTSESATPESGPGSPAGSPTS | |
| | TEEGSPAGSPTSTEEGSPAGSPTSTEEGTSESATPESGPGTSTEPSEGSAPGT | |
| | SESATPESGPGSEPATSGSETPGTSESATPESGPGSEPATSGSETPGTSESAT | |
| | PESGPGTSTEPSEGSAPGSPAGSPTSTEEGTSESATPESGPGSEPATSGSETP | |
| | GTSESATPESGPGSPAGSPTSTEEGSPAGSPTSTEEGTSTEPSEGSAPGTSES | |
| | ATPESGPGTSESATPESGPGTSESATPESGPGSEPATSGSETPGSEPATSGSE | |
| | TPGSPAGSPTSTEEGTSTEPSEGSAPGTSTEPSEGSAPGSEPATSGSETPGTS | |
| | ESATPESGPGTESASR | |
| AE288_3 | SPAGSPTSTEEGTSESATPESGPGTSTEPSEGSAPGTSESATPESGPGSEPAT | 712 |
| | SGSETPGTSESATPESGPGSEPATSGSETPGTSESATPESGPGTSTEPSEGSA | |
| | PGSPAGSPTSTEEGTSESATPESGPGSEPATSGSETPGTSESATPESGPGSPA | |
| | GSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGT | |
| | STEPSEGSAPGTSTEPSEGSAPG | |
| AE284 | GTSESATPESGPGSEPATSGSETPGTSESATPESGPGSEPATSGSETPGTSES | 713 |
| | ATPESGPGTSTEPSEGSAPGSPAGSPTSTEEGTSESATPESGPGSEPATSGSE | |
| | TPGTSESATPESGPGSPAGSPTSTEEGSPAGSPTSTEEGTSTEPSEGSAPGTS | |
| | ESATPESGPGTSESATPESGPGTSESATPESGPGSEPATSGSETPGSEPATSG | |
| | SETPGSPAGSPTSTEEGTSTEPSEGSAPGTSTEPSEGSAPGSEPATSGSETPG | |
| | TSESATPESGPGTSTEPSE | |
| AE292 | SPAGSPTSTEEGTSESATPESGPGTSTEPSEGSAPGTSESATPESGPGSEPAT | 714 |
| | SGSETPGTSESATPESGPGSEPATSGSETPGTSESATPESGPGTSTEPSEGSA | |
| | PGSPAGSPTSTEEGTSESATPESGPGSEPATSGSETPGTSESATPESGPGSPA | |
| | GSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGT | |
| | STEPSEGSAPGTSTEPSEGSAPGGSAP | |
| AE864_2 | AGSPTSTEEGTSESATPESGPGTSTEPSEGSAPGSPAGSPTSTEEGTSTEPSE | 715 |
| | GSAPGTSTEPSEGSAPGTSESATPESGPGSEPATSGSETPGSEPATSGSETPG | |
| | SPAGSPTSTEEGTSESATPESGPGTSTEPSEGSAPGTSTEPSEGSAPGSPAGS | |
| | PTSTEEGTSTEPSEGSAPGTSTEPSEGSAPGTSESATPESGPGTSTEPSEGSA | |
| | PGTSESATPESGPGSEPATSGSETPGTSTEPSEGSAPGTSTEPSEGSAPGTSE | |
| | SATPESGPGTSESATPESGPGSPAGSPTSTEEGTSESATPESGPGSEPATSGS | |
| | ETPGTSESATPESGPGTSTEPSEGSAPGTSTEPSEGSAPGTSTEPSEGSAPGT | |
| | STEPSEGSAPGTSTEPSEGSAPGTSTEPSEGSAPGSPAGSPTSTEEGTSTEPS | |
| | EGSAPGTSESATPESGPGSEPATSGSETPGTSESATPESGPGSEPATSGSETP | |
| | GTSESATPESGPGTSTEPSEGSAPGTSESATPESGPGSPAGSPTSTEEGSPAG | |
| | SPTSTEEGSPAGSPTSTEEGTSESATPESGPGTSTEPSEGSAPGTSESATPES | |
| | GPGSEPATSGSETPGTSESATPESGPGSEPATSGSETPGTSESATPESGPGTS | |
| | TEPSEGSAPGSPAGSPTSTEEGTSESATPESGPGSEPATSGSETPGTSESATP | |
| | ESGPGSPAGSPTSTEEGSPAGSPTSTEEGTSTEPSEGSAPGTSESATPESGPG | |
| | TSESATPESGPGTSESATPESGPGSEPATSGSETPGSEPATSGSETPGSPAGS | |
| | PTSTEEGTSTEPSEGSAPGTSTEPSEGSAPGSEPATSGSETPGTSESATPESG | |
| | PGTSTEPSEGAAEPEA | |
| AE867 | GSPAGSPTSTEEGTSESATPESGPGTSTEPSEGSAPGSPAGSPTSTEEGTSTE | 716 |
| | PSEGSAPGTSTEPSEGSAPGTSESATPESGPGSEPATSGSETPGSEPATSGSE | |
| | TPGSPAGSPTSTEEGTSESATPESGPGTSTEPSEGSAPGTSTEPSEGSAPGSP | |
| | AGSPTSTEEGTSTEPSEGSAPGTSTEPSEGSAPGTSESATPESGPGTSTEPSE | |
| | GSAPGTSESATPESGPGSEPATSGSETPGTSTEPSEGSAPGTSTEPSEGSAPG | |
| | TSESATPESGPGTSESATPESGPGSPAGSPTSTEEGTSESATPESGPGSEPAT | |
| | SGSETPGTSESATPESGPGTSTEPSEGSAPGTSTEPSEGSAPGTSTEPSEGSA | |
| | PGTSTEPSEGSAPGTSTEPSEGSAPGTSTEPSEGSAPGSPAGSPTSTEEGTST | |
| | EPSEGSAPGTSESATPESGPGSEPATSGSETPGTSESATPESGPGSEPATSGS | |
| | ETPGTSESATPESGPGTSTEPSEGSAPGTSESATPESGPGSPAGSPTSTEEGS | |
| | PAGSPTSTEEGSPAGSPTSTEEGTSESATPESGPGTSTEPSEGSAPGTSESAT | |
| | PESGPGSEPATSGSETPGTSESATPESGPGSEPATSGSETPGTSESATPESGP | |
| | GTSTEPSEGSAPGSPAGSPTSTEEGTSESATPESGPGSEPATSGSETPGTSES | |
| | ATPESGPGSPAGSPTSTEEGSPAGSPTSTEEGTSTEPSEGSAPGTSESATPES | |
| | GPGTSESATPESGPGTSESATPESGPGSEPATSGSETPGSEPATSGSETPGSP | |
| | AGSPTSTEEGTSTEPSEGSAPGTSTEPSEGSAPGSEPATSGSETPGTSESATP | |
| | ESGPGTSTEPSEGAAEPEA | |
| AE867_2 | SPGSPAGSPTSTEEGTSESATPESGPGTSTEPSEGSAPGSPAGSPTSTEEGTS | 717 |
| | TEPSEGSAPGTSTEPSEGSAPGTSESATPESGPGSEPATSGSETPGSEPATSG | |
| | SETPGSPAGSPTSTEEGTSESATPESGPGTSTEPSEGSAPGTSTEPSEGSAPG | |
| | SPAGSPTSTEEGTSTEPSEGSAPGTSTEPSEGSAPGTSESATPESGPGTSTEP | |
| | SEGSAPGTSESATPESGPGSEPATSGSETPGTSTEPSEGSAPGTSTEPSEGSA | |
| | PGTSESATPESGPGTSESATPESGPGSPAGSPTSTEEGTSESATPESGPGSEP | |
| | ATSGSETPGTSESATPESGPGTSTEPSEGSAPGTSTEPSEGSAPGTSTEPSEG | |
| | SAPGTSTEPSEGSAPGTSTEPSEGSAPGTSTEPSEGSAPGSPAGSPTSTEEGT | |
| | STEPSEGSAPGTSESATPESGPGSEPATSGSETPGTSESATPESGPGSEPATS | |
| | GSETPGTSESATPESGPGTSTEPSEGSAPGTSESATPESGPGSPAGSPTSTEE | |
| | GSPAGSPTSTEEGSPAGSPTSTEEGTSESATPESGPGTSTEPSEGSAPGTSES | |
| | ATPESGPGSEPATSGSETPGTSESATPESGPGSEPATSGSETPGTSESATPES | |
| | GPGTSTEPSEGSAPGSPAGSPTSTEEGTSESATPESGPGSEPATSGSETPGTS | |
| | ESATPESGPGSPAGSPTSTEEGSPAGSPTSTEEGTSTEPSEGSAPGTSESATP | |
| | ESGPGTSESATPESGPGTSESATPESGPGSEPATSGSETPGSEPATSGSETPG | |
| | SPAGSPTSTEEGTSTEPSEGSAPGTSTEPSEGSAPGSEPATSGSETPGTSESA | |
| | TPESGPGTSTEPSEGSAPG | |
| | PGSPAGSPTSTEEGTSESATPESGPGTSTEPSEGSAPGSPAGSPTSTEEGTST | 718 |
| | ETPGSPAGSPTSTEEGTSESATPESGPGTSTEPSEGSAPGTSTEPSEGSAPGS | |
| | PAGSPTSTEEGTSTEPSEGSAPGTSTEPSEGSAPGTSESATPESGPGTSTEPS | |
| | EGSAPGTSESATPESGPGSEPATSGSETPGTSTEPSEGSAPGTSTEPSEGSAP | |
| | GTSESATPESGPGTSESATPESGPGSPAGSPTSTEEGTSESATPESGPGSEPA | |
| | TSGSETPGTSESATPESGPGTSTEPSEGSAPGTSTEPSEGSAPGTSTEPSEGS | |
| | APGTSTEPSEGSAPGTSTEPSEGSAPGTSTEPSEGSAPGSPAGSPTSTEEGTS | |
| | TEPSEGSAPGTSESATPESGPGSEPATSGSETPGTSESATPESGPGSEPATSG | |
| | SETPGTSESATPESGPGTSTEPSEGSAPGTSESATPESGPGSPAGSPTSTEEG | |
| | SPAGSPTSTEEGSPAGSPTSTEEGTSESATPESGPGTSTEPSEGSAPGTSESA | |
| | TPESGPGSEPATSGSETPGTSESATPESGPGSEPATSGSETPGTSESATPESG | |
| | PGTSTEPSEGSAPGSPAGSPTSTEEGTSESATPESGPGSEPATSGSETPGTSE | |
| | SATPESGPGSPAGSPTSTEEGSPAGSPTSTEEGTSTEPSEGSAPGTSESATPE | |
| | SGPGTSESATPESGPGTSESATPESGPGSEPATSGSETPGSEPATSGSETPGS | |
| | PAGSPTSTEEGTSTEPSEGSAPGTSTEPSEGSAPGSEPATSGSETPGTSESAT | |
| | PESGPGTSTEPSEGAAEPEA | |
| AE144_7A | GSPAGSPTSTEEGTSESATPESGPGTSTEPSEGSAPGSPAGSPTSTEEGTSTE | 922 |
| | PSEGSAPGTSTEPSEGSAPGTSESATPESGPGSEPATSGSETPGSEPATSGSE | |
| | TPGSPAGSPTSTEEGTSESATPESGPGTSTEPSEGSAP | |
| AE292 | SPAGSPTSTEEGTSESATPESGPGTSTEPSEGSAPGTSESATPESGPGSEPAT | 923 |
| | SGSETPGTSESATPESGPGSEPATSGSETPGTSESATPESGPGTSTEPSEGSA | |
| | PGSPAGSPTSTEEGTSESATPESGPGSEPATSGSETPGTSESATPESGPGSPA | |
| | GSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGT | |
| | STEPSEGSAPGTSTEPSEGSAPGGSAP | |
| AE293 | PGSPAGSPTSTEEGTSESATPESGPGTSTEPSEGSAPGSPAGSPTSTEEGTST | 924 |
| | EPSEGSAPGTSTEPSEGSAPGTSESATPESGPGSEPATSGSETPGSEPATSGS | |
| | ETPGSPAGSPTSTEEGTSESATPESGPGTSTEPSEGSAPGTSTEPSEGSAPGS | |
| | PAGSPTSTEEGTSTEPSEGSAPGTSTEPSEGSAPGTSESATPESGPGTSTEPS | |
| | EGSAPGTSESATPESGPGSEPATSGSETPGTSTEPSEGSAPGTSTEPSEGSAP | |
| | GTSESATPESGPGTSESATPEGAAEPEA | |
| AE300 | PGSPAGSPTSTEEGTSESATPESGPGTSTEPSEGSAPGSPAGSPTSTEEGTST | 925 |
| | EPSEGSAPGTSTEPSEGSAPGTSESATPESGPGSEPATSGSETPGSEPATSGS | |
| | ETPGSPAGSPTSTEEGTSESATPESGPGTSTEPSEGSAPGTSTEPSEGSAPGS | |
| | PAGSPTSTEEGTSTEPSEGSAPGTSTEPSEGSAPGTSESATPESGPGTSTEPS | |
| | EGSAPGTSESATPESGPGSEPATSGSETPGTSTEPSEGSAPGTSTEPSEGSAP | |
| | GTSESATPESGPGTSESATPESGPGSPAGAAEPEA | |
| AE584 | PGSPAGSPTSTEEGTSESATPESGPGTSTEPSEGSAPGSPAGSPTSTEEGTST | 926 |
| | EPSEGSAPGTSTEPSEGSAPGTSESATPESGPGSEPATSGSETPGSEPATSGS | |
| | ETPGSPAGSPTSTEEGTSESATPESGPGTSTEPSEGSAPGTSTEPSEGSAPGS | |
| | PAGSPTSTEEGTSTEPSEGSAPGTSTEPSEGSAPGTSESATPESGPGTSTEPS | |
| | EGSAPGTSESATPESGPGSEPATSGSETPGTSTEPSEGSAPGTSTEPSEGSAP | |
| | GTSESATPESGPGTSESATPESGPGSPAGSPTSTEEGTSESATPESGPGSEPA | |
| | TSGSETPGTSESATPESGPGTSTEPSEGSAPGTSTEPSEGSAPGTSTEPSEGS | |
| | APGTSTEPSEGSAPGTSTEPSEGSAPGTSTEPSEGSAPGSPAGSPTSTEEGTS | |
| | TEPSEGSAPGTSESATPESGPGSEPATSGSETPGTSESATPESGPGSEPATSG | |
| | SETPGTSESATPESGPGTSTEPSEGSAPGTSESATPESGPGSPAGSPTSTEEG | |
| | SPAGSPTSTEEGSPAGSPTSTEEGTSESATPESGPGTSTEPSEGSAPGAAEPE | |
| | A | |
| AE870 | PGSPAGSPTSTEEGTSESATPESGPGTSTEPSEGSAPGSPAGSPTSTEEGTST | 927 |
| | EPSEGSAPGTSTEPSEGSAPGTSESATPESGPGSEPATSGSETPGSEPATSGS | |
| | ETPGSPAGSPTSTEEGTSESATPESGPGTSTEPSEGSAPGTSTEPSEGSAPGS | |
| | PAGSPTSTEEGTSTEPSEGSAPGTSTEPSEGSAPGTSESATPESGPGTSTEPS | |
| | EGSAPGTSESATPESGPGSEPATSGSETPGTSTEPSEGSAPGTSTEPSEGSAP | |
| | GTSESATPESGPGTSESATPESGPGSPAGSPTSTEEGTSESATPESGPGSEPA | |
| | TSGSETPGTSESATPESGPGTSTEPSEGSAPGTSTEPSEGSAPGTSTEPSEGS | |
| | APGTSTEPSEGSAPGTSTEPSEGSAPGTSTEPSEGSAPGSPAGSPTSTEEGTS | |
| | TEPSEGSAPGTSESATPESGPGSEPATSGSETPGTSESATPESGPGSEPATSG | |
| | SETPGTSESATPESGPGTSTEPSEGSAPGTSESATPESGPGSPAGSPTSTEEG | |
| | SPAGSPTSTEEGSPAGSPTSTEEGTSESATPESGPGTSTEPSEGSAPGTSESA | |
| | TPESGPGSEPATSGSETPGTSESATPESGPGSEPATSGSETPGTSESATPESG | |
| | PGTSTEPSEGSAPGSPAGSPTSTEEGTSESATPESGPGSEPATSGSETPGTSE | |
| | SATPESGPGSPAGSPTSTEEGSPAGSPTSTEEGTSTEPSEGSAPGTSESATPE | |
| | SGPGTSESATPESGPGTSESATPESGPGSEPATSGSETPGSEPATSGSETPGS | |
| | PAGSPTSTEEGTSTEPSEGSAPGTSTEPSEGSAPGSEPATSGSETPGTSESAT | |
| | PESGPGTSTEPSEGSAPGEPEA | |

The disclosure contemplates compositions of any of the embodiments described herein comprising XTEN of intermediate lengths to those of Table 7, as well as XTEN of longer lengths than those of Table 7, such as those in which motifs of 12 amino acids of Table 6 are added to the N- or C-terminus of an XTEN of Table 7.

In another embodiment, the disclosure contemplates polypeptide compositions of any of the embodiments described herein comprising an XTEN1 and an XTEN2 that can further comprise a His tag of HHHHHH (SEQ ID NO: 1150) or HHHHHHHH (SEQ ID NO: 1151) at the N-terminus and/or the sequence EPEA (SEQ ID NO: 1149) at the C-terminus, respectively, of the polypeptide composition to facilitate the purification of the composition to at least 90%, 91%, 92%, 93%, 94%, 95%, 96%, 97%, 98%, or at least 99% purity by chromatography methods known in the art, including but not limited to IMAC chromatography, C-tagXL affinity matrix, and other such methods, including but not limited to those described in the Examples, below.

Additional examples of XTEN sequences that can be used according to the present disclosure and are disclosed in US Patent Publication Nos. 2010/0239554 A1, 2010/0323956 A1, 2011/0046060 A1, 2011/0046061 A1, 2011/0077199 A1, or 2011/0172146 A1, or International Patent Publication Nos. WO 2010091122 A1, WO 2010144502 A2, WO 2010144508 A1, WO 2011028228 A1, WO 2011028229 A1, WO 2011028344 A2, WO 2014/011819 A2, or WO 2015/023891.

### V). Target Cell Marker Antigen Binding Fragments

In another aspect, the present disclosure relates to antigen binding fragments that have specific binding affinity for target cell marker antigens other than CD3 that can be incorporated into any of the subject composition embodiments described herein. The resulting bispecific compositions—having a first antigen binding fragment (AF1) with binding affinity to CD3 linked to a second antigen binding fragment (AF2) with binding affinity to a second non-CD3 antigen by a short, flexible peptide linker—are bispecific, with each antigen binding fragment having specific binding affinity to their respective ligands. It will be understood that in such compositions, an antigen binding fragment directed against a target cell marker of a disease tissue is used in combination with a second antigen binding fragment directed towards an effector cell marker in order to bring an effector cell in close proximity to the cell of a disease tissue in order to effect the cytolysis of the cell of the diseased tissue. Further, the AF1 and AF2 can be incorporated into the specifically designed polypeptides comprising cleavable release segments and XTEN in order to confer prodrug characteristics on the compositions that becomes activated by release of the fused AF1 and AF2 upon the cleavage of the release segments when in proximity to the disease tissue having proteases capable of cleaving the release segments in one or more locations in the release segment sequence.

In one embodiment, the polypeptides of any of the subject composition embodiments described herein can comprise an AF2 having specific binding affinity for a target cell marker expressed on a cell surface, in the cytoplasmic membrane, or within a target cell associated with cancers, autoimmune diseases, inflammatory diseases and other conditions where localized activation of the polypeptide is desirable. In one embodiment, the antigens against which the AF2 has specific binding affinity are selected from antigens that include, but are not limited to, 1-40-β-amyloid, 4-1BB, 5AC, 5T4, 707-AP, A kinase anchor protein 4 (AKAP-4), activin receptor type-2B (ACVR2B), activin receptor-like kinase 1 (ALK1), adenocarcinoma antigen, adipophilin, adrenoceptor β3 (ADRB3), AGS-22M6, α folate receptor, α-fetoprotein (AFP), AIM-2, anaplastic lymphoma kinase (ALK), androgen receptor, angiopoietin 2, angiopoietin 3, angiopoietin-binding cell surface receptor 2 (Tie 2), anthrax toxin, AOC3 (VAP-1), B cell maturation antigen (BCMA), B7-H3 (CD276), Bacillus anthracis anthrax, B-cell activating factor (BAFF), B-lymphoma cell, bone marrow stromal cell antigen 2 (BST2), Brother of the Regulator of Imprinted Sites (BORIS), C242 antigen, C5, CA-125, cancer antigen 125 (CA-125 or MUC16), Cancer/testis antigen 1 (NY-ESO-1), Cancer/testis antigen 2 (LAGE-1a), carbonic anhydrase 9 (CA-IX), Carcinoembryonic antigen (CEA), cardiac myosin, CCCTC-Binding Factor (CTCF), CCL11 (eotaxin-1), CCR4, CCR5, CCR6, CCR7, CCR8, CCR9, CD11, CD123, CD125, CD140a, CD147 (basigin), CD15, CD152, CD154 (CD40L), CD171, CD179a, CD18, CD19, CD2, CD20, CD200, CD22, CD221, CD23 (IgE receptor), CD24, CD25 (α chain of IL-2 receptor), CD27, CD274, CD28, CD3, CD3 ε, CD30, CD300 molecule-like family member f (CD300LF), CD319 (SLAMF7), CD33, CD37, CD38, CD4, CD40, CD40 ligand, CD41, CD44 v7, CD44 v8, CD44 v6, CD5, CD51, CD52, CD56, CD6, CD70, CD72, CD74, CD79A, CD79B, CD80, CD97, CEA-related antigen, CFD, ch4D5, chromosome X open reading frame 61 (CXORF61), claudin 18.2 (CLDN18.2), claudin 6 (CLDN6), Clostridium difficile, clumping factor A, CLCA2, colony stimulating factor 1 receptor (CSF1R), CSF2, CTLA-4, C-type lectin domain family 12 member A (CLEC12A), C-type lectin-like molecule-1 (CLL-1 or CLECL1), C-X-C chemokine receptor type 4, cyclin B1, cytochrome P4501B1 (CYP1B1), cyp-B, cytomegalovirus, cytomegalovirus glycoprotein B, dabigatran, DLL4, DPP4, DR5, E. coli shiga toxin type-1, E. coli shiga toxin type-2, ecto-ADP-ribosyltransferase 4 (ART4), EGF-like module-containing mucin-like hormone receptor-like 2 (EMR2), EGF-like-domain multiple 7 (EGFL7), elongation factor 2 mutated (ELF2M), endotoxin, Ephrin A2, Ephrin B2, ephrin type-A receptor 2, epidermal growth factor receptor (EGFR), epidermal growth factor receptor variant III (EGFRvIII), episialin, epithelial cell adhesion molecule (EpCAM), epithelial glycoprotein 2 (EGP-2), epithelial glycoprotein 40 (EGP-40), ERBB2, ERBB3, ERBB4, ERG (transmembrane protease, serine 2 (TMPRSS2) ETS fusion gene), Escherichia coli, ETS translocation-variant gene 6, located on chromosome 12p (ETV6-AML), F protein of respiratory syncytial virus, FAP, Fc fragment of IgA receptor (FCAR or CD89), Fc receptor-like 5 (FCRLS), fetal acetylcholine receptor, fibrin II β chain, fibroblast activation protein α (FAP), fibronectin extra domain-B, FGF-5, Fms-Like Tyrosine Kinase 3 (FLT3), folate binding protein (FBP), folate hydrolase, folate receptor 1, folate receptor α, folate receptor β, Fos-related antigen 1, Frizzled receptor, Fucosyl GM1, G250, G protein-coupled receptor 20 (GPR20), G protein-coupled receptor class C group 5, member D (GPRC5D), ganglioside G2 (GD2), GD3 ganglioside, glycoprotein 100 (gp100), glypican-3 (GPC3), GMCSF receptor α-chain, GPNMB, GnT-V, growth differentiation factor 8, GUCY2C, heat shock protein 70-2 mutated (mut hsp70-2), hemagglutinin, Hepatitis A virus cellular receptor 1 (HAVCR1), hepatitis B surface antigen, hepatitis B virus, HER1, HER2/neu, HER3, hexasaccharide portion of globoH glycoceramide (GloboH), HGF, HHGFR, high molecular weight-melanoma-associated antigen (HMW-MAA), histone complex, HIV-1, HLA-DR, HNGF, Hsp90, HST-2 (FGF6), human papilloma virus E6 (HPV E6), human papilloma virus E7 (HPV E7), human scatter factor receptor kinase, human Telomerase reverse transcriptase (hTERT), human TNF, ICAM-1 (CD54), iCE, IFN-α, IFN-β, IFN-γ, IgE, IgE Fc region, IGF-1, IGF-1 receptor, IGHE, IL-12, IL-13, IL-17, IL-17A, IL-17F, IL-1β, IL-20, IL-22, IL-23, IL-31, IL-31RA, IL-4, IL-5, IL-6, IL-6 receptor, IL-9, immunoglobulin lambda-like polypeptide 1 (IGLL1), influenza A hemagglutinin, insulin-like growth factor 1 receptor (IGF-I receptor), insulin-like growth factor 2 (ILGF2), integrin α4β7, integrin β2, integrin α2, integrin α4, integrin α5β1, integrin α7β7, integrin αIIbβ3, integrin αvβ3, interferon α/β receptor, interferon γ-induced protein, Interleukin 11 receptor α (IL-11Rα), Interleukin-13 receptor subunit α-2 (IL-13Ra2 or CD213A2), intestinal carboxyl esterase, kinase domain region (KDR), KIR2D, KIT (CD117), L1-cell adhesion molecule (L1-CAM), legumain, leukocyte immunoglobulin-like receptor subfamily A member 2 (LILRA2), leukocyte-associated immunoglobulin-like receptor 1 (LAIR1), lymphocyte antigen 6 (Ly-6), Lewis-Y antigen, LFA-1 (CD11a), LINGO-1, lipoteichoic acid, LOXL2, L-selectin (CD62L), lymphocyte antigen 6 complex, locus K 9 (LY6K), lymphocyte antigen 75 (LY75), lymphocyte-specific protein tyrosine kinase (LCK), lymphotoxin-α (LT-α) or Tumor necrosis factor-β (TNF-β), Lysosomal Associated Membrane Protein 1 (LAMP1), macrophage migration inhibitory factor (MIF or MMIF), M-CSF, mammary gland differentiation antigen (NY-BR-1), MCP-1, melanoma cancer testis antigen-1 (MAD-CT-1), melanoma cancer testis antigen-2 (MAD-CT-2), melanoma inhibitor of apoptosis (ML-IAP), melanoma-associated antigen 1 (MAGE-A1), mesothelin, mucin 1, cell surface associated (MUC1), MUC-2, MUC3, MUC4, MUC5AC, MUC5B, MUC7, MUC16, mucin CanAg, myelin-associated glycoprotein, myostatin, N-Acetyl glucosaminyl-transferase V (NA17), NCA-90 (granulocyte antigen), Nectin 4, nerve growth factor (NGF), neural apoptosis-regulated proteinase 1, neural cell adhesion molecule (NCAM), neurite outgrowth inhibitor (e.g., NOGO-A, NOGO-B, NOGO-C), neuropilin-1 (NRP1), N-glycolylneuraminic acid, NKG2D, Notch receptor, o-acetyl-GD2 ganglioside (OAcGD2), olfactory receptor 51E2 (OR51E2), oncofetal antigen (h5T4), oncogene fusion protein consisting of breakpoint cluster region (BCR) and Abelson murine leukemia viral oncogene homolog 1 (Abl) (bcr-abl), Oryctolagus cuniculus, OX-40, oxLDL, p53 mutant, paired box protein Pax-3 (PAX3), paired box protein Pax-5 (PAXS), pannexin 3 (PANX3), P-cadherin, phosphate-sodium co-transporter, phosphatidylserine, placenta-specific 1 (PLAC1), platelet-derived growth factor receptor α (PDGF-R α), platelet-derived growth factor receptor β (PDGFR-β), polysialic acid, proacrosin binding protein sp32 (OY-TES1), programmed cell death protein 1 (PD-1), Programmed death-ligand 1 (PD-L1), proprotein convertase subtilisin/kexin type 9 (PCSK9), prostase, prostate carcinoma tumor antigen-1 (PCTA-1 or Galectin 8), melanoma antigen recognized by T cells 1 (MelanA or MART1), P15, P53, PRAME, prostate stem cell antigen (PSCA), prostate-specific membrane antigen (PSMA), prostatic acid phosphatase (PAP), prostatic carcinoma cells, prostein, Protease Serine 21 (Testisin or PRSS21), Proteasome (Prosome, Macropain) Subunit, β Type, 9 (LMP2), Pseudomonas aeruginosa, rabies virus glycoprotein, RAGE, Ras Homolog Family Member C (RhoC), receptor activator of nuclear factor kappa-B ligand (RANKL), Receptor for Advanced Glycation Endproducts (RAGE-1), receptor tyrosine kinase-like orphan receptor 1 (ROR1), renal ubiquitous 1 (RU1), renal ubiquitous 2 (RU2), respiratory syncytial virus, Rh blood group D antigen, Rhesus factor, sarcoma translocation breakpoints, sclerostin (SOST), selectin P, sialyl Lewis adhesion molecule (sLe), sperm protein 17 (SPA17), sphingosine-1-phosphate, squamous cell carcinoma antigen recognized by T Cells 1, 2, and 3 (SART1, SART2, and SART3), stage-specific embryonic antigen-4 (SSEA-4), Staphylococcus aureus, STEAP1, syndecan 1 (SDC1)+A314, SOX10, survivin, survivin-2B, synovial sarcoma, X breakpoint 2 (SSX2), T-cell receptor, TCR F Alternate Reading Frame Protein (TARP), telomerase, TEM1, tenascin C, TGF-β (e.g., TGF-β1, TGF-β2, TGF-β3), thyroid stimulating hormone receptor (TSHR), tissue factor pathway inhibitor (TFPI), Tn antigen ((Tn Ag) or (GalNAcα-Ser/Thr)), TNF receptor family member B cell maturation (BCMA), TNF-α, TRAIL-R1, TRAIL-R2, TRG, transglutaminase 5 (TGS5), tumor antigen CTAA16.88, tumor endothelial marker 1 (TEM1/CD248), tumor endothelial marker 7-related (TEM7R), tumor protein p53 (p53), tumor specific glycosylation of MUC1, tumor-associated calcium signal transducer 2 (TROP-2), tumor-associated glycoprotein 72 (TAG72), tumor-associated glycoprotein 72 (TAG-72)+A327, TWEAK receptor, tyrosinase, tyrosinase-related protein 1 (TYRP1 or glycoprotein 75), tyrosinase-related protein 2 (TYRP2), uroplakin 2 (UPK2), vascular endothelial growth factor (e.g., VEGF-A, VEGF-B, VEGF-C, VEGF-D, PIGF), vascular endothelial growth factor receptor 1 (VEGFR1), vascular endothelial growth factor receptor 2 (VEGFR2), vimentin, v-myc avian myelocytomatosis viral oncogene neuroblastoma derived homolog (MYCN), von Willebrand factor (VWF), Wilms tumor protein (WT1), X Antigen Family, Member 1A (XAGE1), β-amyloid, κ-light chain, Fibroblast Growth Factor Receptor 2 (FGFR2), LIV-1 Protein, estrogen regulated (LIV1, aka SLC39A6), Neurotrophic Receptor Tyrosine Kinase 1 (NTRK1, aka TRK), Ret Proto-Oncogene (RET), B Cell Maturation Antigen (BCMA, aka TNFRSF17), Transferrin Receptor (TFRC, aka CD71), Activated Leukocyte Cell Adhesion Molecule (ALCAM, aka CD166), Somatostatin Receptor 2 (SSTR2), KIT Proto-Oncogene Receptor Tyrosine Kinase (cKIT), V-Set Immunoregulatory Receptor (VSIR, aka VISTA), Glycoprotein Nmb (GPNMB), Delta Like Canonical Notch Ligand 3 (DLL3), Interleukin 3 Receptor Subunit Alpha (IL3RA, aka CD123), Lysosomal Associated Membrane Protein 1 (LAMP1), Cadherin 3, Type 1, P-Cadherin (CDH3), Ephrin A4 (EFNA4), Protein Tyrosine Kinase 7 (PTK7), Solute Carrier Family 34 Member 2 (SLC34A2, aka NaPi-2b), Guanylyl Cyclase C (GCC), PLAUR Domain Containing 3 (LYPD3, aka LY6 or C4.4a), Mucin 17, Cell Surface Associated (MUC17), Fms Related Receptor Tyrosine Kinase 3 (FLT3), NKG2D ligands (e.g. ULBP1, ULBP2, ULBP3, H60, Rae-1α, Rae-1β, Rae-1δ, Rae-1γ, MICA, MICB, hHLA-A), SLAM Family Member 7 (SLAMF7), Interleukin 13 Receptor Subunit Alpha 2 (IL13RA2), C-Type Lectin Domain Family 12 Member A (CLEC12A aka CLL-1), CEA Cell Adhesion Molecule 5 (CEACAM aka CD66e), Interleukin 3 Receptor Subunit Alpha (IL3RA), CD5 Molecule (CD5), UL16 Binding Protein 1 (ILBP1), V-Set Domain Containing T Cell Activation Inhibitor 1 (VTCN1 aka B7-H4), Chondroitin Sulfate Proteoglycan 4 (CSPG4), Syndecan 1 (SDC1 aka CD138), Interleukin 1 Receptor Accessory Protein (IL1RAP), Baculoviral IAP Repeat Containing 5 (BIRC5 aka Survivin), CD74 Molecule (CD74), Hepatitis A Virus Cellular Receptor 1 (HAVCR1 aka TIM1), SLIT and NTRK Like Family Member 6 (SILTRK6), CD37 Molecule (CD37), Coagulation Factor III, Tissue Factor (CD142 aka F3), AXL Receptor Tyrosine Kinase (AXL), Endothelin Receptor Type B (EDNRB aka ETBR), Cadherin 6 (CDH6), Fibroblast Growth Factor Receptor 3 (FGFR3), Carbonic Anhydrase 6 (CA6), CanAg glycoform of MUC1, Integrin Subunit Alpha V (ITGAV), Teratocarcinoma-Derived Growth Factor 1 (TDGF1, aka Crypto 1), SLAM Family Member 6 (SLAMF6 aka CD352), and Notch Receptor 3 (NOTCH3).

Therapeutic monoclonal antibodies from which the AF2 can be derived for incorporation into any of the polypeptide embodiments of the subject compositions described herein are known in the art. Such therapeutic antibodies can include, but are not limited to, rituximab, IDEC/Genentech/Roche (see, e.g., U.S. Pat. No. 5,736,137), a chimeric anti-CD20 antibody used in the treatment of many lymphomas, leukemias, and some autoimmune disorders; ofatumumab, an anti-CD20 antibody approved for use for chronic lymphocytic leukemia, and under development for follicular non-Hodgkin's lymphoma, diffuse large B cell lymphoma, rheumatoid arthritis and relapsing remitting multiple sclerosis; lucatumumab (HCD122), an anti-CD40 antibody for Non-Hodgkin's or Hodgkin's Lymphoma (see, for example, U.S. Pat. No. 6,899,879), AME-133, an antibody which binds to cells expressing CD20 to treat non-Hodgkin's lymphoma, veltuzumab (hA20), an antibody which binds to cells expressing CD20 to treat immune thrombocytopenic purpura, HumaLYM developed for the treatment of low-grade B-cell lymphoma, and ocrelizumab, which is an anti-CD20 monoclonal antibody for treatment of rheumatoid arthritis (see, e.g., U.S. Patent Application 20090155257), trastuzumab (see, e.g., U.S. Pat. No. 5,677,171), a humanized anti-HER2/neu antibody approved to treat breast cancer; pertuzumab, an anti-HER2 dimerization inhibitor antibody developed for use in treatment of in prostate, breast, and ovarian cancers; (see, e.g., U.S. Pat. No. 4,753,894); cetuximab, an anti-EGFR antibody used to treat epidermal growth factor receptor (EGFR)-expressing, KRAS wild-type metastatic colorectal cancer and head and neck cancer (see U.S. Pat. No. 4,943,533; PCT WO 96/40210); panitumumab, a fully human monoclonal antibody specific to the epidermal growth factor receptor (also known as EGF receptor, EGFR, ErbB-1 and HER1, currently marketed for treatment of metastatic colorectal cancer (see U.S. Pat. No. 6,235,883); zalutumumab, a fully human IgG1 monoclonal antibody that is directed towards the epidermal growth factor receptor (EGFR) for the treatment of squamous cell carcinoma of the head and neck (see, e.g., U.S. Pat. No. 7,247,301); nimotuzumab, a chimeric antibody to EGFR developed for the treatment of squamous cell carcinomas of the head and neck, nasopharyngeal cancer and glioma (see, e.g., U.S. Pat. No. 5,891,996; U.S. Pat. No. 6,506,883); matuzumab, a humanized monoclonal that is directed towards the epidermal growth factor receptor (EGFR) that was developed for the treatment of colorectal, lung, esophageal and stomach cancer (see, e.g., U.S. Patent Application 20090175858A1); cetuximab, a chimeric (mouse/human) monoclonal antibody that is directed to epidermal growth factor receptor (EGFR) used for the treatment of metastatic colorectal cancer, metastatic non-small cell lung cancer and head and neck cancer (see, e.g., U.S. Pat. No. 6,217,866); alemtuzumab, a humanized monoclonal antibody to CD52 marketed for the treatment of chronic lymphocytic leukemia (CLL), cutaneous T-cell lymphoma (CTCL) and T-cell lymphoma; ibritumomab tiuxetan, an anti-CD20 monoclonal antibody developed for treatment for some forms of B cell non-Hodgkin's lymphoma; gemtuzumab ozogamicin, an anti-CD33 (p67 protein) antibody linked to a cytotoxic chelator tiuxetan, to which a radioactive isotope is attached, used to treat acute myelogenous leukemia; ABX-CBL, an anti-CD147 antibody; ABX-IL8, an anti-IL8 antibody, ABX-MA1, an anti-MUC18 antibody, Pemtumomab (R1549, 90Y-muHMFG1), an anti-MUC1 in development, Therex (R1550), an anti-MUC1 antibody, AngioMab (AS1405), developed by Antisoma, HuBC-1, developed by Antisoma, Thioplatin (AS1407) developed by Antisoma, ANTEGREN (natalizumab), an anti-alpha-4-beta-1 (VLA4) and alpha-4-beta-7 antibody, VLA-1 mAb, an anti-VLA-1 integrin antibody, LTBR mAb, an anti-lymphotoxin beta receptor (LTBR) antibody, CAT-152, an anti-TGF-02 antibody, J695, an anti-IL-12 antibody, CAT-192, an anti-TGFβ1 antibody developed, CAT-213, an anti-Eotaxin1 antibody developed, LYMPHOSTAT-B, an anti-Blys antibody, TRAIL-R1mAb, an anti-TRAIL-R1 antibody; Herceptin, an anti-HER receptor family antibody; Anti-Tissue Factor (ATF), an anti-Tissue Factor antibody; Xolair (Omalizumab), an anti-IgE antibody, MLN-02 Antibody (formerly LDP-02); HuMax CD4®, an anti-CD4 antibody; tocilizuma, and anti-IL6R antibody; HuMax-IL15, an anti-IL15 antibody, HuMax-Inflam; HuMax-Cancer, an anti-Heparanase I antibody; HuMax-Lymphoma, HuMax-TAC; IDEC-131, an anti-CD40; IDEC-151 (Clenoliximab), an anti-CD4 antibody; IDEC-114, an anti-CD80 antibody; IDEC-152, an anti-CD23; an anti-KDR antibody, DC101, an anti-flk-1 antibody; anti-VE cadherin antibodies developed by Imclone; CEA-CIDE (labetuzumab), an anti-carcinoembryonic antigen (CEA) antibody developed by Immunomedics; Yervoy (ipilimumab), an anti-CTLA4 antibody used in the treatment of melanoma; Lumphocide® (Epratuzumab), an anti-CD22 antibody, AFP-Cide, developed by Immunomedics; MyelomaCide, developed by Immunomedics; LkoCide, developed by Immunomedics; ProstaCide, developed by Immunomedics; MDX-010, an anti-CTLA4 antibody; MDX-060, an anti-CD30 antibody; MDX-070; MDX-018 developed by Medarex; OSIDEM (IDM-1), an anti-HER2 antibody; HuMax®-CD4, an anti-CD4 antibody; HuMax-IL15, an anti-IL15 antibody; anti-intercellular adhesion molecule-1 (ICAM-1) (CD54) antibodies, MOR201; tremelimumab, an anti-CTLA-4 antibody; Anti-a 5β1 Integrin, developed by Protein Design Labs; anti-IL-12, developed by Protein Design Labs; ING-1, an anti-Ep-CAM antibody developed by Xoma; and MLN01, an anti-Beta2 integrin antibody; all of the above-cited antibody references in this paragraph are expressly incorporated herein by reference. The sequences for the above antibodies can be obtained from publicly available databases, patents, or literature references. In addition, non-limiting examples of monoclonal antibodies and VH and VL sequences (and, in some cases, with indicated CDR sequences that can be incorporated into the AF2) to cancer, tumor, or target cell markers suitable for incorporation into the subject compositions of the disclosure are presented in Table 8.

In accordance with the antigen binding fragment embodiments referred to above, it may be advantageous if the binding site recognizing the target cell marker antigen has a high binding affinity in order to capture the target cells to be destroyed with high efficiency. The subject polypeptides of any of the embodiments of the disclosure have the advantage that they may be used a number of times for killing tumor cells since, in preferred embodiments, the AF2 target cell antigen binding fragment has an affinity with a K_{d} value in the range of 10⁻⁷ to 10⁻¹⁰ M, as determined in an vitro binding assay. If the affinity of a bispecific antigen binding fragment for binding a target cell marker is too high, the composition binds the expressing target cell and remains on its surface, making it unable to release and bind to another cell. In one embodiment, a polypeptide of any of the subject composition embodiments described herein comprises an AF2, wherein the AF2 specifically binds the target cell marker with a K_{d} between about 0.1 nM and about 100 nM, or about 0.5 to about 50 nM, or about 1.0 to about 10 nM, as determined in an in vitro antigen-binding assay comprising the target cell marker. In another embodiment, the AF2 specifically binds the target cell marker with a binding affinity (as determined by the K_{d} in an in vitro binding assay) of less than about 0.1 nM, or less than about 0.5 nM, or less than about 1.0 nM, or less than about 10 nM, or less than about 50 nM, or less than about 100 nM. In another embodiment, the present disclosure provides polypeptides comprising an AF2, wherein the binding affinity of the AF2 to the target cell marker is at least 10-fold greater, or at least 100-fold greater, or at least 1000-fold greater than the binding affinity of the AF1 to CD3, as measured in an in vitro antigen-binding assay. In another embodiment, the AF1 antigen binding fragment of any of the subject embodiments of the disclosure has a lower binding affinity to the CD3 antigen of at least one order, at least two orders, or at least three orders of magnitude lower compared to the greater binding affinity of the AF2 to the target cell marker antigen, as determined as K_{d} constants in an in vitro assay. It will be understood that a greater binding affinity means a lower K_{d} value; e.g., 1×10⁻⁹ M is a greater binding affinity than 1×10⁻⁸ M.

In another embodiment, the present disclosure provides polypeptides comprising an AF2, wherein the AF2 comprises CDR of a monoclonal antibody having binding affinity to the target cell marker antigen. In another embodiment, the polypeptides of any of the subject composition embodiments described herein comprise an AF2, wherein the AF2 comprises CDR derived from a monoclonal antibody having binding affinity to the target cell marker antigen wherein the CDR of the AF2 are selected from the CDRs within the VL and VH sequences of SEQ ID NOs:719-918.

In some aspects of any of the embodiments disclosed herein, a subject polypeptide comprises an AF2, wherein the AF2 comprises a VL and VH of a monoclonal antibody having binding affinity to the target cell marker antigen. In some cases, the polypeptides of any of the subject composition embodiments described herein can comprise an AF2 wherein the AF2 comprises VL and VH of a monoclonal antibody having binding affinity to the target cell marker antigen wherein the VL comprises an amino acid sequence having at least 90%, 91%, 92%, 93%, 94%, 95%, 96%, 97%, 98%, 99% sequence identity or is identical to an amino acid sequence of SEQ ID NOs:719-918, and the VH comprises an amino acid sequence having at least 90%, 91%, 92%, 93%, 94%, 95%, 96%, 97%, 98%, 99% sequence identity or is identical to an amino acid sequence of SEQ ID NOs:719-818.

It will be understood that use of the term “antigen binding fragment” for the composition embodiments disclosed herein is intended to include portions or fragments of antibodies that retain the ability to bind the antigens that are the ligands of the corresponding intact antibody. In such embodiments, the antigen binding fragment can be, but is not limited to, CDRs and intervening framework regions, variable or hypervariable regions of light and/or heavy chains of an antibody (VL, VH), variable fragments (Fv), Fab′ fragments, F(ab′)2 fragments, Fab fragments, single chain antibodies (scAb), VHH camelid antibodies, single chain variable fragment (scFv), linear antibodies, a single domain antibody, complementarity determining regions (CDR), domain antibodies (dAbs), single domain heavy chain immunoglobulins of the BHH or BNAR type, single domain light chain immunoglobulins, or other polypeptides known in the art containing a fragment of an antibody capable of binding an antigen. The VL and VH of two antigen binding fragments can also be configured in a single chain diabody configuration; i.e., the VL and VH of the AF1 and AF2 configured with linkers of an appropriate length to permit arrangement as a diabody.

In certain embodiments, the VL and VH of the antigen binding fragments are fused by relatively long linkers, consisting 25, 26, 27, 28, 29, 30, 31, 32, 33, 34, or 35 hydrophilic amino acids that, when joined together, have a flexible characteristic. In one embodiment, the VL and VH of any of the scFv embodiments described herein are linked by linkers of hydrophilic amino acids selected from the sequences GSGEGSEGEGGGEGSEGEGSGEGGEGEGSG (SEQ ID NO: 1142), TGSGEGSEGEGGGEGSEGEGSGEGGEGEGSGT (SEQ ID NO: 1143), GATPPETGAETESPGETTGGSAESEPPGEG (SEQ ID NO: 1144), or GSAAPTAGTTPSASPAPPTGGSSAAGSPST (SEQ ID NO: 1145). In other cases, the AF1 and AF2 of the subject compositions are linked together by a short linker of hydrophilic amino acids having 3, 4, 5, 6, or 7 amino acids. In one embodiment, the short linker sequences are selected from the group of sequences SGGGGS (SEQ ID NO: 1146), GGGGS (SEQ ID NO: 1147), GGSGGS (SEQ ID NO: 1148), GGS, or GSP. In another embodiment, the disclosure provides compositions comprising a single chain diabody in which after folding, the first domain (VL or VH) is paired with the last domain (VH or VL) to form one scFv and the two domains in the middle are paired to form the other scFv in which the first and second domains, as well as the third and last domains, are fused together by one of the foregoing short linkers and the second and the third variable domains are fused by one of the foregoing long linkers. The selection of the short linker and long linker may prevent the incorrect pairing of adjacent variable domains, thereby facilitating the formation of the single chain diabody configuration comprising the VL and VH of the first antigen binding fragment and the second antigen binding fragment.

TABLE 8
Target cell marker antibodies and sequences

| | | Target | | SEQ | | SEQ |
|---|---|---|---|---|---|---|
| Trade | Antibody | Cell | | ID | | ID |
| Name | Name | Marker | VH Sequence | NO: | VL Sequence | NO: |
| Tysabri™ | natalizumab | Alpha 4 | QVQLVQSGAEV | 719 | DIQMTQSPSSL | 819 |
| | | Integrin | KKPGASVKVSC | | SASVGDRVTIT | |
| | | | KASGFNIKDTY | | CKTSQDINKYM | |
| | | | IHWVRQAPGQR | | AWYQQTPGKAP | |
| | | | LEWMGRIDPAN | | RLLIHYTSALQ | |
| | | | GYTKYDPKFQG | | PGIPSRFSGSG | |
| | | | RVTITADTSAS | | SGRDYTFTISS | |
| | | | TAYMELSSLRS | | LQPEDIATYYC | |
| | | | EDTAVYYCARE | | LQYDNLWTFGQ | |
| | | | GYYGNYGVYAM | | GTKVEIK | |
| | | | DYWGQGTLVTV | | | |
| | | | SS | | | |
| REGN910 | nesvacumab | Ang2 | EVQLVESGGGL | 720 | EIVLTQSPGTL | 820 |
| | | | VQPGGSLRLSC | | SLSPGERATLS | |
| | | | AASGFTFSSYD | | CRASQSVSSTY | |
| | | | IHWVRQATGKG | | LAWYQQKPGQA | |
| | | | LEWVSAIGPAG | | PRLLIYGASSR | |
| | | | DTYYPGSVKGR | | ATGIPDRFSGS | |
| | | | FTISRENAKNS | | GSGTDFTLTIS | |
| | | | LYLQMNSLRAG | | RLEPEDFAVYY | |
| | | | DTAVYYCARGL | | CQHYDNSQTFG | |
| | | | ITFGGLIAPFD | | QGTKVEIK | |
| | | | YWGQGTLVTVS | | | |
| | | | S | | | |
| hMFE23 | | CEA | QVKLEQSGAEV | 721 | ENVLTQSPSSM | 821 |
| | | | VKPGASVKLSC | | SASVGDRVNIA | |
| | | | KASGFNIKDSY | | CSASSSVSYMH | |
| | | | MHWLRQGPGQR | | WFQQKPGKSPK | |
| | | | LEWIGWIDPEN | | LWIYSTSNLAS | |
| | | | GDTEYAPKFQG | | GVPSRFSGSGS | |
| | | | KATFTTDTSAN | | GTDYSLTISSM | |
| | | | TAYLGLSSLRP | | QPEDAATYYCQ | |
| | | | EDTAVYYCNEG | | QRSSYPLTFGG | |
| | | | TPTGPYYFDYW | | GTKLEIK | |
| | | | GQGTLVTVSS | | | |
| M5A | | CEA | EVQLVESGGGL | 722 | DIQLTQSPSSL | 822 |
| (humanized | | | VQPGGSLRLSC | | SASVGDRVTIT | |
| T84.66) | | | AASGFNIKDTY | | CRAGESVDIFG | |
| | | | MHWVRQAPGKG | | VGFLHWYQQKP | |
| | | | LEWVARIDPAN | | GKAPKLLIYRA | |
| | | | GNSKYADSVKG | | SNLESGVPSRF | |
| | | | RFTISADTSKN | | SGSGSRTDFTL | |
| | | | TAYLQMNSLRA | | TISSLQPEDFA | |
| | | | EDTAVYYCAPF | | TYYCQQTNEDP | |
| | | | GYYVSDYAMAY | | YTFGQGTKVEI | |
| | | | WGQGTLVTVSS | | K | |
| M5B | | CEA | EVQLVESGGGL | 723 | DIQLTQSPSSL | 823 |
| (humanized | | | VQPGGSLRLSC | | SASVGDRVTIT | |
| T84.66) | | | AASGFNIKDTY | | CRAGESVDIFG | |
| | | | MHWVRQAPGKG | | VGFLHWYQQKP | |
| | | | LEWVARIDPAN | | GKAPKLLIYRA | |
| | | | GNSKYVPKFQG | | SNLESGVPSRF | |
| | | | RATISADTSKN | | SGSGSRTDFTL | |
| | | | TAYLQMNSLRA | | TISSLQPEDFA | |
| | | | EDTAVYYCAPF | | TYYCQQTNEDP | |
| | | | GYYVSDYAMAY | | YTFGQGTKVEI | |
| | | | WGQGTLVTVSS | | K | |
| CEA-Cide | Labetuzumab | CEACAM5 | EVQLVESGGGV | 724 | DIQLTQSPSSL | 824 |
| | (MN-14) | | VQPGRSLRLSC | | SASVGDRVTIT | |
| | | | SASGFDFTTYW | | CKASQDVGTSV | |
| | | | MSWVRQAPGKG | | AWYQQKPGKAP | |
| | | | LEWIGEIHPDS | | KLLIYWTSTRH | |
| | | | STINYAPSLKD | | TGVPSRFSGSG | |
| | | | RFTISRDNAKN | | SGTDFTFTISS | |
| | | | TLFLQMDSLRP | | LQPEDIATYYC | |
| | | | EDTGVYFCASL | | QQYSLYRSFGQ | |
| | | | YFGFPWFAYWG | | GTKVEIK | |
| | | | QGTPVTVSS | | | |
| CEA-Scan | arcitumomab | CEACAM5 | EVKLVESGGGL | 725 | QTVLSQSPAIL | 825 |
| | | | VQPGGSLRLSC | | SASPGEKVTMT | |
| | | | ATSGFTFTDYY | | CRASSSVTYIH | |
| | | | MNWVRQPPGKA | | WYQQKPGSSPK | |
| | | | LEWLGFIGNKA | | SWIYATSNLAS | |
| | | | NGYTTEYSASV | | GVPARFSGSGS | |
| | | | KGRFTISRDKS | | GTSYSLTISRV | |
| | | | QSILYLQMNTL | | EAEDAATYYCQ | |
| | | | RAEDSATYYCT | | HWSSKPPTFGG | |
| | | | RDRGLRFYFDY | | GTKLEIKR | |
| | | | WGQGTTLTVSS | | | |
| MT110 | | CEACAM5 | EVQLVESGGGL | 726 | QAVLTQPASLS | 826 |
| | | | VQPGRSLRLSC | | ASPGASASLTC | |
| | | | AASGFTVSSYW | | TLRRGINVGAY | |
| | | | MHWVRQAPGKG | | SIYWYQQKPGS | |
| | | | LEWVGFIRNKA | | PPQYLLRYKSD | |
| | | | NGGTTEYAASV | | SDKQQGSGVSS | |
| | | | KGRFTISRDDS | | RFSASKDASAN | |
| | | | KNTLYLQMNSL | | AGILLISGLQS | |
| | | | RAEDTAVYYCA | | EDEADYYCMIW | |
| | | | RDRGLRFYFDY | | HSGASAVFGGG | |
| | | | WGQGTTVTVSS | | TKLTVL | |
| MT103 | blinatumomab | CD19 | QVQLQQSGAEL | 727 | DIQLTQSPASL | 827 |
| | | | VRPGSSVKISC | | AVSLGQRATIS | |
| | | | KASGYAFSSYW | | CKASQSVDYDG | |
| | | | MNWVKQRPGQG | | DSYLNWYQQIP | |
| | | | LEWIGQIWPGD | | GQPPKLLIYDA | |
| | | | GDTNYNGKFKG | | SNLVSGIPPRF | |
| | | | KATLTADESSS | | SGSGSGTDFTL | |
| | | | TAYMQLSSLAS | | NIHPVEKVDAA | |
| | | | EDSAVYFCARR | | TYHCQQSTEDP | |
| | | | ETTTVGRYYYA | | WTFGGGTKLEI | |
| | | | MDYWGQGTTVT | | K | |
| | | | VSS | | | |
| Arzerra | ofatummnab | CD20 | EVQLVESGGGL | 728 | EIVLTQSPATL | 828 |
| | | | VQPGRSLRLSC | | SLSPGERATLS | |
| | | | AASGFTFNDYA | | CRASQSVSSYL | |
| | | | MHWVRQAPGKG | | AWYQQKPGQAP | |
| | | | LEWVSTISWNS | | RLLIYDASNRA | |
| | | | GSIGYADSVKG | | TGIPARFSGSG | |
| | | | RFTISRDNAKK | | SGTDFTLTISS | |
| | | | SLYLQMNSLRA | | LEPEDFAVYYC | |
| | | | EDTALYYCAKD | | QQRSNWPITFG | |
| | | | IQYGNYYYGMD | | QGTRLEIK | |
| | | | VWGQGTTVTVS | | | |
| | | | S | | | |
| Bexxar™ | tositumomab | CD20 | QAYLQQSGAEL | 729 | QIVLSQSPAIL | 829 |
| | | | VRPGASVKMSC | | SASPGEKVTMT | |
| | | | KASGYTFTSYN | | CRASSSVSYMH | |
| | | | MHWVKQTPRQG | | WYQQKPGSSPK | |
| | | | LEWIGAIYPGN | | PWIYAPSNLAS | |
| | | | GDTSYNQKFKG | | GVPARFSGSGS | |
| | | | KATLTVDKSSS | | GTSYSLTISRV | |
| | | | TAYMQLSSLTS | | EAEDAATYYCQ | |
| | | | EDSAVYFCARV | | QWSFNPPTFGA | |
| | | | VYYSNSYWYFD | | GTKLELK | |
| | | | VWGTGTTVTVS | | | |
| | | | G | | | |
| GAZYVA | Obinutuzumab | CD20 | QVQLVQSGAEV | 730 | DIVMTQTPLSL | 830 |
| | | | KKPGSSVKVSC | | PVTPGEPASIS | |
| | | | KASGYAFSYSW | | CRSSKSLLHSN | |
| | | | INWVRQAPGQG | | GITYLYWYLQK | |
| | | | LEWMGRIFPGD | | PGQSPQLLIYQ | |
| | | | GDTDYNGKFKG | | MSNLVSGVPDR | |
| | | | RVTITADKSTS | | FSGSGSGTDFT | |
| | | | TAYMELSSLRS | | LKISRVEAEDV | |
| | | | EDTAVYYCARN | | GVYYCAQNLEL | |
| | | | VFDGYWLVYWG | | PYTFGGGTKVE | |
| | | | QGTLVTVSS | | IK | |
| | Ocrelizumab/ | CD20 | EVQLVESGGGL | 731 | DIQMTQSPSSL | 831 |
| | 2H7 v16 | | VQPGGSLRLSC | | SASVGDRVTIT | |
| | | | AASGYTFTSYN | | CRASSSVSYMH | |
| | | | MHWVRQAPGKG | | WYQQKPGKAPK | |
| | | | LEWVGAIYPGN | | PLIYAPSNLAS | |
| | | | GDTSYNQKFKG | | GVPSRFSGSGS | |
| | | | RFTISVDKSKN | | GTDFTLTISSL | |
| | | | TLYLQMNSLRA | | QPEDFATYYCQ | |
| | | | EDTAVYYCARV | | QWSFNPPTFGQ | |
| | | | VYYSNSYWYFD | | GTKVEIK | |
| | | | VWGQGTLVTVS | | | |
| | | | S | | | |
| Rituxan™ | rituximab | CD20 | QVQLQQPGAEL | 732 | QIVLSQSPAIL | 832 |
| | | | VKPGASVKMSC | | SASPGEKVTMT | |
| | | | KASGYTFTSYN | | CRASSSVSYIH | |
| | | | MHWVKQTPGRG | | WFQQKPGSSPK | |
| | | | LEWIGAIYPGN | | PWIYATSNLAS | |
| | | | GDTSYNQKFKG | | GVPVRFSGSGS | |
| | | | KATLTADKSSS | | GTSYSLTISRV | |
| | | | TAYMQLSSLTS | | EAEDAATYYCQ | |
| | | | EDSAVYYCARS | | QWTSNPPTFGG | |
| | | | TYYGGDWYFNV | | GTKLEIK | |
| | | | WGAGTTVTVSA | | | |
| Zevalin™ | ibritumomab | CD20 | QAYLQQSGAEL | 733 | QIVLSQSPAIL | 833 |
| | tieuxetan | | VRPGASVKMSC | | SASPGEKVTMT | |
| | | | KASGYTFTSYN | | CRASSSVSYMH | |
| | | | MHWVKQTPRQG | | WYQQKPGSSPK | |
| | | | LEWIGAIYPGN | | PWIYAPSNLAS | |
| | | | GDTSYNQKFKG | | GVPARFSGSGS | |
| | | | KATLTVDKSSS | | GTSYSLTISRV | |
| | | | TAYMQLSSLTS | | EAEDAATYYCQ | |
| | | | EDSAVYFCARV | | QWSFNPPTFGA | |
| | | | VYYSNSYWYFD | | GTKLELK | |
| | | | VWGTGTTVTVS | | | |
| | | | A | | | |
| Mylotarg | Gemtuzumab | CD33 | QLVQSGAEVKK | 734 | DIQLTQSPSTL | 834 |
| | (hP67.6) | | PGSSVKVSCKA | | SASVGDRVTIT | |
| | | | SGYTITDSNIH | | CRASESLDNYG | |
| | | | WVRQAPGQSLE | | IRFLTWFQQKP | |
| | | | WIGYIYPYNGG | | GKAPKLLMYAA | |
| | | | TDYNQKFKNRA | | SNQGSGVPSRF | |
| | | | TLTVDNPTNTA | | SGSGSGTEFTL | |
| | | | YMELSSLRSED | | TISSLQPDDFA | |
| | | | TDFYYCVNGNP | | TYYCQQTKEVP | |
| | | | WLAYWGQGTLV | | WSFGQGTKVEV | |
| | | | TVSS | | K | |
| Daratuniumab | | CD38 | EVQLLESGGGL | 735 | EIVLTQSPATL | 835 |
| | | | VQPGGSLRLSC | | SLSPGERATLS | |
| | | | AVSGFTFNSFA | | CRASQSVSSYL | |
| | | | MSWVRQAPGKG | | AWYQQKPGQAP | |
| | | | LEWVSAISGSG | | RLLIYDASNRA | |
| | | | GGTYYADSVKG | | TGIPARFSGSG | |
| | | | RFTISRDNSKN | | SGTDFTLTISS | |
| | | | TLYLQMNSLRA | | LEPEDFAVYYC | |
| | | | EDTAVYFCAKD | | QQRSNWPPTFG | |
| | | | KILWFGEPVFD | | QGTKVEIK | |
| | | | YWGQGTLVTVS | | | |
| | | | S | | | |
| | 1F6 | CD70 | QIQLVQSGPEV | 736 | DIVLTQSPASL | 836 |
| | | | KKPGETVKISC | | AVSLGQRATIS | |
| | | | KASGYTFTNYG | | CRASKSVSTSG | |
| | | | MNWVKQAPGKG | | YSFMHWYQQKP | |
| | | | LKWMGWINTYT | | GQPPKLLIYLA | |
| | | | GEPTYADAFKG | | SNLESGVPARF | |
| | | | RFAFSLETSAS | | SGSGSGTDFTL | |
| | | | TAYLQINNLKN | | NIHPVEEEDAA | |
| | | | EDTATYFCARD | | TY | |
| | | | YGDYGMDYWGQ | | YCQHSREVPWT | |
| | | | GTSVTVSS | | FGGGTKLEIK | |
| | 2F2 | CD70 | QVQLQQSGTEL | 737 | DIVLTQSPASL | 837 |
| | | | MTPGASVTMSC | | TVSLGQKTTIS | |
| | | | KTSGYTFSTYW | | CRASKSVSTSG | |
| | | | IEWVKQRPGHG | | YSFMHWYQLKP | |
| | | | LEWIGEILGPS | | GQSPKLLIYLA | |
| | | | GYTDYNEKFKA | | SDLPSGVPARF | |
| | | | KATFTADTSSN | | SGSGSGTDFTL | |
| | | | TAYMQLSSLAS | | KIHPVEEEDAA | |
| | | | EDSAVYYCARW | | TY | |
| | | | DRLYAHDYWGG | | YCQHSREIPYT | |
| | | | GTSVTVSS | | FGGGTKLEIT | |
| | 2H5 | CD70 | QVQLVESGGGV | 738 | EIVLTQSPATL | 838 |
| | | | VQPGRSLRLSC | | SLSPGERATLS | |
| | | | AASGFTFSSYI | | CRASQSVSSYL | |
| | | | MHWVRQAPGKG | | AWYQQKPGQAP | |
| | | | LEWVAVISYDG | | RLLIYDASNRA | |
| | | | RNKYYADSVKG | | TGIPARFSGSG | |
| | | | RFTISRDNSKN | | SGTDFTLTISS | |
| | | | TLYLQMNSLRA | | LEPEDFAVYYC | |
| | | | ED | | QQ | |
| | | | TAVYYCARDTD | | RTNWPLTFGGG | |
| | | | GYDFDYWGQGT | | TKVEIK | |
| | | | LVTVSS | | | |
| | 10B4 | CD70 | QIQLVESGGGV | 739 | AIQLTQSPSSL | 839 |
| | | | VQPGRSLRLSC | | SASVGDRVTIT | |
| | | | AASGFTFGYYA | | CRASQGISSAL | |
| | | | MHWVRQAPGKG | | AWYQQKPGKAP | |
| | | | LEWVAVISYDG | | KFLIYDASSLE | |
| | | | SIKYYADSVKG | | SGVPSRFSGSG | |
| | | | RFTISRDNSKN | | SGTDFTLTISS | |
| | | | TLYLQMNSLRA | | LQPEDFATYYC | |
| | | | ED | | QQ | |
| | | | TAVYYCAREGP | | FNSYPFTFGPG | |
| | | | YSNYLDYWGQG | | TKVDIK | |
| | | | TLVTVSS | | | |
| | 8B5 | CD70 | QVQLVESGGGV | 740 | DIQMTQSPSSL | 840 |
| | | | VQPGRSLRLSC | | SASVGDRVTIT | |
| | | | ATSGFTFSDYG | | CRASQGISSWL | |
| | | | MHWVRQAPGKG | | AWYQQKPEKAP | |
| | | | LEWVAVIWYDG | | KSLIYAASSLQ | |
| | | | SNKYYADSVKG | | SGVPSRFSGSG | |
| | | | RFTISRDNSKK | | SGTDFTLTISS | |
| | | | TLSLQMNSLRA | | LQPEDFATYYC | |
| | | | ED | | QQ | |
| | | | TAVYYCARDSI | | YNSYPLTFGGG | |
| | | | MVRGDYWGQGT | | TKVEIK | |
| | | | LVTVSS | | | |
| | 18E7 | CD70 | QVQLVESGGGV | 741 | DIQMTQSPSSL | 841 |
| | | | VQPGRSLRLSC | | SASVGDRVTIT | |
| | | | AASGFTFSDHG | | CRASQGISSWL | |
| | | | MHWVRQAPGKG | | AWYQQKPEKAP | |
| | | | LEWVAVIWYDG | | KSLIYAASSLQ | |
| | | | SNKYYADSVKG | | SGVPSRFSGSG | |
| | | | RFTISRDNSKN | | SGTDFTLTISS | |
| | | | TLYLQMNSLRA | | LQPEDFATYYC | |
| | | | ED | | QQ | |
| | | | TAVYYCARDSI | | YNSYPLTFGGG | |
| | | | MVRGDYWGQGT | | TKVEIK | |
| | | | LVTVSS | | | |
| | 69A7 | CD70 | QVQLQESGPGL | 742 | EIVLTQSPATL | 842 |
| | | | VKPSETLSLTC | | SLSPGERATLS | |
| | | | TVSGGSVSSDY | | CRASQSVSSYL | |
| | | | YYWSWIRQPPG | | AWYQQKPGQAP | |
| | | | KGLEWLGYIYY | | RLLIFDASNRA | |
| | | | SGSTNYNPSLK | | TGIPARFSGSG | |
| | | | SRVTISVDTSK | | SGTDFTLTISS | |
| | | | NQFSLKLRSVT | | LEPEDFAVYYC | |
| | | | TA | | QQ | |
| | | | DTAVYYCARGD | | RSNWPLTFGGG | |
| | | | GDYGGNCFDYW | | TKVEIK | |
| | | | GQGTLVTVSS | | | |
| CE-355621 | | cMET | QVQLVQSGAEV | 743 | DIQMTQSPSSV | 843 |
| | | | KKPGASVKVSC | | SASVGDRVTIT | |
| | | | KASGYTFTSYG | | CRASQGINTWL | |
| | | | FSWVRQAPGQG | | AWYQQKPGKAP | |
| | | | LEWMGWISASN | | KLLIYAASSLK | |
| | | | GNTYYAQKLQG | | SGVPSRFSGSG | |
| | | | RVTMTTDTSTS | | SGTDFTLTISS | |
| | | | TAYMELRSLRS | | LQPEDFATYYC | |
| | | | DDTAVYYCARV | | QQANSFPLTFG | |
| | | | YADYADYNGQG | | GGTKVEIK | |
| | | | TLVTVSS | | | |
| LY2875358 | emibetuzumab | cMET | QVQLVQSGAEV | 744 | DIQMTQSPSSL | 844 |
| | | | KKPGASVKVSC | | SASVGDRVTIT | |
| | | | KASGYTFTDYY | | CSVSSSVSSIY | |
| | | | MHWVRQAPGQG | | LHWYQQKPGKA | |
| | | | LEWMGRVNPNR | | PKLLIYSTSNL | |
| | | | RGTTYNQKFEG | | ASGVPSRFSGS | |
| | | | RVTMTTDTSTS | | GSGTDFTLTIS | |
| | | | TAYMELRSLRS | | SLQPEDFATYY | |
| | | | DDTAVYYCARA | | CQVYSGYPLTF | |
| | | | NWLDYWGQGTT | | GGGTKVEIK | |
| | | | VTVSS | | | |
| MetMAb | onartuzumab | cMET | EVQLVESGGGL | 745 | DIQMTQSPSSL | 845 |
| | | | VQPGGSLRLSC | | SASVGDRVTIT | |
| | | | AASGYTFTSYW | | CKSSQSLLYTS | |
| | | | LHWVRQAPGKG | | SQKNYLAWYQQ | |
| | | | LEWVGMIDPSN | | KPGKAPKLLIY | |
| | | | SDTRFNPNFKD | | WASTRESGVPS | |
| | | | RFTISADTSKN | | RFSGSGSGTDF | |
| | | | TAYLQMNSLRA | | TLTISSLQPED | |
| | | | EDTAVYYCATY | | FATYYCQQYYA | |
| | | | RSYVTPLDYWG | | YPWTFGQGTKV | |
| | | | QGTLVTVSS | | EIK | |
| | tremelimumab | CTLA4 | QVQLVESGGGV | 746 | DIQMTQSPSSL | 846 |
| | (CP-675206, | | VQPGRSLRLSC | | SASVGDRVTIT | |
| | or 11.2.1) | | AASGFTFSSYG | | CRASQSINSYL | |
| | | | MHWVRQAPGKG | | DWYQQKPGKAP | |
| | | | LEWVAVIWYDG | | KLLIYAASSLQ | |
| | | | SNKYYADSVKG | | SGVPSRFSGSG | |
| | | | RFTISRDNSKN | | SGTDFTLTISS | |
| | | | TLYLQMNSLRA | | LQPEDFATYYC | |
| | | | EDTAVYYCARD | | QQYYSTPFTFG | |
| | | | PRGATLYYYYY | | PGTKVEIK | |
| | | | GMDVKGQGTTV | | | |
| | | | TVSS | | | |
| Yervoy | Ipilimumab | CTLA4 | QVQLVESGGGV | 747 | EIVLTQSPGTL | 847 |
| | 10D1 | | VQPGRSLRLSC | | SLSPGERATLS | |
| | | | AASGFTFSSYT | | CRASQSVGSSY | |
| | | | MHWVRQAPGKG | | LAWYQQKPGQA | |
| | | | LEWVTFISYDG | | PRLLIYGAFSR | |
| | | | NNKYYADSVKG | | ATGIPDRFSGS | |
| | | | RFTISRDNSKN | | GSGTDFTLTIS | |
| | | | TLYLQMNSLRA | | RLEPEDFAVYY | |
| | | | EDTAIYYCART | | CQQYGSSPWTF | |
| | | | GWLGPFDYWGQ | | GQGTKVEIK | |
| | | | GTLVTVSS | | | |
| AGS16F | H16-7.8 | ENPP3 | QVQLQESGPGL | 748 | EIVLTQSPDFQ | 848 |
| | | | VKPSQTLSLTC | | SVTPKEKVTIT | |
| | | | TVSGGSISSGG | | CRASQSIGISL | |
| | | | YYWSWIRQHPG | | HWYQQKPDQSP | |
| | | | KGLEWIGIIYY | | KLLIKYASQSF | |
| | | | SGSTYYNPSLK | | SGVPSRFSGSG | |
| | | | SRVTISVDTSK | | SGTDFTLTINS | |
| | | | NQFSLKLNSVT | | LEAEDAATYYC | |
| | | | AADTAVFYCAR | | HQSRSFPWTFG | |
| | | | VAIVTTIPGGM | | QGTKVEIK | |
| | | | DVWGQGTTVTV | | | |
| | | | SS | | | |
| MT110 | solitomab | EpCAM | EVQLLEQSGAE | 749 | ELVMTQSPSSL | 849 |
| | | | LVRPGTSVKIS | | TVTAGEKVTMS | |
| | | | CKASGYAFTNY | | CKSSQSLLNSG | |
| | | | WLGWVKQRPGH | | NQKNYLTWYQQ | |
| | | | GLEWIGDIFPG | | KPGQPPKLLIY | |
| | | | SGNIHYNEKFK | | WASTRESGVPD | |
| | | | GKATLTADKSS | | RFTGSGSGTDF | |
| | | | STAYMQLSSLT | | TLTISSVQAED | |
| | | | FEDSAVYFCAR | | LAVYYCQNDYS | |
| | | | LRNWDEPMDYW | | YPLTFGAGTKL | |
| | | | GQGTTVTVSS | | EIK | |
| MT201 | Adecatumumab | EpCAM | EVQLLESGGGV | 750 | ELQMTQSPSSL | 850 |
| | | | VQPGRSLRLSC | | SASVGDRVTIT | |
| | | | AASGFTFSSYG | | CRTSQSISSYL | |
| | | | MHWVRQAPGKG | | NWYQQKPGQPP | |
| | | | LEWVAVISYDG | | KLLIYWASTRE | |
| | | | SNKYYADSVKG | | SGVPDRFSGSG | |
| | | | RFTISRDNSKN | | SGTDFTLTISS | |
| | | | TLYLQMNSLRA | | LQPEDSATYYC | |
| | | | EDTAVYYCAKD | | QQSYDIPYTFG | |
| | | | MGWGSGWRPYY | | QGTKLEIK | |
| | | | YYGMDVWGQGT | | | |
| | | | TVTVSS | | | |
| Panorex | Edrecolomab | EpCAM | QVQLQQSGAEL | 751 | NIVMTQSPKSM | 851 |
| | Mab CO17-1A | | VRPGTSVKVSC | | SMSVGERVTLT | |
| | | | KASGYAFTNYL | | CKASENVVTYV | |
| | | | IEWVKQRPGQG | | SWYQQKPEQSP | |
| | | | LEWIGVINPGS | | KLLIYGASNRY | |
| | | | GGTNYNEKFKG | | TGVPDRFTGSG | |
| | | | KATLTADKSSS | | SATDFTLTISS | |
| | | | TAYMQLSSLTS | | VQAEDLADYHC | |
| | | | DDSAVYFCARD | | GQGYSYPYTFG | |
| | | | GPWFAYWGQGT | | GGTKLEIK | |
| | | | LVTVSA | | | |
| | tucotuzumab | EpCAM | QIQLVQSGPEL | 752 | QILLTQSPAIM | 852 |
| | | | KKPGETVKISC | | SASPGEKVTMT | |
| | | | KASGYTFTNYG | | CSASSSVSYML | |
| | | | MNWVRQAPGKG | | WYQQKPGSSPK | |
| | | | LKWMGWINTYT | | PWIFDTSNLAS | |
| | | | GEPTYADDFKG | | GFPARFSGSGS | |
| | | | RFVFSLETSAS | | GTSYSLIISSM | |
| | | | TAFLQLNNLRS | | EAEDAATYYCH | |
| | | | EDTATYFCVRF | | QRSGYPYTFGG | |
| | | | ISKGDYWGQGT | | GTKLEIK | |
| | | | SVTVSS | | | |
| UBS-54 | | EpCAM | VQLQQSDAELV | 753 | DIVMTQSPDSL | 853 |
| | | | KPGASVKISCK | | AVSLGERATIN | |
| | | | ASGYTFTDHAI | | CKSSQSVLYSS | |
| | | | HWVKQNPEQGL | | NNKNYLAWYQQ | |
| | | | EWIGYFSPGND | | KPGQPPKLLIY | |
| | | | DFKYNERFKGK | | WASTRESGVPD | |
| | | | ATLTADKSSST | | RFSGSGSGTDF | |
| | | | AYVQLNSLTSE | | TLTISSLQAED | |
| | | | DSAVYFCTRSL | | VAVYYCQQYYS | |
| | | | NMAYWGQGTSV | | YPLTFGGGTKV | |
| | | | TVSS | | KES | |
| 3622W94 | 323/A3 | EpCAM | EVQLVQSGPEV | 754 | DIVMTQSPLSL | 854 |
| | | | KKPGASVKVSC | | PVTPGEPASIS | |
| | | | KASGYTFTNYG | | CRSSINKKGSN | |
| | | | MNWVRQAPGQG | | GITYLYWYLQK | |
| | | | LEWMGWINTYT | | PGQSPQLLIYQ | |
| | | | GEPTYGEDFKG | | MSNLASGVPDR | |
| | | | RFAFSLDTSAS | | FSGSGSGTDFT | |
| | | | TAYMELSSLRS | | LKISRVEAEDV | |
| | | | EDTAVYFCARF | | GVYYCAQNLEI | |
| | | | GNYVDYWGQGS | | PRTFGQGTKVE | |
| | | | LVTVSS | | IK | |
| 4D5MOCBv2 | | EpCAM | EVQLVQSGPGL | 755 | DIQMTQSPSSL | 855 |
| | | | VQPGGSVRISC | | SASVGDRVTIT | |
| | | | AASGYTFTNYG | | CRSTKSLLHSN | |
| | | | MNWVKQAPGKG | | GITYLYWYQQK | |
| | | | LEWMGWINTYT | | PGKAPKLLIYQ | |
| | | | GESTYADSFKG | | MSNLASGVPSR | |
| | | | RFTFSLDTSAS | | FSSSGSGTDFT | |
| | | | AAYLQINSLRA | | LTISSLQPEDF | |
| | | | EDTAVYYCARF | | ATYYCAQNLEI | |
| | | | AIKGDYWGQGT | | PRTFGQGTKVE | |
| | | | LLTVSS | | IK | |
| 4D5MOCB | | EpCAM | EVQLVQSGPGL | 756 | DIQMTQSPSSL | 856 |
| | | | VQPGGSVRISC | | SASVGDRVTIT | |
| | | | AASGYTFTNYG | | CRSTKSLLHSN | |
| | | | MNWVKQAPGKG | | GITYLYWYQQK | |
| | | | LEWMGWINTYT | | PGKAPKLLIYg | |
| | | | GESTYADSFKG | | MSNLASGVPSR | |
| | | | RFTFSLDTSAS | | FSSSGSGTDFT | |
| | | | AAYLQINSLRA | | LTISSLQPEDF | |
| | | | EDTAVYYCARF | | ATYYCAQNLEI | |
| | | | AIKGDYWGQGT | | PRTFGQGTKVE | |
| | | | LLTVSS | | LK | |
| MEDI-547 | 1C1 | EphA2 | EVQLLESGGGL | 757 | DIQMTQSPSSL | 857 |
| | | | VQPGGSLRLSC | | SASVGDRVTIT | |
| | | | AASGFTFSHYM | | CRASQSISTWL | |
| | | | MAWVRQAPGKG | | AWYQQKPGKAP | |
| | | | LEWVSRIGPSG | | KLLIYKASNLH | |
| | | | GPTHYADSVKG | | TGVPSRFSGSG | |
| | | | RFTISRDNSKN | | SGTEFSLTISG | |
| | | | TLYLQMNSLRA | | LQPDDFATYYC | |
| | | | EDTAVYYCAGY | | QQYNSYSRTFG | |
| | | | DSGYDYVAVAG | | QGTKVEIK | |
| | | | PAEYFQHWGQG | | | |
| | | | TLVTVSS | | | |
| MORAb-003 | farletuzumab | FOLR1 | EVQLVESGGGV | 758 | DIQLTQSPSSL | 858 |
| | | | VQPGRSLRLSC | | SASVGDRVTIT | |
| | | | SASGFTFSGYG | | CSVSSSISSNN | |
| | | | LSWVRQAPGKG | | LHWYQQKPGKA | |
| | | | LEWVAMISSGG | | PKPWIYGTSNL | |
| | | | SYTYYADSVKG | | ASGVPSRFSGS | |
| | | | RFAISRDNAKN | | GSGTDYTFTIS | |
| | | | TLFLQMDSLRP | | SLQPEDIATYY | |
| | | | EDTGVYFCARH | | CQQWSSYPYMY | |
| | | | GDDPAWFAYWG | | TFGQGTKVEIK | |
| | | | QGTPVTVSS | | | |
| M9346A | huMOV19 | FOLR1 | QVQLVQSGAEV | 759 | DIVLTQSPLSL | 859 |
| | (vLCv1.60) | | VKPGASVKISC | | AVSLGQPAIIS | |
| | | | KASGYTFTGYF | | CKASQSVSFAG | |
| | | | MNWVKQSPGQS | | TSLMHWYHQKP | |
| | | | LEWIGRIHPYD | | GQQPRLLIYRA | |
| | | | GDTFYNQKFQG | | SNLEAGVPDRF | |
| | | | KATLTVDKSSN | | SGSGSKTDFTL | |
| | | | TAHMELLSLTS | | NISPVEAEDAA | |
| | | | EDFAVYYCTRY | | TYYCQQSREYP | |
| | | | DGSRAMDYWGQ | | YTFGGGTKLEI | |
| | | | GTTVTVSS | | K | |
| M9346A | huMOV19 | FOLR1 | QVQLVQSGAEV | 760 | DIVLTQSPLSL | 860 |
| | (vLCv1.60) | | VKPGASVKISC | | AVSLGQPAIIS | |
| | | | KASGYTFTGYF | | CKASQSVSFAG | |
| | | | MNWVKQSPGQS | | TSLMHWYHQKP | |
| | | | LEWIGRIHPYD | | GQQPRLLIYRA | |
| | | | GDTFYNQKFQG | | SNLEAGVPDRF | |
| | | | KATLTVDKSSN | | SGSGSKTDFTL | |
| | | | TAHMELLSLTS | | TISPVEAEDAA | |
| | | | EDFAVYYCTRY | | TYYCQQSREYP | |
| | | | DGSRAMDYWGQ | | YTFGGGTKLEI | |
| | | | GTTVTVSS | | K | |
| 26B3.F2 | | FOLR1 | GPELVKPGASV | 761 | PASLSASVGET | 861 |
| | | | KISCKASDYSF | | VTITCRTSENI | |
| | | | TGYFMNWYMQS | | FSYLAWYQQKQ | |
| | | | HGKSLEWIGRI | | GISPQLLVYNA | |
| | | | FPYNGDTFYNQ | | KTLAEGVPSRF | |
| | | | KFKGRATLTVD | | SGSGSGTQFSL | |
| | | | KSSSTAHMELR | | KINSLQPEDFG | |
| | | | SLASEDSAVYF | | SYYCQHHYAFP | |
| | | | CARGTHYFDYW | | WTFGGGSKLEI | |
| | | | GQGTTLTVSS | | K | |
| RG7686 | GC33 | GPC3 | QVQLVQSGAEV | 762 | DWMTQSPLSL | 862 |
| | | | KKPGASVKVSC | | PVTPGEPASIS | |
| | | | KASGYTFTDYE | | CRSSQSLVHSN | |
| | | | MHWVRQAPGQG | | GNTYLHWYLQK | |
| | | | LEWMGALDPKT | | PGQSPQLLIYK | |
| | | | GDTAYSQKFKG | | VSNRFSGVPDR | |
| | | | RVTLTADKSTS | | FSGSGSGTDFT | |
| | | | TAYMELSSLTS | | LKISRVEAEDV | |
| | | | ED | | GV | |
| | | | TAVYYCTRFYS | | YYCSQNTHVPP | |
| | | | YTYWGQGTLVT | | TFGQGTKLEIK | |
| | | | VSS | | | |
| | 4A6 | GPC3 | EVQLVQSGAEV | 763 | EIVLTQSPGTL | 863 |
| | | | KKPGESLKISC | | SLSPGERATLS | |
| | | | KGSGYSFTSYW | | CRAVQSVSSSY | |
| | | | IAWVRQMPGKG | | LAWYQQKPGQA | |
| | | | LEWMGIIFPGD | | PRLLIYGASSR | |
| | | | SDTRYSPSFQG | | ATGIPDRFSGS | |
| | | | QVTISADRSIR | | GSGTDFTLTIS | |
| | | | TAYLQWSSLKA | | RLEPEDFAVYY | |
| | | | SD | | CQQYGSSPTFG | |
| | | | TALYYCARTRE | | GGTKVEIK | |
| | | | GYFDYWGQGTL | | | |
| | | | VTVSS | | | |
| | | | EVQLVQSGAEV | 764 | EIVLTQSPGTL | 864 |
| | | | KKPGESLKISC | | SLSPGERATLS | |
| | | | KGSGYSFTNYW | | CRASQSVSSSY | |
| | | | IAWVRQMPGKG | | LAWYQQKPGQA | |
| | | | LEWMGIIYPGD | | PRLLIYGASSR | |
| | 11E7 | GPC3 | SDTRYSPSFQG | | ATGIPDRFSGS | |
| | | | QVTISADKSIR | | GSGTDFTLTIS | |
| | | | TAYLQWSSLKA | | RLEPEDFAVYY | |
| | | | SD | | CQQYGSSPTFG | |
| | | | TAMYYCARTRE | | GGTKVEIK | |
| | | | GYFDYWGQGTL | | | |
| | | | VTVSS | | | |
| | 16D10 | GPC3 | EVQLVQSGADV | 765 | EILLTQSPGTL | 865 |
| | | | TKPGESLKISC | | SLSPGERATLS | |
| | | | KVSGYRFTNYW | | CRASQSVSSSY | |
| | | | IGWMRQMSGKG | | LAWYQQKPGQA | |
| | | | LEWMGIIYPGD | | PRLLIYGASSR | |
| | | | SDTRYSPSFQG | | ATGIPDRFSGS | |
| | | | HVTISADKSIN | | GSGTDFTLTIS | |
| | | | TAYLRWSSLKA | | RLEPEDFAVYY | |
| | | | SD | | CQQYGSSPTFG | |
| | | | TAIYYCARTRE | | QGTKVEIK | |
| | | | GFFDYWGQGTP | | | |
| | | | VTVSS | | | |
| AMG-595 | | EGFR | QVQLVESGGGV | 766 | DTVMTQTPLSS | 866 |
| | | | VQSGRSLRLSC | | HVTLGQPASIS | |
| | | | AASGFTFRNYG | | CRSSQSLVHSD | |
| | | | MHWVRQAPGKG | | GNTYLSWLQQR | |
| | | | LEWVAVIWYDG | | PGQPPRLLIYR | |
| | | | SDKYYADSVRG | | ISRRFSGVPDR | |
| | | | RFTISRDNSKN | | FSGSGAGTDFT | |
| | | | TLYLQMNSLRA | | LEISRVEAEDV | |
| | | | EDTAVYYCARD | | GVYYCMQSTHV | |
| | | | GYDILTGNPRD | | PRTFGQGTKVE | |
| | | | FDYWGQGTLVT | | IK | |
| | | | VSS | | | |
| Erubitux™ | cetutximab | EGFR | QVQLKQSGPGL | 767 | DILLTQSPVIL | 867 |
| | | | VQPSQSLSITC | | SVSPGERVSFS | |
| | | | TVSGFSLTNYG | | CRASQSIGTNI | |
| | | | VHWVRQSPGKG | | HWYQQRTNGSP | |
| | | | LEWLGVIWSGG | | RLLIKYASESI | |
| | | | NTDYNTPFTSR | | SGIPSRFSGSG | |
| | | | LSINKDNSKSQ | | SGTDFTLSINS | |
| | | | VFFKMNSLQSN | | VESEDIADYYC | |
| | | | DTAIYYCARAL | | QQNNNWPTTFG | |
| | | | TYYDYEFAYWG | | AGTKLELK | |
| | | | QGTLVTVSA | | | |
| GA201 | Imgatuzumab | EGFR | QVQLVQSGAEV | 768 | DIQMTQSPSSL | 868 |
| | | | KKPGSSVKVSC | | SASVGDRVTIT | |
| | | | KASGFTFTDYK | | CRASQGINNYL | |
| | | | IHWVRQAPGQG | | NWYQQKPGKAP | |
| | | | LEWMGYFNPNS | | KRLIYNTNNLQ | |
| | | | GYSTYAQKFQG | | TGVPSRFSGSG | |
| | | | RVTITADKSTS | | SGTEFTLTISS | |
| | | | TAYMELSSLRS | | LQPEDFATYYC | |
| | | | EDTAVYYCARL | | LQHNSFPTFGQ | |
| | | | SPGGYYVMDAW | | GTKLEIK | |
| | | | GQGTTVTVSS | | | |
| Humax | zalutumumab | EGFR | QVQLVESGGGV | 769 | AIQLTQSPSSL | 869 |
| | | | VQPGRSLRLSC | | SASVGDRVTIT | |
| | | | AASGFTFSTYG | | CRASQDISSAL | |
| | | | MHWVRQAPGKG | | VWYQQKPGKAP | |
| | | | LEWVAVIWDDG | | KLLIYDASSLE | |
| | | | SYKYYGDSVKG | | SGVPSRFSGSE | |
| | | | RFTISRDNSKN | | SGTDFTLTISS | |
| | | | TLYLQMNSLRA | | LQPEDFATYYC | |
| | | | EDTAVYYCARD | | QQFNSYPLTFG | |
| | | | GITMVRGVMKD | | GGTKVEIK | |
| | | | YFDYWGQGTLV | | | |
| | | | TVSS | | | |
| IMC-11F8 | necitumumab | EGFR | QVQLQESGPGL | 770 | EIVMTQSPATL | 870 |
| | | | VKPSQTLSLTC | | SLSPGERATLS | |
| | | | TVSGGSISSGD | | CRASQSVSSYL | |
| | | | YYWSWIRQPPG | | AWYQQKPGQAP | |
| | | | KGLEWIGYIYY | | RLLI YDASNRA | |
| | | | SGSTDYNPSLK | | TGIPARFSGSG | |
| | | | SRVTMSVDTSK | | SGTDFTLTISS | |
| | | | NQFSLKVNSVT | | LEPEDFAVYYC | |
| | | | AADTAVYYCAR | | HQYGSTPLTFG | |
| | | | VSIFGVGTFDY | | GGTKAEIK | |
| | | | WGQGTLVTVSS | | | |
| MM-151 | P1X | EGFR | QVQLVQSGAEV | 771 | DIQMTQSPSTL | 871 |
| | | | KKPGSSVKVSC | | SASVGDRVTIT | |
| | | | KASGGTFSSYA | | CRASQSISSWW | |
| | | | ISWVRQAPGQG | | AWYQQKPGKAP | |
| | | | LEWMGSIIPIF | | KLLIYDASSLE | |
| | | | GTVNYAQKFQG | | SGVPSRFSGSG | |
| | | | RVTITADESTS | | SGTEFTLTISS | |
| | | | TAYMELSSLRS | | LQPDDFATYYC | |
| | | | EDTAVYYCARD | | QQYHAHPTTFG | |
| | | | PSVNLYWYFDL | | GGTKVEIK | |
| | | | WGRGTLVTVSS | | | |
| MM-151 | P2X | EGFR | QVQLVQSGAEV | 772 | DIVMTQSPDSL | 872 |
| | | | KKPGSSVKVSC | | AVSLGERATIN | |
| | | | KASGGTFGSYA | | CKSSQSVLYSP | |
| | | | ISWVRQAPGQG | | NNKNYLAWYQQ | |
| | | | LEWMGSIIPIF | | KPGQPPKLLIY | |
| | | | GAANPAQKSQG | | WASTRESGVPD | |
| | | | RVTITADESTS | | RFSGSGSGTDF | |
| | | | TAYMELSSLRS | | TLTISSLQAED | |
| | | | EDTAVYYCAKM | | VAVYYCQQYYG | |
| | | | GRGKVAFDIWG | | SPITFGGGTKV | |
| | | | QGTMVTVSS | | EIK | |
| MM-151 | P3X | EGFR | QVQLVQSGAEV | 773 | EIVMTQSPATL | 873 |
| | | | KKPGASVKVSC | | SVSPGERATLS | |
| | | | KASGYAFTSYG | | CRASQSVSSNL | |
| | | | INWVRQAPGQG | | AWYQQKPGQAP | |
| | | | LEWMGWISAYN | | RLLIYGASTRA | |
| | | | GNTYYAQKLRG | | TGIPARFSGSG | |
| | | | RVTMTTDTSTS | | SGTEFTLTISS | |
| | | | TAYMELRSLRS | | LQSEDFAVYYC | |
| | | | DDTAVYYCARD | | QDYRTWPRRVF | |
| | | | LGGYGSGSVPF | | GGGTKVEIK | |
| | | | DPWGQGTLVTV | | | |
| | | | SS | | | |
| TheraCIM | nimotuzumab | EGFR | QVQLQQSGAEV | 774 | DIQMTQSPSSL | 874 |
| | | | KKPGSSVKVSC | | SASVGDRVTIT | |
| | | | KASGYTFTNYY | | CRSSQNIVHSN | |
| | | | IYWVRQAPGQG | | GNTYLDWYQQT | |
| | | | LEWIGGINPTS | | PGKAPKLLIYK | |
| | | | GGSNFNEKFKT | | VSNRFSGVPSR | |
| | | | RVTITADESST | | FSGSGSGTDFT | |
| | | | TAYMELSSLRS | | FTISSLQPEDI | |
| | | | EDTAFYFCTRQ | | ATYYCFQYSHV | |
| | | | GLWFDSDGRGF | | PWTFGQGTKLQ | |
| | | | DFWGQGTTVTV | | IT | |
| | | | SS | | | |
| Vectibix™ | panitumimab | EGFR | QVQLQESGPGL | 775 | DIQMTQSPSSL | 875 |
| | | | VKPSETLSLTC | | SASVGDRVTIT | |
| | | | TVSGGSVSSGD | | CQASQDISNYL | |
| | | | YYWTWIRQSPG | | NWYQQKPGKAP | |
| | | | KGLEWIGHIYY | | KLLIYDASNLE | |
| | | | SGNTNYNPSLK | | TGVPSRFSGSG | |
| | | | SRLTISIDTSK | | SGTDFTFTISS | |
| | | | TQFSLKLSSVT | | LQPEDIATYFC | |
| | | | AADTAIYYCVR | | QHFDHLPLAFG | |
| | | | DRVTGAFDIWG | | GGTKVEIK | |
| | | | QGTMVTVSS | | | |
| 07D06 | | EGFR | QIQLVQSGPEL | 776 | DWMTQTPLSL | 876 |
| | | | KKPGETVKISC | | PVSLGDQASIS | |
| | | | KASGYTFTEYP | | CRSSQSLVHSN | |
| | | | IHWVKQAPGKG | | GNTYLHWYLQK | |
| | | | FKWMGMIYTDI | | PGQSPKLLIYK | |
| | | | GKPTYAEEFKG | | VSNRFSGVPDR | |
| | | | RFAFSLETSAS | | FSGSGSGTDFT | |
| | | | TAYLQINNLKN | | LKISRVEAEDL | |
| | | | EDTATYFCVRD | | GVYFCSQSTHV | |
| | | | RYDSLFDYWGQ | | PWTFGGGTKLE | |
| | | | GTTLTVSS | | IK | |
| 12D03 | | EGFR | EMQLVESGGGF | 777 | DWMTQTPLSL | 877 |
| | | | VKPGGSLKLSC | | PVSLGDQASIS | |
| | | | AASGFAFSHYD | | CRSSQSLVHSN | |
| | | | MSWVRQTPKQR | | GNTYLHWYLQK | |
| | | | LEWVAYIASGG | | PGQSPKLLIYK | |
| | | | DITYYADTVKG | | VSNRFSGVPDR | |
| | | | RFTISRDNAQN | | FSGSGSGTDFT | |
| | | | TLYLQMSSLKS | | LKISRVEAEDL | |
| | | | EDTAMFYCSRS | | GVYFCSQSTHV | |
| | | | SYGNNGDALDF | | LTFGSGTKLEI | |
| | | | WGQGTSVTVSS | | K | |
| | C1 | HER2 | QVQLVESGGGL | 778 | QSPSFLSAFVG | 878 |
| | | | VQPGGSLRLSC | | DRITITCRASP | |
| | | | AASGFTFSSYA | | GIRNYLAWYQQ | |
| | | | MGWVRQAPGKG | | KPGKAPKLLIY | |
| | | | LEWVSSISGSS | | AASTLQSGVPS | |
| | | | RYIYYADSVKG | | RFSGSGSGTDF | |
| | | | RFTISRDNSKN | | TLTISSLQPED | |
| | | | TLYLQMNSLRA | | FATYYCQQYNS | |
| | | | EDTAVYYCAKM | | YPLSFGGGTKV | |
| | | | DASGSYFNFWG | | EIK | |
| | | | QGTLVTVSS | | | |
| Erbicin | | HER2 | QVQLLQSAAEV | 779 | QAWTQEPSFS | 879 |
| | | | KKPGESLKISC | | VSPGGTVTLTC | |
| | | | KGSGYSFTSYW | | GLSSGSVSTSY | |
| | | | IGWVRQMPGKG | | YPSWYQQTPGQ | |
| | | | LEWMGIIYPGD | | APRTLIYSTNT | |
| | | | SDTRYSPSFQG | | RSSGVPDRFSG | |
| | | | QVTISADKSIS | | SILGNKAALTI | |
| | | | TAYLQWSSLKA | | TGAQADDESDY | |
| | | | SDTAVYYCARW | | YCVLYMGSGQY | |
| | | | RDSPLWGQGTL | | VFGGGTKLTVL | |
| | | | VTVSS | | | |
| Herceptin | trastuzumab | HER2 | EVQLVESGGGL | 780 | DIQMTQSPSSL | 880 |
| | | | VQPGGSLRLSC | | SASVGDRVTIT | |
| | | | AASGFNIKDTY | | CRASQDVNTAV | |
| | | | IHWVRQAPGKG | | AWYQQKPGKAP | |
| | | | LEWVARIYPTN | | KLLIYSASFLY | |
| | | | GYTRYADSVKG | | SGVPSRFSGSR | |
| | | | RFTISADTSKN | | SGTDFTLTISS | |
| | | | TAYLQMNSLRA | | LQPEDFATYYC | |
| | | | EDTAVYYCSRW | | QQHYTTPPTFG | |
| | | | GGDGFYAMDYW | | QGTKVEIK | |
| | | | GQGTLVTVSS | | | |
| MAGH22 | margetuximab | HER2 | QVQLQQSGPEL | 781 | DIVMTQSHKFM | 881 |
| | | | VKPGASLKLSC | | STSVGDRVSIT | |
| | | | TASGFNIKDTY | | CKASQDVNTAV | |
| | | | IHWVKQRPEQG | | AWYQQKPGHSP | |
| | | | LEWIGRIYPTN | | KLLIYSASFRY | |
| | | | GYTRYDPKFQD | | TGVPDRFTGSR | |
| | | | KATITADTSSN | | SGTDFTFTISS | |
| | | | TAYLQVSRLTS | | VQAEDLAVYYC | |
| | | | EDTAVYYCSRW | | QQHYTTPPTFG | |
| | | | GGDGFYAMDYW | | GGTKVEIK | |
| | | | GQGASVTVSS | | | |
| MM-302 | F5 | HER2 | QVQLVESGGGL | 782 | QSVLTQPPSVS | 882 |
| | | | VQPGGSLRLSC | | GAPGQRVTISC | |
| | | | AASGFTFRSYA | | TGSSSNIGAGY | |
| | | | MSWVRQAPGKG | | GVHWYQQLPGT | |
| | | | LEWVSAISGRG | | APKLLIYGNTN | |
| | | | DNTYYADSVKG | | RPSGVPDRFSG | |
| | | | RFTISRDNSKN | | FKSGTSASLAI | |
| | | | TLYLQMNSLRA | | TGLQAEDEADY | |
| | | | EDTAVYYCAKM | | YCQFYDSSLSG | |
| | | | TSNAFAFDYWG | | WVFGGGTKLTV | |
| | | | QGTLVTVSS | | L | |
| Perjeta | pertuzumab | HER2 | EVQLVESGGGL | 783 | DIQMTQSPSSL | 883 |
| | | | VQPGGSLRLSC | | SASVGDRVTIT | |
| | | | AASGFTFTDYT | | CKASQDVSIGV | |
| | | | MDWVRQAPGKG | | AWYQQKPGKAP | |
| | | | LEWVADVNPNS | | KLLIYSASYRY | |
| | | | GGSIYNQRFKG | | TGVPSRFSGSG | |
| | | | RFTLSVDRSKN | | SGTDFTLTISS | |
| | | | TLYLQMNSLRA | | LQPEDFATYYC | |
| | | | EDTAVYYCARN | | QQYYIYPYTFG | |
| | | | LGPSFYFDYWG | | QGTKVEIK | |
| | | | QGTLVTVSS | | | |
| MM-121/ | | HER3 | EVQLLESGGGL | 784 | QSALTQPASVS | 884 |
| SAR256212 | | | VQPGGSLRLSC | | GSPGQSITISC | |
| | | | AASGFTFSHYV | | TGTSSDVGSYN | |
| | | | MAWVRQAPGKG | | VVSWYQQHPGK | |
| | | | LEWVSSISSSG | | APKLIIYEVSQ | |
| | | | GWTLYADSVKG | | RPSGVSNRFSG | |
| | | | RFTISRDNSKN | | SKSGNTASLTI | |
| | | | TLYLQMNSLRA | | SGLQTEDEADY | |
| | | | EDTAVYYCTRG | | YCCSYAGSSIF | |
| | | | LKMATIFDYWG | | VIFGGGTKVTV | |
| | | | QGTLVTVSS | | L | |
| MEHD7945 | Duligotumab | EGFR/HE | EVQLVESGGGL | 785 | DIQMTQSPSSL | 885 |
| A | | R3 | VQPGGSLRLSC | | SASVGDRVTIT | |
| | | | AASGFTLSGDW | | CRASQNIATDV | |
| | | | IHWVRQAPGKG | | AWYQQKPGKAP | |
| | | | LEWVGEISAAG | | KLLIYSASFLY | |
| | | | GYTDYADSVKG | | SGVPSRFSGSG | |
| | | | RFTISADTSKN | | SGTDFTLTISS | |
| | | | TAYLQMNSLRA | | LQPEDFATYYC | |
| | | | EDTAVYYCARE | | QQSEPEPYTFG | |
| | | | SRVSFEAAMDY | | QGTKVEIK | |
| | | | WGQGTLVTVSS | | | |
| MM-111 | | HER2/3 | QVQLQESGGGL | 786 | QSALTQPASVS | 886 |
| | | | VKPGGSLRLSC | | GSPGQSITISC | |
| | | | AASGFTFSSYW | | TGTSSDVGGYN | |
| | | | MSWVRQAPGKG | | FVSWYQQHPGK | |
| | | | LEWVANINRDG | | APKLMIYDVSD | |
| | | | SASYYVDSVKG | | RPSGVSDRFSG | |
| | | | RFTISRDDAKN | | SKSGNTASLII | |
| | | | SLYLQMNSLRA | | SGLQADDEADY | |
| | | | EDTAVYYCARD | | YCSSYGSSSTH | |
| | | | RGVGYFDLWGR | | VIFGGGTKVTV | |
| | | | GTLVTVSS | | L | |
| MM-111 | | HER2/3 | QVQLVQSGAEV | 787 | QSVLTQPPSVS | 887 |
| | | | KKPGESLKISC | | AAPGQ | |
| | | | KGSGYSFTSYW | | KVTISCSGSSS | |
| | | | IAWVRQMPGKG | | NIGNNYVSWYQ | |
| | | | LEYMGLIYPGD | | QLPGTAPKLLI | |
| | | | SDTKYSPSFQG | | YDHTNRPAGVP | |
| | | | QVTISVDKSVS | | DRFSGSKSGTS | |
| | | | TAYLQWSSLKP | | ASLAISGFRSE | |
| | | | SDSAVYFCARH | | DEADYYCASWD | |
| | | | DVGYCTDRTCA | | YTLSGWVFGGG | |
| | | | KWPEWLGVWGQ | | TKLTVL | |
| | | | GTLVTVSS | | | |
| | Hu3S193 | Lewis-Y | EVQLVESGGGV | 788 | DIQMTQSPSSL | 888 |
| | | | VQPGRSLRLSC | | SASVGDRVTIT | |
| | | | STSGFTFSDYY | | CRSSQRIVHSN | |
| | | | MYWVRQAPGKG | | GNTYLEWYQQT | |
| | | | LEWVAYMSNVG | | PGKAPKLLIYK | |
| | | | AITDYPDTVKG | | VSNRFSGVPSR | |
| | | | RFTISRDNSKN | | FSGSGSGTDFT | |
| | | | TLFLQMDSLRP | | FTISSLQPEDI | |
| | | | EDTGVYFCARG | | ATYYCFQGSHV | |
| | | | TRDGSWFAYWG | | PFTFGQGTKLQ | |
| | | | QGTPVTVSS | | IT | |
| BAY 94- | anetumab | Mesothelin | QVELVQSGAEV | 789 | DIALTQPASVS | 889 |
| 9343 | ravtansine | | KKPGESLKISC | | GSPGQSITISC | |
| | | | KGSGYSFTSYW | | TGTSSDIGGYN | |
| | | | IGWVRQAPGKG | | SVSWYQQHPGK | |
| | | | LEWMGIIDPGD | | APKLMIYGVNN | |
| | | | SRTRYSPSFQG | | RPSGVSMRFSG | |
| | | | QVTISADKSIS | | SKSGNTASLTI | |
| | | | TAYLQWSSLKA | | SGLQAEDEADY | |
| | | | SDTAMYYCARG | | YCSSYDIESAT | |
| | | | QLYGGTYMDGW | | PVFGGGTKLTV | |
| | | | GQGTLVTVSS | | L | |
| | SS1 | Mesothelin | QVQLQQSGPEL | 790 | DIELTQSPAIM | 890 |
| | | | EKPGASVKISC | | SASPGEKVTMT | |
| | | | KASGYSFTGYT | | CSASSSVSYMH | |
| | | | MNWVKQSHGKS | | WYQQKSGTSPK | |
| | | | LEWIGLITPYN | | RWIYDTSKLAS | |
| | | | GASSYNQKFRG | | GVPGRFSGSGS | |
| | | | KATLTVDKSSS | | GNSYSLTISSV | |
| | | | TAYMDLLSLTS | | EAEDDATYYCQ | |
| | | | EDSAVYFCARG | | QWSGYPLTFGA | |
| | | | GYDGRGFDYWG | | GTKLEIK | |
| | | | QGTTVTVSS | | | |
| | | Mesothelin | QVYLVESGGGV | 791 | EIVLTQSPATL | 891 |
| | | | VQPGRSLRLSC | | SLSPGERATLS | |
| | | | AASGITFSIYG | | CRASQSVSSYL | |
| | | | MHWVRQAPGKG | | AWYQQKPGQAP | |
| | | | LEWVAVIWYDG | | RLLIYDASNRA | |
| | | | SHEYYADSVKG | | TGIPARFSGSG | |
| | | | RFTISRDNSKN | | SGTDFTLTISS | |
| | | | TLYLLMNSLRA | | LEPEDFAVYYC | |
| | | | ED | | QQ | |
| | | | TAVYYCARDGD | | RSNWPLTFGGG | |
| | | | YYDSGSPLDYW | | TKVEIK | |
| | | | GQGTLVTVSS | | | |
| | | Mesothelin | QVHLVESGGGV | 792 | EIVLTQSPATL | 892 |
| | | | VQPGRSLRLSC | | SLSPGERATLS | |
| | | | VASGITFRIYG | | CRASQSVSSYL | |
| | | | MHWVRQAPGKG | | AWYQQKPGQAP | |
| | | | LEWVAVLWYDG | | RLLIYDASNRA | |
| | | | SHEYYADSVKG | | TGIPARFSGSG | |
| | | | RFTISRDNSKN | | SGTDFTLTISS | |
| | | | TLYLQMNSLRA | | LEPEDFAVYYC | |
| | | | ED | | QQ | |
| | | | TAIYYCARDGD | | RSNWPLTFGGG | |
| | | | YYDSGSPLDYW | | TKVEIK | |
| | | | GQGTLVTVSS | | | |
| | | Mesothelin | EVHLVESGGGL | 793 | EIVLTQSPGTL | 893 |
| | | | VQPGGSLRLSC | | SLSPGERATLS | |
| | | | AASGFTFSRYW | | CRASQSVSSSY | |
| | | | MSWVRQAQGKG | | LAWYQQKPGQA | |
| | | | LEWVASIKQAG | | PRLLIYGASSR | |
| | | | SEKTYVDSVKG | | ATGIPDRFSGS | |
| | | | RFTISRDNAKN | | GSGTDFTLTIS | |
| | | | SLSLQMNSLRA | | RLEPEDFAVYY | |
| | | | ED | | CQ | |
| | | | TAVYYCAREGA | | QYGSSQYTFGQ | |
| | | | YYYDSASYYPY | | GTKLEIK | |
| | | | YYYYSMDVWGQ | | | |
| | | | GTTVTVSS | | | |
| MORAb-009 | amatuximab | Mesothelin | QVQLQQSGPEL | 794 | DIELTQSPAIM | 894 |
| | | | EKPGASVKISC | | SASPGEKVTMT | |
| | | | KASGYSFTGYT | | CSASSSVSYMH | |
| | | | MNWVKQSHGKS | | WYQQKSGTSPK | |
| | | | LEWIGLITPYN | | RWIYDTSKLAS | |
| | | | GASSYNQKFRG | | GVPGRFSGSGS | |
| | | | KATLTVDKSSS | | GNSYSLTISSV | |
| | | | TAYMDLLSLTS | | EAEDDATYYCQ | |
| | | | EDSAVYFCARG | | QWSKHPLTFGS | |
| | | | GYDGRGFDYWG | | GTKVEIK | |
| | | | SGTPVTVSS | | | |
| hPAM4 | | MUC-1 | EVQLQESGPEL | 795 | DIVMTQSPAIM | 895 |
| | | | VKPGASVKMSC | | SASPGEKVTMT | |
| | | | KASGYTFPSYV | | CSASSSVSSSY | |
| | | | LHWVKQKPGQG | | LYWYQQKPGSS | |
| | | | LEWIGYINPYN | | PKLWIYSTSNL | |
| | | | DGTQYNEKFKG | | ASGVPARFSGS | |
| | | | KATLTSDKSSS | | GSGTSYSLTIS | |
| | | | TAYMELSRLTS | | SMEAEDAASYF | |
| | | | ED | | CH | |
| | | | SAVYYCARGFG | | QWNRYPYTFGG | |
| | | | GSYGFAYWGQG | | GTKLEIK | |
| | | | TLITVSA | | | |
| hPAM4- | clivatuzumab | MUC1 | QVQLQQSGAEV | 796 | DIQLTQSPSSL | 896 |
| Cide | | | KKFGASVKVSC | | SASVGDRVTMT | |
| | | | EASGYTFPSYV | | CSASSSVSSSY | |
| | | | LHWVKQAPGQG | | LYWYQQKPGKA | |
| | | | LEWIGYINPYN | | PKLWIYSTSNL | |
| | | | DGTQTNKKFKG | | ASGVPARFSGS | |
| | | | KATLTRDTSIN | | GSGTDFTLTIS | |
| | | | TAYMELSRLRS | | SLQPEDSASYF | |
| | | | DDTAVYYCARG | | CHQWNRYPYTF | |
| | | | FGGSYGFAYNG | | GGGTRLEIK | |
| | | | QGTLVTVSS | | | |
| SAR566658 | huDS6v1.01 | MUC1 | QAQLQVSGAEV | 797 | EIVLTQSPATM | 897 |
| | | | VKPGASVKMSC | | SASPGERVTIT | |
| | | | KASGYTFTSYN | | CSAHSSVSFMH | |
| | | | MHWVKQTPGQG | | WFQQKPGTSPK | |
| | | | LEWIGYIYPGN | | LWIYSTSSLAS | |
| | | | GATNYNQKFQG | | GVPARFGGSGS | |
| | | | KATLTADTSSS | | GTSYSLTISSM | |
| | | | TAYMQISSLTS | | EAEDAATYYCQ | |
| | | | EDSAVYFCARG | | QRSSFPLTFGA | |
| | | | DSVPFAYWGQG | | GTKLELK | |
| | | | TLVTVSA | | | |
| Theragyn | Pemtumomab | MUC1 | QVQLQQSGAEL | 798 | DIVMSQSPSSL | 898 |
| | muHMFG1 | | MKPGASVKISC | | AVSVGEKVTMS | |
| | | | KATGYTFSAYW | | CKSSQSLLYSS | |
| | | | IEWVKQRPGHG | | NQKIYLAWYQQ | |
| | | | LEWIGEILPGS | | KPGQSPKLLIY | |
| | | | NNSRYNEKFKG | | WASTRESGVPD | |
| | | | KATFTADTSSN | | RFTGGGSGTDF | |
| | | | TAYMQLSSLTS | | TLTISSVKAED | |
| | | | EDSAVYYCSRS | | LAVYYCQQYYR | |
| | | | YDFAWFAYWGQ | | YPRTFGGGTKL | |
| | | | GTPVTVSA | | EIK | |
| Therex | Sontuzumab | MUC1 | QVQLVQSGAEV | 799 | DIQMTQSPSSL | 899 |
| | huHMFG1 | | KKPGASVKVSC | | SASVGDRVTIT | |
| | AS1402 | | KASGYTFSAYW | | CKSSQSLLYSS | |
| | R1150 | | IEWVRQAPGKG | | NQKIYLAWYQQ | |
| | | | LEWVGEILPGS | | KPGKAPKLLIY | |
| | | | NNSRYNEKFKG | | WASTRESGVPS | |
| | | | RVTVTRDTSTN | | RFSGSGSGTDF | |
| | | | TAYMELSSLRS | | TFTISSLQPED | |
| | | | EDTAVYYCARS | | IATYYCQQYYR | |
| | | | YDFAWFAYWGQ | | YPRTFGQGTKV | |
| | | | GTLVTVSS | | EIK | |
| MDX-1105 | | PD-L1 | QVQLVQSGAEV | 800 | EIVLTQSPATL | 900 |
| or BMS- | | | KKPGSSVKVSC | | SLSPGERATLS | |
| 936559 | | | KTSGDTFSTYA | | CRASQSVSSYL | |
| | | | ISWVRQAPGQG | | AWYQQKPGQAP | |
| | | | LEWMGGIIPIF | | RLLIYDASNRA | |
| | | | GKAHYAQKFQG | | TGIPARFSGSG | |
| | | | RVTITADESTS | | SGTDFTLTISS | |
| | | | TAYMELSSLRS | | LEPEDFAVYYC | |
| | | | EDTAVYFCARK | | QQRSNWPTFGQ | |
| | | | FHFVSGSPFGM | | GTKVEIK | |
| | | | DVWGQGTTVTV | | | |
| | | | SS | | | |
| MEDI-4736 | durvalumab | PD-L1 | EVQLVESGGGL | 801 | EIVLTQSPGTL | 901 |
| | | | VQPGGSLRLSC | | SLSPGERATLS | |
| | | | AASGFTFSRYW | | CRASQRVSSSY | |
| | | | MSWVRQAPGKG | | LAWYQQKPGQA | |
| | | | LEWVANIKQDG | | PRLLIYDASSR | |
| | | | SEKYYVDSVKG | | ATGIPDRFSGS | |
| | | | RFTISRDNAKN | | GSGTDFTLTIS | |
| | | | SLYLQMNSLRA | | RLEPEDFAVYY | |
| | | | EDTAVYYCARE | | CQQYGSLPWTF | |
| | | | GGWFGELAFDY | | GQGTKVEIK | |
| | | | WGQGTLVTVSS | | | |
| MPDL3280A | atezolizumab | PD-L1 | EVQLVESGGGL | 802 | DIQMTQSPSSL | 902 |
| | | | VQPGGSLRLSC | | SASVGDRVTIT | |
| | | | AASGFTFSDSW | | CRASQDVSTAV | |
| | | | IHWVRQAPGKG | | AWYQQKPGKAP | |
| | | | LEWVAWISPYG | | KLLIYSASFLY | |
| | | | GSTYYADSVKG | | SGVPSRFSGSG | |
| | | | RFTISADTSKN | | SGTDFTLTISS | |
| | | | TAYLQMNSLRA | | LQPEDFATYYC | |
| | | | EDTAVYYCARR | | QQYLYHPATFG | |
| | | | HWPGGFDYWGQ | | QGTKVEIK | |
| | | | GTLVTVSS | | | |
| MSB0010718C | avelumab | PD-L1 | EVQLLESGGGL | 803 | QSALTQPASVS | 903 |
| | | | VQPGGSLRLSC | | GSPGQSITISC | |
| | | | AASGFTFSSYI | | TGTSSDVGGYN | |
| | | | MMWVRQAPGKG | | YVSWYQQHPGK | |
| | | | LEWVSSIYPSG | | APKLMIYDVSN | |
| | | | GITFYADTVKG | | RPSGVSNRFSG | |
| | | | RFTISRDNSKN | | SKSGNTASLTI | |
| | | | TLYLQMNSLRA | | SGLQAEDEADY | |
| | | | EDTAVYYCARI | | YCSSYTSSSTR | |
| | | | KLGTVTTVDYW | | VFGTGTKVTVL | |
| | | | GQGTLVTVSS | | | |
| MLN591 | | PSMA | EVQLVQSGPEV | 804 | DIQMTQSPSSL | 904 |
| | | | KKPGATVKISC | | STSVGDRVTLT | |
| | | | KTSGYTFTEYT | | CKASQDVGTAV | |
| | | | IHWVKQAPGKG | | DWYQQKPGPSP | |
| | | | LEWIGNINPNN | | KLLIYWASTRH | |
| | | | GGTTYNQKFED | | TGIPSRFSGSG | |
| | | | KATLTVDKSTD | | SGTDFTLTISS | |
| | | | TAYMELSSLRS | | LQPEDFADYYC | |
| | | | EDTAVYYCAAG | | QQYNSYPLTFG | |
| | | | WNFDYWGQGTL | | PGTKVDIK | |
| | | | LTVSS | | | |
| MT112 | pasotuxizumab | PSMA | QVQLVESGGGL | 805 | DIQMTQSPSSL | 905 |
| | | | VKPGESLRLSC | | SASVGDRVTIT | |
| | | | AASGFTFSDYY | | CKASQNVDTNY | |
| | | | MYWVRQAPGKG | | AWYQQKPGQAP | |
| | | | LEWVAIISDGG | | KSLIYSASYRY | |
| | | | YYTYYSDIIKG | | SDVPSRFSGSA | |
| | | | RFTISRDNAKN | | SGTDFTLTISS | |
| | | | SLYLQMNSLKA | | VQSEDFATYYC | |
| | | | EDTAVYYCARG | | QQYDSYPYTFG | |
| | | | FPLLRHGAMDY | | GGTKLEIK | |
| | | | WGQGTLVTVSS | | | |
| | | ROR1 | QEQLVESGGRL | 806 | ELVLTQSPSVS | 906 |
| | | | VTPGGSLTLSC | | AALGSPAKITC | |
| | | | KASGFDFSAYY | | TLSSAHKTDTI | |
| | | | MSWVRQAPGKG | | DWYQQLQGEAP | |
| | | | LEWIATIYPSS | | RYLMQVQSDGS | |
| | | | GKTYYATWVNG | | YTKRPGVPDRF | |
| | | | RFTISSDNAQN | | SGSSSGADRYL | |
| | | | TVDLQMNSLTA | | IIPSVQADDEA | |
| | | | AD | | DY | |
| | | | RATYFCARDSY | | YCGADYIGGYV | |
| | | | ADDGALFNIWG | | FGGGTQLTVTG | |
| | | | PGTLVTISS | | | |
| | | ROR1 | EVKLVESGGGL | 807 | DIKMTQSPSSM | 907 |
| | | | VKPGGSLKLSC | | YASLGERVTIT | |
| | | | AASGFTFSSYA | | CKASPDINSYL | |
| | | | MSWVRQIPEKR | | SWFQQKPGKSP | |
| | | | LEWVASISRGG | | KTLIYRANRLV | |
| | | | TTYYPDSVKGR | | DGVPSRFSGGG | |
| | | | FTISRDNVRNI | | SGQDYSLTINS | |
| | | | LYLQMSSLRSE | | LEYEDMGIYYC | |
| | | | DT | | LQ | |
| | | | AMYYCGRYDYD | | YDEFPYTFGGG | |
| | | | GYYAMDYWGQG | | TKLEMK | |
| | | | TSVTVSS | | | |
| | | ROR1 | QSLEESGGRLV | 808 | ELVMTQTPSSV | 908 |
| | | | TPGTPLTLTCT | | SAAVGGTVTIN | |
| | | | VSGIDLNSHWM | | CQASQSIGSYL | |
| | | | SWVRQAPGKGL | | AWYQQKPGQPP | |
| | | | EWIGIIAASGS | | KLLIYYASNLA | |
| | | | TYYANWAKGRF | | SGVPSRFSGSG | |
| | | | TISKTSTTVDL | | SGTEYTLTISG | |
| | | | RIASPTTEDTA | | VQREDAATYYC | |
| | | | TY | | LG | |
| | | | FCARDYGDYRL | | SLSNSDNVFGG | |
| | | | VTFNIWGPGTL | | GTELEIL | |
| | | | VTVSS | | | |
| | | ROR1 | QSVKESEGDLV | 809 | ELVMTQTPSST | 909 |
| | | | TPAGNLTLTCT | | SGAVGGTVTIN | |
| | | | ASGSDINDYPI | | CQASQSIDSNL | |
| | | | SWVRQAPGKGL | | AWFQQKPGQPP | |
| | | | EWIGFINSGGS | | TLLIYRASNLA | |
| | | | TWYASWVKGRF | | SGVPSRFSGSR | |
| | | | TISRTSTTVDL | | SGTEYTLTISG | |
| | | | KMTSLTTDDTA | | VQREDAATYYC | |
| | | | TY | | LG | |
| | | | FCARGYSTYYC | | GVGNVSYRTSF | |
| | | | DFNIWGPGTLV | | GGGTEVWK | |
| | | | TISS | | | |
| CC49 | | TAG-72 | QVQLVQSGAEV | 810 | DIVMSQSPDSL | 910 |
| (Humanized) | | | VKPGASVKISC | | AVSLGERVTLN | |
| | | | KASGYTFTDHA | | CKSSQSLLYSG | |
| | | | IHWVKQNPGQR | | NQKNYLAWYQQ | |
| | | | LEWIGYFSPGN | | KPGQSPKLLIY | |
| | | | DDFKYNERFKG | | WASARESGVPD | |
| | | | KATLTADTSAS | | RFSGSGSGTDF | |
| | | | TAYVELSSLRS | | TLTISSVQAED | |
| | | | EDTAVYFCTRS | | VAVYYCQQYYS | |
| | | | LNMAYWGQGTL | | YPLTFGAGTKL | |
| | | | VTVSS | | ELK | |
| | Murine A1 | TPBG/5T4 | QIQLVQSGPEL | 811 | SIVMTQTPKFL | 911 |
| | | | KKPGETVKISC | | LVSAGDRVTIT | |
| | | | KASGYTFTNFG | | CKASQSVSNDV | |
| | | | MNWVKQGPGEG | | AWYQQKPGQSP | |
| | | | LKWMGWINTNT | | KLLINFATNRY | |
| | | | GEPRYAEEFKG | | TGVPNRFTGSG | |
| | | | RXAFSLETTAS | | YGTDFTFTIST | |
| | | | TAYLQINNLKN | | VQAEDLALYFC | |
| | | | EDTATYFCARD | | QQ | |
| | | | WDGAYFFDYWG | | DYSSPWTFGGG | |
| | | | QGTTLTVSS | | TKLEIK | |
| | Murine A2 | TPBG/5T4 | QVQLQQSRPEL | 812 | SVIMSRGQIVL | 912 |
| | | | VKPGASVKMSC | | TQSPAIMSASL | |
| | | | KASGYTFTDYV | | GERVTLTCTAS | |
| | | | ISWVKQRTGQG | | SSVNSNYLHWY | |
| | | | LEWIGEIYPGS | | QQKPGSSPKLW | |
| | | | NSIYYNEKFKG | | IYSTSNLASGV | |
| | | | RATLTA | | PARFSGSGSGT | |
| | | | DKSSSTAYMQL | | SYSLTISSMEA | |
| | | | SSLTSEDSAVY | | EDAATYYCHQY | |
| | | | FCAMGGNYGFD | | HRSPLTFGAGT | |
| | | | YWGQGTTLTVS | | KLELK | |
| | | | S | | | |
| | Murine A3 | TPBG/5T4 | EVQLVESGGGL | 813 | DIVMTQSHIFM | 913 |
| | | | VQPKGSLKLSC | | STSVGDRVSIT | |
| | | | AASGFTFNTYA | | CKASQDVDTAV | |
| | | | MNWVRQAPGKG | | AWYQQKPGQSP | |
| | | | LEWVARIRSKS | | KLLIYWASTRL | |
| | | | NNYATYYADSV | | TGVPDRFTGSG | |
| | | | KDRFTISRDDS | | SGTDFTLTISN | |
| | | | QSMLYLQMNNL | | VQSEDLADYFC | |
| | | | KTEDTAMYXCV | | QQ | |
| | | | RQWDYDVRAMN | | YSSYPYTFGGG | |
| | | | YWGQGTSVTVS | | TKLEIK | |
| | | | S | | | |
| IMMU-132 | hRS-7 | TROP-2 | QVQLQQSGSEL | 814 | DIQLTQSPSSL | 914 |
| | | | KKPGASVKVSC | | SASVGDRVSIT | |
| | | | KASGYTFTNYG | | CKASQDVSIAV | |
| | | | MNWVKQAPGQG | | AWYQQKPGKAP | |
| | | | LKWMGWINTYT | | KLLIYSASYRY | |
| | | | GEPTYTDDFKG | | TGVPDRFSGSG | |
| | | | RFAFSLDTSVS | | SGTDFTLTISS | |
| | | | TAYLQISSLKA | | LQPEDFAVYYC | |
| | | | DDTAVYFCARG | | QQHYITPLTFG | |
| | | | GFGSSYWYFDV | | AGTKVEIK | |
| | | | WGQGSLVTVSS | | | |
| IMC-18F1 | icrucumab | VEGFR1 | QAQWESGGGV | 815 | EIVLTQSPGTL | 915 |
| | | | VQSGRSLRLSC | | SLSPGERATLS | |
| | | | AASGFAFSSYG | | CRASQSVSSSY | |
| | | | MHWVRQAPGKG | | LAWYQQKPGQA | |
| | | | LEWVAVIWYDG | | PRLLIYGASSR | |
| | | | SNKYYADSVRG | | ATGIPDRFSGS | |
| | | | RFTISRDNSEN | | GSGTDFTLTIS | |
| | | | TLYLQMNSLRA | | RLEPEDFAVYY | |
| | | | EDTAVYYCARD | | CQQYGSSPLTF | |
| | | | HYGSGVHHYFY | | GGGTKVEIK | |
| | | | YGLDVWGQGTT | | | |
| | | | VTVSS | | | |
| Cyramza | ramucirumab | VEGFR2 | EVQLVQSGGGL | 816 | DIQMTQSPSSV | 916 |
| | | | VKPGGSLRLSC | | SASIGDRVTIT | |
| | | | AASGFTFSSYS | | CRASQGIDNWL | |
| | | | MNWVRQAPGKG | | GWYQQKPGKAP | |
| | | | LEWVSSISSSS | | KLLIYDASNLD | |
| | | | SYIYYADSVKG | | TGVPSRFSGSG | |
| | | | RFTISRDNAKN | | SGTYFTLTISS | |
| | | | SLYLQMNSLRA | | LQAEDFAVYFC | |
| | | | EDTAVYYCARV | | QQAKAFPPTFG | |
| | | | TDAFDIWGQGT | | GGTKVDIK | |
| | | | MVTVSSA | | | |
| | | | EVQLVESGGGL | 817 | DIQMTQSPSSL | 917 |
| | | | VQPGGSLRLSC | | SASVGDRVTIT | |
| | | | AASGFTFSSYG | | CRASQDIAGSL | |
| | | | MSWVRQAPGKG | | NWLQQKPGKAI | |
| | | | LEWVATITSGG | | KRLIYATSSLD | |
| g165DFM- | alacizumabpegol | VEGFR2 | SYTYYVDSVKG | | SGVPKRFSGSR | |
| PEG | | | RFTISRDNAKN | | SGSDYTLTISS | |
| | | | TLYLQMNSLRA | | LQPEDFATYYC | |
| | | | EDTAVYYCVRI | | LQYGSFPPTFG | |
| | | | GEDALDYWGQG | | QGTKVEIK | |
| | | | TLVTVSS | | | |
| Imclone | | VEGFR2 | KVQLQQSGTEL | 818 | DIVLTQSPASL | 918 |
| 6.64 | | | VKPGASVKVSC | | AVSLGQRATIS | |
| | | | KASGYIFTEYI | | CRASESVDSYG | |
| | | | IHWVKQRSGQG | | NSFMHWYQQKP | |
| | | | LEWIGWLYPES | | GQPPKLLIYRA | |
| | | | NIIKYNEKFKD | | SNLESGIPARF | |
| | | | KATLTADKSSS | | SGSGSRTDFTL | |
| | | | TVYMELSRLTS | | TINPVEADDVA | |
| | | | EDSAVYFCTRH | | TYYCQQSNEDP | |
| | | | DGTNFDYWGQG | | LTFGAGTKLEL | |
| | | | TTLTVSSA | | K | |

### VI). Bispecific Antigen Binding Compositions—Configurations and Functional Properties

In another aspect, the present disclosure relates to novel chimeric, bispecific antigen binding compositions that bind to an antigen or epitope of the CD3 protein complex of effector cells (e.g., a T cell) and a second target cell marker associated with a diseased cell or tissue. Thus, they can be referred to as T-cell engagers. As described more fully, below, the bispecific antigen binding compositions are configured in an activatable prodrug form that confer advantages over bispecific T-cell engagers and related compounds known in the art. Various compositions of the disclosure have properties that include enhanced stability during their production and purification, enhanced stability and increased half-life in circulation when administered to a subject, the ability to become activated at intended sites of therapy but not in normal, healthy tissue, and, when activated by proteolytic cleavage of the release segments and release of the fused AF1 and AF2, exhibit binding affinity to target and effector cells that is at least comparable to a corresponding conventional bispecific IgG antibody. Upon the binding of the effector cell and target cell by the AF1 and AF2, an immunological synapse is formed that effects activation of the effector cell and promotes the subsequent destruction of the target cell via apoptosis or cytolysis.

Various subject bispecific antigen binding compositions of the disclosure described herein are specifically designed to be in a prodrug form in that the XTEN component(s) shield the antigen binding fragments, reducing their ability to bind their ligands until released from the composition by protease cleavage of any of the protease cleavage sites located within the release segments. Proteases known to be associated with diseased cells or tissues include but are not limited to serine proteases, cysteine proteases, aspartate proteases, and metalloproteases, including but not limited to the specific proteases described herein. This prodrug property of the bispecific antigen binding compositions improves the specificity of the composition towards diseased tissues or cells compared to bispecific T-cell engager therapeutics that are not in a prodrug format. In contrast, by activating the bispecific antigen binding compositions specifically in the microenvironment of the target cell or diseased tissue where the target cell marker and proteases capable of cleaving the release segments are highly expressed, the bispecific antigen binding fragments and XTEN of the constructs are released upon cleavage of the release segment, and the fused antigen binding fragments can crosslink cytotoxic effector cells with cells expressing a target cell marker in a highly specific fashion, thereby directing the cytotoxic potential of the T cell towards the target cell. After protease cleavage, the antigen binding fragments are no longer shielded and effectively regain their full potential to bind to target cells bearing a target cell marker and an effector cell such as a cytotoxic T cell via binding to the CD3 antigen, which forms part of the T cell receptor complex, causing T cell activation that mediates the subsequent lysis of the target cell expressing the particular target cell marker. Thus, the bispecific antigen binding compositions of the disclosure are contemplated to display strong, specific and efficient target cell killing. In such case, cells are eliminated selectively, thereby reducing the potential for toxic side effects.

In one aspect, the disclosure provides activatable bispecific antigen binding fragment compositions comprising two antigen binding fragments, with a first antigen binding fragment that targets an effector cell and a second antigen binding fragment that targets a cell marker associated with a disease tissue or cell; both of which have specific binding affinity for their respective ligands. The design of the subject compositions having a first and a second antigen binding fragment (AF1 and AF2, respectively) was informed by consideration of at least three properties: 1) compositions having bispecific antigen binding fragments with the capability to bind to and link together an effector cell and a target cell with the resultant formation of an immunological synapse; 2) compositions with a XTEN that i) shields both of the antigen binding fragments and reduces their ability to bind the target and effector cell ligands when the composition is in an intact prodrug form, ii) provides enhanced half-life when administered to a subject, iii) reduces extravasation of the intact composition from the circulation in normal tissues and organs compared to diseased tissues (e.g., tumor), and iv) confers an increased safety profile compared to conventional bispecific cytotoxic antibody therapeutics; and 3) is activated when the RS is cleaved by one or more mammalian proteases in proximity of diseased tissues, thereby releasing the fused bispecific antigen binding fragments such that they regain their full binding affinity potential for the target ligands. The design of the subject compositions takes advantage of the properties of XTEN and the release segment (RS) components, and their positioning relative to the bispecific antigen binding fragments achieves the foregoing properties, as evidenced by the results in the illustrative Examples, below.

In one embodiment, the polypeptides of any of the bispecific antigen binding fragment composition embodiments described herein having two antigen binding fragments (AF1 and AF2), a single RS, and a single XTEN, the polypeptide can have, in an uncleaved state, a structural arrangement from N-terminus to C-terminus of AF2-AF1-RS1-XTEN1, AF1-AF2-RS1-XTEN1, XTEN1-RS1-AF2-AF1, XTEN1-RS1-AF1-AF2, or diabody-RS1-XTEN1, or XTEN1-RS1-diabody, wherein the diabody comprises VL and VH of the AF1 and AF2.

In other aspects, the disclosure provides bispecific antigen binding compositions having two antigen binding fragments (AF1 and AF2), two RS, and two XTEN. The design of these compositions was informed by considerations of further reducing the binding affinity of the uncleaved compositions to the respective ligands of the AF1 and AF2 antibody fragments by the addition of the second XTEN in order to further reduce the unintended binding of the compositions to healthy tissues or cells when administered to a subject, thereby further improving the therapeutic index of the subject compositions compared to compositions having only one RS and one XTEN. The addition of the second RS and second XTEN resulted in a surprising reduction of binding affinity of the intact, uncleaved polypeptide to the respective ligands of the AF1 and AF2 antibody fragments relative to those compositions having a single RS and XTEN, when assayed in vitro, and also resulted in reduced toxicity in animal models of disease when administered as therapeutically-effective doses, as described in the Examples, below. In embodiments of the subject compositions having two antigen binding fragments, two RS, and two XTEN, the compositions can have, in an uncleaved state, a structural arrangement from N-terminus to C-terminus of XTEN1-RS1-AF2-AF1-RS2-XTEN2, XTEN1-RS1-AF1-AF2-RS2-XTEN2, XTEN2-RS2-AF2-AF1-RS1-XTEN1, XTEN2-RS2-AF1-AF2-RS1-XTEN1, XTEN2-RS2-diabody-RS1-XTEN1, wherein the diabody comprises VL and VH of the AF1 and AF2, or XTEN1-RS1-diabody-RS2-XTEN2, wherein the diabody comprises VL and VH of the AF1 and AF2.

It is a feature of various designed compositions that when the RS of the bispecific antigen binding composition is cleaved by a mammalian protease in the environment of the target cell and is converted from the prodrug form to the activated or apoprotein form, upon cleavage and release of the bispecific antigen binding fragments and the XTEN from the composition, the fused AF1 and AF2 bind to and link together an effector cell (e.g., a T cell bearing CD3) and a diseased cell (e.g., a tumor or cancer cell) bearing the target cell marker antigen capable of being bound by the AF2, whereupon the effector cell is activated. In one embodiment, wherein RS of the bispecific antigen binding composition is cleaved and the antigen binding fragments are released, the subsequent concurrent binding of the effector cell and the target cell can result in at least a 3-fold, or a 10-fold, or a 30-fold, or a 100-fold, or a 300-fold, or a 1000-fold activation of the effector cell, wherein the activation is assessed by the production of cytokines, cytolytic proteins, or lysis of the target cell, assessed in an in vitro cell-based assay. In another embodiment, the concurrent binding of a T cell bearing the CD3 antigen and a diseased cell bearing the target cell marker antigen by the released, fused AF1 and AF2 forms an immunologic synapse, wherein the binding results in the release of T cell-derived effector molecules capable of lysing the diseased cell. Non-limiting examples of the in vitro assay for measuring effector cell activation and/or cytolysis include cell membrane integrity assay, mixed cell culture assay, FACS based propidium Iodide assay, trypan Blue influx assay, photometric enzyme release assay, ELISA, radiometric 51Cr release assay, fluorometric Europium release assay, CalceinAM release assay, photometric MTT assay, XTT assay, WST-1 assay, alamar Blue assay, radiometric 3H-Thd incorporation assay, clonogenic assay measuring cell division activity, fluorometric Rhodamine123 assay measuring mitochondrial transmembrane gradient, apoptosis assay monitored by FACS-based phosphatidylserine exposure, ELISA-based TUNEL test assay, caspase activity assay, and cell morphology assay, or other assays known in the art for the assay of cytokines, cytolytic proteins, or lysis of cells, or the methods described in the Examples, below.

Without being bound to a particular theory, it is believed that using the bispecific antigen binding composition formats as described above, upon cleavage of the RS, the released fused AF1 and AF2 are capable of killing target cells by recruitment of cytotoxic effector cells without any need for pre- and/or co-stimulation. Further, the independence from pre- and/or co-stimulation of the effector cell may substantially contribute to the exceptionally high cytotoxicity mediated by the released, fused AF1 and AF2 antigen binding fragments. In some embodiments, the released AF1 and AF2, wherein the AF1 remains fused to the AF2 by a linker peptide, is designed with binding specificities such that it has the capability to bind and link together in close proximity cytotoxic effector cells (e.g., T cells, NK cells, cytokine induced killer cell (CIK cell)), to preselected target cell markers by the AF2 that has binding specificity to target cell markers associated with tumor cells, cancer cells, or cells associated with diseased tissues, thereby effecting an immunological synapse and a selective, directed, and localized effect of released cytokines and effector molecules against the target tumor or cancer cell, with the result that tumor or cancer cells are damaged or destroyed, resulting in therapeutic benefit to a subject. The released AF1 that binds to an effector cell antigen is capable of modulating one or more functions of an effector cell, resulting in or contributing to the cytolytic effect on the target cell to which the AF2 is bound; e.g., a tumor cell. The effector cell antigen can by expressed by the effector cell or other cells. In one embodiment, the effector cell antigen is expressed on cell surface of the effector cell. Non-limiting examples of effector cell antigens are CD3, CD4, CD8, CD16, CD25, CD38, CD45RO, CD56, CD57, CD69, CD95, CD107, and CD154. Thus, it will be understood by one of skill in the art that the configurations of the subject compositions are intended to selectively or disproportionately deliver the active form of the composition to the target tumor tissue or cancer cell, compared to healthy tissue or healthy cells in a subject in which the composition is administered, with resultant therapeutic benefit. As is evident from the foregoing, the disclosure provides a large family of polypeptides in designed configurations to effect the desired properties.

It is an object of the disclosure that the design of the subject bispecific antigen binding compositions, with the shielding effect imparted by the XTEN of the intact, circulating composition and the concomitant reduced potential to bind to effector cells and target tissues, results in reduced production of Th1 T-cell associated cytokines or other proinflammatory mediators during systemic exposure when administered to a subject such that the overall side-effect and safety profile (e.g., the therapeutic index) is improved compared to bispecific antigen binding compositions not linked to a shielding moiety such as an XTEN. As an important component of cellular immunity, the production of IL-2, TNF-alpha, and IFN-gamma are hallmarks of a Th1 response (Romagnani S. T-cell subsets (Th1 versus Th2). Ann Allergy Asthma Immunol. 2000. 85(1):9-18), particularly in T cells stimulated by anti-CD3 (Yoon, S. H. Selective addition of CXCR3+CCR4-CD4+ Th1 cells enhances generation of cytotoxic T cells by dendritic cells in vitro. Exp Mol Med. 2009. 41(3):161-170), and 11-4, IL-6, and IL-10 are also proinflammatory cytokines important in a cytotoxic response for bispecific antibody compositions (Zimmerman, Z., et al. Unleashing the clinical power of T cells: CD19/CD3 bi-specific T cell engager (BiTE®) antibody composition blinatumomab as a potential therapy. Int. Immunol. (2015) 27(1): 31-37). In one embodiment, an intact, uncleaved bispecific antigen binding composition of the embodiments described herein can exhibit at least 3-fold, or at least 4-fold, or at least 5-fold, or at least 6-fold, or at least 7-fold, or at least 8-fold, or at least 9-fold, or at least 10-fold, or at least 20-fold, or at least 30-fold, or at least 50-fold, or at least 100-fold, or at least 1000-fold reduced potential to result in the production of Th1 and/or proinflammatory cytokines when the intact, uncleaved polypeptide is in contact with the effector cell and a target cell in an in vitro cell-based cytokine stimulation assay compared to the Th1 and/or cytokine levels stimulated by the corresponding released AF1 and AF2 (which remain fused together after release by proteolysis of the RS) of a corresponding protease-treated composition in the in vitro cell-based stimulation cytokine assay performed under comparable conditions, e.g., equivalent molar concentrations. Non-limiting examples of Th1 and/or proinflammatory cytokines are IL-2, IL-4, IL-6, IL-10, TNF-alpha and IFN-gamma. In one embodiment of the foregoing, the production of the Th1 cytokine is assayed in an in vitro assay comprising effector cells such as PBMC or CD3+ T cells and target cells having a target cell marker antigen disclosed herein. In another embodiment, the cytokines can be assessed from a blood, fluid, or tissue sample removed from a subject in which the polypeptide composition has been administered. In the foregoing embodiment, the subject can be mouse, rat, monkey, and human. In an advantage of the subject bispecific antigen binding compositions of the embodiments described herein, however, it has been discovered that the cytolytic properties of the compositions do not require prestimulation by cytokines; that formation of the immunological synapse of the effector cell bound to the target cell by the antigen binding fragments is sufficient to effect cytolysis or apoptosis in the target cell. Nevertheless, the production of proinflammatory cytokines are useful markers to assess the potency or the effects of the subject polypeptide compositions; whether by in vitro assay or in the monitoring of treatment of a subject with a disease tissue (e.g., such as a tumor) after administration of a subject bispecific antigen binding composition.

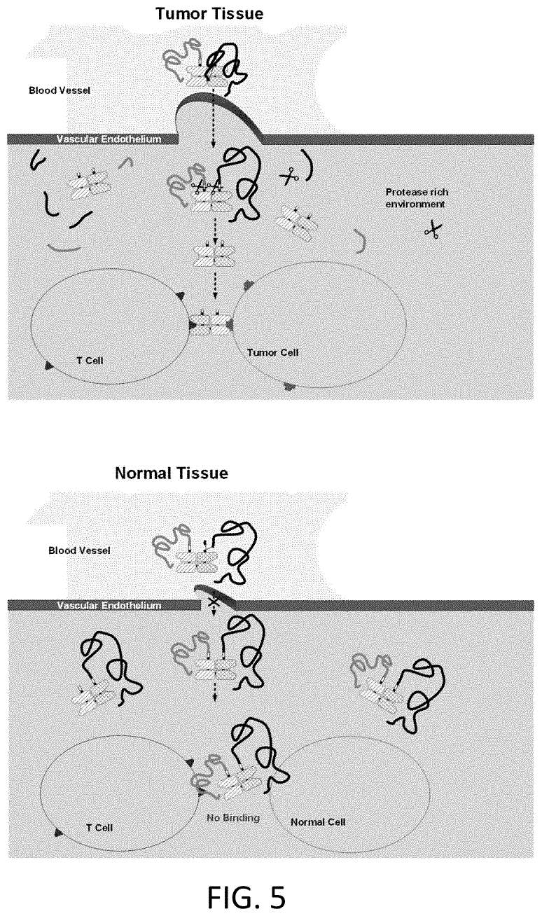

In the context of use of the bispecific antigen binding compositions in a subject, in an object of the disclosure, the subject bispecific antigen binding compositions were designed to take advantage of the differential in pore size of the vasculature in tumor or inflamed tissues compared to healthy vasculature by the addition of the XTEN, such that extravasation of the intact bispecific antigen binding composition in normal tissue is reduced, but in the leaky environment of the tumor vasculature or other areas of inflammation in diseased tissues, the intact assembly can extravasate and be activated by the proteases in the diseased tissue environment, releasing the antigen binding fragments to the effector and target cells (see, e.g., FIG. 5). In the case of the RS of the bispecific antigen binding compositions, the design takes advantage of the circumstance that when a bispecific antigen binding composition is in proximity to diseased tissues; e.g., a tumor, that elaborates one or more proteases, the RS sequences that are susceptible to the one or more proteases expressed by the tumor are capable of being cleaved by the proteases (described more fully, above). The action of the protease cleaves the release segment (RS) of the composition, separating the antigen binding fragments from the XTEN, resulting in components with reduced molecular weight and hydrodynamic radii, particularly for the released fused AF1 and AF2. As will be appreciated, the decrease in molecular weight and hydrodynamic radius of the composition also confers the property that the released, fused AF1 and AF2 are able to more freely move in solution, move through smaller pore spaces in tissue and tumors, and extravasate more readily from the larger pores of the tumor vasculature and more readily penetrate into the tumor, resulting in an increased ability to attach to and link together the effector cell and the tumor cell. Such property can be measured by different assays. Thus, it will be appreciated by one of skill in the art that in the context of treatment of a subject using the subject compositions, the bispecific antigen binding compositions are present in a prodrug form and are converted to a more active form when entering a certain cellular environment by the action of proteases co-localized with the disease tissue or cell. Upon release from the composition by the action of the protease(s) in the target tissue, the AF1 with binding specificity to an effector cell antigen and the fused AF2 with binding specificity to an target cell marker antigen of a diseased cell regain their full capability to bind to and link together the effector cell to the target cell, forming an immunological synapse. The formation of the immunological synapse causes the effector cell to become activated, with various signal pathways turning on new gene transcription and the release, by exocytosis, the effector molecule contents of its vesicles. Depending on the type of effector cell, different cytokines and lymphokines are released; e.g., Type 1 helper T cells (Th1) release cytokines like IFN-gamma, IL-2 and TNF-alpha while Type 2 helper T cells (Th2) release cytokines like IL-4, IL-5, IL-10, and IL-13 that stimulate B cells, and cytotoxic T Lymphocytes (CTLs) release cytotoxic molecules like perforin and granzymes that kill the target (collectively, “effector molecules”). It is specifically contemplated that upon the concurrent binding to and linking together the effector cell to the target tumor cell by the released bispecific antigen binding fragments of the bispecific antigen binding composition, at very low effector to target (E:T) ratios the tumor cell is acted upon by the effector molecules released by the effector cell into the immunological synapse between the cells, resulting in damage, perforin-mediated lysis, granzyme B-induced cell death and/or apoptosis of the tumor cell. Thus, in another aspect, it is a feature of various designed compositions that when the activatable bispecific antigen binding fragment composition is administered to a subject with a disease such as a tumor, the prodrug form remains in the circulatory system in normal tissue but is able to extravasate in the more permeable vasculature of the tumor such that the prodrug form of the assembly is activated by the proteases co-localized with the tumor and that the released antigen binding fragments bind together and link an effector cell (e.g., a T cell) and a tumor cell expressing the target cell marker targeted by the AF2 of the composition, whereupon the effector cell is activated and lysis of the tumor cell is effected. Stated differently, in some cases, the more permeable vasculature in the tumor tissue may permit the bispecific antigen-binding polypeptide to extravasate into the tissue where the tumor-associated proteases can act on a release segment (RS), cleaving it and releasing the binding moieties, which in turn can bind to and link together the effector cell and the tumor associate cell. In the case of the normal tissue, the extravasation may be blocked by the tighter vasculature barriers or, in the case where the bispecific antigen binding polypeptide does extravasate to some extent, the bispecific antigen binding polypeptide may primarily remain in the “pro” form, as insufficient proteases may be present in the healthy tissue to release the binding moieties, with the net effect that an immunological synapse is not formed. In some cases, the released, fused AF1 and AF2 in the tumor of the subject bound to both a tumor cell and an effector cell exhibits an increased ability to activate effector cells of at least 10-fold, or at least 30-fold, or at least 100-fold, or at least 200-fold, or at least 300-fold, or at least 400-fold, or at least 500-fold, or at least 1000-fold compared to the corresponding intact, uncleaved bispecific antigen binding composition. In other cases, the released, linked AF1 and AF2 in the tumor of the subject bound to both a tumor cell and an effector cell exhibits an increased ability to lyse the tumor cell of at least 10-fold, or at least 30-fold, or at least 100-fold, or at least 200-fold, or at least 300-fold, or at least 400-fold, or at least 500-fold, or at least 1000-fold compared to the corresponding intact bispecific antigen binding composition that has not been cleaved in the tumor. In the foregoing embodiments, the effector cell activation and/or the cytotoxicity can be assayed by conventional methods known in the art, such as cytometric measurement of activated effector cells, assay of cytokines, measurement of tumor size, or by histopathology. In the foregoing embodiments, the subject can be mouse, rat, dog, monkey, and human. In particular, it is specifically contemplated that the subject compositions are designed such that upon administration to a subject with a disease having a target cell marker to which the AF2 can bind, the bispecific antigen binding composition exhibits an enhanced therapeutic index and reduced incidence of side effects, compared to conventional bispecific antibodies known in the art, achieved by a combination of the shielding effect and steric hindrance of XTEN on binding affinity over the antigen binding fragments in the prodrug form, yet are able to release the bispecific AF1 and AF2 (achieved by inclusion of the cleavage sequences in the RS) in proximity to or within a target tissue (e.g., a tumor) that produces a protease for which the RS is a substrate.

### VII). Methods and Uses of Bispecific Antigen Binding Compositions

In another aspect, the present disclosure provides activatable bispecific antigen binding compositions and pharmaceutical compositions comprising a bispecific antigen binding composition that are particularly useful in medical settings; for example, in the prevention, treatment and/or the amelioration of certain diseases such as, but not limited to, cancers, tumors or inflammatory diseases. For use of treatment of diseases, bispecific antigen binding compositions of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

A number of therapeutic strategies have been used to design the polypeptide compositions for use in methods of treatment of a subject with a cancerous disease, including the modulation of T cell responses by targeting TcR signaling, particularly using VL and VH portions of anti-human CD3 monoclonal antibodies that are widely used clinically in immunosuppressive regimes. The CD3-specific monoclonal OKT3 was the first such monoclonal approved for use in humans (Sgro, Toxicology 105 (1995), 23-29) and is widely used clinically as an immunosuppressive agent in transplantation (Chatenoud L: Immunologic monitoring during OKT3 therapy. Clin Transplant 7:422-430, 1993). Moreover, anti-CD3 monoclonals can induce partial T cell signaling and clonal anergy (Smith, J. Exp. Med. 185 (1997), 1413-1422). The OKT3 reacts with and blocks the function of the CD3 complex in the membrane of T cells; the CD3 complex being associated with the antigen recognition structure of T cells (TCR), which is essential for signal transduction. These and other such CD3 specific antibodies are able to induce various T cell responses, including cytokine production (Von Wussow, Human gamma interferon production by leukocytes induced with monoclonal antibodies recognizing T cells. J. Immunol. 127:1197-1200 (1981)), proliferation and suppressor T-cell induction. In cancer, attempts have been made to utilize cytotoxic T cells to lyse cancer cells. Without being bound by theory, to effect target cell lysis, cytotoxic T cells are believed to require direct cell-to-cell contact; the TCR on the cytotoxic T cell must recognize and engage the appropriate antigen on the target cell. This creates the immunologic synapse that, in turn initiates a signaling cascade within the cytotoxic T cell, causing T-cell activation and the production of a variety of cytotoxic cytokines and effector molecules. Perforin and granzymes are highly toxic molecules that are stored in preformed granules that reside in activated cytotoxic T cells. After recognition of the target cell, the cytoplasmic granules of the engaged cytotoxic T cells migrate toward the cytotoxic T-cell membrane, ultimately fusing with it and releasing their contents in directed fashion into the immunological synapse to form a pore within the membrane of the target cell, disrupting the tumor cell plasma membrane. The created pore acts as a point of entry for granzymes; a family of serine proteases that that induce apoptosis of the tumor cells.

The subject bispecific antigen binding compositions described herein, with an AF1 with specific binding affinity to the CD3 of a T cell closely fused to an AF2 with specific binding affinity to a target cell marker are T-cell engagers with the ability, once released from the intact prodrug form of the composition by cleavage of the release segments, regain their full potential to bind a T cell and target cell, forming an immunological synapse that promotes activation of the T-cell and promotes the subsequent destruction of the tumor cell via apoptosis or cytolysis.

The disclosure contemplates methods of use of bispecific antigen binding compositions that are engineered to target a range of malignant cells, such as tumors, in addition to the effector cells, in order to initiate target cell lysis and to effect a beneficial therapeutic outcome in that the bispecific antigen binding compositions are designed such that the AF1 binds and engages CD3 to activate the cytotoxic T cell while the AF2 can be designed to target a variety of different target cell markers that are characteristic of specific malignancies; bridging them together for the creation of the immunological synapse. In a particular advantage of the design, the physical binding of the cytotoxic effector cell and the cell bearing the target cell marker eliminates the need for antigen processing, MHCI/ß2-microglobulin, as well as co-stimulatory molecules. Examples of important target cell markers include but are not limited to the markers disclosed herein. Because of the range of such target cell markers (more extensively described, above) that can be engineered into the various embodiments of the subject bispecific antigen binding compositions, it will be appreciated that the resulting compositions will have utility against a variety of diseases, including hematological cancers and solid tumors. In one embodiment, the disclosure provides a method of treatment of a subject with a tumor. The tumor being treated can comprise tumor cells arising from a cell selected from the group consisting of stromal cell, fibroblasts, myofibroblasts, glial cells, epithelial cells, fat cells, lymphocytic cells, vascular cells, smooth muscle cells, mesenchymal cells, breast tissue cells, prostate cells, kidney cells, brain cells, colon cells, ovarian cells, uterine cells, bladder cells, skin cells, stomach cells, genito-urinary tract cells, cervix cells, uterine cells, small intestine cells, liver cells, pancreatic cells, gall bladder cells, bile duct cells, esophageal cells, salivary gland cells, lung cells, and thyroid cells. In a further advantage of the compositions, as the cytotoxic effector cells are not consumed during the damage/destruction of the bridged target cancer cell, after causing lysis of one target cell, an activated effector cell can release and move on through the local tissue towards other target cancer cells, bind again to the AF1-AF2 and the target antigen, and initiate additional cell lysis. In addition, it is contemplated that in a localized environment like a solid tumor, the release of effector cell molecules such as perform and granzymes will result in damage to tumor cells that are adjacent but not bound by a given molecule of the bispecific binding domains, resulting in stasis of growth or regression of the tumor.

Accordingly, a utility of the disclosure will be understood that after administration of a therapeutically effective dose of pharmaceutical composition comprising a bispecific antigen binding composition described herein to a subject with a cancer or tumor having the target cell marker, the composition can be acted upon by proteases in association with or co-localized with the cancer or tumor cells, releasing the fused AF1 and AF2 such that an immunological synapse can be created by the linking of the target cell and a effector cell, with the result that effector cell-derived effector molecules capable of lysing the target cell are released into the synapse, leading to apoptosis, cytolysis, or death of the target cancer or tumor cell. Furthermore, it will be appreciated by one of skill in the art that use of the bispecific antigen binding compositions can result in a sustained and more generalized beneficial therapeutic effect than a “single kill” once the immunological synapse is formed by the binding of the released binding domains to the effector cell and target cancer cell.

In one aspect, the disclosure relates to methods of treating a disease in a subject, such as a cancer or an inflammatory disorder. In some embodiments, the disclosure provides a method of treating a disease in a subject, comprising administering to the subject in need thereof a therapeutically effective amount of a pharmaceutical composition comprising a bispecific antigen binding composition of any of the embodiments described herein. A therapeutically effective amount of the pharmaceutical composition may vary according to factors such as the disease state, age, sex, and weight of the individual, and the ability of the antibody or antibody portion to elicit a desired response in the individual. A therapeutically effective amount is also one in which any toxic or detrimental effects of the subject compositions are outweighed by the therapeutically beneficial effects. A prophylactically effective amount refers to an amount of pharmaceutical composition required for the period of time necessary to achieve the desired prophylactic result.

A therapeutically effective dose of the bispecific antigen binding compositions described herein will generally provide therapeutic benefit without causing substantial toxicity. Toxicity and therapeutic efficacy of a bispecific antigen binding composition can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Bispecific antigen binding compositions that exhibit large therapeutic indices are preferred. In one aspect, the bispecific antigen binding molecule according to the present disclosure exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1). A skilled artisan readily recognizes that in many cases the bispecific antigen binding composition may not provide a cure but may only provide partial benefit. In some aspects, a physiological change having some benefit is also considered therapeutically beneficial. Thus, in some aspects, an amount of bispecific antigen binding composition that provides a physiological change is considered an “effective amount” or a “therapeutically effective amount”. The subject, patient, or individual in need of treatment is typically a mouse, rat, dog, monkey, or a human.

The bispecific antigen binding compositions of the invention may be administered in combination with one or more other agents in therapy. For instance, a bispecific antigen binding molecule of any of the embodiments described herein may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain aspects, an additional therapeutic agent is an immunomodulatory agent, an immuno-oncologic antibody, a cytostatic agent, an inhibitor of cell adhesion, a cytotoxic agent, an activator of cell apoptosis, or an agent that increases the sensitivity of cells to apoptotic inducers. In a particular aspect, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

In one embodiment of the method of treating a disease in a subject, the disease for treatment can be carcinomas, Hodgkin's lymphoma, non-Hodgkin's lymphoma, B cell lymphoma, diffuse large B cell lymphoma, T-cell lymphoma, follicular lymphoma, mantle cell lymphoma, blastoma, breast cancer, colon cancer, prostate cancer, head and neck cancer, any form of skin cancer, melanoma, genito-urinary tract cancer, ovarian cancer, ovarian cancer with malignant ascites, vaginal cancer, vulvar cancer, Ewing sarcoma, peritoneal carcinomatosis, uterine serous carcinoma, parathyroid cancer, endometrial cancer, cervical cancer, colorectal cancer, an epithelia intraperitoneal malignancy with malignant ascites, uterine cancer, mesothelioma in the peritoneum kidney cancers, lung cancer, laryngeal cancer, small-cell lung cancer, non-small cell lung cancer, gastric cancer, esophageal cancer, stomach cancer, small intestine cancer, liver cancer, hepatocarcinoma, retinoblastoma, hepatoblastoma, liposarcoma, pancreatic cancer, gall bladder cancer, testicular cancer, cancers of the bile duct, cancers of the bone, salivary gland carcinoma, thyroid cancer, craniopharyngioma, carcinoid tumor, epithelial cancer, arrhenoblastoma, adenocarcinoma, sarcomas of any origin, primary hematologic malignancies including acute or chronic lymphocytic leukemias, acute or chronic myelogenous leukemias, B-cell derived chronic lymphatic leukemia, hairy cell leukemia, myeloproliferative neoplastic disorders, or myelodysplastic disorders, myasthenia gravis, Morbus Basedow, Kaposi sarcoma, neuroblastoma, Hashimoto thyroiditis, Wilms tumor, or Goodpasture syndrome. The therapeutically effective amount can produce a beneficial effect in helping to treat (e.g., cure or reduce the severity) or prevent (e.g., reduce the likelihood of recurrence) of a cancer or a tumor. In another embodiment of the method of treating the disease in a subject, the pharmaceutical composition is administered to the subject as two or more therapeutically effective doses administered twice weekly, once a week, every two weeks, every three weeks, every four weeks, or monthly. In another embodiment of the method, the pharmaceutical composition is administered to the subject as two or more therapeutically effective doses over a period of at least two weeks, or at least one month, or at least two months, or at least three months, or at least four months, or at least five months, or at least six months. In another embodiment of the method, a first low priming dose is administered to the subject, followed by one or more higher maintenance doses over the dosing schedule of at least two weeks, or at least one month, or at least two months, or at least three months, or at least four months, or at least five months, or at least six months. The initial priming dose administered is selected from the group consisting of at least about 0.005 mg/kg, at least about 0.01 mg/kg, at least about 0.02 mg/kg, at least about 0.04 mg/kg, at least about 0.08 mg/kg, at least about 0.1 mg/kg, and one or more subsequent maintenance dose(s) administered is selected from the group consisting of at least about 0.02 mg/kg, at least about 0.05 mg/kg, at least about 0.1 mg/kg, at least about 0.16 mg/kg, at least about 0.18 mg/kg, at least about 0.20 mg/kg, at least about 0.22 mg/kg, at least about 0.24 mg/kg, at least about 0.26 mg/kg, at least about 0.27 mg/kg, at least about 0.28 mg/kg, at least 0.3 mg/kg, at least 0.4. mg/kg, at least about 0.5 mg/kg, at least about 0.6 mg/kg, at least about 0.7 mg/kg, at least about 0.8 mg/kg, at least about 0.9 mg/kg, at least about 1.0 mg/kg, at least about 1.5 mg/kg, or at least about 2.0 mg/kg, or at least 5.0 mg/kg. In another embodiment of the method, the pharmaceutical composition is administered to the subject intradermally, subcutaneously, intravenously, intra-arterially, intra-abdominally, intraperitoneally, intrathecally, or intramuscularly. In another embodiment of the method, the pharmaceutical composition is administered to the subject as one or more therapeutically effective bolus doses or by infusion of 5 minutes to 96 hours as tolerated for maximal safety and efficacy. In another embodiment of the method, the pharmaceutical composition is administered to the subject as one or more therapeutically effective bolus doses or by infusion of 5 minutes to 96 hours, wherein the dose is selected from the group consisting of at least about 0.005 mg/kg, at least about 0.01 mg/kg, at least about 0.02 mg/kg, at least about 0.04 mg/kg, at least about 0.08 mg/kg, at least about 0.1 mg/kg, at least about 0.12 mg/kg, at least about 0.14 mg/kg, at least about 0.16 mg/kg, at least about 0.18 mg/kg, at least about 0.20 mg/kg, at least about 0.22 mg/kg, at least about 0.24 mg/kg, at least about 0.26 mg/kg, at least about 0.27 mg/kg, at least about 0.28 mg/kg, at least 0.3 mg/kg, at least 0.4. mg/kg, at least about 0.5 mg/kg, at least about 0.6 mg/kg, at least about 0.7 mg/kg, at least about 0.8 mg/kg, at least about 0.9 mg/kg, at least about 1.0 mg/kg, at least about 1.5 mg/kg, or at least about 2.0 mg/kg, or at least about 5.0 mg/kg. In another embodiment of the method, the pharmaceutical composition is administered to the subject as one or more therapeutically effective bolus doses or by infusion over a period of 5 minutes to 96 hours, wherein the administration to the subject results in a Cmax plasma concentration of the intact, uncleaved bispecific antigen binding composition of at least about 0.1 ng/mL to at least about 2 μg/mL or more in the subject that is maintained for at least about 3 days, at least about 7 days, at least about 10 days, at least about 14 days, or at least about 21 days. The therapeutically effective dose is at least about 0.005 mg/kg, at least about 0.01 mg/kg, at least about 0.02 mg/kg, at least about 0.04 mg/kg, at least about 0.08 mg/kg, at least about 0.1 mg/kg, at least about 0.12 mg/kg, at least about 0.14 mg/kg, at least about 0.16 mg/kg, at least about 0.18 mg/kg, at least about 0.20 mg/kg, at least about 0.22 mg/kg, at least about 0.24 mg/kg, at least about 0.26 mg/kg, at least about 0.27 mg/kg, at least about 0.28 mg/kg, at least 0.3 mg/kg, at least 0.4 mg/kg, at least about 0.5 mg/kg, at least about 0.6 mg/kg, at least about 0.7 mg/kg, at least about 0.8 mg/kg, at least about 0.9 mg/kg, at least about 1.0 mg/kg, at least about 1.5 mg/kg, or at least about 2.0 mg/kg. In one embodiment, an initial dose is selected from the group consisting of at least about 0.005 mg/kg, at least about 0.01 mg/kg, at least about 0.02 mg/kg, at least about 0.04 mg/kg, at least about 0.08 mg/kg, at least about 0.1 mg/kg, and a subsequent dose is selected from the group consisting of at least about 0.1 mg/kg, at least about 0.12 mg/kg, at least about 0.14 mg/kg, at least about 0.16 mg/kg, at least about 0.18 mg/kg, at least about 0.20 mg/kg, at least about 0.22 mg/kg, at least about 0.24 mg/kg, at least about 0.26 mg/kg, at least about 0.27 mg/kg, at least about 0.28 mg/kg, at least 0.3 mg/kg, at least 0.4. mg/kg, at least about 0.5 mg/kg, at least about 0.6 mg/kg, at least about 0.7 mg/kg, at least about 0.8 mg/kg, at least about 0.9 mg/kg, at least about 1.0 mg/kg, at least about 1.5 mg/kg, or at least about 2.0 mg/kg. In the foregoing embodiments, the administration to the subject results in a plasma concentration of the polypeptide of at least about 0.1 ng/mL to at least about 2 ng/mL or more in the subject for at least about 3 days, at least about 7 days, at least about 10 days, at least about 14 days, or at least about 21 days. In the foregoing embodiments of the method, the subject can be a mouse, rat, dog, monkey, or a human.

### VIII). Nucleic Acid Sequences

In another aspect, the present invention relates to isolated polynucleotide sequences encoding the polypeptides or bispecific antigen binding compositions of any of the embodiments described herein and sequences complementary to polynucleotide molecules encoding the polypeptide composition embodiments.

In some embodiments, the invention provides isolated polynucleotide sequences encoding the AF1 sequences, or the AF2 sequences, or the release segment sequences (RS1 and RS2), or the XTEN sequences of any of the embodiments described herein, or the complement of the polynucleotide sequences. In one embodiment, the invention provides an isolated polynucleotide sequence encoding a polypeptide or bispecific antigen binding composition of any of the embodiments described herein, or the complement of the polynucleotide sequence. In one embodiment, the invention provides an isolated polynucleotide sequence encoding a polypeptide or bispecific antigen binding composition wherein the polynucleotide sequence has at least 90%, 91%, 92%, 93%, 94%, 95%, 96%, 97%, 98%, 99%, or 100% sequence identity to a polynucleotide sequence set forth in Table 9.

In another aspect, the disclosure relates to methods to produce polynucleotide sequences encoding the polypeptides or bispecific antigen binding compositions of any of the embodiments described herein, or sequences complementary to the polynucleotide sequences, including homologous variants thereof, as well as methods to express the proteins expressed by the polynucleotide sequences. In general, the methods include producing a polynucleotide sequence coding for the proteinaceous polypeptides or bispecific antigen binding compositions of any of the embodiments described herein and incorporating the encoding gene into an expression vector appropriate for a host cell. For production of the encoded polypeptides or bispecific antigen binding compositions of any of the embodiments described herein, the method includes transforming an appropriate host cell with the expression vector, and culturing the host cell under conditions causing or permitting the resulting polypeptide or bispecific antigen binding composition of any of the embodiments described herein to be expressed in the transformed host cell, thereby producing the polypeptide or bispecific antigen binding composition, which is recovered by methods described herein or by standard protein purification methods known in the art. Standard recombinant techniques in molecular biology are used to make the polynucleotides and expression vectors of the present disclosure.

In accordance with the disclosure, nucleic acid sequences that encode the polypeptides or bispecific antigen binding compositions of any of the embodiments described herein (or their complement) are used to generate recombinant DNA molecules that direct the expression in appropriate host cells. Several cloning strategies are suitable for performing the present disclosure, many of which are used to generate a construct that comprises a gene coding for a composition of the present disclosure, or its complement. In one embodiment, the cloning strategy is used to create a gene that encodes a construct that comprises nucleotides encoding the polypeptide or bispecific antigen binding composition that is used to transform a host cell for expression of the composition. In the foregoing embodiments hereinabove described in this paragraph, the genes can comprise nucleotides encoding the antigen binding fragments, release segments, and the XTEN in the configurations disclosed herein.

In one approach, a construct is first prepared containing the DNA sequence encoding a polypeptide or bispecific antigen binding composition construct. Exemplary methods for the preparation of such constructs are described in the Examples. The construct is then used to create an expression vector suitable for transforming a host cell, such as a prokaryotic or eukaryotic host cell for the expression and recovery of the polypeptide construct. Where desired, the host cell is an E. coli. In another embodiment, the host cell is selected from BHK cells, NS0 cells, SP2/0 cells, YO myeloma cells, P3X63 mouse myeloma cells, PER cells, PER.C6 cells, hybridoma cells, NIH3T3 cells, COS, HeLa, CHO, or yeast cells. Exemplary methods for the creation of expression vectors, the transformation of host cells and the expression and recovery of XTEN are described in the Examples.

The gene encoding for the polypeptide or bispecific antigen binding composition construct can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate sequences of polynucleotides encoding the various components (e.g., binding domains, linkers, release segments, and XTEN) genes of a desired length and sequence. Genes encoding polypeptide compositions are assembled from oligonucleotides using standard techniques of gene synthesis. The gene design can be performed using algorithms that optimize codon usage and amino acid composition appropriate for the E. coli or mammalian host cell utilized in the production of the polypeptide or bispecific antigen binding composition. In one method of the disclosure, a library of polynucleotides encoding the components of the constructs is created and then assembled, as described above. The resulting genes are then assembled, and the resulting genes used to transform a host cell and produce and recover the polypeptide compositions for evaluation of its properties, as described herein.

The resulting polynucleotides encoding the polypeptide or bispecific antigen binding composition sequences can then be individually cloned into an expression vector. The nucleic acid sequence is inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available. The vector may, for example, be in the form of a plasmid, cosmid, viral particle, or phage that may conveniently be subjected to recombinant DNA procedures, and the choice of vector will often depend on the host cell into which it is to be introduced. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. Once introduced into a suitable host cell, expression of the antigen binding fragments or bispecific antigen binding compositions can be determined using any nucleic acid or protein assay known in the art. For example, the presence of transcribed mRNA of light chain CDRs or heavy chain CDRs, the antigen binding fragment, or the bispecific antigen binding composition can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to any region of antigen binding unit polynucleotide.

The disclosure provides for the use of plasmid expression vectors containing replication and control sequences that are compatible with and recognized by the host cell and are operably linked to the gene encoding the polypeptide for controlled expression of the polypeptide. The vector ordinarily carries a replication site, as well as sequences that encode proteins that are capable of providing phenotypic selection in transformed cells. Such vector sequences are well known for a variety of bacteria, yeast, and viruses. Useful expression vectors that can be used include, for example, segments of chromosomal, non-chromosomal and synthetic DNA sequences. “Expression vector” refers to a DNA construct containing a DNA sequence that is operably linked to a suitable control sequence capable of effecting the expression of the DNA encoding the polypeptide in a suitable host. The requirements are that the vectors are replicable and viable in the host cell of choice. Low- or high-copy number vectors may be used as desired.

Suitable vectors include, but are not limited to, derivatives of SV40 and pcDNA and known bacterial plasmids such as col EI, pCR1, pBR322, pMal-C2, pET, pGEX as described by Smith, et al., Gene 57:31-40 (1988), pMB9 and derivatives thereof, plasmids such as RP4, phage DNAs such as the numerous derivatives of phage I such as NM98 9, as well as other phage DNA such as M13 and filamentous single stranded phage DNA; yeast plasmids such as the 2 micron plasmid or derivatives of the 2m plasmid, as well as centomeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like. Yeast expression systems that can also be used in the present disclosure include, but are not limited to, the non-fusion pYES2 vector (Invitrogen), the fusion pYESHisA, B, C (Invitrogen), pRS vectors and the like. The control sequences of the vector include a promoter to effect transcription, an optional operator sequence to control such transcription, a sequence encoding suitable mRNA ribosome binding sites, and sequences that control termination of transcription and translation. The promoter may be any DNA sequence, which shows transcriptional activity in the host cell of choice and may be derived from genes encoding proteins either homologous or heterologous to the host cell. Promoters suitable for use in expression vectors with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)], all is operably linked to the DNA encoding XTEN polypeptides. Promoters for use in bacterial systems can also contain a Shine-Dalgarno (S.D.) sequence, operably linked to the DNA encoding polypeptide polypeptides.

Expression of the vector can also be determined by examining the antigen binding fragment or a component of the bispecific antigen binding composition expressed. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

### IX). Methods of Making the Polypeptides and Bispecific Antigen Binding Compositions

In another aspect, the disclosure provides methods of manufacturing the subject compositions. In one embodiment, the method comprises culturing a host cell comprising a nucleic acid construct that encodes a polypeptide or a bispecific antigen binding composition of any of the embodiments described herein under conditions that promote the expression of the polypeptide or bispecific antigen binding composition, followed by recovery of the polypeptide or bispecific antigen binding composition using standard purification methods (e.g., column chromatography, HPLC, and the like) wherein the composition is recovered wherein at least 70%, or at least 80%, or at least 90%, or at least 95%, or at least 97%, or at least 99% of the binding fragments of the expressed polypeptide or bispecific antigen binding composition are correctly folded. In another embodiment of the method of making, the expressed polypeptide or bispecific antigen binding composition is recovered in which at least or at least 90%, or at least 95%, or at least 97%, or at least 99% of the polypeptide or bispecific antigen binding composition is recovered in monomeric, soluble form.

In another aspect, the disclosure relates to methods of making the polypeptide and bispecific antigen binding compositions at high fermentation expression levels of functional protein using an E. coli or mammalian host cell, as well as providing expression vectors encoding the constructs useful in methods to produce the cytotoxically active polypeptide construct compositions at high expression levels. In one embodiment, the method comprises the steps of 1) preparing the polynucleotide encoding the polypeptides of any of the embodiments disclosed herein, 2) cloning the polynucleotide into an expression vector, which can be a plasmid or other vector under control of appropriate transcription and translation sequences for high level protein expression in a biological system, 3) transforming an appropriate host cell with the expression vector, and 4) culturing the host cell in conventional nutrient media under conditions suitable for the expression of the polypeptide composition. Where desired, the host cell is E. coli. By the method, the expression of the polypeptide results in fermentation titers of at least 0.05 g/L, or at least 0.1 g/L, or at least 0.2 g/L, or at least 0.3 g/L, or at least 0.5 g/L, or at least 0.6 g/L, or at least 0.7 g/L, or at least 0.8 g/L, or at least 0.9 g/L, or at least 1 g/L of the expression product of the host cell and wherein at least 70%, or at least 80%, or at least 90%, or at least 95%, or at least 97%, or at least 99% of the expressed protein are correctly folded. As used herein, the term “correctly folded” means that the antigen binding fragments component of the composition have the ability to specifically bind its target ligand. In another embodiment, the disclosure provides a method for producing a polypeptide or bispecific antigen binding composition, the method comprising culturing in a fermentation reaction a host cell that comprises a vector encoding a polypeptide comprising the polypeptide or bispecific antigen binding composition under conditions effective to express the polypeptide product at a concentration of more than about 10 milligrams/gram of dry weight host cell (mg/g), or at least about 250 mg/g, or about 300 mg/g, or about 350 mg/g, or about 400 mg/g, or about 450 mg/g, or about 500 mg/g of said polypeptide when the fermentation reaction reaches an optical density of at least 130 at a wavelength of 600 nm, and wherein the antigen binding fragments of the expressed protein are correctly folded. In another embodiment, the disclosure provides a method for producing a polypeptide or bispecific antigen binding composition, the method comprising culturing in a fermentation reaction a host cell that comprises a vector encoding the composition under conditions effective to express the polypeptide product at a concentration of more than about 10 milligrams/gram of dry weight host cell (mg/g), or at least about 250 mg/g, or about 300 mg/g, or about 350 mg/g, or about 400 mg/g, or about 450 mg/g, or about 500 mg/g of said polypeptide when the fermentation reaction reaches an optical density of at least 130 at a wavelength of 600 nm, and wherein the expressed polypeptide product is soluble.

The following are examples of compositions and evaluations of compositions of the disclosure. It is understood that various other embodiments may be practiced, given the general description provided above.

### EXAMPLES

### Example 1: Construction of Bispecific Antigen Binding Polypeptides with Two Release Segments

In order to generate a plasmid where the individual scFvs can be removed by restriction digest, pCW1700, which encodes for an anti-EpCAM-anti-CD3 (UCHT1) bispecific tandem scFv, with an RSR2486 release segment, an AE866 XTEN and a 6×His tag affinity tag (SEQ ID NO: 1150), was digested with SacII and BstXI, removing the 3′ end of the anti-EpCAM binding domain, the linker between the anti-EpCAM and anti-CD3 domains and the 5′ end of the anti-CD3 domain. A fragment of DNA encoding the same region was synthesized with silent point mutations at the junction between the anti-EpCAM binding domain and the linker to introduce a Bsu36I site. Synthetic DNA fragments were cloned into digested backbone using the In-Fusion kit (New England Biolabs) to assemble pJB0035. pJB0035 was subsequently digested with NheI and BsaI to remove the BSRS1 release segment sequence. Overlapping single stranded oligonucleotides encoding RSR2486 were synthesized with single stranded tails that anneal to the NheI and BsaI overhangs. The oligonucleotides were annealed together and ligated into the digested pJB0035, resulting in pCW1880, which encodes for an anti-EpCAM-anti-CD3 (UCHT1) bispecific tandem scFv, RSR2486, XTEN866 and a 6×His tag affinity tag (SEQ ID NO: 1150).

In order to generate plasmids with various CD3 binding domain variants, pCW1880 was digested with Bsu36I and NheI to remove the UCHT1 anti-CD3 scFv. DNA fragments encoding the designed CD3 variants were synthesized. Each gene fragment included 30 nucleotides 5′ and 3′ of the restriction sites to serve as DNA overlaps for Gibson DNA Assembly. Synthetic DNA fragments were cloned into digested backbone using the Gibson Cloning Kit (SGI-DNA, Carlsbad, Calif.) to assemble pJB0205, pJB0206, pJB0207 and pJB0208.

In order to generate a bispecific antigen binding polypeptide with both an N-terminal and C-terminal XTEN, the AE292 XTEN was PCR amplified from a plasmid using primers including a 17-21 bp 5′ homology region to backbone DNA on the N-terminus and to an uncleavable release segment (RSR3058, amino acid sequence TTGEAGEAAGATSAGATGP (SEQ ID NO: 100)) on the C-terminus. A second PCR product encoding the light and part of the heavy chain of the anti-EpCAM antibody 4D5MOCB was amplified using primers that included a 16-21 bp 5′ homology region to RSR3058 on the N-terminus and the heavy chain of 4D5MOCB on the C-terminus. These PCR fragments were cloned into a backbone vector digested with BsiWI-SacII that encoding the remainder of the 4D5MOCB heavy chain/anti-CD3 tandem scFv, a second copy of the RSR3058 uncleavable release segment and AE837 XTEN with a 6×HIS (SEQ ID NO: 1150) affinity tag using the In-Fusion Plasmid Assembly Kit (Takara Bio). The final vector encodes the bispecific antigen binding polypeptide with the components (in the N- to C-terminus) of AE292 XTEN, the uncleavable RSR3058 release segment, anti-EpCAM-anti-CD3 bispecific tandem scFv, with RSR3058 fused to AE867 XTEN with a 6×HIS (SEQ ID NO: 1150) affinity tag under the control of a PhoA promoter and STII secretion leader. The resulting construct is pJB0084 (Table 9).

pJB0084 was used as a template to create a bispecific antigen binding polypeptide construct encoding AE292 XTEN, the cleavable release segment RSR2295, anti-EpCAM-anti-CD3 bispecific tandem scFv, with RSR2295 fused to AE868 XTEN. The plasmid utilized two PCR products using pJB0084 as a template; the first encoding a 6×HIS (SEQ ID NO: 1150) affinity tag and AE292 XTEN with an 5′ homology region to the vector backbone and the 3′ homology region encoding the first RSR2295, the second encoding the anti-EpCAM-anti-CD3 bispecific tandem scFv with 5′ and 3′ homology regions encoding the RSR2295 release segments 5′ and 3′ of the tandem scFvs. The third fragment encoded AE868 XTEN having the C-Tag affinity tag (amino acid sequence EPEA (SEQ ID NO: 1149)) with a 5′ homology region encoding the second RSR2295 and a 3′ homology region to the backbone vector. The three PCR fragments were cloned into pJB0084 that had been digested with BsiWI-NotI using the In-Fusion Plasmid Assembly Kit. The final vector, pJB0169, encodes the bispecific antigen binding polypeptide molecule with the components (in the N- to C-terminus) of 6×HIS affinity tag (SEQ ID NO: 1150), AE292 XTEN, RSR2295 release segment, anti-EGFR-anti-CD3 bispecific tandem scFv, RSR2295, AE868 XTEN with the C-Tag affinity tag under the control of a PhoA promoter and STII secretion leader with the DNA sequence.

To construct pJB0163 and pJB0179, pJB0169 was digested with DraIII and BtsI to remove the 5′ RSR2295, anti-EGFR-anti-CD3 bispecific tandem scFv, RSR2295, and the first 72 amino acids of the AE868XTEN. For pJB0163, a fragment of DNA was synthesized encoding RSR3058, the anti-CD3 light chain, anti-EGFR light and heavy chain, the anti-CD3 heavy chain, RSR3058 and the first 72 amino acids of AE868 XTEN. For pJB0179, a fragment of DNA was synthesized encoding RSR2295, the anti-CD3 light chain, anti-EGFR light and heavy chain, the anti-CD3 heavy chain, RSR2295 and the first 72 amino acids of AE868 XTEN. The gene fragments also included 30 nucleotides 5′ and 3′ of the restriction sites to serve as DNA overlaps for Gibson DNA Assembly. Synthetic DNA fragments were cloned into the digested pJB0169 backbone using the Gibson Cloning Kit (SGI-DNA, Carlsbad, Calif.) to assemble pJB0163 and pJB0179.

pJB0179 was digested with BsaI and BbvCI to remove the anti-CD3 and anti-EGFR binding domain encoding sequences. A PCR product encoding an anti-HER2 light chain and heavy chain with primers including an 18 bp 5′ homology region to backbone DNA on the N-terminus and a 21 bp 3′ homology region to a second PCR product was amplified. A second PCR product encoding an anti-CD3 scFv sequence variant (CD3.23) with primers including an 18 bp 5′ homology region to the first PCR product on the N-terminus and a 23 bp 3′ homology region the vector backbone was amplified using pJB0205 as a template. The two PCR products were cloned into the digested backbone using the Gibson Cloning Kit (SGI-DNA, Carlsbad, Calif.) to assemble pAH0011.

pJB0163 was digested with BsaI and BstEII to remove the anti-CD3 and anti-EGFR binding domain encoding sequences. A PCR product encoding an anti-HER2 light chain and heavy chain with primers including an 18 bp 5′ homology region to backbone DNA on the N-terminus and a 21 bp 3′ homology region to a second PCR product was amplified. A second PCR product encoding an anti-CD3 scFv sequence variant (CD3.23) with primers including an 18 bp 5′ homology region to the first PCR product on the N-terminus and a 23 bp 3′ homology region the vector backbone was amplified using pJB0205 as a template. The two PCR products were cloned into the digested backbone using the Gibson Cloning Kit (SGI-DNA, Carlsbad, Calif.) to assemble pAH0013.

In order to generate pJB0244 and pJB0245, pAH0011 and pAH0013 were digested with BsaI and BsrDI to remove the anti-Her2 (Her2.1) light and heavy chains encoding sequences. PCR products encoding the anti-Her2 (Her2.2) light and heavy chains was amplified with primers including an 25 bp 5′ homology region to the 3′ end of the respective vector backbone on the N-terminus and a 25 bp 3′ homology region to the 5′ end of the vector backbone. The PCR product for pJB0244 was cloned into the digested pAH0011 backbone using the Gibson Cloning Kit (SGI-DNA, Carlsbad, Calif.) to assemble pJB0244, which encodes for a 6×HIS affinity tag (SEQ ID NO: 1150), AE292 XTEN, RSR2295, anti-HER2-anti-CD3 bispecific tandem scFv, RSR2295, AE868 XTEN868 having a C-Tag affinity tag under the control of a PhoA promoter and STII secretion leader with the DNA sequence and encoded amino acid sequence provided in Table 9. The PCR product for pJB0245 was cloned into the pAH0013 backbone to generate pJB0245, which encodes for a 6×HIS affinity tag (SEQ ID NO: 1150), AE292 XTEN, RSR3058 release segment, anti-HER2-anti-CD3 bispecific tandem scFv, RSR3058, AE868 XTEN having a C-Tag affinity tag under the control of a PhoA promoter and STII secretion leader.

In order to introduce a new CD3 scFv with alterations to the isoelectric point and removal of potential aggregation sites in the amino acid sequence, pJB0244 was digested with BsaI and BbvCI to remove both the HER2 and CD3 scFvs. DNA fragments encoding anti-EGFR scFv variants paired with CD3.33 were synthesized that included 40 bp of homology to the digested vector at both the 5′ and 3′ ends to facilitate Gibson DNA Assembly. Plasmids pJB0358-pJB0372 were assembled with the structure of 6×HIS affinity tag (SEQ ID NO: 1150), AE292 XTEN, RSR2295, and individually, a total of 15 anti-EGFR scFv variants paired with an anti-CD3 scFv, RSR2295, AE868 XTEN having a C-Tag affinity tag.

pAH0025 and pAH0026 were created by initially digesting pJB0368 and pJB0373 with BtsI to remove the anti-CD3 scFv. DNA fragments were ordered encoding the anti-CD3.32 scFv flanked with 40 bp homology regions to the digested backbone. These fragments were introduced into pJB0368 and pJB0373 by Gibson Assembly to create plasmids encoding a 6×HIS affinity tag (SEQ ID NO: 1150), AE292 XTEN, RSR2295, anti-EGFR-anti-CD3 bispecific tandem scFv, RSR2295, AE868 XTEN having a C-Tag affinity tag constructed with two different anti-EGFR binding domains, EGFR.23 and EGFR.2 to result in the pAH0025 and pAH0026 constructs with the DNA sequence and encoded amino acid sequence provided in Table 9.

To generate bispecific antigen binding polypeptide constructs with a shortened C-terminal XTEN, pJB0244 was digested with BtsI and EcoRI to remove the C-terminal XTEN and the C-tag. A PCR fragment encoding for an AE584 XTEN sequence and C-tag was amplified from pJB0244. A second fragment encoding vector backbone with 40 bp of homology past the EcoRI site was synthesized with a 34 base tail overlapping the first fragment. These two fragments were cloned into the digested pJB0244 backbone using the Gibson Assembly Kit to create plasmid pJB0354, which encodes a 6×HIS affinity tag (SEQ ID NO: 1150), AE292, RSR2295, anti-HER2-anti-CD3 bispecific tandem scFv, RSR2295, AE584 XTEN and a C-Tag affinity tag. To generate pJB0355, a PCR fragment encoding for an AE293 XTEN sequence and C-tag was amplified from pJB0244. This was cloned, along with the second fragment described above, into the digested pJB0244 backbone using the Gibson Assembly Kit to create plasmid pJB0355, which encodes a 6×HIS affinity tag (SEQ ID NO: 1150), XTEN292, RSR2295, anti-Her2-anti-CD3 bispecific tandem scFv, RSR2295, AE300 XTEN and a C-Tag affinity tag (DNA and amino acid sequences in Table 9). Uncleavable variants of pJB0354 and pJB0355 (pJB0377 and pJB0378 respectively) were also constructed substituting RSR2295 with the sequence EAGRSANHTPAGLTGP (SEQ ID NO: 88).

To generate protein with shortened N- and C-terminal XTENs, three PCR products were amplified. The first PCR product consisted of the N-terminal His tag and AE144_7A XTEN amplified from pCW1199. The second PCR products consisted of the N-terminal release site 2295, the anti-HER2-anti-CD3 bispecific tandem scFv, and the C-terminal release site 2295 and 286 amino acids of XTEN sequence. These two fragments were cloned into a backbone that was generated by PCR amplification that includes the last 17 XTEN amino acids on its 5′ end including 30 bp of homology to the second PCR product and the STII signal peptide, 6×His tag (SEQ ID NO: 1150) and 5 XTEN residues on its 3′ end, which includes 39 bp of homology to the 5′ end of the first PCR product via Gibson Assembly to form pJB0380. pJB0380 encodes for a 6×HIS affinity tag (SEQ ID NO: 1150), AE144_7A XTEN, RSR2295, anti-HER2-anti-CD3 bispecific tandem scFv, RSR2295, AE293 XTEN and a C-Tag affinity tag (DNA and amino acid sequences in Table 9). An uncleavable variants of pJB0380 (pJB0379) was also constructed substituting RSR2295 with the sequence EAGRSANHTPAGLTGP (SEQ ID NO: 88). The same methodologies would be employed to make constructs having CD3.24, CD3.30, CD3.31, CD3.33 scFv, and scFv for antigen binding fragments against target cell markers described herein, in any combination or orientation (i.e., AF1-AF2 or AF2-AF1 in an N- to C-terminal orientation).

Lengthy table referenced here
US20210054077A1-20210225-T00001
Please refer to the end of the specification for access instructions.

Lengthy table referenced here
US20210054077A1-20210225-T00002
Please refer to the end of the specification for access instructions.

### Example 2: Evaluation of CD3 scFv Sequence Variants in Comparison to Parental CD3 scFv

The purpose of the experiments was to evaluate four CD3 sequence variants to determine if the variants had enhanced properties in comparison to the CD3.9 parental scFv.

1. Determination of Melting Temperature (Tₘ)

The melting temperature of each scFv variant was measured to determine its thermal stability. Briefly, a uniform quantity of scFv in 200 μL of 1% BSA-PBST was aliquoted into PCR tubes. Tubes were incubated for one hour at several different temperatures (50° C., 51.4° C., 53.7° C., 57.3° C., 61.7° C., 65.5° C., and 68° C.). 50 μL of each sample was added to an ELISA plate coated with CD3CEμ target antigen (Creative Biomart) or BSA (reference to address stickiness). The wells of the ELISA plate were prefilled with 1% BSA-PBST (50 μl/well). Plates were incubated for 1 hour at room temperature. Plates were washed three times with water with 0.05% TWEEN to remove unbound scFv. Bound scFv was detected by adding an anti-YOL antibody (Thermo Scientific # MA180189) (1:500 diluted in 1% BSA-PBST (0.05%)) that detects a porcine alpha-tubulin motif in the linker between the heavy and light chain. Samples were incubated at room temperature for 1 hour. Plates were washed three times with water with 0.05% TWEEN to remove unbound scFv. The anti-YOL antibody was detected by adding an anti-rat-HRP antibody (Thermo Scientific #31470) (1:7500 diluted in 1% BSA-PBST (0.05%)) [100 μl/well) and incubating at room temperature for 1 hour. Plates were washed three times with water with 0.05% TWEEN to remove unbound antibody. Plates were developed using TMB (3,3′,5,5′-tetramethylbenzidine) substrate (100 μL/well for 6 minutes at room temperature. The reactions were stopped with H2504 (0.5M, 100 μL/well). The relative activity was measured as the absorbance reading at 450 nM. The absorbance at each temperature was graphed. The melting temperature was determined to be the EC50 of each sample, the temperature at which the binding of the scFv was reduced to 50% of maximal signal. The results are presented in Table 11.

Results: The assay results demonstrate that the CD3 scFv 3.23 and 3.24 had a Tm of 5° C. higher than the parental CD3.9, while the CD3.25 and CD3.26 (sequences shown in Table 12) scFv had Tₘ that were equivalent to the parental CD3.9.

2. Determination of Binding Affinity to CD3

The binding affinity of each scFv was measured using the ForteBio BLItz instrument. A dilution series of each scFv was prepared in PBS (300 μL/tube) starting from 1000 nM to 62.5 nM in one to one dilution steps for CD3.24-26, 400 nM to 25 nM in one to one dilution steps for CD3.23. Biotinylated CD3CEμ antigen (Creative Biomart) was diluted in PBS to a final concentration of 30 ug/ml. Streptavidin Biosensors (ForteBio) were activated in PBS for 10 minutes. To perform the measurements, the streptavidin biosensors were applied to the BLItz instrument. A tube containing 300 μL of PBS was transferred to the BLItz instrument for 30 seconds. A tube containing biotinylated CD3CEμ (30 ug/ml, 300 μL/tube) was transferred to the BLItz instrument to measure capture of antigen to sensor for 120 seconds. A tube containing 300 μL of PBS was transferred to the BLItz instrument for 30 seconds to measure the baseline signal. A tube containing test scFv (30 ug/ml, 300 μL/tube) was transferred to the BLItz instrument to measure association of the scFv to antigen-loaded biosensor for 120 seconds. A tube containing 300 μL of PBS was transferred to the BLItz instrument for 120 seconds to measure dissociation of the scFv from the antigen-loaded biosensor. The protocol was repeated for each scFv dilution. The KD of each antibody was determined using the BLI software (ForteBio). The results are presented in Table 11, which shows melting temp and binding affinity of the CD3 binding variants, demonstrating that variants such as CD3.23 have reduced binding affinity for CD3.

The binding affinity of bivalent anti-HER2, anti-CD3 XTENylated binder AC2275 (see Example 24) was measured against targets (HER2 and CD3) using a ForteBio Octet Red instrument. The assay was performed in a PBSTB buffer (10 mM sodium phosphate dibasic, 1.8 mM potassium phosphate monobasic, 137 mM sodium chloride, 2.7 mM potassium chloride, 0.5% BSA, 0.005% Tween-20). For binding to human HER2 or cynomolgus monkey HERa2, a dilution series of each analyte was prepared in PBSTB buffer (500 μL/tube) starting from 64 nM to 1 nM in one to one dilution steps. For binding to human CD3 or cynomolgus monkey CD3, a dilution series of each analyte was prepared in PBSTB buffer (500 μL/tube) starting from 1010 nM to 16 nM in one to one dilution steps. Targets were diluted in PBSTB to a final concentration of 33 ug/ml. Anti-human Fc biosensors (ForteBio) were activated in PBSTB buffer for 10 minutes. To perform the measurements, a set of anti-human Fc biosensors were placed on the sensor rack and were transferred to Octet Red instrument. A 96-well non-binding opaque plate containing 200 μL of PBSTB buffer, glycine buffer, targets and analytes were transferred to Octet Red instrument. Biosensors were transferred to the PBSTB buffer for 600 seconds for equilibration. For activation, biosensors were transferred to a 10 mM glycine buffer, pH 1.5 for 10 seconds and were transferred to PBSTB buffer for 10 seconds. The activation step was repeated for additional 2 times. Biosensors were transferred to the target well for 100 seconds for loading step. Biosensors were transferred to PBSTB buffer for 600 seconds for baseline measurement. Biosensors were transferred to well of analyte for 200-400 seconds for association step. Biosensors were transferred to well of analyte for 300-400 seconds for disassociation step. The protocol was repeated for each target. The binding affinity of each antibody was determined using the Octet Data Analysis software (ForteBio). The results are presented in Table 11A.

Results: The assay results demonstrate that the CD3 sequence variants all had reduced binding affinity to CD3 in comparison to the parental CD3.9.

TABLE 11
Tₘ and Binding Affinity Results

| scFv Construct | Melting temp (° C.) | Binding Affinity (nM) |
|---|---|---|
| CD3.9 | 57 | 75 |
| CD3.23 | 62 | 175 |
| CD3.24 | 62 | 296 |
| CD3.25 | 57 | 215 |
| CD3.26 | 57 | 221 |

TABLE 11A
Binding Affinity Results of AC2275

| Target | Binding Affinity (nM) |
|---|---|
| Human HER2-Fc | 4.5 |
| Cyno HER2-Fc | 1.2 |
| Human CD3 ϵ -Fc | 111 |
| Cyno CD3 ϵ -Fc | 170 |

TABLE 12
scFv sequences

| | | SEQ ID |
|---|---|---|
| Construct | Amino Acid Sequence | NO: |
| 3.25 | ELVVTQEPSHTVSPGGTVTLTCRSSTGAVTS | 1153 |
| | SNYANWVQQKPGQAPRGLIGGTIKRAPGTPA | |
| | RFSGSLLGGKAALTLSGVQPEDEAEYYCALW | |
| | YPNLWVFGGGTKLTVLGATPPETGAETESPG | |
| | ETTGGSAESEPPGEGEVQLLESGGGIVQPGG | |
| | SLKLSCAASGFTENTYAMNWVRQAPGKGLEW | |
| | VARIRSKYNNYATYYADSVKDRETISRDDSK | |
| | NTVYLQMNNLKTEDTAVYYCVRHENFGNSYV | |
| | SWFAHWGQGTLVTVSS | |
| 3.26 | ELVVTQEPSHTVSPGGTVTLTCRSSTGEVTT | 1154 |
| | SNYANWVQQKPGQAPRGLIGGTIKRAPGTPA | |
| | RFSGSLLGGKAALTLSGVQPEDEAEYYCALW | |
| | YPNLWVFGGGTKLTVLGATPPETGAETESPG | |
| | ETTGGSAESEPPGEGEVQLLESGGGIVQPGG | |
| | SLKLSCAASGFTENTYAMNWVRQAPGKGLEW | |
| | VARIRSKYNNYATYYADSVKDRETISRDDSK | |
| | NTVYLQMNNLKTEDTAVYYCVRHENFGNSYV | |
| | SWFAHWGQGTLVTVSS | |

Conclusions: Two new anti-CD3 scFvs have been identified that have improved thermal stability. Each new scFv has 8 to 9 mutations relative to CD3.9, residing primarily in the CDRs. These mutations have reduced affinity of the scFvs for their target (CD3) compared to the parental CD3.9 but bispecific T cell engagers utilizing CD3.23 are still efficacious in cell killing assays and in vivo.

### Example 3: Fermentation and Purification of Stable and Unstable Chimeric Fusion Polypeptides Comprising Bispecific Antigen Binding Fragments, Release Segments, and XTEN

The following example describes production of two highly-similar chimeric bispecific antigen binding fragment compositions, differing only in the anti-CD3 antigen binding fragment utilized, the observed incongruity of aggregation tendency between the two constructs, and the discovery that the sequence of the anti-CD3 antigen binding fragment had a significant impact on production, recovery, and purification of stable, soluble product.

Construct ID pJB0169 is a molecule having eight distinct domains. From the N-terminus to the C-terminus, the molecule consists of an N-terminal polyhistidine tag (His6 (SEQ ID NO: 1150)), an unstructured 292 amino acid chain (XTEN_AE293), a protease cleavable release segment (RS), an anti-EGFR scFv (aEGFR.2), an anti-CD3 scFv (aCD3.9), another protease cleavable release segment (RS), an unstructured 864 amino acid chain, and four C-terminal residues—glutamic acid, proline, glutamic acid, alanine (C-tag) (XTEN_AE868).

Construct ID pJB0231 is a molecule configured similarly; from the N-terminus to the C-terminus, the molecule consists of an N-terminal polyhistidine tag (His6 (SEQ ID NO: 1150)), an unstructured 292 amino acid chain (XTEN_AE292), a protease cleavable release segment (RS), an anti-EGFR scFv (aEGFR.2), an anti-CD3 scFv (aCD3.23), another protease cleavable release segment (RS), an unstructured 864 amino acid chain, and four C-terminal residues—glutamic acid, proline, glutamic acid, alanine (C-tag) (XTEN_AE868).

EXPRESSION: Both molecules (pJB0169 and pJB0231) were expressed in a proprietary E. coli AmE098 strain and partitioned into the periplasm via an N-terminal secretory leader sequence (MKKNIAFLLASMFVFSIATNAYA-(SEQ ID NO: 1155)), which was cleaved during translocation. Fermentation cultures were grown with animal-free complex medium at 37° C. and temperature shifted to 26° C. prior to phosphate depletion with continued fermentation for 12 hours following phosphate depletion. During harvest, fermentation whole broth was centrifuged to pellet the cells. At harvest, the total volume and the wet cell weight (WCW; ratio of pellet to supernatant) were recorded, and the pelleted cells were collected and frozen at −80° C.

CLARIFICATION: Frozen cell pellet of each molecule (pJB0169 and pJB0231) was resuspended 3-fold in lysis buffer (60 mM acetic acid, 350 mM NaCl) at pH 4.5, and the cells were lysed via homogenization. The homogenate was flocculated overnight at pH 4.5 and 2-8° C. The flocculated homogenate was centrifuged, and the supernatant was retained. The supernatant was diluted approximately 3-fold with water, then adjusted to 7±1 mS/cm with NaCl. The supernatant was then adjusted to 0.1% (m/m) diatomaceous earth and mixed via impeller. The supernatant was filtered through a filter train ending with a 0.22 μm filter. The filtrate was adjusted to pH 7.0 with sodium phosphate dibasic.

PURIFICATION: Each molecule (pJB0169 and pJB0231) was initially captured from clarified lysate and purified by Protein-L Chromatography (TOYOPEARL AF-rProtein L-650F). Subsequently, IMAC chromatography (GE IMAC Sepharose 6 FF) was used to select for the N-terminal His6-tag (SEQ ID NO: 1150), then C-tag affinity chromatography (CaptureSelect C-tagXL Affinity Matrix) was used to select for the C-terminal EPEA-tag (SEQ ID NO: 1149). Anion exchange chromatography (BIA CIMmultus QA monolith) was used to remove HMWCs and to polish to final purity.

ANALYTICS: The aggregation state of the process intermediates was monitored by SEC-HPLC. The SEC-HPLC method was performed using a Phenomenex 3 μm SEC-4000 300×7.8 mm (P/N 00H-4514-K0), a 20-minute isocratic method, at 1 mL/min, while monitoring the absorbance at 220 nm. pJB0169 monomer elutes from the analytical column at 6.2 minutes, and HMWC elute from 4.8-6.0 minutes. SEC-HPLC quality was measured as the relative area under the curve at 6.2 minutes versus the total area under the curve from 4.8-6.4 minutes.

Results: Aggregation summary (SEC-HPLC % monomer) for construct pJB0169 and construct pJB0231 following each unit operation are presented in Table 13. Recovery of ≥95% monomer was the quality threshold upon final polishing as the criterion for considering a molecule stable or processable.

TABLE 13
Analytic Results

| Unit | Unit | | |
|---|---|---|---|
| Operation | Operation | pJB0169 | pJB0231 |
| Number | Description | αEGFR.2-αCD3.9 | αEGFR.2-αCD3.23 |
| 1 | Clarified | 26.7% | 4.4% |
| | lysate | | |
| 2 | Protein L | 40.7% | 13.0% |
| | eluate | | |
| 3 | IMAC eluate | 52.8% | 36.9% |
| 4 | C-tag eluate | 71.0% | 53.8% |
| 5 | AEX eluate | 99.9% | 79.2% |
| | | Stable | unstable |

CONCLUSIONS: Construct pJB0169 was purified to the target monomeric quality by SEC-HPLC (≥95% monomer), indicating that the construct is stable and compatible with both recovery and purification operations. However, construct pJB0231 could not be purified to the target monomeric quality, achieving only 79% monomer upon final polishing, indicating that the construct is either unstable or incompatible with recovery or purification operations (or a combination thereof). Because the only difference between pJB0169 and pJB0231 is the anti-CD3 scFv sequence, it was hypothesized that the anti-CD3.23 scFv is incompatible within the context of the bispecific molecule as composed with the various components.

STABILITY IMPROVEMENT AND ASSESSMENT: New scFvs (anti-EGFR.23 and anti-CD3.32) were designed for improved stability via (1) reduction of surface hydrophobicity and (2) reduction of isoelectric point differences between the paired scFv molecules (fused by a short peptide linker) by substitution of amino acids at select locations. Constructs pAH0025 and pAH0026 represent design iterations on pJB0169, where pAH0025 contains the anti-CD3.32 scFv variant, and pAH0026 contains both the anti-CD3.32 scFv variant and the EGFR.23 scFv variant. Constructs pAH0025 and pAH0026 would be expressed, clarified, purified, and analyzed as above; the SEC-HPLC results throughout the purification would be monitored and compared to pJB0169 and pJB0231 to assess relative stability. New design pairings are anticipated to be more stable than pJB0231 and would be expected to show concomitant improvement in percent monomer content, as measured by SEC-HPLC, following the unit operations tabulated below in Table 14 (or a subset thereof). Any construct that meets the purity target of >95% monomer would be considered stable or processable.

TABLE 14
analytic results

| | Unit | SEC-HPLC quality (% monomer) | | | |
|---|---|---|---|---|---|
| Unit | Operation | pJB0169 | pJB0231 | pAH0025 | pAH0026 |
| Operation | Descrip- | αEGFR.2- | αEGFR.2- | αEGFR.2- | αEGFR.23- |
| Number | tion | αCD3.9 | αCD3.23 | αCD3.32 | αCD3.32 |
| 1 | Clarified | 26.7% | 4.4% | TBD | TBD |
| | lysate | | | | |
| 2 | Protein L | 40.7% | 13.0% | TBD | TBD |
| | eluate | | | | |
| 3 | IMAC | 52.8% | 36.9% | TBD | TBD |
| | eluate | | | | |
| 4 | C-tag | 71.0% | 53.8% | TBD | TBD |
| | eluate | | | | |
| 5 | AEX | 99.9% | 79.2% | TBD | TBD |
| | eluate | | | | |
| | | stable | unstable | TBD | TBD |

### Example 4: Binding Affinity of Anti-EpCAM×Anti-CD3 Bispecific Antigen Binding Polypeptide Composition

The binding affinity of anti-EpCAM×anti-CD3 bispecific antigen binding polypeptide constructs to human EpCAM and human CD3 was measured using flow cytometry with huEp-CHO 4-12B (CHO cell line transfected with human EpCAM) and Jurkat cells.

The binding constants for anti-EpCAM×anti-CD3 bispecific antigen binding polypeptide binding to EpCAM-expressing and CD3-expressing cells was measured by competition binding with a fluorescently-labeled, protease-treated bispecific antigen binding polypeptide. The fluorescently-labeled, protease-treated bispecific antigen binding polypeptide was made by conjugation of Alexa Fluor 647 C2 maleimide (Thermo Fisher, cat#A20347) to a cysteine-containing, protease-treated bispecific antigen binding polypeptide mutant (MMP-9 treated pCW1645). Binding experiments were performed on 10,000 cells at 4° C. for 1 hour in a total volume of 100 μL of binding buffer (2% FCS, 5 mM EDTA, HBSS). Cells were washed once with cold binding buffer, then re-suspended in 1% formaldehyde in phosphate-buffered saline and immediately analyzed on a Millipore Guava easyCyte flow cytometer. Binding of the fluorescently-labeled, protease-treated pCW1645 was found to have an apparent K_{d} value of 1 nM to hEp-CHO 4-12B and 4 nM to CD3+Jurkat cells.

Competition binding experiments were performed on 10,000 hEp-CHO 4-12B cells with 1.5 nM fluorescently-labeled, protease-treated pCW1645 at 4° C. for 1 hour in a total volume of 100 μL of binding buffer (2% FCS, 5 mM EDTA, HBSS). Cells were washed once with cold binding buffer, then re-suspended in 1% formaldehyde in phosphate-buffered saline and immediately analyzed on a Millipore Guava easyCyte flow cytometer. Competition binding of fluorescently labeled, protease-treated pCW1645 to hEp-CHO 4-12B cells with cleaved bispecific antigen binding polypeptide (pJB0189 hEp.2-hCD3.9 or AC1984 hEp.2-hCD3.23) resulted in apparent binding constants of 0.5 nM for hEp.2 (panitumumab).

Competition binding experiments were performed on 10,000 Jurkat cells with 10 nM fluorescently-labeled, protease-treated pCW1645 at 4° C. for 1 hour in a total volume of 100 μL of binding buffer (2% FCS, 5 mM EDTA, HBSS). Cells were washed once with cold binding buffer, then re-suspended in 1% formaldehyde in phosphate-buffered saline and immediately analyzed on a Millipore Guava easyCyte flow cytometer. Competition binding of fluorescently-labeled, protease-treated pCW1645 to Jurkat cells with cleaved bispecific antigen binding polypeptide (pJB0189 hEp.2-hCD3.9 or AC1984 hEp.2-hCD3.23) resulted in apparent binding constants of 75 nM for hCD3.9 and 300 nM for hCD3.23 for CD3 binding and 0.5 nM for EpCAM binding.

Conclusions: The binding affinity of CD3.23 for CD3 on Jurkat cells is 300 nM, which is 4-fold weaker than the affinity of CD3.9. The binding affinity of hEp.2 for EpCAM on Jurkat cells is 0.5 nM.

### Example 5: Binding Affinity of Anti-HER2×Anti-CD3 Bispecific Antigen Binding Polypeptide Composition

The binding affinity of anti-HER2×anti-CD3 bispecific antigen binding polypeptide constructs to human HER2 and human CD3 are measured using flow cytometry with hHER2-CT26 (CT26 cell line transfected with human HER2) and Jurkat cells.

The binding constants for anti-HER2×anti-CD3 bispecific antigen binding polypeptide binding to HER2-expressing and CD3-expressing cells are measured by competition binding with a fluorescently labeled, protease-treated bispecific antigen binding polypeptide. The fluorescently labeled bispecific antigen binding polypeptide is made by conjugation of Alexa Fluor 647 C2 maleimide (Thermo Fisher, cat#A20347) to a cysteine-containing bispecific antigen binding polypeptide mutant (MMP-9 treated pJB0297 (see Table 14B)) with hHER2.2-hCD3.23 and two XTEN. The fluorescently-labeled, protease-treated bispecific antigen binding polypeptide is made by conjugation of Alexa Fluor 647 C2 maleimide (Thermo Fisher, cat#A20347) to a cysteine-containing, protease-treated bispecific antigen binding polypeptide mutant (MMP-9 treated pJB0297). Binding experiments are performed on 10,000 cells at 4° C. for 1 hour in a total volume of 100 μL of binding buffer (2% FCS, 5 mM EDTA, HBSS). Cells are washed once with cold binding buffer, then re-suspended in 1% formaldehyde in phosphate-buffered saline and immediately analyzed on a Millipore Guava easyCyte flow cytometer. Binding of the fluorescently-labeled, protease-treated pJB0297 is expected to have an apparent K_{d} value in the low nM concentration to hHER2-CT26 and about 300 nM to CD3+ Jurkat cells. Binding of the fluorescently-labeled pJB0297 with two XTEN is expected to have an apparent K_{d} value about 10- to 100-fold weaker than for fluorescently-labeled, protease-treated bispecific antigen binding polypeptide to hHER2-CT26 and CD3+ Jurkat cells.

Competition binding experiments are performed on 10,000 hHER2-CT26 cells at a concentration of fluorescently-labeled, protease-treated pJB0297 close to the K_{d} from the previously described binding experiment at 4° C. for 1 hour in a total volume of 100 μL of binding buffer (2% FCS, 5 mM EDTA, HBSS). Cells are washed once with cold binding buffer, then re-suspended in 1% formaldehyde in phosphate-buffered saline and immediately analyzed on a Millipore Guava easyCyte flow cytometer. Competition binding of fluorescently-labeled, protease-treated pJB0297 to hHER2-CT26 cells with pJB0244 bispecific antigen binding polypeptide is expected to have an apparent binding constant similar to the direct binding constant of fluorescently-labeled pJB0297.

Competition binding experiments are performed on 10,000 Jurkat cells with about 300 nM (or a concentration close to the K_{d} from the previously described binding experiment) of fluorescently-labeled, protease-treated pJB0297 at 4° C. for 1 hour in a total volume of 100 μL of binding buffer (2% FCS, 5 mM EDTA, HBSS). Cells are washed once with cold binding buffer, then re-suspended in 1% formaldehyde in phosphate-buffered saline and immediately analyzed on a Millipore Guava easyCyte flow cytometer. Competition binding of fluorescently-labeled, protease-treated pJB0297 to Jurkat cells with pJB0244 bispecific antigen binding polypeptide is expected to have an apparent binding constant similar to the direct binding constant of fluorescently-labeled pJB0297, which is expected to be in the low micromolar to nanomolar concentration range.

TABLE 14B
pJB0297-DNA and Amino Acid Sequence

| Construct | | SEQ ID | Amino Acid | SEQ ID |
|---|---|---|---|---|
| Name | DNA Sequence | NO: DNA | Sequence (AA) | NO: AA |
| pJB0297 | CACCATCATCACCATCACTCCCCA | 1137 | HHHHHHSPAGSPTSTEEGTSES | 1138 |
| | GCAGGCAGCCCGACCAGCACCGAG | | ATPESGPGTSTEPSEGSAPGTS | |
| | GAGGGTACGAGCGAGTCGGCTACT | | ESATPESGPGSEPATSGSETPG | |
| | CCAGAGAGCGGTCCGGGTACCTCT | | TSESATPESGPGSEPATSGSET | |
| | ACGGAACCGTCCGAAGGTAGCGCT | | PGTSESATPESGPGTSTEPSEG | |
| | CCAGGCACGTCTGAAAGCGCGACG | | SAPGSPAGSPTSTEEGTSESAT | |
| | CCGGAAAGCGGTCCAGGCAGCGAG | | PESGPGSEPATSGSETPGTSES | |
| | CCGGCGACCTCCGGTAGCGAAACG | | ATPESGPGSPAGSPTSTEEGSP | |
| | CCTGGTACCTCGGAGTCAGCGACT | | AGSPTSTEEGTSTEPSEGSAPG | |
| | CCGGAAAGCGGTCCGGGTAGCGAA | | TSESATPESGPGTSESATPESG | |
| | CCTGCAACGAGCGGTAGCGAGACT | | PGTSESATPESGPGSEPATSGS | |
| | CCAGGCACTAGCGAATCCGCAACT | | ETPGSEPATSGSETPGSPAGSP | |
| | CCGGAGTCGGGTCCGGGCACCTCT | | TSTEEGTSTEPSEGSAPGTSTE | |
| | ACGGAGCCTAGCGAGGGCTCAGCA | | PSEGSAPGGSAPEAGRSANHTP | |
| | CCAGGTAGCCCTGCAGGTTCCCCG | | AGLTGPATSGSETPGTDIQMTQ | |
| | ACGTCAACCGAGGAAGGTACAAGC | | SPSSLSASVGDRVTITCKASQD | |
| | GAAAGCGCCACCCCTGAGTCGGGC | | VSIGVAWYQQKPGKAPKLLIYS | |
| | CCTGGCAGCGAACCGGCAACTAGC | | ASYRYTGVPSRFSGSGSGTDFT | |
| | GGCAGCGAGACTCCGGGTACCAGC | | LTISSLQPEDFATYYCQQYYIY | |
| | GAGTCTGCTACGCCAGAGAGCGGC | | PYTFGQGTKVEIKGATPPETGA | |
| | CCAGGTTCGCCAGCGGGTTCGCCG | | ETESPGETTGGSAESEPPGEGE | |
| | ACTAGCACGGAGGAGGGCAGCCCA | | VQLVESGGGLVQPGGSLRLSCA | |
| | GCGGGTAGCCCGACCAGCACTGAG | | ASGFTFTDYTMDWVRQAPGKGL | |
| | GAGGGTACGTCCACCGAACCGAGC | | EWVADVNPNSGGSIYNQRFKGR | |
| | GAAGGTAGCGCACCAGGTACCTCC | | FTLSVDRSKNTLYLQMNSLRAE | |
| | GAGTCTGCCACCCCTGAATCCGGT | | DTAVYYCARNLGPSFYFDYWGQ | |
| | CCAGGTACCAGCGAATCAGCCACC | | GTLVTVSSGGGGSELVVTQEPS | |
| | CCGGAGTCGGGTCCAGGTACGAGC | | LTVSPGGTVTLTCRSSNGAVTS | |
| | GAATCTGCTACCCCGGAATCCGGC | | SNYANWVQQKPGQAPRGLIGGT | |
| | CCAGGCAGCGAACCTGCTACTAGC | | NKRAPGTPARFSGSLLGGKAAL | |
| | GGCAGCGAAACGCCGGGCAGCGAA | | TLSGVQPEDEAVYYCALWYPNL | |
| | CCTGCCACGTCAGGCAGCGAGACG | | WVFGGGTKLTVLGATPPETGAE | |
| | CCGGGTTCCCCTGCAGGCTCCCCG | | TESPGETTGGSAESEPPGEGEV | |
| | ACCAGCACTGAGGAGGGCACCTCC | | QLLESGGGIVQPGGSLKLSCAA | |
| | ACCGAACCATCAGAAGGTAGCGCG | | SGFTFNTYAMNWVRQAPGKGLE | |
| | CCTGGTACGTCAACCGAACCTTCC | | WVARIRSKYNNYATYYADSVKD | |
| | GAGGGCAGCGCACCGGGTGGCTCA | | RFTISRDDSKNTVYLQMNNLKT | |
| | GCGCCTGAGGCAGGTCGTTCTGCT | | EDTAVYYCVRHENFGNSYVSWF | |
| | AACCATACCCCTGCAGGATTAACT | | AHWGQGTLVTVSSGTAEAASAC | |
| | GGCCCCGCCACCAGCGGGAGCGAG | | GEAGRSANHTPAGLTGPPGSPA | |
| | ACCCCCGGGACTGACATTCAGATG | | GSPTSTEEGTSESATPESGPGT | |
| | ACTCAGTCTCCGTCCTCCCTGTCT | | STEPSEGSAPGSPAGSPTSTEE | |
| | GCGAGCGTGGGCGACCGTGTGACT | | GTSTEPSEGSAPGTSTEPSEGS | |
| | ATTACCTGTAAAGCCTCCCAGGAC | | APGTSESATPESGPGSEPATSG | |
| | GTGTCTATCGGTGTGGCATGGTAT | | SETPGSEPATSGSETPGSPAGS | |
| | CAACAAAAGCCGGGTAAGGCACCT | | PTSTEEGTSESATPESGPGTST | |
| | AAACTGCTGATCTACTCCGCTTCT | | EPSEGSAPGTSTEPSEGSAPGS | |
| | TACCGTTACACGGGCGTTCCGTCC | | PAGSPTSTEEGTSTEPSEGSAP | |
| | CGTTTTAGCGGTTCCGGTAGCGGT | | GTSTEPSEGSAPGTSESATPES | |
| | ACTGATTTTACCCTGACTATTTCC | | GPGTSTEPSEGSAPGTSESATP | |
| | TCCCTGCAACCAGAAGACTTTGCG | | ESGPGSEPATSGSETPGTSTEP | |
| | ACCTATTACTGTCAGCAATACTAT | | SEGSAPGTSTEPSEGSAPGTSE | |
| | ATTTACCCGTATACCTTCGGCCAG | | SATPESGPGTSESATPESGPGS | |
| | GGCACTAAGGTTGAAATTAAAGGT | | PAGSPTSTEEGTSESATPESGP | |
| | GCAACGCCTCCGGAGACTGGTGCT | | GSEPATSGSETPGTSESATPES | |
| | GAAACTGAGTCCCCGGGCGAGACG | | GPGTSTEPSEGSAPGTSTEPSE | |
| | ACCGGTGGCTCTGCTGAATCCGAA | | GSAPGTSTEPSEGSAPGTSTEP | |
| | CCACCGGGCGAAGGCGAGGTTCAG | | SEGSAPGTSTEPSEGSAPGTST | |
| | CTGGTGGAGTCTGGCGGCGGTCTG | | EPSEGSAPGSPAGSPTSTEEGT | |
| | GTACAGCCGGGTGGTAGCCTGCGT | | STEPSEGSAPGTSESATPESGP | |
| | CTGAGCTGCGCGGCGTCCGGTTTC | | GSEPATSGSETPGTSESATPES | |
| | ACTTTCACCGATTATACCATGGAC | | GPGSEPATSGSETPGTSESATP | |
| | TGGGTTCGCCAGGCACCGGGCAAG | | ESGPGTSTEPSEGSAPGTSESA | |
| | GGTCTGGAATGGGTGGCGGACGTG | | TPESGPGSPAGSPTSTEEGSPA | |
| | AACCCGAACTCCGGTGGTTCTATC | | GSPTSTEEGSPAGSPTSTEEGT | |
| | TACAACCAGCGTTTCAAAGGTCGT | | SESATPESGPGTSTEPSEGSAP | |
| | TTCACGCTGAGCGTAGATCGTAGC | | GTSESATPESGPGSEPATSGSE | |
| | AAAAACACTCTGTACCTGCAGATG | | TPGTSESATPESGPGSEPATSG | |
| | AACTCCCTGCGCGCAGAAGACACC | | SETPGTSESATPESGPGTSTEP | |
| | GCGGTGTATTACTGTGCACGTAAC | | SEGSAPGSPAGSPTSTEEGTSE | |
| | CTGGGCCCGTCCTTCTATTTCGAC | | SATPESGPGSEPATSGSETPGT | |
| | TACTGGGGTCAAGGTACTCTGGTA | | SESATPESGPGSPAGSPTSTEE | |
| | ACTGTTTCCTCTGGTGGTGGCGGC | | GSPAGSPTSTEEGTSTEPSEGS | |
| | AGCGAGTTAGTTGTGACCCAAGAG | | APGTSESATPESGPGTSESATP | |
| | CCGAGCCTGACCGTTAGCCCGGGT | | ESGPGTSESATPESGPGSEPAT | |
| | GGTACGGTCACCCTGACGTGCCGT | | SGSETPGSEPATSGSETPGSPA | |
| | AGCAGCAACGGTGCGGTCACGAGC | | GSPTSTEEGTSTEPSEGSAPGT | |
| | AGCAACTATGCCAATTGGGTCCAG | | STEPSEGSAPGSEPATSGSETP | |
| | CAGAAACCGGGTCAAGCACCGCGT | | GTSESATPESGPGTSTEPSEGA | |
| | GGCCTGATCGGCGGCACCAATAAA | | AEPEA | |
| | CGTGCCCCGGGTACTCCTGCGCGT | | | |
| | TTCTCCGGTAGCCTGCTGGGCGGC | | | |
| | AAAGCCGCTCTGACCCTGAGCGGT | | | |
| | GTCCAGCCGGAAGATGAAGCGGTG | | | |
| | TACTACTGCGCGCTGTGGTATCCG | | | |
| | AATCTGTGGGTTTTTGGCGGCGGT | | | |
| | ACCAAGCTGACCGTATTGGGTGCT | | | |
| | ACGCCACCGGAGACTGGCGCAGAA | | | |
| | ACGGAAAGCCCGGGTGAGACTACG | | | |
| | GGTGGCTCTGCGGAGAGCGAACCT | | | |
| | CCGGGTGAGGGTGAGGTCCAACTG | | | |
| | CTGGAGTCTGGTGGTGGCATTGTT | | | |
| | CAACCGGGTGGCTCGTTGAAGCTG | | | |
| | AGCTGTGCAGCTAGCGGCTTTACC | | | |
| | TTCAACACCTATGCGATGAATTGG | | | |
| | GTTCGTCAGGCACCGGGTAAGGGC | | | |
| | CTGGAATGGGTGGCGCGTATCCGC | | | |
| | TCCAAGTACAACAACTACGCGACC | | | |
| | TACTACGCGGATAGCGTTAAAGAC | | | |
| | CGCTTCACGATTAGCCGTGACGAT | | | |
| | TCCAAGAATACGGTGTATCTGCAA | | | |
| | ATGAACAATCTGAAAACCGAAGAT | | | |
| | ACCGCGGTGTATTACTGTGTGCGC | | | |
| | CACGAAAATTTCGGCAACAGCTAC | | | |
| | GTGAGCTGGTTTGCACATTGGGGT | | | |
| | CAGGGCACCCTGGTTACGGTGAGC | | | |
| | TCCGGGACTGCTGAGGCGGCTAGC | | | |
| | GCCTGTGGAGAAGCTGGAAGAAGC | | | |
| | GCCAATCACACACCAGCTGGACTT | | | |
| | ACAGGCCCGCCTGGTAGCCCCGCG | | | |
| | GGGAGCCCTACAAGCACTGAGGAG | | | |
| | GGCACATCTGAGTCCGCTACCCCT | | | |
| | GAGAGTGGACCCGGGACAAGCACT | | | |
| | GAGCCTAGCGAAGGAAGCGCACCA | | | |
| | GGTTCCCCCGCTGGGAGCCCCACA | | | |
| | AGCACAGAAGAGGGAACTTCTACC | | | |
| | GAGCCCTCTGAGGGCTCAGCCCCT | | | |
| | GGAACTAGCACAGAGCCCTCCGAA | | | |
| | GGCAGTGCACCGGGTACTTCCGAA | | | |
| | AGCGCAACTCCGGAATCCGGCCCT | | | |
| | GGTTCTGAGCCTGCTACTTCCGGC | | | |
| | TCTGAAACTCCAGGTAGCGAGCCA | | | |
| | GCGACTTCTGGTTCTGAAACTCCA | | | |
| | GGTTCACCGGCGGGTAGCCCGACG | | | |
| | AGCACGGAGGAAGGTACCTCTGAG | | | |
| | TCGGCCACTCCTGAGTCCGGTCCG | | | |
| | GGCACGAGCACCGAGCCGAGCGAG | | | |
| | GGTTCAGCCCCGGGTACCAGCACG | | | |
| | GAGCCGTCCGAGGGTAGCGCACCG | | | |
| | GGTTCTCCGGCGGGCTCCCCTACG | | | |
| | TCTACGGAAGAGGGTACGTCCACT | | | |
| | GAACCTAGCGAGGGCAGCGCGCCA | | | |
| | GGCACCAGCACTGAACCGAGCGAA | | | |
| | GGCAGCGCACCTGGCACTAGCGAG | | | |
| | TCTGCGACTCCGGAGAGCGGTCCG | | | |
| | GGTACGAGCACGGAACCAAGCGAA | | | |
| | GGCAGCGCCCCAGGTACCTCTGAA | | | |
| | TCTGCTACCCCAGAATCTGGCCCG | | | |
| | GGTTCCGAGCCAGCTACCTCTGGT | | | |
| | TCTGAAACCCCAGGTACTTCCACT | | | |
| | GAACCAAGCGAAGGTAGCGCTCCT | | | |
| | GGCACTTCTACTGAACCATCCGAA | | | |
| | GGTTCCGCTCCTGGTACGTCTGAA | | | |
| | AGCGCTACCCCTGAAAGCGGCCCA | | | |
| | GGCACCTCTGAAAGCGCTACTCCT | | | |
| | GAGAGCGGTCCAGGCTCTCCAGCA | | | |
| | GGTTCTCCAACCTCCACTGAAGAA | | | |
| | GGCACCTCTGAGTCTGCTACCCCT | | | |
| | GAATCTGGTCCTGGCTCCGAACCT | | | |
| | GCTACCTCTGGTTCCGAAACTCCA | | | |
| | GGTACCTCGGAATCTGCGACTCCG | | | |
| | GAATCTGGCCCGGGCACGAGCACG | | | |
| | GAGCCGTCTGAGGGTAGCGCACCA | | | |
| | GGTACCAGCACTGAGCCTTCTGAG | | | |
| | GGCTCTGCACCGGGTACCTCCACG | | | |
| | GAACCTTCGGAAGGTTCTGCGCCG | | | |
| | GGTACCTCCACTGAGCCATCCGAG | | | |
| | GGTTCAGCACCAGGTACTAGCACG | | | |
| | GAACCGTCCGAGGGCTCTGCACCA | | | |
| | GGTACGAGCACCGAACCGTCGGAG | | | |
| | GGTAGCGCTCCAGGTAGCCCAGCG | | | |
| | GGCTCTCCGACAAGCACCGAAGAA | | | |
| | GGCACCAGCACCGAGCCGTCCGAA | | | |
| | GGTTCCGCACCAGGTACAAGCGAG | | | |
| | AGCGCGACTCCTGAATCTGGTCCG | | | |
| | GGTAGCGAGCCTGCAACCAGCGGT | | | |
| | TCTGAGACGCCGGGCACTTCCGAA | | | |
| | TCTGCGACCCCGGAGTCCGGTCCA | | | |
| | GGTTCAGAGCCGGCGACGAGCGGT | | | |
| | TCGGAAACGCCGGGTACGTCTGAA | | | |
| | TCAGCCACGCCGGAGTCTGGTCCG | | | |
| | GGTACCTCGACCGAACCAAGCGAA | | | |
| | GGTTCGGCACCGGGTACTAGCGAG | | | |
| | AGCGCAACCCCTGAAAGCGGTCCG | | | |
| | GGCAGCCCGGCAGGTTCTCCAACC | | | |
| | AGCACCGAAGAAGGTTCCCCTGCT | | | |
| | GGTAGCCCGACCTCTACGGAGGAA | | | |
| | GGTAGCCCTGCAGGTTCCCCAACT | | | |
| | TCTACTGAGGAAGGTACTTCTGAG | | | |
| | TCCGCTACCCCAGAAAGCGGTCCT | | | |
| | GGTACCTCCACTGAACCGTCTGAA | | | |
| | GGCTCTGCACCAGGCACTTCTGAG | | | |
| | TCTGCTACTCCAGAAAGCGGCCCA | | | |
| | GGTTCTGAACCAGCAACTTCTGGC | | | |
| | TCTGAGACTCCAGGCACTTCTGAG | | | |
| | TCCGCAACGCCTGAATCCGGTCCT | | | |
| | GGTTCTGAACCAGCTACTTCCGGC | | | |
| | AGCGAAACCCCAGGTACCTCTGAG | | | |
| | TCTGCGACTCCAGAGTCTGGTCCT | | | |
| | GGTACTTCCACTGAGCCTAGCGAG | | | |
| | GGTTCCGCACCAGGTTCTCCGGCT | | | |
| | GGTAGCCCGACCAGCACGGAGGAG | | | |
| | GGTACGTCTGAATCTGCAACGCCG | | | |
| | GAATCGGGCCCAGGTTCGGAGCCT | | | |
| | GCAACGTCTGGCAGCGAAACCCCG | | | |
| | GGTACCTCCGAATCTGCTACACCG | | | |
| | GAAAGCGGTCCTGGCAGCCCTGCT | | | |
| | GGTTCTCCAACCTCTACCGAGGAG | | | |
| | GGTTCACCGGCAGGTAGCCCGACT | | | |
| | AGCACTGAAGAAGGTACTAGCACG | | | |
| | GAGCCGAGCGAGGGTAGTGCTCCG | | | |
| | GGTACGAGCGAGAGCGCAACGCCA | | | |
| | GAGAGCGGTCCAGGCACCAGCGAA | | | |
| | TCGGCCACCCCTGAGAGCGGCCCA | | | |
| | GGTACTTCTGAGAGCGCCACTCCT | | | |
| | GAATCCGGCCCTGGTAGCGAGCCG | | | |
| | GCAACCTCCGGCTCAGAAACTCCT | | | |
| | GGTTCGGAACCAGCGACCAGCGGT | | | |
| | TCTGAAACTCCGGGTAGCCCGGCA | | | |
| | GGCAGCCCAACGAGCACCGAAGAG | | | |
| | GGTACCAGCACGGAACCGAGCGAG | | | |
| | GGTTCTGCCCCGGGTACTTCCACC | | | |
| | GAACCATCGGAGGGCTCTGCACCT | | | |
| | GGTAGCGAACCTGCGACGTCTGGT | | | |
| | TCTGAAACGCCGGGTACCAGCGAA | | | |
| | AGCGCTACCCCAGAATCCGGTCCG | | | |
| | GGCACTAGCACCGAGCCATCGGAG | | | |
| | GGCGCCGCAGAACCAGAGGCG | | | |

### Example 6: Human/Cynomolgus Monkey Cross-Reactivity of Anti-HER2×Anti-CD3 Bispecific Antigen Binding Polypeptide Composition

Redirected cellular cytotoxicity of an activated, cleaved (such that the masking XTEN are removed) anti-HER2×anti-CD3 bispecific antigen binding polypeptide composition (protease treatment of pJB0244 to result in pJB0244A) was compared in both human and cynomolgus monkey cell-based assay systems to investigate whether the cynomolgus monkey can serve as relevant species for pharmacologic and toxicological safety.

Human and cynomolgus monkey peripheral blood mononuclear cells (PBMC) were used as effector cells and HER2 transfected CT26 cells as targets. Human PBMC were isolated from screened, healthy donors by ficoll density gradient centrifugation from either whole blood or from lymphocyte-enriched buffy coat preparations obtained from local blood banks or Bioreclamation IVT. Cryopreserved normal cynomolgus monkey PBMCs were obtained from IQ Biosciences. PBMCs were thawed quickly in a 37° C. water bath and centrifuged with culture media (RPMI+FBS 10%) at 1300 rpm for 5 minutes and then the supernatant was removed. Both human and cynomolgus monkey PBMCs were resuspended and cultured at appropriate cell density as discussed below in RPMI-1640/FBS 10% at 37° C. in a 5% CO₂ humidified incubator until use. CT26 cells stably expressing human (CT26-huHER2) or cynomolgus monkey HER2 (CT26-cyHER2) were generated by transfecting full length huHER2 or cyno HER2 cDNA into mouse CT26 tumor cells and selecting for puromycin resistant clones. Selection of clones and amplification of expression was conducted in the presence of puromycin.

Caspase Glo 3/7 assay was used for the determination of the cytolytic activity of protease-treated anti-HER2×anti-CD3 cleavable bispecific antigen binding polypeptide composition (pJB0244). Caspase 3/7 assay utilizes a proluminescent caspase-3/7 DEVD-aminoluciferin substrate (“DEVD” disclosed SEQ ID NO: 1156) and a thermostable luciferase in a reagent optimized for caspase-3/7 activity, luciferase activity and cell lysis. Adding the reagent results in cell lysis, followed by caspase cleavage of the substrate. This liberates free aminoluciferin, which is consumed by the luciferase, generating a “glow-type” luminescent signal that is proportional to caspase-3/7 activity/cell lysis.

The cytotoxic performance of the protease-treated anti-HER2×anti-CD3 bispecific antigen binding polypeptide compositions with CT26-huHER2 and CT26-cyHER2 transfected cells was analyzed as follows: cell density of human and cyno PBMCs was first adjusted 2×10⁶ cells/mL, respectively, in assay medium comprised of RPMI/FBS 10%. CT26-huHER2 and CT26-cyHER2 transfected cells were resuspended at 5×10⁵ cells/mL assay medium comprised of RPMI/FBS 10% to achieve an effector to target ratio of 5:1. 50 μL aliquots of PBMC were co-cultured with 40 μL aliquots of CT26-huHER2/CT26-cyHER2 transfected cells per assay well in a 96-well round-bottom plate. Unmasked (protease treated) anti-HER2×anti-CD3 composition sample was diluted in assay medium to the desired dose concentration and added in 10 μL to the respective experimental wells bringing the total assay volume to 100 μL. The unmasked (no flanking XTEN) anti-HER2×anti-CD3 composition was evaluated as an 8-point, 5× serial diluted dose concentration starting at 1 nM to obtain a final dose range of 0.00006 to 1 nM. An assay control for background had an intact, untreated anti-HER2×anti-CD3 composition (pJB0244), only PBMC cells with CT26 transfected cells was also set up at this time. The plate containing experimental wells of unmasked protease-treated anti-HER2×anti-CD3 bispecific antigen binding polypeptide composition and the respective assay controls, all tested in duplicates, was then allowed to incubate overnight in a 37° C., 5% CO₂ humidified incubator.

The amount of Caspase 3/7 released into the supernatant as a result of cell lysis was measured using the Promega Caspase-Glo 3/7 Assay kit and following manufacturer's instructions. Before starting the assay, Caspase-Glo 3/7 Reagent was allowed to thaw and equilibrate to room temperature. A 96-well plate containing treated cells was removed from the incubator and allowed to equilibrate to room temperature. To each well in the enzymatic plate, 100 μl of Caspase-Glo 3/7 Reagent was added. The plate was then covered, protected from light and allowed to incubate at room temperature for 30 min. After the desired incubation period, the contents of wells were gently mixed using a plate shaker at 300-500 rpm for 30 seconds. Luminescence of each sample was measured in a plate-reading luminometer as directed by the luminometer manufacturer.

Data analysis was then performed as follows: dose concentration of unmasked, protease treated anti-HER2×anti-CD3 composition was then plotted against cytotoxicity (Relative Luminescence Units) measured, and the concentration of protein that gave half maximal response (EC₅₀) was derived with a 4-parameter logistic regression equation using GraphPad prism software.

Exposure of CT26-huHER2 transfected cells to unmasked protease treated anti-HER2×anti-CD3 composition in the presence of human PBMCs yielded concentration-dependent cytotoxic dose curves, with an EC₅₀ of 0.5 pM. With CT26-cyHER2 transfected cells and cynomolgus PBMCs, protease-treated anti-HER2×anti-CD3 composition yielded concentration-dependent cytotoxic dose curve with an EC50 of 1.2 pM.

Conclusions: The data indicate that protease-treated anti-HER2×anti-CD3 (pJB0244A) is cross-reactive with cyno HER2 (as in the CT26-cyHER2 transfected cells and cyno CD3 (as in cynomolgus PBMCs). Target cells expressing human HER2 were more potently lysed by human PBMC cells than those expressing cynomolgus monkey HER2. Human PBMC cells showed a 2.4-fold higher potency of redirected lysis with protease-treated anti-HER2×anti-CD3 composition than cynomolgus monkey PBMCs cells. Taken together, unmasked, protease-treated anti-HER2×anti-CD3 composition showed dose-dependent activity for redirected lysis with human and cynomolgus monkey PBMC cells, and reacted with HER2 antigen from both human and cynomolgus monkey species.

### Example 7: Cytotoxicity Assays of Anti-HER2×Anti-CD3 Bispecific Antigen Binding Polypeptide Composition

Redirected cellular cytotoxicity of unmasked (pJB0244A, with XTEN removed by proteolysis by MMP-9), masked (pJB0244A, having 2 XTEN and 2 release segments cleavable by proteolysis), and uncleavable (pJB0245, with 2 XTEN and the release segments replaced by a peptide not susceptible to proteolysis) anti-HER2×anti-CD3 bispecific antigen binding polypeptide compositions were assessed by using human peripheral blood mononuclear cells (PBMC) as effectors and HER2 positive human carcinoma cells such as BT-474, SK-Br-3, SK-OV-3, JIMT-1, MDA-MB-231 and MCF-7 (based on the levels of HER2 expression) as targets. PBMC were isolated from screened, healthy donors by ficoll density gradient centrifugation from either whole blood or from lymphocyte-enriched buffy coat preparations obtained from local blood banks or Bioreclamation IVT. Human PBMC cells were resuspended and cultured at appropriate cell density, as discussed below, in RPMI-1640/FBS 10% at 37° C. in a 5% CO₂ humidified incubator until use. Tumor cell lines were obtained from ATCC and grown in culture media as recommended by ATCC. A caspase Glo 3/7 assay was used for the determination of the cytolytic activity of unmasked anti-HER2×anti-CD3 composition (pJB0244A), masked (having 2 XTEN attached to antigen binding fragments via release segments) and uncleavable anti-HER2×anti-CD3 compositions (pJB0244 and pJB0245 respectively).

Caspase 3/7 assay utilizes a proluminescent caspase-3/7 DEVD-aminoluciferin substrate (“DEVD” disclosed SEQ ID NO: 1156) and a thermostable luciferase in a reagent optimized for caspase-3/7 activity, luciferase activity and cell lysis. Adding the reagent results in cell lysis, followed by caspase cleavage of the substrate. This liberates free aminoluciferin, which is consumed by the luciferase, generating a “glow-type” luminescent signal that is proportional to caspase-3/7 activity.

The cytotoxic performance of the unmasked, masked, or uncleavable anti-HER2×anti-CD3 bispecific antigen binding polypeptide compositions in all the human carcinoma cell lines was analyzed as follows: cell density of human carcinoma cells (target) and human PBMC cells(effector) was first adjusted to 5×10⁵ cells/mL and 2×10⁶ cells/mL respectively in assay medium comprised of RPMI and 10% FBS. To achieve an effector to target ratio of 5:1, 50 μL aliquots of human PBMC cells were co-cultured with 40 μL aliquots of human carcinoma cells per assay well in a 96-well round-bottom plate. Unmasked, masked, and uncleavable anti-HER2×anti-CD3 composition samples were diluted in assay medium to the desired dose concentration and added in 10 μL to the respective experimental wells, bringing the total assay volume to 100 μL. The unmasked aHER2×antiCD3 composition (e.g. pJB0244A) was evaluated as a 12-point, 5× serial diluted dose concentration starting at 1 nM to obtain a final dose range of 0.0000001 to 1 nM. The masked (e.g. pJB0244) and uncleavable (pJB0245) bispecific antigen binding polypeptide compositions were analyzed as a 12 point, 5× serial diluted dose concentration starting at 200 nM to derive at a final dose range of 0.00002 to 200 nM. Assay control for background, with no anti-HER2×anti-CD3 composition, having only target and effector cells, was also included in the assay. The plate containing experimental wells of unmasked, masked and uncleavable anti-HER2×anti-CD3 bispecific antigen binding polypeptide compositions and the respective assay controls, all tested in duplicates, was then allowed to incubate overnight in a 37° C., 5% CO₂ humidified incubator.

The amount of caspase 3/7 released into the supernatant as a result of cell lysis was measured using the Promega Caspase-Glo 3/7 Assay kit, following manufacturer's instructions. Before starting the assay, Caspase-Glo 3/7 Reagent was allowed to thaw and equilibrate to room temperature. The 96-well plate containing treated cells was removed from the incubator and allowed to equilibrate to room temperature, then 100 μl of Caspase-Glo 3/7 Reagent was added to each well in the plate. The plate was then covered, protected from light and allowed to incubate at room temperature for 30 min. After the incubation period, the contents of the wells were gently mixed using a plate shaker at 300-500 rpm for 30 seconds. Luminescence of each sample was measured in a plate-reading luminometer as directed by the luminometer manufacturer.

Data analysis was then performed as follows: dose concentrations of unmasked, masked, and uncleavable anti-HER2×anti-CD3 bispecific antigen binding polypeptide compositions were then plotted against cytotoxicity (measured in Relative Luminescence Units), and the concentration of protein that gave a half maximal response (EC50) was derived with a 4-parameter logistic regression equation using GraphPad prism software.

As shown in Table 15, when evaluated in HER2 high BT-474, SK-Br-3 and SK-OV-3 cell lines, the EC₅₀ activity of the unmasked anti-HER2×anti-CD3 bispecific antigen binding composition (pJB0244A) was in the range of 1 to 4 pM. The activity of the unmasked composition was at least 14,000-fold more active than the masked pJB0244 composition, which had an EC₅₀ activity in the range of 14,140 to 66,020 pM.

When evaluated using the HER2 mid-expression JIMT-1 cell line, the EC₅₀ activity of the unmasked pJB0244A was 52 pM, compared to an EC₅₀ activity of >200,000 pM for the masked pJB0244 and uncleavable pJB0245.

When evaluated in HER2 low-expressing cell lines such as MDA-MB-231 and MCF-7, the EC₅₀ activity of the unmasked pJB0244A was 124 pM and 139 pM respectively. Masked pJB0244 and the uncleavable pJB0245 were observed to have an EC₅₀ activity of >200,000 pM.

Conclusions: The results demonstrated that activity of unmasked anti-HER2×anti-CD3 bispecific antigen binding composition is HER2-receptor density dependent with a robust magnitude of killing in HER2-high- and HER2-mid expressing cell lines and a lower degree of killing in HER2 low-expressing cell lines. In line with the activity trend of the unmasked bispecific, the masked bispecific anti-HER2×anti-CD3 ProTIAs bearing two XTEN (pJB0244) offered strong blocking of cytotoxicity activity, with a reduced EC₅₀ activity of at least greater than 14,000-fold.

TABLE 15
Cytotoxicity determination in human carcinoma cell lines

| | | EC50 (pM) | | | | | |
|---|---|---|---|---|---|---|---|
| | Release | | | | | MDA- | |
| Construct | Segment | BT-474 | SK-Br3 | SKOV-3 | JIMT-1 | MB231 | MCF-7 |
| pJB0244A | none | 1.5 | 4 | 1 | 52 | 124 | 139 |
| pJB0244 | RSR-2295 | 66020 | 19530 | 14140 | ND | ND | ND |
| pJB0245 | RSR-3058 | ND | ND | ND | ND | ND | ND |

ND: below limits of detection

### Example 8: Cytotoxicity of Anti-HER2×Anti-CD3 Bispecific Antigen Binding Polypeptide Composition in Normal Human Breast, Skin and Lung Cell Lines

The unmasked anti-HER2×anti-CD3 bispecific antigen binding polypeptide (pJB0244A) was also evaluated in normal human primary cardiomyocytes and normal breast, normal skin and normal lung cell lines. In this experiment, effector PBMC were mixed independently with target normal human breast, skin or lung cells in a ratio of 5:1 in the same manner as described above. The HER2 high BT-474 cell line was used as a positive assay control. The unmasked anti-HER2×anti-CD3 bispecific antigen binding polypeptide was tested as a 8-point, 5× serial dilution dose curve concentration starting at 1 nM to obtain a final dose range of 0.000064 to 1 nM. The Caspase-Glo 3/7 assay was performed as described above. As expected, unmasked pJB0244A gave a robust cytotoxic activity in HER2 high BT-474 cell line with an EC5c) of 1.5 pM. Human cardiomyocytes, known to express some level of HER2, gave an EC₅₀ of 26 pM. In contrast, unmasked anti-HER2×anti-CD3 bispecific antigen binding polypeptide elicited no detectable cytotoxicity in normal breast, skin and lung cell lines using the concentrations tested (Table 16).

TABLE 16
Cytotoxicity determination in cell lines

| | EC50 (pM) | | | | |
|---|---|---|---|---|---|
| | | Normal | Normal human | Normal human | Normal human |
| Construct | BT-474 | cardiomyocytes | breast | skin | lung |
| pJB0244A | 1.5 | 26 | No cytotoxic | No cytotoxic | No cytotoxic |
| | | | activity observed | activity observed | activity observed |

### Example 9: In Vitro Caspase 3/7 Assay of Anti-EGFR×Anti-CD3 Bispecific Antigen Binding Polypeptide Compositions

Redirected cellular cytotoxicity of unmasked (with XTEN removed by proteolysis), masked (having 2 XTEN and 2 release segments cleavable by proteolysis), and uncleavable (with 2 XTEN and the release segments replaced by a peptide not susceptible to proteolysis) anti-EGFR×anti-CD3 bispecific antigen binding polypeptide compositions was assessed in an in vitro cell-based assay of caspase 3/7 activities of apoptotic cells. Similar to the caspase cytotoxicity assay described in the Examples, above, PBMC were mixed with EGFR positive tumor target cells in a ratio of 10 effector cells to 1 target cell. All anti-EGFR×anti-CD3 bispecific antigen binding polypeptide compositions were tested using a 10-point, 5× serial dilution of dose concentrations. The unmasked anti-EGFR×anti-CD3 composition was evaluated at a final dose range of 0.000012 to 10 nM. The masked and uncleavable bispecific antigen binding polypeptide compositions were analyzed at a final dose range of 0.00064 to 250 nM. Appropriate EGFR positive human tumor target cell lines included FaDu (squamous cell carcinoma of the head and neck, SCCHN), SCC-9 (SCCHN), HCT-116 (colorectal bearing KRAS mutation), NCI-H1573 (colorectal bearing KRAS mutation), HT-29 (colorectal bearing BRAF mutation) and NCI-H1975 (EGFR T790M mutation). The cell lines were selected to represent colorectal and SCCHN tumors with wild type EGFR and T790M, KRAS and BRAF mutations.

Upon cell lysis, released caspase 3/7 in culture supernatants was measured by the amount of luminogenic caspase 3/7 substrate cleavage by caspase 3/7 to generate the “glow-type” luminescent signal (Promega Caspase-Glo 3/7 cat#G8091). The amount of luminescence is proportional to the amount of caspase activities.

Results: As shown in Table 17, when evaluated in EGFR KRAS mutant HCT-116 cell line, the EC₅₀ activity of the masked anti-EGFR×anti-CD3 bispecific antigen binding polypeptide was 3,408 pM. The EC₅₀ of the uncleavable variant activity was >100,000 pM and the unmasked EC50 activity of the unmasked compositions was 0.8 pM.

When evaluated in EGFR BRAF mutant HT-29 cell line, the EC₅₀ activity of the masked anti-EGFR×anti-CD3 bispecific antigen binding polypeptide was 10,930 pM. The EC₅₀ activity of the uncleavable and unmasked compositions was >100,000 pM and 0.8 pM respectively.

The masked anti-EGFR×anti-CD3 bispecific antigen binding polypeptide was 4,000 to 14,000-fold less active than the unmasked anti-EGFR×anti-CD3 bispecific antigen binding polypeptide in the two EGFR mutant cell lines tested. As expected, the activity of the uncleavable variant was the least active of the 3 versions evaluated, with an EC50 of greater than 100,000 pM.

Conclusions: The results demonstrated that anti-EGFR×anti-CD3 bispecific antigen binding polypeptide are cytotoxically active against EGFR KRAS- and BRAF-mutant cell lines. Masked anti-EGFR×anti-CD3 bispecific antigen binding polypeptide bearing two XTEN offered strong blocking of cytotoxicity activity, with 4000- to 14,000-fold less cytotoxicity compared to the unmasked form.

TABLE 17
In vitro cytotoxicity activity of unmasked, masked/cleavable
and uncleavable anti-EGFR × anti-CD3 variants
in HT-29 and HCT-116 human cell lines

EC50 (pM)

| ProTIA | HCT-116 | HT-29 |
|---|---|---|
| Unmasked pJB0169 | 0.8 | 0.8 |
| Masked pJB0169 | 3408 | 10930 |
| Uncleavable pJB0169 | >100000 | >100000 |

### Example 10: Enzyme Activation, Storage and Digestion of RSR-1517-Containing XTEN AC1611

This example demonstrates that RSR-1517-containing XTEN construct AC1611, can be cleaved by various tumor-associated proteases including recombinant human uPA, matriptase, legumain, MMP-2, MMP-7, MMP-9, and MMP-14, in test tubes. The amino acid sequence of AC1611 is presented in Table 18, below.

1. Enzyme Activation

All enzymes used were obtained from R&D Systems. Recombinant human u-plasminogen activator (uPA) and recombinant human matriptase were provided as activated enzymes and stored at −80° C. until use. Recombinant mouse MMP-2, recombinant human MMP-7, and recombinant mouse MMP-9 were supplied as zymogens and required activation by 4-aminophenylmercuric acetate (APMA). APMA was first dissolved in 0.1M NaOH to a final concentration of 10 mM before the pH was readjusted to neutral using 0.1M HCl. Further dilution of the APMA stock to 2.5 mM was done in 50 mM Tris pH 7.5, 150 mM NaCl, 10 mM CaCl2. To activate pro-MMP, 1 mM APMA and 100 pg/mL of pro-MMP in 50 mM Tris pH 7.5, 150 mM NaCl, 10 mM CaCl2 were incubated at 37° C. for 1 hour (MMP-2, MMP-7) or 24 hours (MMP-9). To activate MMP-14, 0.86 μg/mL recombinant human furin and 40 μg/mL pro-MMP-14 in 50 mM Tris pH 9, 1 mM CaCl2 were incubated at 37° C. for 1.5 hours. To activate legumain, 100 μg/mL pro-legumain in 50 mM sodium acetate pH 4, 100 mM NaCl were incubated at 37° C. for 2 hours. 100% Ultrapure glycerol were added to all activated enzymes (including uPA and MTSP1) to a final concentration of 50% glycerol, then be stored at −20° C. for several weeks.

2. Enzymatic Digestion

A panel of enzymes was tested to determine cleavage efficiency of each enzyme for AC1611. 6 μM of the substrate was incubated with each enzyme in the following enzyme-to-substrate molar ratios and conditions: uPA (1:25 in 50 mM Tris pH 8.5), matriptase (1:25 in 50 mM Tris pH 9, 50 mM NaCl), legumain (1:20 in 50 mM MES pH 5, 250 mM NaCl), MMP-2 (1:1200 in 50 mM Tris pH 7.5, 150 mM NaCl, 10 mM CaCl2), MMP-7 (1:1200 in 50 mM Tris pH 7.5, 150 mM NaCl, 10 mM CaCl2), MMP-9 (1:2000 in 50 mM Tris pH 7.5, 150 mM NaCl, 10 mM CaCl2), and MMP-14 (1:30 in 50 mM Tris pH 8.5, 3 mM CaCl2, 1 μM ZnCl2) in 20 μL reactions. Reactions were incubated at 37° C. for two hours before stopped by adding EDTA to 20 mM in the case of MMP reactions, heating at 85° C. for 15 minutes in the case of uPA and matriptase reactions and adjusting pH to 8.5 in the case of legumain.

3. Analysis of Cleavage Efficiency.

Analysis of the samples to determine percentage of cleaved product was performed by loading 2 μL of undigested substrate (at 12 μM) and 4 μL digested (at 6 μM) reaction mixture on SDS-PAGE and staining with Stains-All (Sigma Aldrich). ImageJ software was used to analyze corresponding band intensity and determine percent of cleavage. Upon cleavage by various proteases at the release segment, the substrate RSR-1517-containing XTEN would yield two fragments, and the larger fragment was utilized in % cleavage calculations (quantity of reaction product divided by total initial substrate went into the reaction) while band intensity of the smaller product is too low to quantify. The percentage of cleavage of AC1611 under the current standard experimental conditions is 31%, 14%, 16%, 40%, 51%, 38%, 30%, for uPA, matriptase, legumain, MMP-2, MMP-7, MMP-9, MMP-14, respectively.

Conclusions: We selected a particular release segment RSR-1517 (amino acid sequence EAGRSANHEPLGLVAT (SEQ ID NO: 42)) and determined its cleavage profile as defined by percentage of cleavage under current standard experimental condition for all seven enzymes. This release segment has intermediate cleavage efficiency for all enzymes so that during screening, cleavage of faster or slower variants will fall within the assay window to allow accurate ranking.

TABLE 18
Amino acid sequence of AC1611
with Release Segment RSR-1517

| Construct | | SEQ ID |
|---|---|---|
| Name | Amino Acid Sequence* | NO: |
| AC1611 | MKNPEQAEEQAEEQREETGKPIPNP | 1139 |
| | LLGLDSTEGTSTEPSEGSAPGTSES | |
| | ATPESGPGTSESATPESGPGTSESA | |
| | TPESGPGSEPATSGSETPGSEPATS | |
| | GSETPGSPAGSPTSTEEGTSTEPSE | |
| | GSAPGTSTEPSEGSAPGSEPATSGS | |
| | ETPGTSESATPESGPGTAEAASASG | |
| | EAGRSANHEPLGLVATPGSPAGSPT | |
| | STEEGTSESATPESGPGTSTEPSEG | |
| | SAPGSPAGSPTSTEEGTSTEPSEGS | |
| | APGTSTEPSEGSAPGTSESATPESG | |
| | PGSEPATSGSETPGSEPATSGSETP | |
| | GSPAGSPTSTEEGTSESATPESGPG | |
| | TSTEPSEGSAPGTSTEPSEGSAPGS | |
| | PAGSPTSTEEGTSTEPSEGSAPGTS | |
| | TEPSEGSAPGTSESATPESGPGTST | |
| | EPSEGSAPGTSESATPESGPGSEPA | |
| | TSGSETPGTSTEPSEGSAPGTSTEP | |
| | SEGSAPGTSESATPESGPGTSESAT | |
| | PESGPGSPAGSPTSTEEGTSESATP | |
| | ESGPGSEPATSGSETPGTSESATPE | |
| | SGPGTSTEPSEGSAPGTSTEPSEGS | |
| | APGTSTEPSEGSAPGTSTEPSEGSA | |
| | PGTSTEPSEGSAPGTSTEPSEGSAP | |
| | GSPAGSPTSTEEGTSTEPSEGSAPG | |
| | TSESATPESGPGSEPATSGSETPGT | |
| | SESATPESGPGSEPATSGSETPGTS | |
| | ESATPESGPGTSTEPSEGSAPGTSE | |
| | SATPESGPGSPAGSPTSTEEGSPAG | |
| | SPTSTEEGSPAGSPTSTEEGTSESA | |
| | TPESGPGTSTEPSEGSAPGTSESAT | |
| | PESGPGSEPATSGSETPGTSESATP | |
| | ESGPGSEPATSGSETPGTSESATPE | |
| | SGPGTSTEPSEGSAPGSPAGSPTST | |
| | EEGTSESATPESGPGSEPATSGSET | |
| | PGTSESATPESGPGSPAGSPTSTEE | |
| | GSPAHHHHHHHH | |

### Example 11: Screening Release Segment Using RSR-1517 (AC1611) as Control

Here we select uPA as the example to show how the release segment screening was performed. The same procedure was applied to all seven tumor-associated proteases to define the relative cleavage profile for each substrate, which is a seven-number array to describe how well it can be cleaved for each enzyme, when compared to the control substrate RSR-1517. All polypeptides of Table 19 had the amino acid sequence of AC1611 but with the substitution of the release segment peptide of the indicated construct swapped in for the EAGRSANHEPLGLVAT sequence of AC1611 (SEQ ID NO: 42); e.g., BSRS-4 has a release segment sequence of LAGRSDNHSPLGLAGS (SEQ ID NO: 1157) but otherwise has complete sequence identity to AC1611.

1. Enzymatic Digestion

All release segment-containing XTEN variants and the control AC1611 were diluted to 12 μM in 50 mM Tris pH 7.5, 150 mM NaCl, 10 mM CaCl2 in individual Eppendorf tubes. A master mix of uPA was prepared so that after 1:1 mixing with each substrate, the total reaction volume is 20 μL, the initial substrate concentration is 6 μM, and the enzyme-to-substrate ratio was varied between 1:20 to 1:3000, depending on the enzyme, in order to have reaction products and uncleaved substrate that could be visualized at the endpoint. All reactions were incubated at 37° C. for 2 hours before stopped by adding EDTA to a final concentration of 20 mM. All products were analyzed by non-reducing SDS-PAGE followed by Stains-All. For each gel, AC1611 digestion product was always included as the staining control to normalize differential staining between different gels.

2. Relative Cleavage Efficiency Calculation

Percentage of cleavage for individual substrate was analyzed by ImageJ software and calculated as described before. For each variant, the relative cleavage efficiency is calculated as follows:

$${Log}_{2} ( \frac{\% Cleaved for substrate of interest}{\% cleaved for AC 1611 in the same experiment} )$$

A +1 value in relative cleavage efficiency indicates the substrate yielded twice as much product when compared to the AC1611 control while a −1 value in relative cleavage efficiency indicates the substrate yielded only 50% as much product when compared to the AC1611 control, under the experimental condition specified above.

In this experiment, the percentage of cleavage (% cleaved) for AC1611 is 20%, as quantified by ImageJ. The substrates being screened in this experiment demonstrated 21%, 39%, 1%, 58%, 24%, 6%, 15%, 1%, 1%, and 25% cleavage, where 1% essentially represents below detection limit and does not indicate accurate values. The relative cleavage efficiencies calculated based on the formula above were 0.08, 0.95, −4.34, 1.51, 0.26, −1.76, −0.47, −4.34, −4.34, and 0.32, respectively.

Conclusions: We determined relative cleavage efficiencies of 10 release segment variants when subject to uPA when compared to AC1611 in the same experiment. Following similar procedures, we determined the cleavage profiles of 134 release segments, the results of which are listed in Table 19, using RSR1517 (AC1611) as the reference control. These release segments covers a wide range of cleavage efficiency for individual enzyme as well as combinations. For example, RSR-1478 has a −2.00 value for MMP-14, meaning that this substrate yielded only 25% of product compared to the reference control RSR-1517 when digested with MMP-14. Certain release segments, such as RSR-1951, appear to be better substrate for all seven proteases tested. These faster release segments may prove to be useful in the clinic if the systemic toxicity is low/manageable while efficacy (partially depending on how fast cleavage happens to render bispecific antigen binding polypeptide as the activated form) needs improvement.

TABLE 19
Cleavage profiles of Release Segment when subjected to seven
human proteases using RSR-1517 as control

| | | | SEQ | | | | | | | |
|---|---|---|---|---|---|---|---|---|---|---|
| | | | ID | | | | | | | |
| RS ID | AC# | AA Sequence | NO: | uPA | Matriptase | Legumain | MMP-2 | MMP-7 | MMP-9 | MMP-14 |
| RSR-1517 | AC1611 | EAGRSANHEPLGLVAT | 42 | 0 | 0 | 0 | 0 | 0 | 0 | 0 |
| BSRS-4 | AC1602 | LAGRSDNHSPLGLAGS | 1157 | −0.99 | 1.69 | 0.48 | 0.09 | −0.49 | −0.04 | −0.58 |
| BSRS-5 | AC1603 | LAGRSDNHVPLSLSMG | 1158 | −1.4 | 1.76 | 0.56 | −0.52 | 0 | −0.75 | −0.21 |
| BSRS-6 | AC1604 | LAGRSDNHEPLELVAG | 1159 | −2.71 | 0.47 | 0.23 | −1.26 | 0 | −1.16 | −2.79 |
| BSRS-A1 | AC1605 | ASGRSTNAGPSGLAGP | 43 | 1.43 | 2.77 | 0.09 | −0.16 | −2.18 | 0.03 | −1.24 |
| BSRS-A2 | AC1606 | ASGRSTNAGPQGLAGQ | 44 | 1.36 | 2.77 | −0.14 | 0.09 | −2.64 | 0.03 | −1.03 |
| BSRS-A3 | AC1607 | ASGRSTNAGPPGLTGP | 45 | 1.49 | 2.77 | 0.05 | −1.07 | −3.47 | −1.82 | −3.59 |
| VP-1 | AC1608 | ASSRGTNAGPAGLTGP | 46 | −2.19 | 1.16 | 0.9 | 0.09 | −1.22 | 0.23 | 0 |
| RSR-1752 | AC1609 | ASSRTTNTGPSTLTGP | 47 | −0.55 | 0.7 | 0.29 | −0.34 | −1.29 | −0.94 | −5.38 |
| RSR-1512 | AC1610 | AAGRSDNGTPLELVAP | 48 | −2.96 | 1.51 | 0.56 | −1.43 | −0.45 | −1.09 | −3.91 |
| RSR-1517 | AC1611 | EAGRSANHEPLGLVAT | 42 | 0 | 0 | 0 | 0 | 0 | 0 | 0 |
| VP-2 | AC1612 | ASGRGTNAGPAGLTGP | 49 | −0.7 | 1.38 | 1.12 | 0 | −0.58 | 0.23 | −0.15 |
| RSR-1018 | AC1613 | LFGRNDNHEPLELGGG | 50 | −4.62 | −0.53 | 1.36 | −0.73 | −0.43 | −2.56 | −1.79 |
| RSR-1053 | AC1614 | TAGRSDNLEPLGLVFG | 51 | −3.21 | −0.12 | −0.13 | 0.09 | −0.03 | 0.25 | −0.19 |
| RSR-1059 | AC1615 | LDGRSDNFHPPELVAG | 52 | −4.62 | −0.89 | 0.56 | −3.1 | −2.62 | −5.14 | −6.49 |
| RSR-1065 | AC1616 | LEGRSDNEEPENLVAG | 53 | −4.62 | −2.7 | 0.43 | −1.84 | −1 | −3.14 | −1.85 |
| RSR-1167 | AC1617 | LKGRSDNNAPLALVAG | 54 | −4.62 | 3.35 | 1.32 | 0.09 | 0.22 | 1.18 | 0.06 |
| RSR-1201 | AC1618 | VYSRGTNAGPHGLTGR | 55 | −3.02 | 2.35 | 1.25 | 0.09 | −1.3 | 0.79 | −0.3 |
| RSR-1218 | AC1619 | ANSRGTNKGFAGLIGP | 56 | −0.52 | 2.66 | 1.74 | −0.3 | 0 | −1.33 | −1.6 |
| RSR-1226 | AC1620 | ASSRLTNEAPAGLTIP | 57 | −0.98 | 0.29 | 0.58 | 0.07 | −0.43 | −2.33 | −0.42 |
| RSR-1254 | AC1621 | DQSRGTNAGPEGLTDP | 58 | −1.27 | −1.17 | 1 | −3.1 | −2.32 | −4.14 | −2.92 |
| RSR-1256 | AC1622 | ESSRGTNIGQGGLTGP | 59 | −1.65 | −0.58 | 0.27 | −2.26 | −3.32 | −5.14 | −5.51 |
| RSR-1261 | AC1623 | SSSRGTNQDPAGLTIP | 60 | −1.77 | −0.36 | 0.62 | −1.14 | −1.25 | −3.56 | −0.98 |
| RSR-1293 | AC1624 | ASSRGQNHSPMGLTGP | 61 | −4.69 | 2.15 | 0.91 | −0.7 | −0.01 | 1.3 | −0.67 |
| RSR-1309 | AC1625 | AYSRGPNAGPAGLEGR | 62 | −4.69 | 0.53 | 0.74 | −0.7 | −2.25 | 0.86 | 0.02 |
| RSR-1326 | AC1626 | ASERGNNAGPANLTGF | 63 | −0.27 | 1.27 | 1.64 | −0.85 | −0.74 | 0.28 | −0.13 |
| RSR-1345 | AC1627 | ASHRGTNPKPAILTGP | 64 | 0.42 | ND | ND | ND | ND | −0.5 | ND |
| RSR-1354 | AC1628 | MSSRRTNANPAQLTGP | 65 | 1.07 | 2.82 | 0.36 | −0.77 | −0.64 | −1.82 | −1.87 |
| RSR-1426 | AC1629 | GAGRTDNHEPLELGAA | 66 | −2.36 | −0.65 | −0.19 | −2.82 | −0.18 | −0.11 | −4.07 |
| RSR-1478 | AC1630 | LAGRSENTAPLELTAG | 67 | −2.06 | 1.18 | 0.54 | −0.82 | 0 | 1.73 | −2 |
| RSR-1479 | AC1631 | LEGRPDNHEPLALVAS | 68 | −3.47 | −3.46 | 0.12 | −0.74 | 0 | 2.05 | 0.02 |
| RSR-1496 | AC1632 | LSGRSDNEEPLALPAG | 69 | −3.48 | −1.46 | 0.22 | −2.81 | −5.06 | −3.2 | −3.22 |
| RSR-1508 | AC1633 | EAGRTDNHEPLELSAP | 70 | −2.74 | −1.46 | −0.26 | −1.48 | 0 | 0.56 | −2.93 |
| RSR-1513 | AC1634 | EGGRSDNHGPLELVSG | 71 | −2.81 | −1.87 | 0.27 | −0.9 | 0 | 1.29 | −3.81 |
| RSR-1516 | AC1635 | LSGRSDNEAPLELEAG | 72 | −3.71 | −1.87 | 0.7 | −1.69 | −0.09 | −1.39 | −3.22 |
| RSR-1524 | AC1636 | LGGRADNHEPPELGAG | 73 | −0.84 | 1.12 | 0.95 | −1.22 | −2.84 | −0.74 | −2.57 |
| RSR-1622 | AC1637 | PPSRGTNAEPAGLTGE | 74 | −4.66 | −3.46 | 0.62 | −0.7 | −1.09 | 0.93 | −0.78 |
| RSR-1629 | AC1638 | ASTRGENAGPAGLEAP | 75 | −4.66 | −1.14 | 1.09 | −0.7 | −1.74 | 0.19 | −0.25 |
| RSR-1664 | AC1639 | ESSRGTNGAPEGLTGP | 76 | −4.66 | −3.46 | 0.32 | −1.18 | −0.76 | −0.76 | −2.31 |
| RSR-1667 | AC1640 | ASSRATNESPAGLTGE | 77 | −3.05 | 2 | 0.46 | −0.93 | −1.25 | −0.97 | −0.83 |
| RSR-1709 | AC1641 | ASSRGENPPPGGLTGP | 78 | −2.64 | 0.77 | −1 | −0.93 | −2.06 | −0.76 | −1.72 |
| RSR-1712 | AC1642 | AASRGTNTGPAELTGS | 79 | −4.07 | −0.51 | 0.66 | −0.93 | −0.64 | 0.29 | −0.19 |
| RSR-1727 | AC1643 | AGSRTTNAGPGGLEGP | 80 | −3.55 | −0.51 | 0.32 | −1.58 | −4.84 | −3.08 | −1.78 |
| RSR-1754 | AC1644 | APSRGENAGPATLTGA | 81 | −4.68 | −3.32 | 1.06 | 0.19 | −1.4 | −1.5 | −0.17 |
| RSR-1819 | AC1645 | ESGRAANTGPPTLTAP | 82 | 1.2 | 0.79 | −0.7 | −3.41 | −5.64 | −4.67 | −6.92 |
| RSR-1832 | AC1646 | NPGRAANEGPPGLPGS | 83 | −3.62 | 0.58 | 0.81 | −4.39 | −6.64 | −4.67 | −6.48 |
| RSR-1855 | AC1647 | ESSRAANLTPPELTGP | 84 | −0.08 | −1.62 | 0.77 | −3.07 | −3.47 | −4.67 | −2.92 |
| RSR-1911 | AC1648 | ASGRAANETPPGLTGA | 85 | 0.99 | 2.2 | 0.56 | −1.29 | −3.84 | −1.39 | −3.11 |
| RSR-1929 | AC1649 | NSGRGENLGAPGLTGT | 86 | −1.68 | ND | ND | ND | ND | −3.08 | ND |
| RSR-1951 | AC1650 | TTGRAANLTPAGLTGP | 87 | 1.94 | 2.57 | 0.39 | 0.09 | −0.09 | 0.13 | −0.42 |
| RSR-2295 | AC1761 | EAGRSANHTPAGLTGP | 88 | 0.4 | 1.48 | 0.01 | 1.2 | 0.35 | 0.13 | 0.97 |
| RSR-2298 | AC1762 | ESGRAANTTPAGLTGP | 89 | 1.01 | 0.86 | 0.55 | 1.24 | 0.24 | 0.07 | 1.03 |
| RSR-2038 | AC1679 | TTGRATEAANLTPAGLTGP | 90 | 4.75 | 1 | 0.81 | 0.86 | 0.1 | 0.15 | 0.27 |
| RSR-2072 | AC1680 | TTGRAEEAANLTPAGLTGP | 91 | 0 | −0.49 | 1 | 0.86 | 0.11 | −0.12 | 0.27 |
| RSR-2089 | AC1681 | TTGRAGEAANLTPAGLTGP | 92 | 3.91 | 2.05 | 0.32 | 0.85 | 0.02 | −0.04 | 0.27 |
| RSR-2302 | AC1682 | TTGRATEAANATPAGLTGP | 93 | 4.73 | 0.65 | 0 | 0.74 | −0.48 | −0.35 | 0.1 |
| RSR-3047 | AC1697 | TTGRAGEAEGATSAGATGP | 94 | | | | | | | |
| RSR-3052 | AC1698 | TTGEAGEAANATSAGATGP | 95 | | | | | | | |
| RSR-3043 | AC1699 | TTGEAGEAAGLTPAGLTGP | 96 | | | | | | | |
| RSR-3041 | AC1700 | TTGAAGEAANATPAGLTGP | 97 | | | | | | | |
| RSR-3044 | AC1701 | TTGRAGEAAGLTPAGLTGP | 98 | | | | | | | |
| RSR-3057 | AC1702 | TTGRAGEAANATSAGATGP | 99 | | | | | | | |
| RSR-3058 | AC1703 | TTGEAGEAAGATSAGATGP | 100 | | | | | | | |
| RSR-2485 | AC1763 | ESGRAANTEPPELGAG | 101 | 0.61 | −0.9 | 0.15 | −5.82 | −6.27 | −5.36 | −5.64 |
| RSR-2486 | AC1764 | ESGRAANTAPEGLTGP | 102 | 1.03 | 0.24 | 0.95 | 0.3 | 0.37 | −0.33 | −0.74 |
| RSR-2488 | AC1688 | EPGRAANHEPSGLTEG | 103 | −3.27 | −1.21 | −1.3 | −0.73 | −0.91 | −1.86 | −0.88 |
| RSR-2599 | AC1706 | ESGRAANHTGAPPGGLTGP | 104 | 1.7 | 1.02 | 0.36 | 0.68 | −1.49 | −0.71 | −2.04 |
| RSR-2706 | AC1716 | TTGRTGEGANATPGGLTGP | 105 | 0.07 | 0.83 | 1.17 | −0.04 | −2.25 | −2.25 | 0 |
| RSR-2707 | AC1717 | RTGRSGEAANETPEGLEGP | 106 | 1.95 | 3.25 | 0.96 | −1.96 | −2.75 | −5 | −1.39 |
| RSR-2708 | AC1718 | RTGRTGESANETPAGLGGP | 107 | 1.24 | 3.25 | 0.88 | −0.37 | −3.55 | −4 | −0.49 |
| RSR-2709 | AC1719 | STGRTGEPANETPAGLSGP | 108 | −0.14 | 0.38 | 0.4 | 0.35 | −1.03 | −1.68 | 1.86 |
| RSR-2710 | AC1720 | TTGRAGEPANATPTGLSGP | 109 | −0.21 | 2.04 | 0.56 | 0.15 | −3.23 | −1.83 | −0.07 |
| RSR-2711 | AC1721 | RTGRPGEGANATPTGLPGP | 110 | 0.58 | 3.22 | 1.45 | −6.04 | −5.55 | −5 | −4.39 |
| RSR-2712 | AC1722 | RTGRGGEAANATPSGLGGP | 111 | 0.86 | 3.15 | 1.21 | −0.34 | −3.97 | −2.68 | −1.58 |
| RSR-2713 | AC1723 | STGRSGESANATPGGLGGP | 112 | 0.96 | 2.22 | 0.78 | −5.04 | −5.25 | −5.25 | −3.32 |
| RSR-2714 | AC1724 | RTGRTGEEANATPAGLPGP | 113 | 0.83 | 3.23 | 0.96 | −4.46 | −5.55 | −5 | −4.39 |
| RSR-2715 | AC1725 | ATGRPGEPANTTPEGLEGP | 114 | −4.32 | −3.17 | 0.46 | −1.34 | −1.93 | −1.93 | −1.32 |
| RSR-2716 | AC1726 | STGRSGEPANATPGGLTGP | 115 | 1 | 2.41 | 0.51 | −0.46 | −3.55 | −2.68 | −1.22 |
| RSR-2717 | AC1727 | PTGRGGEGANTTPTGLPGP | 116 | −0.21 | 1.54 | 1.28 | −6.04 | −5.55 | −5 | −4.39 |
| RSR-2718 | AC1728 | PTGRSGEGANATPSGLTGP | 117 | 1.54 | 3.4 | 1.29 | 1.3 | −0.2 | −0.2 | 1.63 |
| RSR-2719 | AC1729 | TTGRASEGANSTPAPLTEP | 118 | 0.26 | 1.15 | 1.3 | −1.46 | −0.16 | −0.16 | 1.68 |
| RSR-2720 | AC1730 | TYGRAAEAANTTPAGLTAP | 119 | −1.65 | 2.14 | 1.21 | 0.56 | 0.45 | 0.21 | 2.25 |
| RSR-2721 | AC1731 | TTGRATEGANATPAELTEP | 120 | 0.77 | −0.85 | 1.25 | −2.44 | 0 | −4.91 | −3.75 |
| RSR-2722 | AC1732 | TVGRASEEANTTPASLTGP | 121 | −1.74 | −1.17 | 0.39 | 1.08 | 1 | 1 | 2.14 |
| RSR-2723 | AC1733 | TTGRAPEAANATPAPLTGP | 122 | −0.42 | −3.17 | 1.32 | 0.76 | 0.66 | 0.66 | 2.17 |
| RSR-2724 | AC1734 | TWGRATEPANATPAPLTSP | 123 | −4.32 | 1 | 0.55 | 0.81 | 0.42 | 0.42 | 2.58 |
| RSR-2725 | AC1735 | TVGRASESANATPAELTSP | 124 | −4.32 | −0.17 | 0.86 | −0.02 | 0.45 | −1.74 | −2.17 |
| RSR-2726 | AC1736 | TVGRAPEGANSTPAGLTGP | 125 | −4.32 | −3.17 | 1.39 | 1.22 | 0.24 | 0.24 | 2.1 |
| RSR-2727 | AC1737 | TWGRATEAPNLEPATLTTP | 126 | −4.32 | 0 | −0.3 | −0.5 | 0.17 | −3.91 | −1.95 |
| RSR-2728 | AC1738 | TTGRATEAPNLTPAPLTEP | 127 | 0.32 | 0.83 | −0.61 | −0.8 | 0.45 | 0.45 | 2 |
| RSR-2729 | AC1739 | TQGRATEAPNLSPAALTSP | 128 | −4.52 | 1.73 | 0.37 | 1.75 | 0.93 | 0.93 | 2.85 |
| RSR-2730 | AC1740 | TCGRAAEAPNLTPATLTAP | 129 | −2.2 | 2.73 | 0.22 | 1.19 | 0.51 | 0.51 | 1.29 |
| RSR-2731 | AC1741 | TSGRAPEATNLAPAPLTGP | 130 | −1.72 | −2.7 | 1.22 | 1.57 | 0.92 | 0.92 | 2.32 |
| RSR-2732 | AC1742 | TQGRAAEAANLTPAGLTEP | 131 | −2.52 | 2.49 | 1.44 | 0.32 | −0.21 | −0.21 | 2.29 |
| RSR-2733 | AC1743 | TTGRAGSAPNLPPTGLTTP | 132 | 1.09 | 2.91 | 0.32 | 0.48 | −2.32 | −2.32 | −3.17 |
| RSR-2734 | AC1744 | TTGRAGGAENLPPEGLTAP | 133 | 0.83 | 2 | 0.66 | 0.55 | 0.55 | 0.55 | 1.83 |
| RSR-2735 | AC1745 | TTSRAGTATNLTPEGLTAP | 134 | 0.38 | 2.34 | 0.32 | 0.48 | 0.26 | 0.26 | 2.12 |
| RSR-2736 | AC1746 | TTGRAGTATNLPPSGLTTP | 135 | 1.03 | 2.91 | 0.17 | 1.34 | −1.1 | −1.1 | 1.42 |
| RSR-2737 | AC1747 | TTARAGEAENLSPSGLTAP | 136 | −0.2 | 0.3 | 0.37 | 1.57 | −0.03 | −0.03 | 2.35 |
| RSR-2738 | AC1748 | TTGRAGGAGNLAPGGLTEP | 137 | 1.68 | 3.37 | 1.03 | −1.32 | −1.65 | −2.1 | −1.05 |
| RSR-2739 | AC1749 | TTGRAGTATNLPPEGLTGP | 138 | 1 49 | 3.43 | 0.31 | −0.12 | 0.71 | −0.58 | −0.67 |
| RSR-2740 | AC1750 | TTGRAGGAANLAPTGLTEP | 139 | 1.77 | 3.38 | 1.49 | −1.02 | −0.75 | −1.32 | −0.43 |
| RSR-2741 | AC1751 | TTGRAGTAENLAPSGLTTP | 140 | 0.68 | 3.1 | 0.56 | 0.58 | −0.51 | −0.91 | 0.42 |
| RSR-2742 | AC1752 | TTGRAGSATNLGPGGLTGP | 141 | 1.43 | 3.42 | 0.51 | −0.27 | −3.23 | −2.32 | −0.17 |
| RSR-2743 | AC1753 | TTARAGGAENLTPAGLTEP | 142 | 1.63 | 2.19 | 0.78 | −0.5 | −0.13 | −2.58 | 1.18 |
| RSR-2744 | AC1754 | TTARAGSAENLSPSGLTGP | 143 | 1.04 | 2.32 | 0.65 | 0.59 | 0 | −0.15 | 0.49 |
| RSR-2745 | AC1755 | TTARAGGAGNLAPEGLTTP | 144 | 1.12 | 2.77 | 0.4 | −0.77 | −0.58 | −2.28 | −1 |
| RSR-2746 | AC1756 | TTSRAGAAENLTPTGLTGP | 145 | −0.81 | 1.54 | 0.18 | 0.42 | −0.85 | −1.5 | −0.26 |
| RSR-2747 | AC1757 | TYGRTTTPGNEPPASLEAE | 146 | −1.49 | 1.26 | 0.06 | −0.2 | −0.36 | −2.77 | −2.1 |
| RSR-2748 | AC1758 | TYSRGESGPNEPPPGLTGP | 147 | −4.81 | −2.32 | −0.76 | −0.28 | −2.68 | −2.28 | −2.91 |
| RSR-2749 | AC1759 | AWGRTGASENETPAPLGGE | 148 | −4.81 | 3.15 | 0.24 | −1.28 | −3.91 | −5.09 | −2.58 |
| RSR-2750 | AC1760 | RWGRAETTPNTPPEGLETE | 149 | −1.49 | 3.28 | −0.29 | −3.17 | −3.91 | −5.09 | −4.91 |
| RSR-2751 | AC1765 | ESGRAANHTGAEPPELGAG | 150 | 1.04 | 0.37 | 0.4 | −1.59 | −5.67 | −5.26 | −4.93 |
| RSR-2754 | AC1801 | TTGRAGEAANLTPAGLTES | 151 | | | | −0.15 | −0.82 | −3.61 | 0.45 |
| RSR-2755 | AC1802 | TTGRAGEAANLTPAALTES | 152 | | | | 0.06 | 0.29 | −2.91 | 0.62 |
| RSR-2756 | AC1803 | TTGRAGEAANLTPAPLTES | 153 | | | | −0.58 | −0.39 | −2.58 | 0.49 |
| RSR-2757 | AC1804 | TTGRAGEAANLTPEPLTES | 154 | | | | −1.59 | −0.27 | −1.89 | −0.52 |
| RSR-2758 | AC1805 | TTGRAGEAANLTPAGLTGA | 155 | | | | 0.7 | −0.43 | 0.17 | 0.85 |
| RSR-2759 | AC1806 | TTGRAGEAANLTPEGLTGA | 156 | | | | 0.04 | −0.72 | −1.06 | −0.18 |
| RSR-2760 | AC1807 | TTGRAGEAANLTPEPLTGA | 157 | | | | −0.06 | −0.12 | −1.9 | −0.15 |
| RSR-2761 | AC1808 | TTGRAGEAANLTPAGLTEA | 158 | | | | −0.06 | −0.55 | −3.71 | 0.69 |
| RSR-2762 | AC1809 | TTGRAGEAANLTPEGLTEA | 159 | | | | −2.14 | −0.69 | −4.3 | −0.59 |
| RSR-2763 | AC1810 | TTGRAGEAANLTPAPLTEA | 160 | | | | −0.76 | −0.31 | −5.28 | 0.64 |
| RSR-2764 | AC1811 | TTGRAGEAANLTPEPLTEA | 161 | | | | −2.18 | −0.06 | −5.28 | −0.11 |
| RSR-2765 | AC1812 | TTGRAGEAANLTPEPLTGP | 162 | | | | −0.31 | 0.07 | −5.28 | −5.63 |
| RSR-2766 | AC1813 | TTGRAGEAANLTPAGLTGG | 163 | | | | 0.77 | −0.61 | −5.28 | −5.63 |
| RSR-2767 | AC1814 | TTGRAGEAANLTPEGLTGG | 164 | | | | −0.2 | −0.85 | −1.26 | −0.25 |
| RSR-2768 | AC1815 | TTGRAGEAANLTPEALTGG | 165 | | | | −0.5 | 0.13 | −1.8 | −0.43 |
| RSR-2769 | AC1816 | TTGRAGEAANLTPEPLTGG | 166 | | | | −0.44 | −0.26 | −2.4 | −0.39 |
| RSR-2770 | AC1817 | TTGRAGEAANLTPAGLTEG | 167 | | | | −0.07 | −0.47 | −3.18 | 0.4 |
| RSR-2771 | AC1818 | TTGRAGEAANLTPEGLTEG | 168 | | | | −3.05 | −0.93 | −5.28 | −0.99 |
| RSR-2772 | AC1819 | TTGRAGEAANLTPAPLTEG | 169 | | | | −0.53 | −0.24 | −2.19 | 0.39 |
| RSR-2773 | AC1820 | TTGRAGEAANLTPEPLTEG | 170 | | | | −3.8 | −0.42 | −5.28 | −0.81 |
| BSRS-1 | AC1601 | LSGRSDNHSPLGLAGS | 1152 | 0.89 | 1.94 | 0.1 | −0.67 | −2.12 | −0.5 | −1.92 |

ND = not determined

### Example 12: Competitive Digestion Using RSR-1517 as Internal Control

This competitive assay is developed to minimize any variability in enzyme concentration or reaction condition between reactions in different vials within the same experiment. In order to resolve both the control substrate and the RS of interest in the same example, new control plasmids are constructed.

1. Molecular Cloning of RSR-1517-Containing Internal Control

Two internal control plasmids, AC1830 (HD2-V5-AE144-RSR-1517-XTEN288) and AC1840 (HD2-V5-AE144-RSR-1517-XTEN432), are constructed in a similar fashion as AC1611 described in Example 10, with the only difference in the length of the C-terminal XTEN.

2. Enzymatic Digestion

2× substrate solution is prepared by mixing and diluting purified AC1830 or AC1840 and the RS of interest in assay buffer so that the final concentrations of individual substrates are 6 μM. An enzyme master mix is prepared so that after 1:1 mixing with 2× substrate solution, the total reaction volume is 20 μL, the final substrate concentration of each component is 3 μM, and the enzyme-to-substrate ratio is as selected in assay development. The reaction is incubated at 37° C. for 2 hours before stopped by procedures as described above.

3. Relative Cleavage Efficiency Calculation

The reaction mixture is analyzed by non-reducing 4-12% SDS-PAGE. Since the internal control and the substrate of interest have different molecular weight, once cleaved, four bands should be visible in the same sample lane. Percentage of cleavage for both can be calculated and the relative cleavage efficiency can be derived from the same formula in Example 10:

$${Log}_{2} ( \frac{\% Cleaved for substrate of interest}{\% cleaved for AC 1611 in the same experiment} )$$

The only difference is now both values are calculated from the reaction mixture in the same vial, while previously from two reactions sharing the same enzyme mix.

Conclusions: We expect this competitive digestion assay with RSR-1517 as internal control to have less assay-to-assay variability when compared to the assay described in Example 10. We anticipate adopting this method for future release segment screening.

### Example 11A: Anti-Tumor Properties of Anti-EpCAM x Anti-CD3 Bispecific Antigen Binding Polypeptide Bearing One or Two XTEN in Established Breast Tumor Model

In the established breast tumor model, BT-474 tumor cells were independently implanted, in the presence of matrigel, subcutaneously into NOG (NOD/Shi-scid/IL-2Rγ^{null}) or NSG (NOD.Cg-Prkdc^{scid}.IL2rg^{tmlWjl}/SzJ) mice on day 0. (The NOG or NSG mice are NOD/SCID mice bearing IL-2Rγ mutation resulting in the mice lacking T, B and NK cells, dysfunctional macrophage, dysfunctional dendritic cells and reduced complement activity.) Human PBMCs were then intravenously introduced when BT-474 tumor volume reached 100-200 mm³. Treatment with vehicle, protease-untreated anti-EpCAM×anti-CD3 bispecific antigen binding polypeptide carrying one XTEN polymer (e.g. pJB0189) and an anti-EpCAM×anti-CD3 bispecific antigen binding polypeptide bearing two XTEN polymers (e.g. pJB0176) was initiated intravenously as three doses per week for four weeks. Cohort 1 was the vehicle-treated group, cohort 2 was the pJB0189-treated group at 0.5 mg/kg, and cohort 3 was the pJB0176-treated group at 0.5 mg/kg.

Tumors were measured twice per week for a projected 45 days with a caliper in two perpendicular dimensions and tumor volumes were calculated by applying the (width²×length)/2 formula. Body weight, general appearance and clinical observations such as seizures, tremors, lethargy, hyper-reactivity, pilo-erection, labored/rapid breathing, coloration and ulceration of tumor and death were also closely monitored as a measure of treatment related toxicity. Percent tumor growth inhibition index (% TGI) was calculated for each of the treatment group by applying the formula: ((Mean tumor volume of Group 2 vehicle control—Mean tumor volume of bispecific antigen binding polypeptide treatment)/mean tumor volume of Group 2 vehicle control)×100. Treatment group with % TGI≥60% is considered therapeutically active.

Results: At interim day 27, vehicle-treated cohort 1 mice did not inhibit tumor progression having a tumor burden of 219±30 mm³, demonstrating that human effector cells alone as such could not elicit an anti-tumor effect. As expected, treatment with pJB0189 anti-EpCAM×anti-CD3 bispecific antigen binding polypeptide at 0.5 mg/kg (cohort 2) in the presence of human effector cells exhibited clear anti-tumor regression with % TGI of 68%. Importantly, treatment with pJB0176 anti-EpCAM×anti-CD3 bispecific antigen binding polypeptide at 0.5 mg/kg (cohort 3) in the presence of human effector cells also elicited a robust anti-tumor response yielding a % TGI of 76%.

Conclusions: Interim data suggest that at 0.5 mg/kg in the in vivo BT-474 tumor environment, protease-untreated anti-EpCAM×anti-CD3 bispecific antigen binding polypeptide bearing two XTENs (i.e., pJB0176) is as efficacious as protease-untreated anti-EpCAM×anti-CD3 bispecific antigen binding polypeptide bearing one XTEN polymer (i.e., pJB0189). Of note, no significant body weight loss was observed in all bispecific antigen binding polypeptide treatment groups and vehicle control indicating that all treatments were well tolerated.

### Example 13: Cell Binding Assessed by Flow Cytometry

Bispecific binding of the anti-EGFR×anti-CD3 bispecific antigen binding polypeptide composition is also evaluated by flow cytometry-based assays utilizing CD3 positive human Jurkat cells and EGFR positive human cells selected from HT-29, HCT-116, NCI-H1573, NCI-H1975, FaDu, and SCC-9 or a stable CHO cell line expressing EGFR. CD3⁺ and EGFR⁺ cells are incubated with a dose range of untreated anti-EGFR×anti-CD3 bispecific antigen binding polypeptide (PJB0169, comprising 2 XTEN and 2 RS), protease-treated PJB0169, and anti-CD3 scFv and anti-EGFR scFv positive controls for 30 min at 4° C. in binding buffer containing HBSS with 2% BSA and 5 mM EDTA. After washing with binding buffer to remove unbound test material, cells are incubated with FITC-conjugated anti-His tag antibody (Abcam cat #ab1206) for 30 min at 4° C. Unbound FITC-conjugated antibody is removed by washing with binding buffer and cells resuspended in binding buffer for acquisition on a FACS Calibur flow cytometer (Becton Dickerson) or equivalent instrument. All flow cytometry data are analyzed with FlowJo software (FlowJo LLC) or equivalent.

While anti-EGFR scFv is not expected to bind to Jurkat cells, anti-CD3 scFv, untreated PJB0169 and protease-treated PJB0169 are all expected to bind to Jurkat cells as indicated by an increase in fluorescence intensity when compared to Jurkat cells incubated with FITC-conjugated anti-His tag antibody alone. Similarly, anti-EGFR scFv, protease-treated and untreated PJB0169 are all expected to bind to EGFR positive cells, while anti-CD3 scFv is not expected to bind to EGFR positive cells. It is expected that these data will reflect the bispecific binding ability of the anti-EGFR×anti-CD3 bispecific antigen binding polypeptide composition to recognize both the CD3 and EGFR antigen expressed respectively on Jurkat and the panel of EGFR expressing human cell lines. Furthermore, due to the XTEN polymer providing some interference in surface binding, the untreated anti-EGFR×anti-CD3 bispecific antigen binding polypeptide is expected to bind at a lower affinity than the protease-treated bispecific antigen binding polypeptide for both the CD3 and EGFR antigens.

### Example 14: Cell Lysis Assessed by Flow Cytometry

Cell lysis by the anti-EGFR×anti-CD3 bispecific antigen binding polypeptide composition is evaluated by flow cytometry utilizing human PBMCs and an EGFR positive cell line. EGFR positive HCT-116 target cells (or target cells selected from HT-29, NCI-H1573, NCI-H1975, FaDu, and SCC-9 or a stable CHO cell line expressing EGFR) are labeled with the fluorescent membrane dye CelIVue Maroon dye (Affymetrix/eBioscience, cat #88-0870-16) according to manufacturer's instructions. Alternatively PKH26 (Sigma, cat #MINI26 and PKH26GL) can also be used. In brief, HCT-116 cells are washed twice with PBS followed by resuspension of 2×10⁶ cells in 0.1 mL Diluent C provided with the CelIVue Maroon labeling kit. In a separate tube, 2 μL of CelIVue Maroon dye is mixed with 0.5 mL diluent C, and then 0.1 mL added to the HCT-116 cell suspension. The cell suspension and CelIVue Maroon dye are mixed and incubated for 2 min at room temperature. The labeling reaction is then quenched by the addition of 0.2 mL of fetal bovine serum (FCS). Labeled cells are washed twice with complete cell culture medium (RPMI-1640 containing 10% FCS) and the total number of viable cells determined by trypan blue exclusion. For an effector to target ratio of 10:1 in a total volume of 200 μL per well, 1×10⁵ PBMC are co-cultured with 1×10⁴ CelIVue Maroon-labeled HCT-116 cells per well in a 96-well round-bottom plate in the absence or presence of the indicated dose range concentration of protease-treated and untreated anti-EGFR×anti-CD3 bispecific antigen binding polypeptide (PJB0169, comprising 2 XTEN and 2 RS) samples. After 24 h, cells are harvested with Accutase (Innovative Cell Technologies, cat #AT104) and washed with 2% FCS/PBS. Before cell acquisition on a Guava easyCyte flow cytometer (Millipore), cells are resuspended in 100 μL 2% FCS/PBS supplemented with 2.5 micrograms/mL 7-AAD (Affymetrix/eBioscience, cat #00-6993-50) to discriminate between alive (7-AAD-negative) and dead (7-AAD-positive) cells. FACS data are analyzed with guavaSoft software (Millipore); and percentage of dead target cells is calculated by the number of 7-AAD-positive/CelIVue Maroon-positive cells divided by the total number of CelIVue Maroon-positive cells.

Dose response kill curves of percent cytotoxicity against bispecific antigen binding polypeptide concentration are analyzed by 4 parameter-logistic regression equation using GraphPad Prism; and the concentration of bispecific antigen binding polypeptide that induced half maximal percent cell cytotoxicity is thus determined.

Cytotoxicity results utilizing flow cytometry are expected to be in-line with results obtained with other cytotoxicity assays, including LDH and caspase. Exposure of HCT-116 cells to protease-cleaved and uncleaved anti-EGFR×anti-CD3 bispecific antigen binding polypeptide compositions in the absence of PBMC are expected to have no effect. Similarly, PBMC are not expected to be activated in the presence of bispecific antigen binding polypeptide without target cells. These results are expected to indicate that bispecific antigen binding polypeptide compositions need to be clustered on the surface of target cells in order to stimulate PBMC for cytotoxicity activity. In the presence of PBMC and target cells, there would be a concentration-dependent cytotoxic effect due to bispecific antigen binding polypeptide pretreated or untreated with protease. Further, results are expected to show that exposure of HCT-116 cells to untreated bispecific antigen binding polypeptide (no protease) in the presence of PBMC would show reduced cytotoxicity as compared to protease-cleaved bispecific antigen binding polypeptide composition.

### Example 15: T-Cell Activation Marker Assays of Anti-EGFR×Anti-CD3 Bispecific Antigen Binding Polypeptide Composition

To measure the anti-EGFR×anti-CD3 bispecific antigen binding polypeptide induced activation markers (CD69 and CD25), 1×10⁵ PBMC or purified CD3+ cells are co-cultured in RPMI-1640 containing 10% FCS with 1×10⁴ HCT-116 or HT-29 cells per assay well (i.e., effector to target ratio of 10:1) in the presence of anti-EGFR×anti-CD3 bispecific antigen binding polypeptide (PJB0169, comprising 2 XTEN and 2 RS) in a 96-well round-bottom plate with total final volume of 200 μL. After 20 h incubation in a 37° C., 5% CO₂ humidified incubator, cells are stained with PECy5-conjugated anti-CD4, APC-conjugated anti-CD8, PE-conjugated anti-CD25, and FITC-conjugated anti-CD69 (all antibodies from BioLegend) in FACS buffer (1% BSA/PBS) at 4° C., washed twice with FACS buffer, and then re-suspended in FACS buffer for acquisition on a Guava easyCyte flow cytometer (Millipore).

The T-cell activation marker expression trend of the three bispecific antigen binding polypeptide molecules is expected to be similar to that observed by cytotoxicity assays, including LDH and caspase. Activation of CD69 on CD8 and CD4 populations of PBMC or CD3+ cells by untreated anti-EGFR×anti-CD3 bispecific antigen binding polypeptide (pJB0169) is expected to be less active than protease-treated pJB0169 bispecific antigen binding polypeptide; and the non-cleavable anti-EGFR×anti-CD3 bispecific antigen binding polypeptide (pJB0172) is expected to be less active than the untreated pJB0169.

### Example 16: Cytometric Bead Array Analysis for Human Th1/Th2 Cytokines Using Stimulated Normal Healthy Human PBMCs and Intact and Protease-Treated Anti-EGFR×Anti-CD3 Bispecific Antigen Binding Polypeptide

As a safety assessment of the ability of intact versus cleaved anti-EGFR×anti-CD3 bispecific antigen binding polypeptide (pJB0169, comprising 2 XTEN and 2 RS) to stimulate release of T-cell related cytokines in a cell-based in vitro assay, a panel of cytokines including IL-2, IL-4, IL-6, IL-10, TNF-alpha, IFN-gamma are analyzed using the cytometric bead array (CBA) on supernatants from cultured human PBMC stimulated with protease-treated and untreated anti-EGFR×anti-CD3 bispecific antigen binding polypeptide samples. The anti-human CD3 antibody, OKT3, is used as positive control and untreated wells serve as negative control.

Briefly, OKT3 (0, 10 nM, 100 nM and 1000 nM) and protease-treated and untreated anti-EGFR×anti-CD3 bispecific antigen binding polypeptide (pJB0169 at 10 nM, 100 nM, 1000 nM and 2000 nM) are dry-coated onto a 96-well flat-bottomed plate by allowing the wells to evaporate overnight in the biosafety hood. Wells are then washed once gently with PBS and 1×10⁶ PBMC in 200 μL were added to each well. The plate is then incubated at 37° C., 5% CO₂ for 24 h, after which tissue culture supernatant is collected from each well and analyzed for cytokine released using the validated commercial CBA kit (BD CBA human Th1/Th2 cytokine kit, cat #551809) by flow cytometry following manufacturer's instructions.

OKT3, but not untreated wells, is expected to induce robust secretion of all cytokines (IL-2, IL-4, IL-6, IL-10, TNF-alpha, IFN-gamma) evaluated, thereby confirming the performance of the CBA cytokine assay. Stimulation with protease-treated anti-EGFR×anti-CD3 bispecific antigen binding polypeptide is expected to trigger significant cytokine expression, especially at concentrations higher than 100 nM for all of the cytokines tested. In contrast, baseline levels of IL-2, IL-6, IL-10, TNF-alpha and IFN-gamma are expected when the intact non-cleaved anti-EGFR×anti-CD3 bispecific antigen binding polypeptide molecule is the stimulant at a concentration range of 10 to 2000 nM. These data support that the XTEN polymer of the intact bispecific antigen binding polypeptide composition provides considerable shielding effect and hinders PBMC stimulated cytokine responses compared to the protease-treated bispecific antigen binding polypeptide in which the EGFR×anti-CD3 portion is released from the composition.

### Example 17: Cytotoxicity Assays of Anti-EGFR×Anti-CD3 Bispecific Antigen Binding Polypeptide Composition in the Presence of Purified CD3 Positive T Cells

To demonstrate that cytotoxic activity of bispecific antigen binding polypeptide molecules is mediated by CD3 positive T cells, non-cleavable anti-EGFR×anti-CD3 bispecific antigen binding polypeptide without the release segment (pJB0172, comprising 2 XTEN) and protease-treated and untreated anti-EGFR×anti-CD3 bispecific antigen binding polypeptide (pJB0169, comprising 2 XTEN and 2 RS) are evaluated in EGFR+ human cell lines (e.g. HCT-116 or HT-29) in the presence of purified human CD3 positive T cells. Purified human CD3 positive T cells are purchased from BioreclamationIV, where they are isolated by negative selection using MagCellect Human CD3+ T cell isolation kit from whole blood of healthy donors. In this experiment, purified human CD3 positive T cells are mixed with an EGFR+ cell line in a ratio of about 10:1 and all three bispecific antigen binding polypeptide molecules were tested as a 12-point, 5× serial dilution dose curve in the LDH assay as described above. The activity trend of the three bispecific antigen binding polypeptide molecules profiled with CD3+ cells is expected to be similar to the profile of the same cell line with PBMCs. Untreated pJB0169 is expected to be less active than protease-treated pJB0169; and the non-cleavable pJB0172 is expected to be less active than untreated pJB0169. Such results would demonstrate that cytotoxic activity of bispecific antigen binding polypeptide molecules is indeed mediated by CD3 positive T cells. The susceptibility of the release segment contained within the cleavable anti-EGFR×anti-CD3 bispecific antigen binding polypeptide molecule to proteases postulated to be released from the tumor cells and/or activated CD3 positive T cells in the assay mixture is likely to differ between cell lines.

### Example 18: T-Cell Activation Marker and Cytokine Release Assays of Anti-EGFR×Anti-CD3 Bispecific Antigen Binding Polypeptide Composition

To measure the anti-EGFR×anti-CD3 bispecific antigen binding polypeptide induced expression of cytokines, purified CD3+ cells are co-cultured with HCT-116 cells per assay well (i.e., effector to target ratio of about 10:1) in the presence of anti-EGFR×anti-CD3 bispecific antigen binding polypeptide (pJB0169, comprising 2 XTEN and 2 RS) in a 96-well round-bottom plate with total final volume of 200 μL. After 20 h incubation in a 37° C., 5% CO₂ humidified incubator, cell supernatant is harvested for cytokine measurements. This assay can also be performed with other target cells selected from HT-29, NCI-H1573, NCI-H1975, FaDu, and SCC-9 as well as PBMC in place of purified CD3+ cells.

Cytokine analysis of interleukin (IL)-2, IL-4, IL-6, IL-10, tumor necrosis factor (TNF)-alpha and interferon (IFN)-gamma secreted into the cell culture supernatant is quantitated using the Human Th1/Th2 Cytokine Cytometric Bead Array (CBA) kit (BD Biosciences cat #550749) following manufacturer's instruction. In the absence of bispecific antigen binding polypeptide, no cytokine secretion above background is expected from purified CD3+ cells. pJB0169 in the presence of EGFR-positive target cells and purified CD3+ cells is expected to activate T cells and secrete a pattern of T cell cytokines with a high proportion of Th1 cytokines such as IFN-gamma and TNF-alpha. Compared to intact pJB0169, lower concentrations of protease-treated pJB0169 are expected to active T cells and secrete T cell cytokines, supporting the shielding effect of the XTEN polymer in bispecific antigen binding polypeptide.

### Example 19: Anti-Tumor Properties of Anti-EGFR×Anti-CD3 Bispecific Antigen Binding Polypeptide Compositions in Early Treatment HT-29 In Vivo Model

An in vivo efficacy experiment was performed to evaluate an EGFR-CD3 bispecific antigen binding polypeptide composition based on the pJB0169 construct in immunodeficient NOD/SCID mice, characterized by the deficiency of T and B cells and impaired natural killer cells. Mice were maintained in sterile, standardized environmental conditions and the experiment was performed in accordance with US Institutional Animal Care Association for Assessment and Use Committee (IACUC Accreditation of Laboratory Animal Care (AAALAC)) guidelines. The efficacy of protease-treated and protease-untreated anti-EGFR×anti-CD3 bispecific antigen binding polypeptide (e.g. pJB0169) was evaluated using the EGFR BRAF mutant human HT-29 adenocarcinoma xenograft model. Briefly, on day 0, 6 NOD/SCID mice were subcutaneously implanted in the right flank with 3×10⁶ HT-29 cells per mouse (Cohort 1). On the same day, cohort 2 to 7 each consisting of 6 NOD/SCID mice per group were subcutaneously injected in the right flank with a mixture of 6×10⁶ human PBMC and 3×10⁶ HT-29 cells per mouse. Four hours after HT-29 or HT-29/PBMC mixture inoculation, treatments were initiated. Cohort 1 and 2 were injected intravenously with vehicle (PBS+0.05% Tween 80), cohort 3 and 4 were injected with 0.05 mg/kg of the intact anti-EGFR×anti-CD3 bispecific construct and 0.5 mg/kg of the anti-EGFR×anti-CD3 bispecific construct treated with protease to remove the XTEN from the polypeptide, respectively, cohort 5 and 6 were injected with 0.143 mg/kg and 1.43 mg/kg intact anti-EGFR×anti-CD3 bispecific construct, and cohort 7 were injected with 50 mg/kg cetuximab as the positive control. Cohorts 1 to 6 further received seven additional doses administered daily from day 1 to day 7 (total 8 doses). Cohort 7 was dosed with cetuximab twice/week for 4 weeks for a total of 8 doses.

Tumors in the mice were measured twice per week for a projected 33 days with a caliper in two perpendicular dimensions and tumor volumes were calculated by applying the (width²×length)/2 formula. Body weight, general appearance and clinical observations such as seizures, tremors, lethargy, hyper-reactivity, pilo-erection, labored/rapid breathing, coloration and ulceration of tumor and death were also closely monitored as a measure of treatment related toxicity. Percent tumor growth inhibition index (% TGI) was calculated for each of the treatment group by applying the formula: ((Mean tumor volume of Cohort 2 vehicle control−Mean tumor volume of test article treatment)/mean tumor volume of Cohort 2 vehicle control)×100. Treatment results with a % TGI≥60% is considered therapeutically active.

Results: At day 33, vehicle-treated cohort 1 mice bearing tumor cells only had an average tumor burden of 250±113 mm³. Cohort 2 mice treated with vehicle in the presence of human effector cells did not inhibit tumor progression, having an average tumor burden of 238±228 mm³, demonstrating that human effector cells alone, as such, could not elicit an anti-tumor effect. Treatment with the protease-treated anti-EGFR×anti-CD3 construct at 0.05 mg/kg and 0.5 mg/kg (cohort 3 and 4 respectively) in the presence of human effector cells exhibited clear anti-tumor regression with a % TGI of 99% for both treatment groups. Importantly, treatment with anti-EGRF×anti-CD3 bispecific antigen binding composition at 0.143 mg/kg and 1.43 mg/kg (cohort 5 and 6 respectively) in the presence of human effector cells also inhibited tumor growth in a dose-dependent manner with % TGI of 70% for the 0.143 mg/kg dose group and 96% in the 1.43 mg/kg cohort. The data suggest that at 0.143 mg/kg and 1.43 mg/kg dosages, sufficient amounts of the anti-EGFR×anti-CD3 constructs were effectively cleaved by proteases in the in vivo tumor environment into the more active, unXTENylated anti-EGFR×anti-CD3 bispecific antigen binding fragments to yield the observed efficacy. Significantly, cohort 7 treated with 50 mg/kg of cetuximab did not induce tumor regression, with a % TGI of −20%.

Conclusions: The results suggest that the anti-EGFR×anti-CD3 bispecific construct can be effectively cleaved in vivo into the active form and is efficacious in the EGFR BRAF mutant HT-29 tumor environment to inhibit tumor progression. In addition, the anti-EGFR×anti-CD3 bispecific construct was superior to the cetuximab control in anti-tumor activity under the conditions of the experiment. Of note, no significant body weight loss was observed in all test article treatment groups and vehicle.

### Example 20: Anti-Tumor Properties of Protease Activated Anti-Her2×Anti-CD3 Bispecific Antigen Binding Polypeptide Bearing One or Two XTEN in Established Ovarian Tumor Model

In the established murine ovarian tumor model, 5×10⁶ SK-OV-3 tumor cells were independently implanted, in the presence of matrigel, subcutaneously into fifty-eight NOG (NOD/Shi-scid/IL-2Rγ^{null}) mice on day 0. (The NOG mice are NOD/SCID mice bearing IL-2Rγ mutation resulting in the mice lacking T, B and NK cells, dysfunctional macrophage, dysfunctional dendritic cells and reduced complement activity.) When SK-OV-3 tumor volume reached approximately 60 mm³, six NOG mice were intravenously administered with 100 μL PBS and designated as Cohort 1. The remaining unassigned 52 mice were intravenously injected with 5×10⁶ human PBMCs/mouse. When mean tumor volume reached approximately 150 mm³, 36 of the 52 NOG mice were allocated to 6 study groups of 6 mice per group based on tumor volume. These groups were assigned as study Cohort 2 to 7. Treatment with vehicle, a protease-untreated anti-Her2×anti-CD3 bispecific antigen binding polypeptide carrying one XTEN polymer (i.e., pCW1628), an anti-Her2×anti-CD3 bispecific antigen binding polypeptide bearing two XTEN polymers (e.g. pJB0244) and a protease-treated anti-Her2×anti-CD3 bispecific antigen binding polypeptide in which the XTEN are cleaved from the construct was initiated at equimolar concentrations for each group, dosed as three intravenous doses per week for three weeks. Cohort 1 and 2 were the vehicle-treated groups, cohort 3 was the pCW1628-treated group at 0.82 mg/kg (6 nmol/kg), cohort 4 was the protease-treated anti-Her2×anti-CD3 construct (without XTEN)-treated group at 0.35 mg/kg (6 nmol/kg), and cohort 5 to 7 were the pJB0244 bispecific construct-treated group at 1 mg/kg (6 nmol/kg), 2.5 mg/kg (15 nmol/kg) and 6.0 mg/kg (36 nmol/kg) respectively.

Tumors were measured twice per week for up to 35 days with a caliper in two perpendicular dimensions and tumor volumes were calculated by applying the (width²×length)/2 formula. Body weight, general appearance and clinical observations such as seizures, tremors, lethargy, hyper-reactivity, pilo-erection, labored/rapid breathing, coloration and ulceration of tumor and death were also closely monitored as a measure of treatment related toxicity. Percent tumor growth inhibition index (% TGI) was calculated for each of the treatment group by applying the formula: ((Mean tumor volume of Cohort 2 vehicle control−Mean tumor volume of test article treatment)/mean tumor volume of Cohort 2 vehicle control)×100. Treatment group with % TGI≥60% is considered therapeutically active.

Results: At day 35, vehicle-treated cohort 1 and 2 mice did not inhibit tumor progression, having a tumor burden of 1122±243 mm³ and 844±258 mm³ demonstrating that human effector cells alone as such could not elicit an anti-tumor effect. As expected, treatment with protease-treated anti-Her2×anti-CD3 bispecific construct at 0.35 mg/kg (cohort 4) in the presence of human effector cells exhibited clear anti-tumor regression with % TGI of 100%. Treatment with pCW1628, an anti-Her2×anti-CD3 bispecific construct bearing one XTEN polymer at 0.82 mg/kg (cohort 3) in the presence of human effector cells also elicited a robust anti-tumor response yielding a % TGI of 100%. Importantly, a dose-dependent anti-tumor response was observed with treatment of, an anti-Her2×anti-CD3 bearing two XTEN polymers. pJB0244 dosed at 1 mg/kg (cohort 5) was considered therapeutically inactive with a % TGI of 51%. Increasing the dose level of pJB0244 to 2.5 mg/kg yielded a therapeutically active % TGI of 69% and to 6 mg/mL a TGI of 98%.

Conclusions: Data suggest that at 6 mg/kg (36 nmol/kg) in the in vivo SK-OV-3 tumor environment, protease-untreated anti-Her2×anti-CD3 bispecific construct bearing two XTENs (e.g., pJB0244) is as efficacious as protease-untreated anti-Her2×anti-CD3 bearing one XTEN polymer (e.g., pCW1628) at 6 nmol/kg and to protease-treated anti-Her2×anti-CD3 bispecific construct molecule (with XTEN removed) at 6 nmol/kg. Of note, no significant body weight loss was observed in all test article treatment groups as compared to Cohort 2 vehicle control indicating that all treatments were well tolerated.

### Example 21: Single- and Multi-Dose Pharmacokinetic Determination of Anti-EGFR×Anti-CD3 Bispecific Antigen Binding Polypeptide in Non-Human Primates

The pharmacokinetics (PK) and general tolerability of anti-EGFR×anti-CD3 bispecific antigen binding polypeptide bearing 2 XTEN polymers (i.e., pJB0169) following single and multiple intravenous administrations was evaluated in naïve, healthy non-human primates (NHP) (e.g., cynomolgus monkeys). Briefly, one female and one male monkey was intravenously infused with 8.5 μg/kg of the composition via the cephalic vein. Both animals were monitored for two weeks. Following no observable adverse events, animals were subjected to a multi-dose regimen initiated as one dose every three days for three weeks (total 9 doses in study). The multi-dose phase began with Day 15 and ended on Day 36. At specific time points throughout the study, blood was collected for assay of pharmacokinetics, cytokines, hematology and serum chemistries.

Animal monitoring included body weight, body temperature and cage-side observations once or twice daily during the duration of the study. Animals were monitored for general health and appearance; signs of pain and distress, fever, chills, nauseas, vomiting and skin integrity. On dosing days, animals were checked for injection site reactions before and after administration of the compositions. Hematology and serum chemistry were determined at predose and 24 hours after first single dose. Cytokines were evaluated at pre-dose and at appropriate intervals within 72 hours post first single dose and in the multi-dose phase.

The amount of pJB0169 present in plasma was quantitated on a sandwich ELISA using EGFR-biotin captured on an electrochemiluminescence streptavidin plate with sulfo-tagged anti-XTEN-antibody as detection. Pharmacokinetic parameters including Cmax, Tmax, area under the curve, half-life and exposure profile were analyzed using the WinNonLin software.

The cytokine panel included measurement of IFN-gamma, IL-1beta, TNF-alpha, IL-1beta, IL-2, IL-4, IL-6, and IL-10 using the Meso-Scale Discovery platform following manufacturer's instructions. The lower limit of detection for these cytokines are 2.0 μg/mL, 0.32 pg/mL, 0.11 μg/mL, 0.68 μg/mL, 0.04 μg/mL, 0.23 μg/mL and 0.10 μg/mL respectively. The hematology panel included measurement of white blood cells, red blood cells, hemoglobin, hematocrit, mean corpuscular hemoglobin volume, mean corpuscular hemoglobin concentration, red blood cell distribution width, platelet, mean platelet volume, % neutrophils, % lymphocytes, % monocytes, % eosinophils and % basophils. The serum chemistry panel included measurement of alanine aminotransferase, aspartate aminotransferase, total protein, albumin, alkaline phosphatase, globulin, albumin/globulin ratio, γ-glutamyltransferase, glucose, urea, creatinine, calcium, total cholesterol, triglycerides, total bilirubin, sodium, potassium, chlorine and creatine kinase.

Results: pJB0169 was well tolerated at a dose of 8.5 μg/kg. There was no loss in body weight. No chills, fever, nausea, vomiting, skin rash, test artile injection site reaction were observed. All measured cytokine levels except IL-6 were below the limits of detection. Although detectable in the single-dose and multi-dose phase, the level of IL-6 detected is considered to be background with the highest level not exceeding 51 μg/mL in male and 19 μg/mL in female animals in the range of time points evaluated. Hematology and clinical panel were within normal range. Following Day 1 administration, at 8.5 μg/kg, the average Cₘₐₓ value was 372 ng/mL, the averaged AUC₀₋₁₆₈ₕ was 15839 ng*h/mL, the averaged AUC_{0-inf} was 16342 ng*h/mL, the averaged CL value was 0.00886 mL/min/kg and the averaged T_{1/2} value was 24.2 hours. The volume of distribution (Vd) was 0.0238 L/kg. Following Day 36 administration, average Cₘₐₓ value was 410 ng/mL, the averaged AUC₀₋₁₆₈ₕ was 22985 ng*h/mL, the averaged AUC_{0-inf} was 24663 ng*h/mL, the averaged CL value was 0.00578 mL/min/kg and the averaged T_{1/2} value was 44.0 hours. The volume of distribution (Vd) was 0.0196 L/kg. The accumulative index of Cₘₐₓ and AUC₀₋₁₆₈ₕ in monkey following single or multiple IV infusion administration of pJB0169 at 8.5 μg/kg were 1.10 and 1.45. There was no significant difference in systemic exposure between Day 1 and Day 36 administration. Data also suggest no emergence of anti-drug antibodies.

### Example 22: Dose Range Finding of Anti-EGFR×Anti-CD3 Bispecific Antigen Binding Polypeptides in Non-Human Primates

The dose range finding study of the pJB0169 bispecific antigen binding polypeptide in non-human primates was carried out in healthy, naïve cynomolgus monkeys with one female and one male monkey per cohort. Briefly, one female and one male monkey was intravenously infused with pJB0169 via the cephalic vein. Both animals were monitored for two weeks. Following no observable adverse events, animals were subjected to a multi-dose regimen initiated as one dose every three days for three weeks (total 9 doses in study). The multi-dose phase began with Day 15 and ended on Day 36. At specific time points throughout the study, blood was collected for assay of pharmacokinetics, cytokines, hematology and serum chemistries. Twenty-four hours after the last dose (i.e. Day 37), animals were necropsied for histopathology evaluation. When no adverse events were observed one week after the first dose in a cohort, pJB0169 was dose escalated 2- or 3-fold in the next cohort. Dose escalation will proceed until adverse events are observed.

Animal monitoring included body weight, food consumption, body temperature and cage-side observations once or twice daily during the duration of the study. Animals were monitored for general health and appearance; signs of pain and distress; fever, chills, nauseas, vomiting and skin integrity. On dosing days, animals were checked for injection site reaction before and after administration of the test articles.

The amount of pJB0169 present in plasma will be quantitated on a sandwich ELISA using EGFR-biotin captured on an electrochemiluminescence streptavidin plate with sulfo-tagged anti-XTEN-antibody as detection. Pharmacokinetic parameters including Cmax, Tmax, area under the curve, half-life and exposure profile will be analyzed using WinNonLin software.

The cytokine panel will include measurement of IL-2, IL-4, IL-5, IL-6, IL-10, IL-13, IFN-gamma and TNF-alpha using Beckon Dickinson Cytometric Bead Array.

The hematology panel included measurement of white blood cells, red blood cells, hemoglobin, hematocrit, mean corpuscular hemoglobin volume, mean corpuscular hemoglobin concentration, red blood cell distribution width, platelet, mean platelet volume, % neutrophils, % lymphocytes, % monocytes, % eosinophils and % basophils.

The serum chemistry panel included measurement of alanine aminotransferase, aspartate aminotransferase, total protein, albumin, alkaline phosphatase, globulin, albumin/globulin ratio, y-glutamyltransferase, glucose, urea, creatinine, calcium, total cholesterol, triglycerides, total bilirubin, sodium, potassium, chlorine and creatine kinase.

Histopathology evaluation with H&E staining were performed on a panel of tissues including adrenal glands, aorta, bone, brain, epididymides, esophagus, eyes, fallopian tubes (female only), heart, kidney, large intestines, liver with gall bladder, lungs, lymph nodes, mammary glands (female only), ovaries (female only), pancreas, pituitary gland, prostate gland, salivary glands, skeletal muscles, skin, small intestines, spinal cord, spleen, stomach, testes (male only), thymus, thyroid glands, trachea, urinary bladder, uterus and injection site.

Interim results: The starting dose in this dose range finding study was Cohort 1 at 25.5 μg/kg of pJB0169. No observable adverse events such as fever, chills, skin rash, nausea, vomiting, abnormal hematology and serum chemistry were observed in the single-dose and multi-dose phase. pJB0169 was therefore dose-escalated 3-fold to 76.5 μg/kg in Cohort 2. No observable adverse events were observed in Cohort 2 and pJB0169 was dose-escalated 3-fold to 230 μg/kg in Cohort 3. Other than a reversible increase in AST, ALT and total bilirubin readings above the normal range, no other adverse events were observed and pJB0169 was next dose-escalated 2-fold to 460 μg/kg in Cohort 4. No observable adverse events were observed in Cohort 4. Further dose escalation of pJB0169 is ongoing.

There were no found dead and moribund animals during the whole study period. There were no test article-related organ weight changes in any treatment groups. There were no observed gross lesions in all the tested animals. Microscopically, the major findings were subcutaneous hemorrhage, tissue necrosis, neutrophilic infiltration, venous necrosis or thrombosis, and skin crust at the injection sites of some animals. These changes were likely attributed to the intravenous infusion procedure.

Interim conclusions: pJB0169 bispecific antigen binding polypeptide is well-tolerated in non-human primates at doses up to 460 μg/kg. No test article-related organ weight and pathologic changes were observed in all tested dose groups.

### Example 23: Determination of Isoelectric Point (pI) of Antigen Binding Fragments

To determine the isoelectric point various CD3 and EGFR variant antigen binding fragments, each was analyzed using the Protein Titration Curve Panel in the Biologics suite of Maestro (Schrödinger, Germany). The titration curve for a protein is calculated from the pKa values of titratable groups—individual ionizable residues and termini—by summing the fractional charges of each such group at intervals in the pH value. The pKa values are generated with ProPKA (Sondergaard, C. et al. Toxicol Lett. 205(2):116 (2011); Olsson, M. et. al. Proteins 79:3333 (2011)). The titration curves were plotted and the isoelectric point (pI) was determined for each curve, with the results presented in the tables, below.

TABLE 20
Isoelectric points for CD3 variants

| Antibody | Variant | Isoelectric Point (pI) |
|---|---|---|
| CD3 | 3.9 | 6.8 |
| CD3 | CD3.30 | 6.8 |
| CD3 | CD3.31 | 6.2 |
| CD3 | CD3.32 | 6.2 |
| CD3 | CD3.33 | 6.2 |

TABLE 21
Isoelectric points for EGFR variants

| Antibody | Variant | Isoelectric Point (pI) |
|---|---|---|
| EGFR | EGFR.2 | 5.0 |
| EGFR | EGFR.13 | 5.0 |
| EGFR | EGFR.18 | 5.1 |
| EGFR | EGFR.23 | 5.1 |
| EGFR | EGFR.14 | 5.0 |
| EGFR | EGFR.19 | 5.1 |
| EGFR | EGFR.24 | 5.1 |
| EGFR | EGFR.15 | 5.3 |
| EGFR | EGFR.20 | 5.5 |
| EGFR | EGFR.25 | 5.5 |
| EGFR | EGFR.16 | 5.3 |
| EGFR | EGFR.21 | 5.5 |
| EGFR | EGFR.26 | 5.5 |
| EGFR | EGFR.17 | 5.3 |
| EGFR | EGFR.22 | 5.5 |
| EGFR | EGFR.27 | 5.5 |

### Example 24: Assessment of Masking Effect of XTENs on XPATs Via In Vitro Cytotoxicity Assays of HER2-XPAT Vs HER2-PAT

The in vitro T-cell directed cytotoxicity of HER2-XPAT (XTENylated HER2/CD3 binder, prodrug) and HER2-PAT (non-XTENylated HER2/CD3 binder, drug) were compared to assess the protective/masking effect of the XTEN molecules contained in the former using a PBMC-based effector cell assay. The HER2-XPAT construct comprised from N- to C-terminus: (1) an N-terminal XTEN (AE292 SEQ ID NO: 714) with a histidine tag, (2) a release segment, (3) an anti-HER2 scFv (the Her2.2 scFv, SEQ ID NO: 1140), (4) an anti-CD3 scFv (the CD3.23 scFv, SEQ ID NO: 1141), (5) a release segment, and (6) a C-terminal XTEN (AE584, SEQ ID NO:926). The HER2-PAT comprised the same elements as HER2-XPAT, except with the N- and C-terminal XTEN molecules cleaved off by protease treatment at the release segments.

TABLE 22
Protein Components of XPATs Used Herein

| | | SEQ ID |
|---|---|---|
| Construct | Amino Acid Sequence | NO: |
| Her2.2 | DIQMTQSPSSLSASVGDRVTITCKA | 1140 |
| scFv | SQDVSIGVAWYQQKPGKAPKLLIYS | |
| | ASYRYTGVPSRFSGSGSGTDFTLTI | |
| | SSLQPEDFATYYCQQYYIYPYTFGQ | |
| | GTKVEIKGATPPETGAETESPGETT | |
| | GGSAESEPPGEGEVQLVESGGGLVQ | |
| | PGGSLRLSCAASGFTFTDYTMDWVR | |
| | QAPGKGLEWVADVNPNSGGSIYNQR | |
| | FKGRFTLSVDRSKNTLYLQMNSLRA | |
| | EDTAVYYCARNLGPSFYFDYWGQGT | |
| | LVTVSS | |
| CD323 | ELVVTQEPSLTVSPGGTVTLTCRSS | 1141 |
| scFv | NGAVTSSNYANWVQQKPGQAPRGLI | |
| | GGTNKRAPGTPARFSGSLLGGKAAL | |
| | TLSGVQPEDEAVYYCALWYPNLWVF | |
| | GGGTKLTVLGATPPETGAETESPGE | |
| | TTGGSAESEPPGEGEVQLLESGGGI | |
| | VQPGGSLKLSCAASGFTENTYAMNW | |
| | VRQAPGKGLEWVARIRSKYNNYATY | |
| | YADSVKDRFTISRDDSKNTVYLQMN | |
| | NLKTEDTAVYYCVRHENFGNSYVSW | |
| | FAHWGQGTLVTVSS | |

Underlined residues indicate linker sequences, ScFvs are oriented VL-linker-VH, Bolded residues indicate CDRs

Cytotoxicity of both molecules was verified using an in vitro cytotoxicity method which utilized the amount of ATP present in wells of lysed target cells post treatment as a proxy for measuring cell viability. HER2 expressing target cells were seeded on white, clear bottom plates at varying densities (BT474 and NUGC4-20 k cells/well, SKOV3 and RT-112-10 k cells/well, MCF7 and MDA-MB-231-7.5 k cells/well) and allowed to incubate at 37° C., 5% CO2 overnight (18-24 hours). Prior to the end of the overnight incubation, PBMCs were thawed and incubated at 37° C., 5% CO2 for 4 hours. PBMCs were isolated from screened, healthy donors by ficoll density gradient centrifugation from either whole blood or from lymphocyte-enriched buffy coat preparations obtained from BioIVT. 10×HER2-XPAT and HER2-PAT dose-response titrations were prepared using an 11 point, 3-fold titration (12th point is non-treatment) with a starting concentration of 2400 nM for HER2-XPAT and 10 nm for HER2-PAT. PBMCs were seeded in the wells at varying Effector:Target (E:T) ratios (BT474-5:1, MCF7, RT-112, MDA-MB-231 and SKOV3-10:1, NUGC4-˜8:1). 10×HER2-XPAT and AMX-818-P1(PAT) titrations were diluted 10-fold into the well for starting concentrations of 240 nM and 1 nM, respectively. The plates were incubated at 37° C., 5% CO2 for 48 hours. After the 48-hour incubation, the plates were washed 3× with 1×PBS and 100 μL of 1×PBS was added to all wells. For the ATP assay, 100 μL of CellTiter-Glo® luminescent substrate solution was added to all wells and the plates were allowed to incubate at room temperature for 1-5 minutes. The plates were then shaken on a plate shaker at 300-500 rpm for 30-60 seconds to mix the contents of the wells and read in a luminometer using an integration time of 100 ms. The intensity of signal produced correlated to the amount of viable cells present in the wells. Mean of the signal from all non-treatment wells was calculated and used to determine % Live cells from treatment wells ((Treatment Signal/Mean of Non-Treatment Signal)*100=% Live). The % Live was plotted by concentration and half maximal response (IC50) values were derived with a 4-parameter logistic regression equation using GraphPad Prism software.

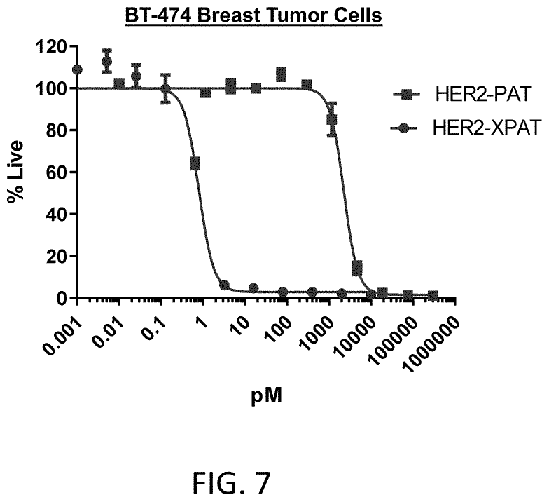

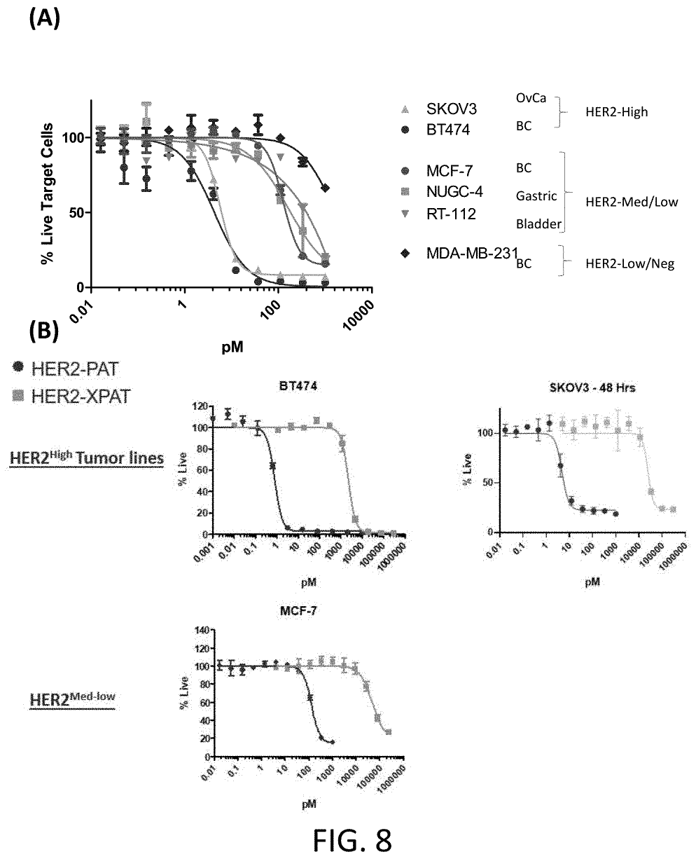

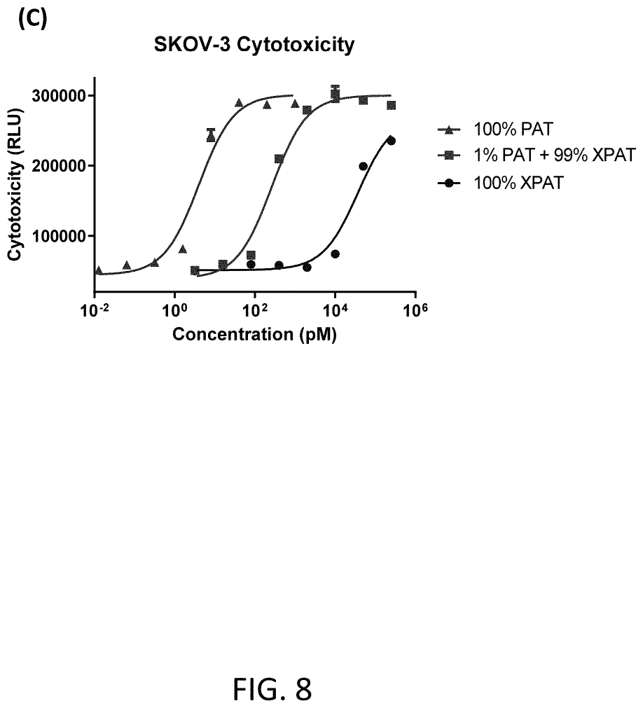

The results of these studies indicated that XTENylation was capable of mitigating cytotoxicity of the XPAT molecule relative to its cleaved HER2-PAT counterpart. HER2-XPAT and HER2-PAT displayed large differentials in potency against all HER2 expressing cell lines tested, confirming that XTENylation results in reduction of cytolytic activity of HER2-XPAT (inactivated state). This can be seen in FIG. 7 and FIG. 8B. FIG. 7 shows results of a dose-response SK-OV3 cell caspase assay where cells are treated with HER2-XPAT or HER2-PAT, demonstrating that there is a large separation between both dose response curves and thus the cytotoxicity of both constructs. FIG. 8B shows similar dose response results of the same HER2-XPAT/HER2-PAT experiment against BT-474 cells, SK-OV-3 cells, and MCF-7 cells, also demonstrating a large separation between dose response curves of the two compounds in additional cell lines.

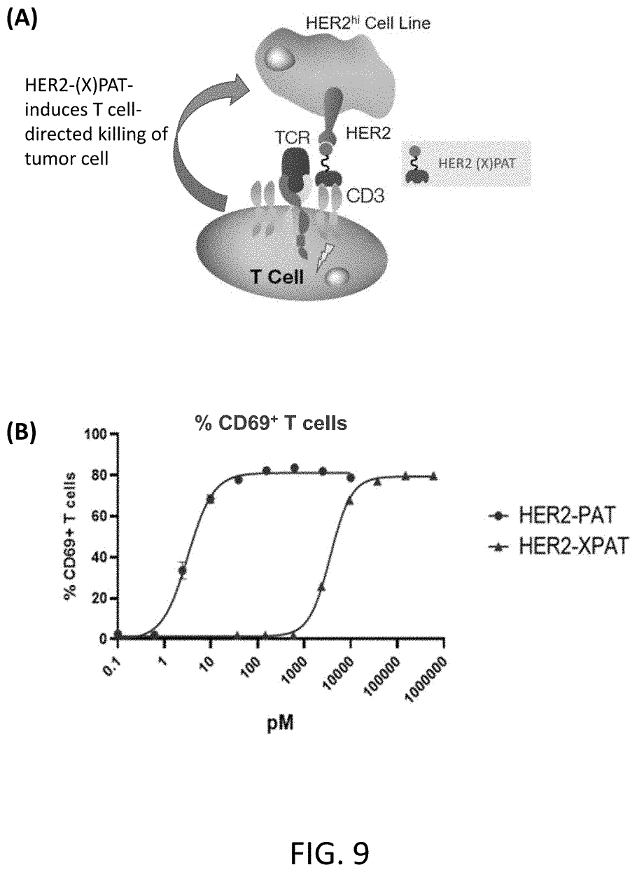

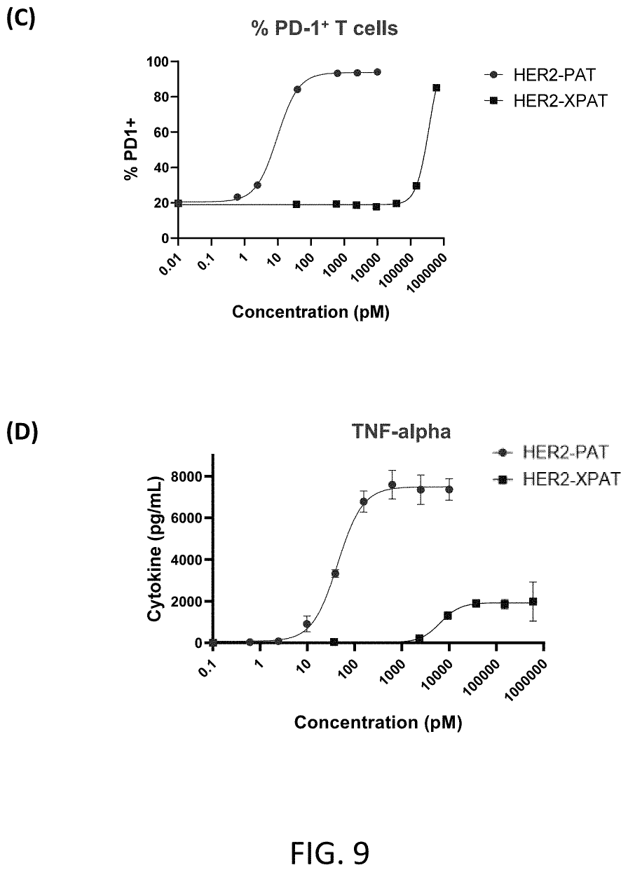

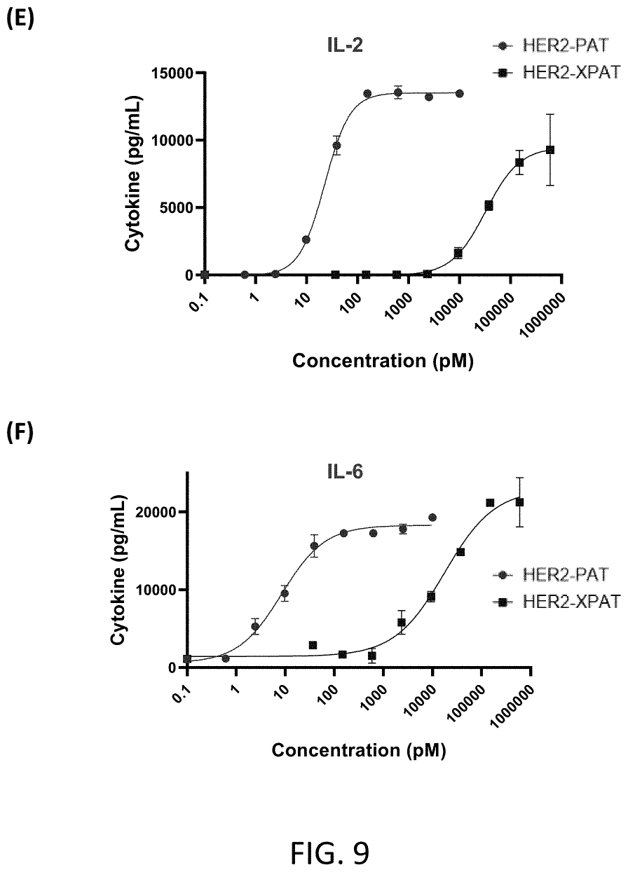

Further, the experiments indicated that the potency of the XPAT molecule without XTEN (HER2-PAT) correlated with HER2 cell expression, indicating the cell killing mechanism is specific for HER2. This can be seen in FIG. 8A and FIG. 8B. FIG. 8A shows a dose response of HER2-PAT in a range of different cell lines that have varying HER2 surface expression; the HER2-PAT molecule shows highest potency/lowest apparent EC50 against the high HER2 expressors (SK-OV-3, BT-474), intermediate potency against the intermediate expressors (MCF-7, NUGC-4, RT-112), and low potency against the low/negative expressor (MDA-MB-231). FIG. 8B shows a different presentation of the same data against select HER2^{High} (BT-474, SK-OV-3) and HER2^{med-low} (MCF-7) cell lines only alongside potency of the HER2-XPAT molecule, demonstrating that the potency of both forms (+ and − XTEN molecule) of the XPAT molecule depend on HER2 expression, as the EC50 of the XTENylated molecule was lower (indicating greater potency) against HER2High cell lines than the HER2Med-low cell line. Cytotoxicity of HER2-PAT on RT-112 and NUGC4 was observed in a dose-dependent manner, with maximal killing of −80% observed at 1 nM of HER2-PAT. HER2-PAT displayed an estimated IC50 of 127.3 pM on MCF7 cells, while the IC50 for HER2-XPAT is >26,667 pM (>200-fold difference in potency). On HER2 amplified cell lines, HER2-PAT obtained IC50 values of 4.4 pM (BT474) and 5.71 pM (SKOV3), while AMX-818(P1) obtained IC50 values of 1,364 pM (BT474) and 15,256 pM (SKOV3). This variation in potency according to HER2 surface expression leads us to conclude that HER2-PAT has cytotoxic potential on cell lines with low to high HER2 expression, and XTENylation masks (or shields) the ability of the XPAT molecule to form an immune synapse between T-cells and target cancer cells, resulting in reduction of potency of the XPAT molecule (an illustration of the proposed mechanism of action appears in FIG. 9A).

### Example 25: Assessment of Toxicity of XTENylated Anti-CD3/Anti-HER2 XPAT in Cardiomyocytes

Normal human cardiomyocytes express low levels of HER2 and as a result, rare cases of cardiac toxicity have been observed in patients treated with some HER2-targeted therapies. Accordingly, the T-cell directed cytotoxicity of XTENylated (HER2-XPAT, prodrug) and non-XTENylated (HER2-PAT, drug) PATs from Example 24 were assessed in normal human cardiomyocytes again using a PBMC effector cell assay. In the assay, normal human cardiomyocytes purchased from FujiFilm Cellular Dynamics were used. icell cardiomyocytes (Cellular Dynamics International) were revived from liquid nitrogen and plated at 20,000 cells per 96-well for 7 days and treated as per the manufacturer's instructions. Human peripheral blood lymphocytes were added onto icell cardiomyocytes at a 10:1 Effector:Target ratio with increasing 3-fold concentrations of HER2-XPAT (starting at 300 nM) or HER2-PAT (starting at 1 nM) and incubated for 48 hours at 37° C., 5% CO2. The assay was performed in RPMI and 10% heat-inactivated fetal bovine serum. Cardiomyocyte cell viability was determined via ATP quantification and was performed with the Cell Titer-Glo Luminescent Cell Viability Assay System (Promega). Cell supernatant was aspirated, and cells were washed twice with phosphate buffered saline (PBS), aspirated, and followed by addition of PBS (100 μl per well). Automated plate washing was carried out using an LS405 microplate washer dispenser (BioTek). Cell Titer-Glo reagent was added (100 n1 per well), and assay plates were incubated for 5 minutes at room temperature. Luminescence was quantified with a multi-label reader (Molecular Devices) with an luminescence detector. For analysis of cytotoxicity, % viable cells was calculated from relative luminescence units (RLU). % live=(Test well RLU/Target cell only RLU)*100. For EC50 determination, data were transformed in Microsoft Excel and analyzed with Graph Pad Prism 8.3.1 software log(agonist)vs.response-variable slope (four parameters).

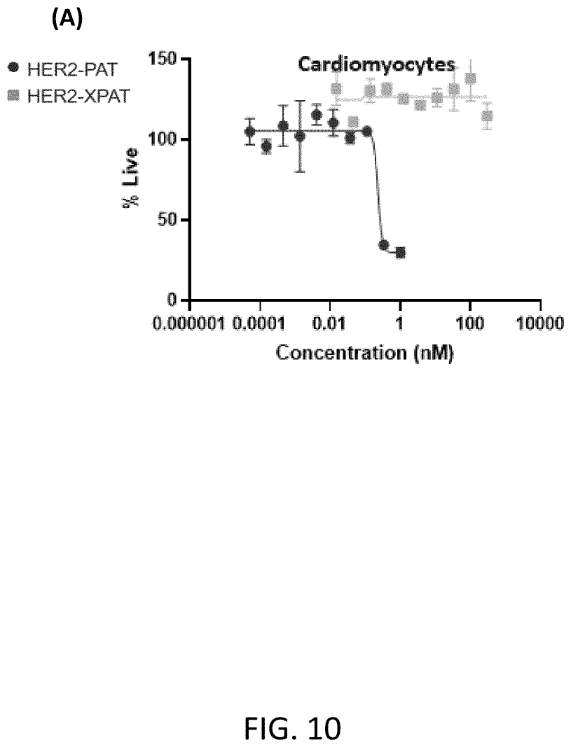

Results indicated that the XTENylation was effective at protecting the cardiomyocytes from T-cell directed cytotoxicity due to the XPAT molecule. This can be seen in FIG. 10A. FIG. 10A shows a dose-response curve of both HER2-XPAT and HER2-PAT, wherein the active drug HER2-PAT demonstrates an apparent EC50 of less than ˜1 nM and the XTENylated prodrug HER2-XPAT shows no significant cytotoxicity at greater than 100 nM concentration.

### Example 26: In Vitro T Cell Activation by HER2-XPAT and its Proteolytic Metabolites Demonstrates Masking Effect of XTENs on XPATs (XTENylated Protease-Activated T Cell Engagers)

Having observed the efficacy of the HER2-CD3 platform and the efficacy of the XTEN molecule at protecting it from unregulated activity, experiments were next performed to assess: (a) dependence of T-cell activation by XPATs on HER2 engagement; and (b) efficacy of single-XTENylation (N- or C-terminal) to mitigate cytotoxic activity due to the XPAT molecule.

For (a) and (b), the activation of CD3 positive T cells by HER2-XPAT and its proteolytic metabolites was verified using an in vitro T cell activation method. Jurkat T Cells genetically engineered to express a luciferase reporter driven by a NFAT-response element were utilized to ascertain the level of T Cell activation via measurement of luciferase present in the wells post treatment. For both (a) and (b), HER2 expressing target cells were seeded on white, clear bottom plates at varying densities (BT474-20 k cells/well, SKOV3-10 k cells/well) and allowed to incubate at 37° C., 5% CO₂ overnight (18-24 hours).

To assess the dependence of the XPAT molecule on HER2 cell surface engagement for T-cell activation (a), Jurkat T cell activation assays were performed where a portion of the wells were plated with only media (e.g. without HER2-expressing cells). Prior to the end of the overnight incubation above, 7.5×HER2-XPAT and HER2-PAT dose-response titrations were prepared using a 7 point, 6-fold titration (8th point is non-treatment) with a starting concentration of 6000 nM for HER2-XPAT and 150 nm for HER2-PAT. Jurkat reporter cells were seeded in the wells at a 5:1 Effector:Target (E:T) ratio (BT474-100 k cells/well). 7.5×HER2-XPAT and HER2-XPAT(PAT) titrations were diluted 7.5-fold into the well for starting concentrations of 800 nM and 20 nM, respectively. 7.5×HER2-XPAT at 6000 nM and HER2-PAT at 150 nM were diluted 7.5-fold into wells containing only Jurkat cells (no BT474s). The plates were incubated at 37° C., 5% CO2 for 6 hours. After the 6-hour incubation, 75 μL of Bio-Glo® luminescent substrate solution was added to all wells and the plates were incubated at room temperature for 5-10 minutes. The plates were then shaken on a plate shaker at 300-500 rpm for 30 seconds to mix the contents of the wells and read in a luminometer using an integration time of 500 ms. The intensity of signal produced correlates to the amount of luciferase from lysed Jurkats present in the wells. The signal was plotted by concentration and half maximal response (EC50) values were derived with a 4-parameter logistic regression equation using GraphPad Prism software.

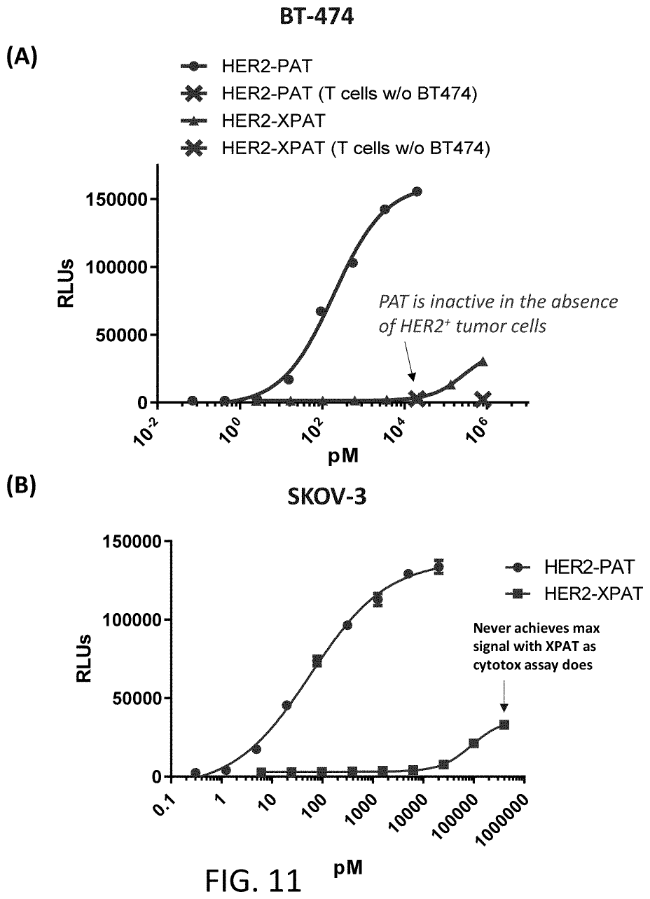

The results indicated that activation of the Jurkat cells depended on engagement of HER2 on the surface of target cells. This can be seen in FIG. 11. FIG. 11A shows a dose response of Jurkat cell activation/NFAT transactivation (measured in RLUs of luciferase activity) for both the drug HER2-PAT (“HER2-PAT”) and prodrug HER2-XPAT (“HER2-XPAT”) in the presence and absence of HER2-bearing BT-474 cells. FIG. 11B shows similar data comparing HER2-PAT (“HER2-PAT”) and HER2-XPAT (“HER2-XPAT”)-induced T cell activation with HER2-bearing SK-OV-3 cells. In FIG. 11A T cells show a lack of activation by both HER2-PAT (“HER2-PAT”) and HER2-XPAT (“HER2-XPAT”) in the absence of HER2-expressing cells, as shown by the failure of the non-HER2 cell containing conditions to reach meaningful levels of T cell activation at maximum concentration compared to the maximum concentration HER2-PAT (“HER2-PAT”) condition in the presence of BT-474 cells. In both FIGS. 11A and 11B, HER2-PAT (“HER2-PAT”) and HER2-XPAT (“HER2-XPAT”) continue to show the previously observed shift in dose-response activity, showing that the XTEN molecule also appears to block T cell activation. Interpolated concentration from maximal signal observed for HER2-XPAT at 800 nM (30.47 k RLUs) for HER2-PAT at 25.6 pM indicates that there is a ˜31,257-fold difference in activity between HER2-XPAT and HER2-PAT under these conditions.

As illustrated in FIG. 8C, a relatively low level of unmasking may nonetheless response in a significant increase in cytotoxic activity. For example, at 10 nM total concentration, 1% unmasked PAT (0.1 nM) and 99% masked XPAT yields maximal activity equivalent to maximal activity of 100% PAT.

To evaluate the effects of single-XTENylated intermediates on mitigation of XPAT activity, Jurkat T cell activation assays were performed in the presence of 2×N-/C-terminally XTENylated XPAT prodrug (HER2-XPAT), N-terminal only XTENylated XPAT intermediate (HER2-XPAT(1×-N)), C-terminal only XTENylated XPAT intermediate (HER2-XPAT(1×-C)), and non-XTENylated drug (HER2-PAT). Prior to the end of the overnight incubation above, 10×HER2-XPAT, HER2-XPAT(1×-N), HER2-XPAT(1×-C), and HER2-PAT titrations were prepared using an 11 point, 4-fold titration (12th point is non-treatment) with a starting concentration of 7320 nM for HER2-XPAT, HER2-XPAT(1×-N), HER2-XPAT(1×-C), and 158.3 nm for HER2-PAT. 10×HER2-XPAT, HER2-XPAT(1×-N), HER2-XPAT(1×-C), and AMX-818-P1(PAT) titrations were diluted 10-fold into the well for starting concentrations of 732 nM and 15.83 nm, respectively. Jurkat cells were seeded in the wells at a 5:1 Effector:Target (E:T) ratio (BT474-100 k cells/well, SKOV3-50 k cells/well). The plates were incubated at 37° C., 5% CO2 for 6 hours. After the 6-hour incubation, 100 μL of Bio-Glo® luminescent substrate solution was added to all wells and the plates were incubated at room temperature for 5-10 minutes. The plates were then shaken on a plate shaker at 300-500 rpm for 30 seconds to mix the contents of the wells and read in a luminometer using an integration time of 500 ms. The intensity of signal produced correlates to the amount of luciferase from lysed Jurkat cells present in the wells. The signal was plotted by concentration and half maximal response (EC50) values were derived with a 4-parameter logistic regression equation using GraphPad Prism software.

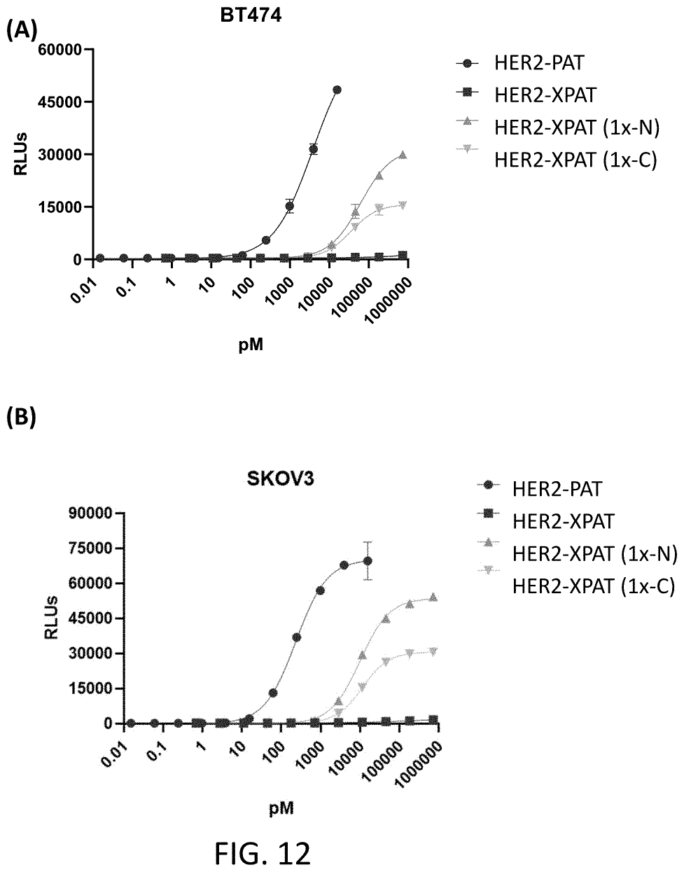

The results indicated that both N-terminal and C-terminal XTENylated intermediates provided partial mitigation of XPAT Jurkat cell activation compared to the both N- and C-terminally XTENylated XPAT prodrug. This can be seen in FIG. 12, which shows dose response curves for 2× N-/C-terminally XTENylated XPAT prodrug (HER-2XPAT, N-terminal only XTENylated XPAT intermediate (“HER2-XPAT (1×-N)”), C-terminal only XTENylated XPAT intermediate (“HER2-XPAT (1×-C)”), and non-XTENylated drug (“HER2-PAT”) in BT-474 (FIG. 12A) and SK-OV-3 (FIG. 12B) cells. In FIG. 12, both the N-terminal only and the C-terminal only (“HER2-XPAT(1×-N)” and “HER2-XPAT (1×-C)”) constructs exhibit EC50 values that are: (a) approximately equal to each other, and (b) intermediate between the fully XTENylated prodrug (“HER2-XPAT”) and the non-XTENylated drug (“HER2-PAT”). This is true when tested against both BT-474 and SK-OV-3 cells. For BT-474 cells, numerical differences in potency could be estimated by comparing the concentration associated with the maximal value obtained for HER2-XPAT to the concentration associated with the signal for the other molecule at the signal obtained with HER2-XPAT for each pair of conditions: (a) HER2-XPAT at 732 nM and HER2-XPAT(1×-N) at 2,494.9 pM—an approximately 293-fold difference, (b) HER2-XPAT at 732 nM and HER2-XPAT(1×-C) at 3,917.4 pM—an approximately 187-fold difference, and (c) HER2-XPAT at 732 nM and HER2-PAT at 33.4 pM—an approximately 21,924-fold difference. For SK-OV-3 cells, potency differences could be estimated by from the curve (a) HER2-XPAT was the least potent, (b) HER2-XPAT(1×-N) was intermediate potency, (c) HER2-XPAT(1×-C) was intermediate potency, and (d) HER2-PAT was the highest potency. Thus, molecules with only N- or C-terminal XTEN cleaved still showed attentuated Jurkat T cell activation, suggesting that cleavage of both XTEN moieties was necessary for maximal activity.

### Example 27: In Vitro Activation of PBMCs in the Presence of XTENylated and Non-XTENylated Anti-HER2/Anti-CD3 Constructs

Having observed activation of Jurkat T-cells by the HER2-XPAT/HER2-PAT constructs, an assay was constructed to directly measure whether the HER2-XPAT/HER2-PAT constructs were capable of inducing conventional phenotypes of T-cell activation in primary cells.

Accordingly, a flow-cytometry based SK-OV-3/PBMC model using the CD69 early marker of T-cell activation was constructed to evaluate the activity of these constructs in vitro. SKOV3 cells were purchased from ATCC (catalog #HTB-77) and the cells were cultured in McCoy's 5A medium (Life technologies, 16600-082) supplemented with 10% heat-inactivated fetal bovine serum (Life technologies, 10082147). Frozen human peripheral blood mononuclear cells (PBMCs) were purchased from BioIVT. Four hours prior to performing the cytotoxicity assay, frozen PBMCs were thawed and cultured in a T75 tissue-culture flask in RPMI (Life technologies, 72400-047) supplemented with 10% FBS; SKOV3 cells were detached by Trypsin (Life technologies, 25200114), and 40,000 cells were plated in each well of a 48-well flat bottom tissue culture plate. After the 4 h incubation, 200,000 PBMCs were added to the SKOV3 cells (an effector-to-target cell ratio of 5:1), followed by addition of increasing concentrations of HER2-PAT and HER2-XPAT as indicated in the table below. The cells were co-cultured for 72 h, followed by assessment of surface CD69 expression on CD3-gated cells by flow cytometry. For cell surface receptor staining, the cells were first blocked with anti-human Fc receptor blocking solution (Biolegend, 422302) for 10 mins at 4C, followed by a 1 hour incubation at 4C in the presence of AF488-labled anti-CD3 (Biolegend, 317310) and APC/Fire750-labeled anti-CD69 antibodies (Biolegend, 310945). Prior to sample acquisition, 7-AAD (Life technologies, 00-6993-50) was added to exclude dead cells. Data were analyzed by Flowjo software and graphed in Prism.

Alternatively, into Each Well of a 96-Well Tissue Culture Plate, SKOV3 Cells were Co-Cultured with Human PBMCs at an Effector to Target Ratio of 10:1. 10 nM Concentration of HER2 PAT or 600 nM of HER2 XPAT, Followed by 4-Fold Serial Dilutions, were Added to the Co-Culture. after 48 h of Incubation, the Plate was Centrifuged at 1200 Rpm for 5 Minutes and Supernatants Collected and Stored at −80C Until Use. The Cytokines were Measured According to Biolegend's Protocol (Th1 LegendPlex 5-Plex Kit Biolegend 740723). Data were Acquired by Flow Cytometry (Beckman Coulter).

The results indicated that the HER2-XPAT and HER2-PAT constructs were able to induce conventional markers of T-cell activation on the PBMCs in the presence of HER2+ cells (SK-OV-3 cells), and that the HER2-XPAT and HER2-PAT constructs exhibited the same difference in potencies observed in the other model systems. This can be seen in FIGS. 9B-9C, which respectively display dose-response curves of % CD69+ (“activated”) T cells versus XPAT/PAT agent concentration (FIG. 9B) and PD-1+ T cells versus XPAT/PAT agent concentration (FIG. 9C) as assessed by flow cytometry. As illustrated in FIGS. 9B-9C, both HER2-PAT and HER2-XPAT constructs were capable of inducing high levels of T-cell activation (˜80% activation at saturating concentrations). In FIG. 9B, the HER2-PAT construct exhibited a dramatically lower EC50 (3.19 pM) than did the HER2-XPAT construct (3634 pM), indicating that the N-/C-terminal XTEN molecules on the HER2-XPAT construct effectively reduced activation of T-cells when present.

As illustrated in FIGS. 9D-9F, HER2 XPAT shifts the dose response for cytokine secretion, e.g., TNF-alpha (FIG. 9D), IL-2 (FIG. 9E), and IL-6 (FIG. 9F), by at least 3000-fold vs HER2-PAT.

### Example 28: Anti-Tumor Efficacy and Intratumoral T Cell Activation in BT-474 Xenograft Model

After observing the effect of the HER2-CD3 XTENylated XPAT (HER2-XPAT) in in vitro models, a BT-474 xenograft/human PBMC model was established to assess the ability of the molecule to induce tumor regression in an in vivo setting.

Mouse Model

BT-474 cells (ATCC cat # HTB-20) were grown as monolayer at 37° C. in a humidified atmosphere (5% CO₂, 95% air). The culture medium was DMEM containing 2 mM L-glutamine (ref. L0104-500, Lonza, Belgium) supplemented with 10% fetal bovine serum (ref. P30-3306, Pan Biotech). The cells are adherent to plastic flasks. For experimental use, tumor cells were detached from the culture flask by a 5-minute treatment with trypsin-versene (ref. X0930, Dutscher), in Hanks' medium without calcium or magnesium (ref. L0611-500, Dutscher) and neutralized by addition of complete culture medium. The cells were counted in a hemocytometer and their viability was assessed by 0.25% trypan blue exclusion assay.

Peripheral blood mononuclear cells (PBMCs) were collected as buffy coat samples from healthy donors. PBMCs were purified from buffy coat using gradient centrifugation according to the Ficoll-Paque® plus procedure (Ref 07907, StemCell Technologies, Meylan, France) within 24 h of whole blood collection. The viability of PBMCs were assessed by 0.25% trypan blue exclusion assay before in-vivo injection. Only PBMC preparations with viability of ≥90% were acceptable for use in the study.

To establish the xenograft mice, tumors were induced by subcutaneous injection of 2×107 BT-474 cells in 200 μL of RPMI 1640 without phenol red containing 50% (v/v) matrigel into the right flank of female NOG (NOD. Cg-Prkdc^{scid}II2rg^{tm1Sug}/JicTac) mice 6-7 weeks old (Taconic, USA). The day of tumor cell implantation was considered as day 0 (DO). BT-474 tumor cell implantation was performed 24 hours after a whole-body irradiation with a gamma-source (1.44 Gy, 60Co, BioMep, France).

To establish human PBMCs in the xenograft mice, PBMCs were injected on D23, when mean tumor volumes reached 100-200 mm³. A subset of tumor-bearing mice were not humanized and were injected with 200 μL RPMI 1640 without phenol red as a control (“non-humanized mice”). PBMC bearing mice received one single intravenous (IV) injection of 1×10⁷ PBMCs in 200 μL RPMI 1640 without phenol red (“humanized mice”). Animals were randomized on D26, 3 days after PBMC inoculation by mean tumor volume. Non-humanized mice were randomized according to their tumor volume. Humanized mice were randomized according to tumor volume and PBMC donor into treatment groups. Intravenous treatments with vehicle (i.e. Amunix diluent) and test articles were initiated on day of randomization (i.e. Day 13).

Agent Administration/Handling/Measurement

Experimental agents (vehicle, HER2-XPAT, HER2-PAT, or HER2-XTEN [an uncleavable variant of HER2-XPAT]) were administered via intravenous injection (IV) into the caudal vein of the treated mice. The administration volume was 10 mL/kg (IV) adjusted to the most recent individual body weight. Treatment started on D26. Agents were administered according to the following dosing schedule.

TABLE 23
Dosing Schedule for Agents in Humanized BT-474 Xenograft Mice

| | No. | PBMC | | Dose | Dose | | Treatment |
|---|---|---|---|---|---|---|---|
| Group | Animals | (IV) | Treatment | (nmol/kg/inj) | (mg/kg/inj) | Route | Schedule |
| 1 | 8 | No | Amunix | — | — | IV | 3 times a |
| | | | diluent | | | | week for 3 |
| | | | (vehicle) | | | | weeks |
| 2 | 8 | Yes | Amunix | — | — | IV | |
| | | | diluent | | | | |
| | | | (vehicle) | | | | |
| 3 | 8 | Yes | HER2-PAT | 6 | 0.35 | IV | |
| | | | drug | | | | |
| 4 | 5 | Yes | HER2-PAT | 15 | 0.9 | IV | |
| 5 | 10 | Yes | Her2- | 6 | 0.9 | IV | |
| | | | XPAT-2X | | | | |
| | | | (HER2-XPAT | | | | |
| | | | (XTEN-576) | | | | |
| | | | prodrug | | | | |
| 6 | 5 | Yes | Her2-XPAT | 15 | 2.1 | IV | |
| | | | (XTEN-576) | | | | |
| | | | prodrug | | | | |
| 7 | 10 | Yes | Her2-XPAT | 6 | 1.0 | IV | |
| | | | (XTEN-864) | | | | |
| | | | prodrug | | | | |
| 8 | 8 | Yes | Her2-XTEN | 6 | 1.0 | IV | |
| | | | (XTEN-864) | | | | |
| | | | prodrug | | | | |
| | | | uncleavable | | | | |
| | | | variant | | | | |
| | | | HER2-PAT | | | | |

Blood samples were collected periodically throughout the study for treatment groups. Blood was collected by jugular vein from 3 mice (1 mouse per donor) into tubes with anticoagulant (K2EDTA) according to standard procedures before the ninth (9th) treatment (47 hours after the eighth (8th) treatment). Samples were centrifuged to obtain plasma and plasma samples were stored at −80° C. until analysis.

Tumor samples were collected in some cases post treatment. For tumor collection, a central piece of the tumor was cut and fixed in neutral buffered formalin and embedded in paraffin. The remaining part of the tumor was processed and used for flow cytometry analysis. Tumors excised for flow cytometry analysis were dissected into smaller fragments using scalpels, further dissociated into single cell suspensions in a non-enzymatic cell dissociation buffer, incubated at 37° C. for 30 minutes and mechanically separated through a 70 μm cell strainer. Viable cells were then enriched using ficoll-based gradient centrifugation.

Viable cells were processed for flow cytometry analysis by surface staining after minimizing non-specific binding with an FcR blocking reagent (viability dye was also used to allow dead cell exclusion). Fluorescently labeled surface target antibodies were added, according to the procedure described by the supplier for each antibody. The mixture was incubated for 20 minutes at room temperature protected from light, washed and then fixed with 200 μL 1% formaldehyde in PBS containing PKH26 beads. All samples were stored at +4° C. and protected from light until acquisition on cytometer. For identification of positive and negative populations, the fluorescence minus one (“FMO”) principle was used to account for background antibody fluorescence. FMO controls were used for controls, for each organ, using mice from Group 0 (residual mice). Compensation was performed using compensation beads and/or single stained cells. For analysis of viability, CD4, CD8, CD25 markers, CD45 markers, and CD3 markers, Viobility 405/452 Fixable Dye (Miltenyi Biotec), PE anti-human CD4 (BD Biosciences), PE-Vio615 anti-human CD8 (Miltenyi Biotec), PE-Vio770 anti-human CD25 (Miltenyi Biotec), FITC anti-human CD45 (BD Biosciences), and APC anti-human CD3 (BD Biosciences) were used. For analysis of leukocytes, the hCD45 marker was used. For analysis of T-cells, gating on hCDR45 followed by hCD3 was used. For analysis of CD4+ or CD8+ cells, gating on hCD45 followed by hCD3, followed by hCD8 vs hCD4 was used. Activated CD8+ or CD4+ cells were assessed by gating on CD4 or CD8 followed by CD25 analysis.

Tumor growth was monitored throughout the study. Tumor was measured twice per week after randomization in two dimensions using a caliper (Brand: Fowler Sylvac, Model: 699371). Tumor volume was calculated and expressed in mm3 using the formula: V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet. The parameters of tumor volume and tumor growth inhibition were used to evaluate the efficacy of treatment. Additionally, a comparison of tumor volume on the last day of the study with at least 80% of animals remaining was performed.

### Results

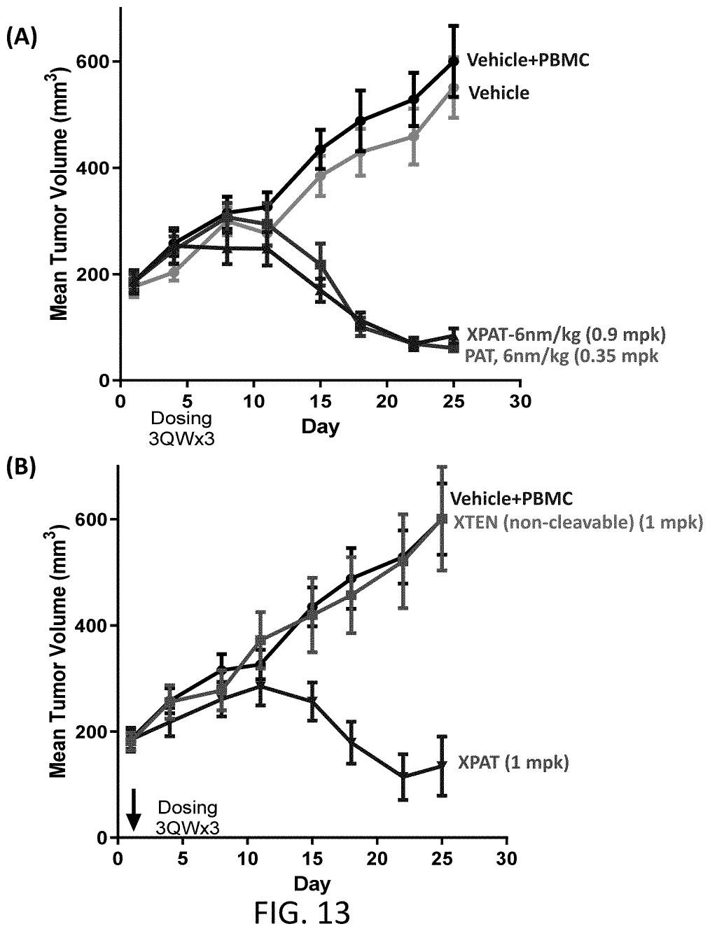

On the 25th day of treatment, mean tumors volumes were similar in the non-humanized and humanized vehicle treated Groups 1 and 2, indicating that humanization alone did not affect the tumor volume. Groups treated with 6 nmol/kg HER2-PAT (Group 3) and 6 nmol/kg Her2-XPAT-XTEN-576 and -XTEN-864 (Group 5 and 7) had significantly lower tumor volumes on treatment day 25 than vehicle-treated Group 2 (see FIGS. 13A and B), indicating that both the drug and prodrugs were effective at reducing tumor burden in mice. In comparison, treatment with the non-cleavable Her2-XTEN did not significantly lower the tumor volume compared to the control Group 2, indicating that the anti-tumor effect of the Her2-XPAT construct was dependent on proteolytic cleavage and release of the XTEN mask (see FIG. 13B). Additionally, both HER2-XPAT and HER2-PAT constructs appeared to have similar efficacy at equimolar doses, indicating that the addition of the cleavable XTEN molecules does not affect efficacy of the drug.

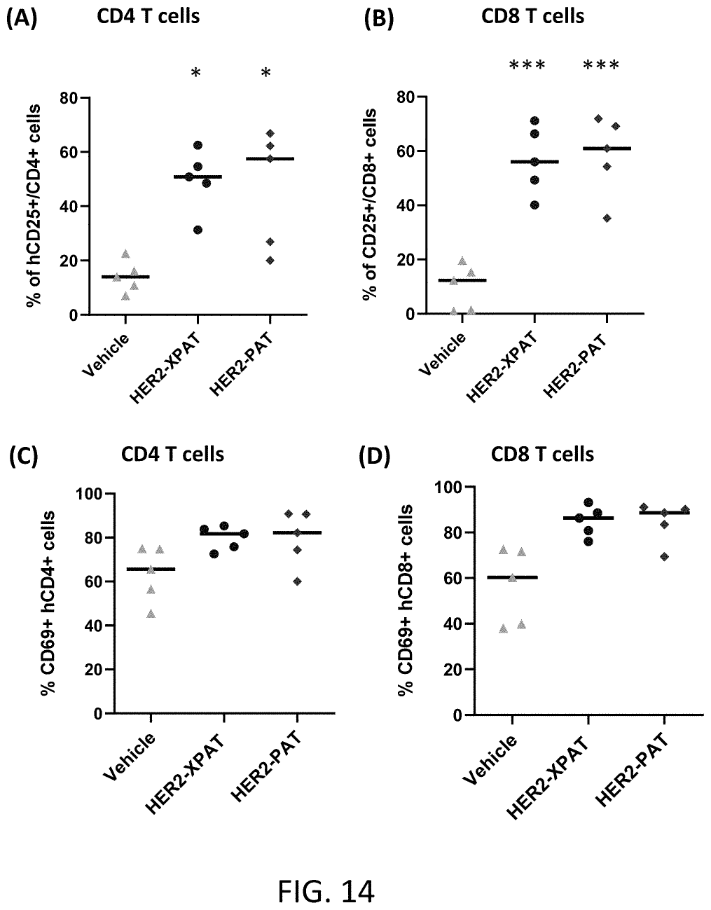

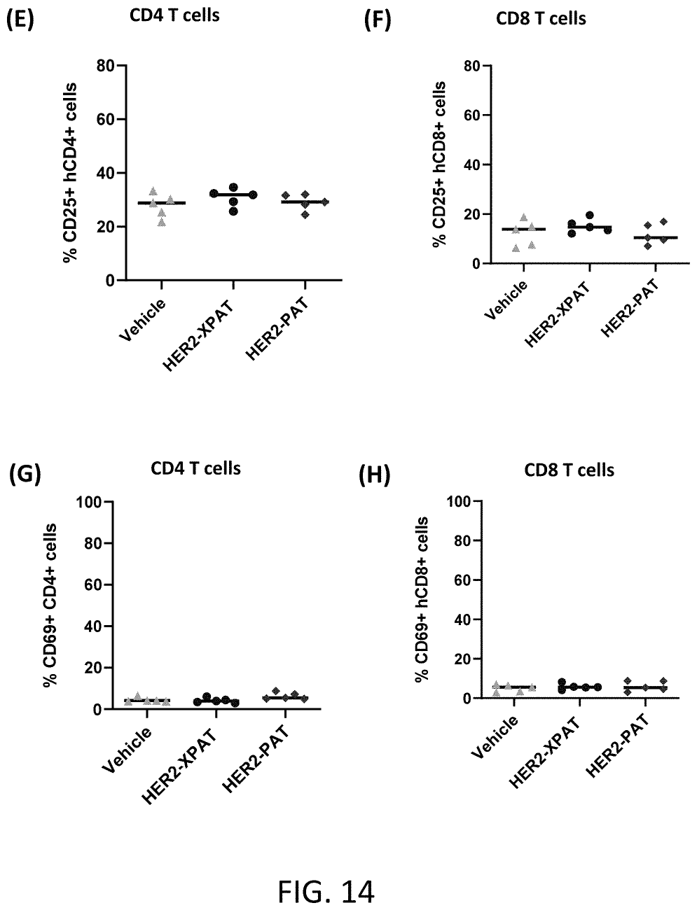

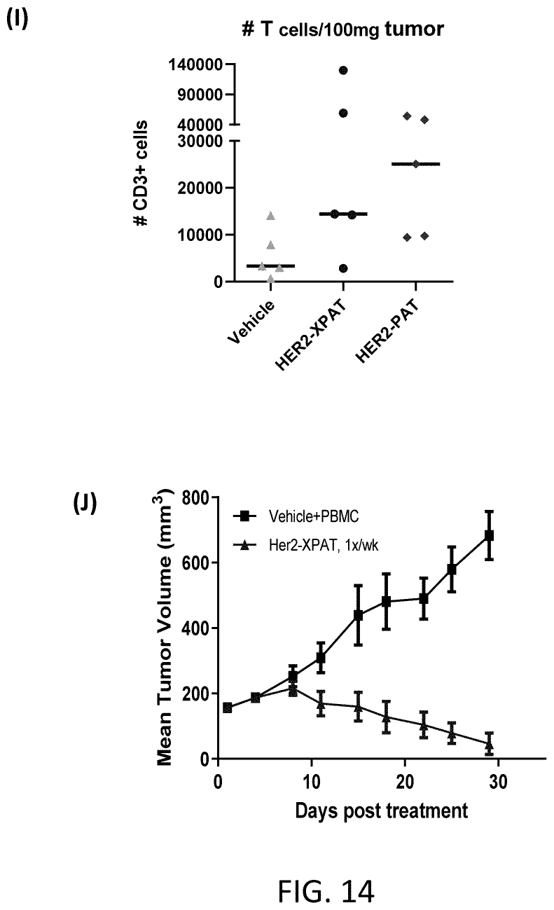

HER2-PAT Further, flow cytometry analysis of tumor infiltrating lymphocytes isolated from tumors of the animals indicated that HER2-PAT and Her2-XPAT were both effective at activating tumor-infiltrating human CD4+ and CD8+ T-cells. This can be seen in FIG. 14, which presents scatter plots of % hCD25+/CD4+ (activated CD4+ T-cells, FIG. 14A), %hCD25+/CD8+ (activated CD8+ T-cells, FIG. 14B), % hCD69+/CD4+ (activated CD4+ T-cells, FIG. 14C), and % hCD69+/CD8+ (activated CD8+ T-cells, FIG. 14D) in tumors isolated from vehicle, HER2-PAT, and HER2-XPAT treated xenograft mice. In FIGS. 14A-14D, both HER2-PAT treatment and HER2-XPAT treatment show comparable activation of CD4+ and CD8+ T-cells relative to vehicle control, with CD4+ cells being elevated at the p<0.05 confidence level, CD25+/CD8+ cells being elevated at the p<0.001 confidence level, and CD69+/CD8+ cells being elevated at the p<0.05 confidence level.

As illustrated in FIGS. 14E-14H, T cell activation is minimal in the periphery (remote from the tumor cells). No appreciable level of activation of the blood T cells was observed after treatment with XTENylated HER2-CD3 molecule (“HER2-XPAT”) or treatment with non-XTENylated HER2-CD3 molecule (“HER2-PAT”) or vehicle.

### Example 29:Anti-Tumor Efficacy Using Altered Dosing Schedule of HER2-XPAT

After observing efficacy in xenograft mice using the dosing schedule in Example 27, further dosing parameters were assessed to determine if less frequent dosing than 3×/week could be utilized. Particularly, a 3×/week dosing at 2.1 mg/kg for HER2-XPAT was investigated, using the same mouse establishment and injection protocol described in Example 27. Tumor cells were removed and the amount of CD3+ T cells were assessed per 100 mg tumor. The results are illustrated in FIG. 14I.

The results indicated that 3×/week dosing could also be sufficient to cause tumor burden regression. This can be seen in FIG. 14J, which presents a plot of tumor volume versus days post-treatment for vehicle+PBMC or HER2-XPAT dosing. In FIG. 14J, mice treated with HER2-XPAT show significantly decreased tumor burden at the endpoint versus day 0 of the same condition and the endpoint for vehicle+PBMC dosing.

### Example 30: HER2-XPAT has Significantly Increased Therapeutic Index Vs. HER2-PAT in Cynomologus Monkeys

Having established that XTENylation of the HER2 XPAT construct enhanced therapeutic index in in vitro but could induce comparable efficacy as HER2 PAT in murine tumor models in vivo settings, the XTENylated prodrug molecule was next evaluated in an cynomologus monkey (NHP) model to determine its safety profile in animals closer to the intended human population.

Cynomolgus monkeys were received from Charles River Laboratories, Houston, Tex., Covance Research Products, Alice, Tex., and Worldwide Primates, Miami, Fla. The animals were between 2.5 and 3.2 years old and weighed between 2.4 and 2.7 kg at the initiation of dosing. For experimental agents, the IV route of exposure was selected because it was the intended route of human exposure.

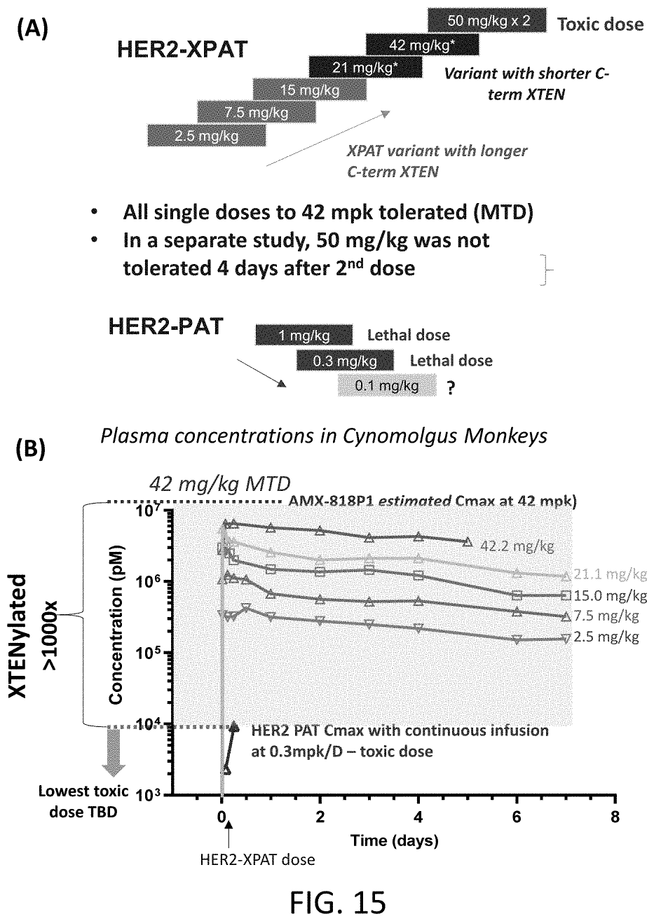

Single-dose tolerance studies were performed with 2.5 mg/kg, 7.5 mg/kg, and 15 mg/kg HER2-XPAT (AC2038, which has altered C-terminal XTEN molecule AE868 instead of the AE584 described in Example 24) and 21 mg/kg, 42 mg/kg, and 50 mg/kg of the HER2-XPAT variant with a shorter C-terminal XTEN molecule (AE584) described in Example 24 (AC2275) to assess toxicity of XTENylated HER2 constructs. Continuous infusion tolerance studies were performed with 1 mg/kg and 0.3 mg/kg HER2-PAT to assess toxicity of non-XTENylated HER2 constructs. These parameters are summarized in FIG. 15A.

For the HER2-XPAT 2038 and short HER2-XPAT 2275 variant, all doses below 50 mg/kg were tolerated, even after multiple days (see FIG. 15B, which shows serum concentrations of the HER2-XPAT molecule over time in the animals after the different doses). In contrast, both 1 mg/kg and 0.3 mg/kg HER2-PAT administered by continuous infusion were not tolerated and resulted in lethality and euthanasia of the animals (see FIG. 15B, which shows the serum concentrations of the HER2-PAT molecule pre-death). Based on the serum concentrations measured, the data indicates HER2-XPAT Provides >1000-fold higher tolerated Cmax vs. lethal Cmax for HER2-PAT, indicating that the XTENylation appears to improve therapeutic index in NHP animals.

### Example 31:Activity of HER2-PAT and HER2-XPAT on T-Cell Populations in Cynomolgus Monkeys

Having determined approximate maximum tolerated doses for the molecules in cynomolgus monkeys, we further analyzed the dosed animals to assess other pharmacodynamic effects of the constructs in the animals treated in Example 30. Particularly, the effect of both molecules on the size of particular subpopulations of T-cells was assessed. Blood samples were collected at 6 and 24 hours on day 1 after dosing and at 24 hours on day 4. The blood samples were manually checked (i.e., stick check) for clots and transferred at room temperature on the day of collection to the appropriate laboratory. Samples were kept at ambient temperature until analysis.

The cellular antigens and cell populations identified in the following table were quantified using flow-cytometry using specific antibodies against the marker antigens to assess effects on various T-cell populations. Below are the antibody combinations used and the cell populations identified.

TABLE 25
Flow-cytometry Marker Combinations for Immune Cell Analysis in Treated Monkeys

| Antigen Marker | Cell Population Identified | Parameters reported |
|---|---|---|
| CD45+/CD3+/CD16− | T-lymphocytes^{a} | % parent |
| CD45+/CD16−/CD8+/CD4− | T-cytotoxic lymphocytes^{a} | % grandparent |
| CD45+/CD16−/CD8+/CD4−/CD69+ | CD69+ T-cytotoxic lymphocytes^{b} | % parent |
| CD45+/CD16−/CD8+/CD4−/CD25+ | CD25+ T-cytotoxic lymphocytes^{c} | % parent |
| CD45+/CD16−/CD8−/CD4+ | T-helper lymphocytes^{a} | % grandparent |
| CD45+/CD16−/CD8−/CD4+/CD69+ | CD69+ T-helper lymphocytes^{b} | % parent |
| CD45+/CD16−/CD8−/CD4+/CD25+ | CD25+ T-helper lymphocytes^{c} | % parent |
| CD45+/CD3−/CD16+ | Natural Killer cells³ | % parent |

^{a}Absolute counts and absolute count percent of baseline were calculated and reported.
^{b}Mean Fluorescence Intensity (MFI) of CD69-BV421 gated positive were reported.
^{c}MFI of CD25-APC gated positive was reported.

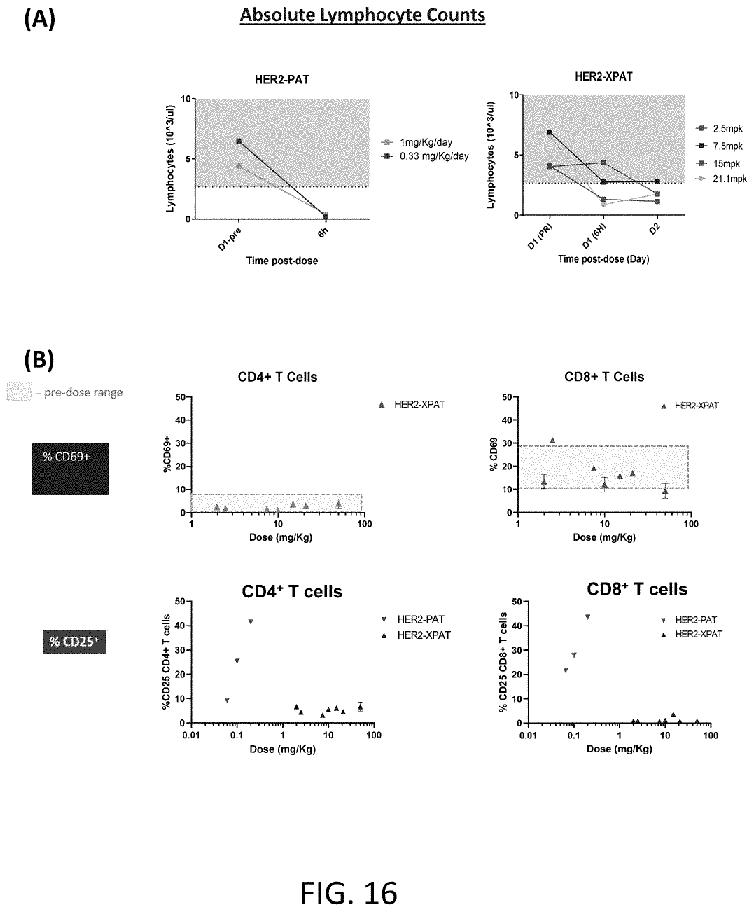

The results indicated that administration of HER2-XPAT 2038 and HER2-XPAT 2275 resulted in effects on systemic lymphocytes and systemic activated lymphocyte subpopulations assessed from blood samples. This can be seen in FIG. 16, which shows effects of agent administration on total blood lymphocytes (A) and effects of AC2275 on particular populations of activated lymphocytes (B). With respect to absolute systemic lymphocyte populations, while HER2-PAT caused apparent lymphocyte margination at all doses (see decreases in lymphocyte populations), HER2-XPAT 2038 only showed margination at the 7.5 mg/kg dose and higher, and HER2-XPAT 2275 showed margination at the 21.1 mg/kg dose (see FIG. 16A). Additionally, with respect to HER2-XPAT 2275, populations of activated CD4+ and CD8+ T-cells expressing CD69 or CD25 markers of activation were largely within pre-dose ranges (see FIG. 16B). The bottom panel of FIG. 16B further illustrates the difference between the effects of XTENylated HER2-XPAT and non-XTENylated HER2-PAT constructs on peripheral CD25+CD4+ and CD25+ CD8+ populations. For example, the exemplary non-XTENylated HER2-PAT constructs caused peripheral T cell activation at a dose of about 0.2 mg/kg or less, whereas the exemplary XTENylated HER2-XPAT did not cause peripheral T cell activation at a dose of about 50 mg/kg or less.

Further analysis was conducted to look at effect of the agents on additional subpopulations of T-cells. The results indicated that while both administration of HER2-PAT and HER2-XPAT resulted in transient decreases in T helper and T cytotoxic lymphocytes due to apparent margination, HER2-XPAT failed to induce increases in CD69 expression on T cytotoxic and T helper lymphocytes at doses as high as 50 mg/kg.

### Example 32: Effect of HER2-PAT and HER2-XPAT on Systemic Cytokine Release in Cynomolgus Monkeys

HER2-PAT and HER2-XPAT (AC2275) were further investigated for their ability to induce deleterious systemic cytokine release in Cynomolgus monkeys. Monkeys prepared as in the previous two examples were injected with escalating intravenous doses of HER2-PAT or HER2-XPAT and plasma concentrations of IL-6, TNFalpha, and IFNgamma were measured by Luminex assay.

All reagents were prepared at room temperature (RT) as stated in the Luminex Performance Assay NHP XL Cytokine premixed kit guidelines. Plasma samples were diluted 2-fold in Calibrator Diluent-RD6-65. Standard Cocktails 1 and 2 were reconstituted with Calibrator Diluent RD6-65 and allowed to sit for 15 minutes at RT. After mixing 1:1, the cocktail was diluted 3-fold in order to generate an 8-point standard curve in polypropylene tubes. 50 μl of standard or sample was then plated in duplicate on the kit-provided Greiner 96 well plate. 50 μl of standard or sample was then plated in duplicate on the kit-provided Greiner 96 well plate. After reconstituting the NHP XL Cytokine Panel Microparticle Cocktail in the mixing bottle provided, 50 μl was added to the top of each well and the plate was left to incubate for 2 hours at RT shaking at 800 rpm. Washing was then performed manually using a magnet provided by R&D systems. After preparing a 1× Wash Buffer solution, 100 μl of wash buffer was added to each well and left to sit for exactly 1 minute. The liquid was removed and washing was performed another two times. After reconstituting the NHP XL Panel Biotin-Antibody Cocktail with assay diluent RD2-1 for 20 min, a 10× dilution was performed of the reconstituted NHP XL panel biotin-antibody cocktail in assay diluent RD2-1, and 50 μl was plated in each well and left to incubate at 1 hour at RT shaking at 800 rpm. After repeating the wash step, 50 μl of diluted Streptavidin-PE was added to each well and left to incubate for 20 minutes at RT on the shaker at 800 rpm. After repeating the wash step for a third time, 100 μl of wash buffer was added to each well and the plate was left to incubate for 2 minutes at RT on the shaker at 800 rpm. The plate was then immediately read using the MAGPIX analyzer.

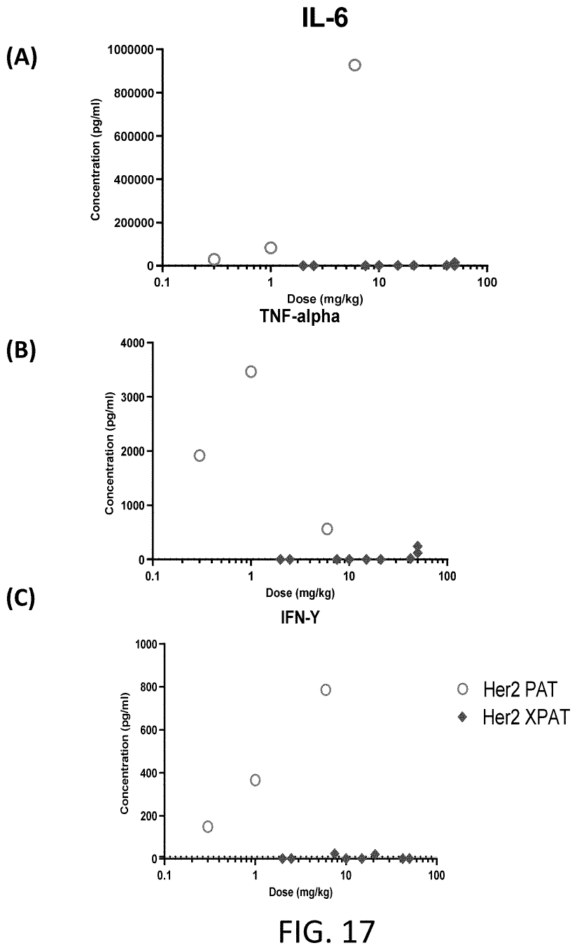

Maximal values of cytokines measured between 6-24 hours at each evaluated dose of HER2-PAT or Her2-XPAT are presented in FIG. 17. FIG. 17 shows concentrations of IL-6 (A), TNFalpha (B), or IFNgamma (C) in pg/ml for increasing dose series of HER2-PAT or HER2-XPAT. While HER2-PAT induced cytokine release of all three cytokines at all tested concentrations, concentrations of all cytokines induced by HER2-XPAT were all near baseline, indicating that the XTEN molecules in the HER2-XPAT mitigated deleterious systemic cytokine release in the context of the prodrug.

LENGTHY TABLES
The patent application contains a lengthy table section. A copy of the table is available in electronic form from the USPTO web site (https://seqdata.uspto.gov/?pageRequest=docDetail&DocID=US20210054077A1). An electronic copy of the table will also be available from the USPTO upon request and payment of the fee set forth in 37 CFR 1.19(b)(3).

1-127. (canceled)

128. A polypeptide comprising an antigen binding fragment, wherein the antigen binding fragment comprises light chain complementarity-determining regions (CDR-L) and heavy chain complementarity-determining regions (CDR-H), and wherein the antigen binding fragment:
a. specifically binds to cluster of differentiation 3 T cell receptor (CD3); and
b. comprises CDR-H1, CDR-H2, and CDR-H3, wherein the CDR-H3 comprises an amino acid sequence of SEQ ID NO:10.

129. The polypeptide of claim 128, wherein the CDR-H1 comprises an amino acid sequence of SEQ ID NO: 8; and wherein the CDR-H2 comprises an amino acid sequence of SEQ ID NO: 9.

130. The polypeptide of claim 128, wherein the antigen binding fragment exhibits a higher thermal stability, as evidenced by in an in vitro assay,
(i) a higher melting temperature (Tₘ) relative to that of an antigen binding fragment consisting of a sequence shown in SEQ ID NO:41, or
(ii) upon incorporating said anti-CD3 antigen binding fragment into an anti-CD3 bispecific antibody, the bispecific antibody exhibits a higher Tm relative to a control bispecific antibody, wherein said anti-CD3 bispecific antibody comprises said anti-CD3 binding fragment and a reference antigen binding fragment that binds to an antigen other than CD3, and wherein said control bispecific antigen binding fragment consists of SEQ ID NO:41 and said reference antigen binding fragment.

131. The polypeptide of claim 130, wherein the Tₘ of the antigen binding fragment is at least 2° C. greater than the Tₘ of an antigen binding fragment consisting of a sequence of SEQ ID NO:41.

132. The polypeptide of claim 130, wherein the CDR-H1 comprises an amino acid sequence of SEQ ID NO: 8; and wherein the CDR-H2 comprises an amino acid sequence of SEQ ID NO: 9.

133. The polypeptide of claim 128, wherein the antigen binding fragment comprises FR-H1, FR-H2, FR-H3, FR-H4, each exhibiting at least 86% sequence identity to an amino acid of SEQ ID NOs: 22, 23, 25, and 26, respectively.

134. The polypeptide of claim 128, wherein the antigen binding fragment comprises FR-L1, FR-L2, FR-L3, FR-L4, each exhibiting at least 86% sequence identity to amino acid sequences of SEQ ID NOs: 12, 13, 18, and 19, respectively.

135. The polypeptide of claim 128, wherein the CDR-L comprises:
a. a CDR-L1 having an amino acid sequence of SEQ ID NOs: 1 or 2,
b. a CDR-L2 having an amino acid sequence of SEQ ID NOs: 4 or 5, and
c. a CDR-L3 having an amino acid sequence of SEQ ID NO:6.

136. The polypeptide of claim 128, wherein the CDR-L comprises:
a. a CDR-L1 having an amino acid sequence of SEQ ID NO:1;
b. a CDR-L2 having an amino acid sequence of any one of SEQ ID NOs: 4 or 5; and
c. a CDR-L3 having an amino acid sequence of SEQ ID NOs: 6 or 7.

137. The polypeptide of claim 128, wherein the CDR-L comprises:
a. a CDR-L1 having an amino acid sequence of SEQ ID NO:2;
b. a CDR-L2 having an amino acid sequence of any one of SEQ ID NOs: 4 or 5; and
c. a CDR-L3 having an amino acid sequence of SEQ ID NO:6.

138. The polypeptide of claim 128, wherein the CDR-L comprises:
a. a CDR-L1 having an amino acid sequence of SEQ ID NO: 1;
b. a CDR-L2 having an amino acid sequence of SEQ ID NO: 4; and
c. a CDR-L3 having an amino acid sequence of SEQ ID NO: 6.

139. The polypeptide of claim 128, wherein the CDR-L comprises:
a. a CDR-L1 having an amino acid sequence of SEQ ID NO:2;
b. a CDR-L2 having an amino acid sequence of SEQ ID NO:5; and
c. a CDR-L3 having an amino acid sequence of SEQ ID NO:6.

140. The polypeptide of claim 128, wherein the antigen binding fragment further comprises a light chain framework region (FR-L) and a heavy chain framework region (FR-H), and wherein the antigen binding fragment comprises:
a. a FR-L1 having an amino acid sequence of SEQ ID NO:12;
b. a FR-L2 having an amino acid sequence of SEQ ID NO:13;
c. a FR-L3 having an amino acid sequence of any one of SEQ ID NOs:14-17;
d. a FR-L4 having an amino acid sequence of SEQ ID NO:19;
e. a FR-H1 having an amino acid sequence of SEQ ID NO:20 or SEQ ID NO:21;
f. a FR-H2 having an amino acid sequence of SEQ ID NO:23;
g. a FR-H3 having an amino acid sequence of SEQ ID NO:24; and
h. a FR-H4 having an amino acid sequence of SEQ ID NO:26.

141. The polypeptide of claim 128, wherein the antigen binding fragment further comprises a light chain framework region (FR-L) and a heavy chain framework region (FR-H), and wherein the antigen binding fragment comprises:
a. a FR-L1 having an amino acid sequence of SEQ ID NO:12;
b. a FR-L2 having an amino acid sequence of SEQ ID NO:13;
c. a FR-L3 having an amino acid sequence of SEQ ID NO:14;
d. a FR-L4 having an amino acid sequence of SEQ ID NO:19;
e. a FR-H1 having an amino acid sequence of SEQ ID NO:20;
f. a FR-H2 having an amino acid sequence of SEQ ID NO:23;
g. a FR-H3 having an amino acid sequence of SEQ ID NO:24; and
h. a FR-H4 having an amino acid sequence of SEQ ID NO:26.

142. The polypeptide of claim 128, wherein the antigen binding fragment further comprises a light chain framework region (FR-L) and a heavy chain framework region (FR-H), and wherein the antigen binding fragment comprises:
a. a FR-L1 having an amino acid sequence of SEQ ID NO:12;
b. a FR-L2 having an amino acid sequence of SEQ ID NO:13;
c. a FR-L3 having an amino acid sequence of SEQ ID NO:15;
d. a FR-L4 having an amino acid sequence of SEQ ID NO:19;
e. a FR-H1 having an amino acid sequence of SEQ ID NO:21;
f. a FR-H2 having an amino acid sequence of SEQ ID NO:23;
g. a FR-H3 having an amino acid sequence of SEQ ID NO:24; and
h. a FR-H4 having an amino acid sequence of SEQ ID NO:26.

143. The polypeptide of claim 128, wherein the antigen binding fragment further comprises a light chain framework region (FR-L) and a heavy chain framework region (FR-H), and wherein the antigen binding fragment comprises:
a. a FR-L1 having an amino acid sequence of SEQ ID NO:12;
b. a FR-L2 having an amino acid sequence of SEQ ID NO:13;
c. a FR-L3 having an amino acid sequence of SEQ ID NO:16;
d. a FR-L4 having an amino acid sequence of SEQ ID NO:19;
e. a FR-H1 having an amino acid sequence of SEQ ID NO:21;
f. a FR-H2 having an amino acid sequence of SEQ ID NO:23;
g. a FR-H3 having an amino acid sequence of SEQ ID NO:24; and
h. a FR-H4 having an amino acid sequence of SEQ ID NO:26.

144. The polypeptide of claim 128, wherein the antigen binding fragment further comprises a light chain framework region (FR-L) and a heavy chain framework region (FR-H), and wherein the antigen binding fragment comprises:
a. a FR-L1 having an amino acid sequence of SEQ ID NO:12;
b. a FR-L2 having an amino acid sequence of SEQ ID NO:13;
c. a FR-L3 having an amino acid sequence of SEQ ID NO:17;
d. a FR-L4 having an amino acid sequence of SEQ ID NO:19;
e. a FR-H1 having an amino acid sequence of SEQ ID NO:21;
f. a FR-H2 having an amino acid sequence of SEQ ID NO:23;
g. a FR-H3 having an amino acid sequence of SEQ ID NO:24; and
h. a FR-H4 having an amino acid sequence of SEQ ID NO:26.

145. The polypeptide of claim 128, wherein the antigen binding fragment comprises a variable heavy (VH) amino acid sequence having at least 90% sequence identity to an amino acid sequence of SEQ ID NO:28 or SEQ ID NO:31.

146. The polypeptide of claim 128, wherein the antigen binding fragment comprises a variable light (VL) amino acid sequence having at least 90% sequence identity to an amino acid sequence of any one of SEQ ID NOs: 27, 29, 30, 32, or 33.

147. The polypeptide of claim 128, wherein the antigen binding fragment comprises an amino acid sequence having at least 95% sequence identity to an amino acid sequence of any one of SEQ ID NOs:36-40.

148. The polypeptide of claim 128, wherein the antigen binding fragment specifically binds human or cynomolgus monkey (cyno) CD3.

149. The polypeptide of claim 128, wherein the antigen binding fragment specifically binds human and cynomolgus monkey (cyno) CD3.

150. The polypeptide of claim 128, wherein the antigen binding fragment binds a CD3 complex subunit selected from CD3 epsilon, CD3 delta, CD3 gamma, CD3 zeta, CD3 alpha and CD3 beta epsilon unit of CD3.

151. The polypeptide of claim 128, wherein the antigen binding fragment binds a CD3 epsilon fragment of CD3.

152. The polypeptide of claim 128, wherein the antigen binding fragment exhibits an isoelectric point (p1) that is less than or equal to 6.6.

153. The polypeptide of claim 128, wherein the antigen binding fragment exhibits a p1 that is between 6.0 and 6.6, inclusive.

154. The polypeptide of claim 128, wherein the antigen binding fragment exhibits a p1 that is at least 0.1 pH units lower than the p1 of a reference antigen binding fragment consisting of a sequence shown in SEQ ID NO: 41.

155. The polypeptide of claim 128, wherein the antigen binding fragment specifically binds human or cyno CD3 with a dissociation constant (K_{d}) constant between about between about 10 nM and about 400 nM, as determined in an in vitro antigen-binding assay comprising a human or cyno CD3 antigen.

156. The polypeptide of claim 128, wherein the antigen binding fragment specifically binds human or cyno CD3 with a dissociation constant (K_{d}) of less than about 10 nM as determined in an in vitro antigen-binding assay.

157. The polypeptide of claim 128, wherein the antigen binding fragment exhibits a binding affinity to CD3 that is at least 2-fold weaker relative to that of an antigen binding fragment consisting of an amino acid sequence of SEQ ID NO:41, as determined by the respective dissociation constants (K_{d}) in an in vitro antigen-binding assay.

158. A pharmaceutical composition comprising the polypeptide of claim 128 and one or more pharmaceutically suitable excipients.

159. A method of treating a disease in a subject, comprising administering to the subject in need thereof one or more therapeutically effective doses of the pharmaceutical composition of claim 158.

160. An isolated nucleic acid, the nucleic acid comprising (a) a polynucleotide encoding a polypeptide of claim 128; or (b) the complement of the polynucleotide of (a).
